(19) United States
(12) Patent Application Publication
(10) Pub. No.: US 2026/0173752 A1
(43) Pub. Date: Jun. 18, 2026

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Wook KIM, Suwon-si (KR); Dalho HUH, Suwon-si (KR); Hyung Sun KIM, Suwon-si (KR); Hanill LEE, Suwon-si (KR); Jiah YOON, Suwon-si (KR); Seonyeong GWAK, Suwon-si (KR); Suheon IM, Suwon-si (KR); Jonghoon KIM, Suwon-si (KR); Jonghoon KO, Suwon-si (KR); Seungjae LEE, Suwon-si (KR)

(21) Appl. No.: 19/411,855

(22) Filed: Dec. 8, 2025

(30) Foreign Application Priority Data
Dec. 13, 2024 (KR) 10-2024-0186277

(51) Int. Cl. H10K 85/60; C07B 59/00; C07D 405/14; C09K 11/02; H10K 50/12

(52) U.S. Cl. CPC H10K 85/654; C07B 59/002; C07D 405/14; C09K 11/02; H10K 85/6572; H10K 85/6574; H10K 50/12

(57) ABSTRACT

A compound for an organic optoelectronic device, a composition for an organic optoelectronic device including the compound, an organic optoelectronic device including the compound or the composition for an organic optoelectronic device, and a display device including the organic optoelectronic device, the compound being represented by a combination of Chemical Formula 1:

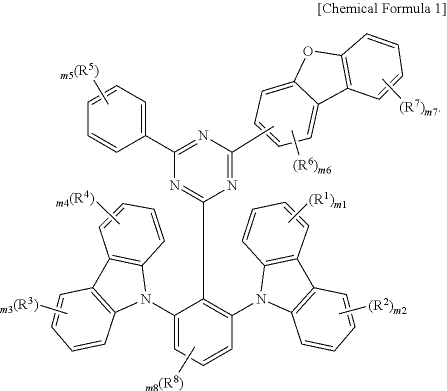

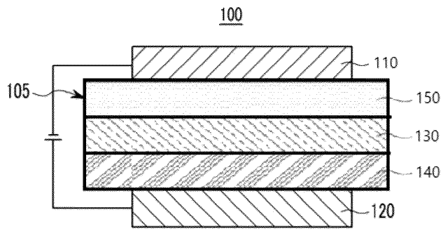

### CROSS-REFERENCE TO RELATED APPLICATION

This application claims priority to and the benefit of Korean Patent Application No. 10-2024-0186277 filed with the Korean Intellectual Property Office on Dec. 13, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively, and the other is light emitting device that generates light energy from electrical energy by supplying voltage or current to the electrodes.

Examples of the organic optoelectronic device may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

Embodiments are directed to a compound for an organic optoelectronic device represented by Chemical Formula 1:

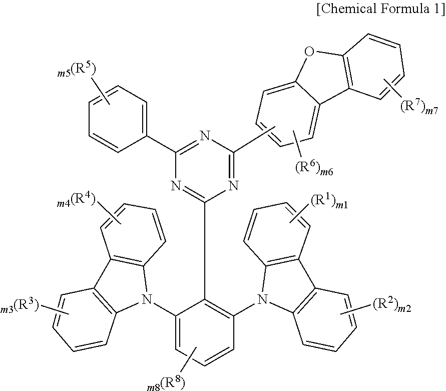

in Chemical Formula 1, R¹ to R⁷ are each independently hydrogen, deuterium, a cyano group, a substituted or unsubstituted C1 to C10 alkyl group, a substituted or unsubstituted C1 to C10 alkylsilyl group, or a substituted or unsubstituted C6 to C12 aryl group, R⁸ is hydrogen, deuterium, a substituted or unsubstituted C1 to C10 alkyl group, or a substituted or unsubstituted C6 to C12 aryl group, m1 to m4 and m7 are each independently an integer of 1 to 4, m5 is an integer of 1 to 5, m6 and m8 are each independently an integer of 1 to 3, when m1 is 2, 3, or 4, each R¹ is the same or different from each other, when m2 is 2, 3, or 4, each R² is the same or different from each other, when m3 is 2, 3, or 4, each R³ is the same or different from each other, when m4 is 2, 3, or 4, each R⁴ is the same or different from each other, when m5 is 2, 3, 4, or 5, each R⁵ is the same or different from each other, when m6 is 2 or 3, each R⁶ is the same or different from each other, when m7 is 2, 3, or 4, each R⁷ is the same or different from each other, and when m8 is 2 or 3, each R⁸ is the same or different from each other.

The embodiments may be realized by providing a composition for an organic optoelectronic device, the composition including a first compound and a second compound, wherein the first compound is the compound for the organic optoelectronic device according to an embodiment, and the second compound is represented by Chemical Formula 2, a combination of Chemical Formula 3 and Chemical Formula 4, or Chemical Formula 5:

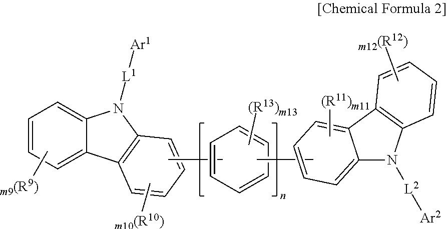

in Chemical Formula 2, R⁹ to R¹³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar¹ and Ar² are each independently a substituted or unsubstituted C6 to C20 aryl group or a substituted or unsubstituted C2 to C30 heterocyclic group, L¹ and L² are each independently a single bond or a substituted or unsubstituted C6 to C20 arylene group, m9, m12, and m13 are each independently integers of 1 to 4, m10 and m11 are each independently integers of 1 to 3, when m9 is 2, 3, or 4, each R⁹ is the same or different from each other, when m10 is 2 or 3, each R¹⁰ is the same or different from each other, when m11 is 2 or 3, each R¹¹ is the same or different from each other, when m12 is 2, 3, or 4, each R¹² is the same or different from each other, when m13 is 2, 3, or 4, each R¹³ is the same or different from each other, and n is an integer of 0 to 2;

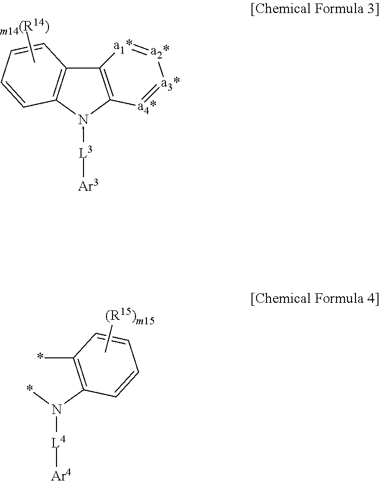

in Chemical Formula 3 and Chemical Formula 4, two adjacent ones of a₁* to a₄* of Chemical Formula 3 are linking carbons linked at * of Chemical Formula 4, the remaining two of a₁* to a₄* of Chemical Formula 3, not linked at * of Chemical Formula 4, are each independently C-L^{a}-R^{a}, L^{a}, L³, and L⁴ are each independently a single bond or a substituted or unsubstituted C6 to C20 arylene group, R^{a}, R¹⁴, and R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar³ and Ar⁴ are each independently a substituted or unsubstituted C6 to C20 aryl group or a substituted or unsubstituted C2 to C30 heterocyclic group, m14 and m15 are each independently an integer of 1 to 4, when m14 is 2, 3, or 4, each R¹⁴ is the same or different from each other, and when m15 is 2, 3, or 4, each R¹⁵ is the same or different from each other;

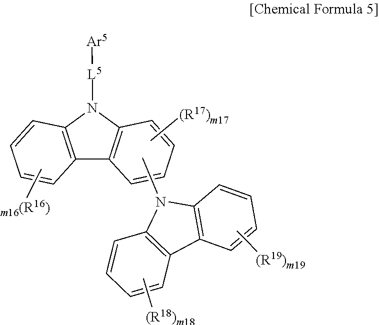

in Chemical Formula 5, L⁵ is a single bond or a substituted or unsubstituted C6 to C20 arylene group, R¹⁶ to R¹⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁵ is a substituted or unsubstituted C6 to C20 aryl group or a substituted or unsubstituted C2 to C30 heterocyclic group, m16, m18, and m19 are each independently an integer of 1 to 4, m17 is an integer of 1 to 3, when m16 is 2, 3, or 4, each R¹⁶ is the same or different from each other, when m17 is 2 or 3, each R¹⁷ is the same or different from each other, when m18 is 2, 3, or 4, each R¹⁸ is the same or different from each other, and when m19 is 2, 3, or 4, each R¹⁹ is the same or different from each other.

The embodiments may be realized by providing an organic optoelectronic device including an anode and a cathode facing each other, and at least one organic layer between the anode and the cathode, wherein the organic layer includes the compound for an organic optoelectronic device according to an embodiment.

The embodiments may be realized by providing an organic optoelectronic device including an anode and a cathode facing each other, and at least one organic layer between the anode and the cathode, wherein the organic layer includes the composition for an organic optoelectronic device according to an embodiment.

The embodiments may be realized by providing a display device including the organic optoelectronic device according to an embodiment.

### BRIEF DESCRIPTION OF THE DRAWING

Features will become apparent to those of skill in the art by describing in detail exemplary embodiments with reference to the attached drawing in which:

the FIGURE is a cross-sectional view showing an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example of the present embodiments, the “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In a specific example of the present embodiments, the “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In a specific example of the present embodiments, the “substituted” refers to replacement of at least one hydrogen of a substituent or a compound by deuterium, a cyano group, a methyl group, an ethyl group, an n-propyl group, an iso-propyl group, an n-butyl group, an iso-butyl group, a sec-butyl group, a tert-butyl group, a trimethylsilyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen (—H) substituted” may also include “deuterium substituted (-D)” or “tritium substituted (-T).” For example, any hydrogen in any compound described herein may be protium, deuterium, or tritium (e.g., based on natural or artificial substitution). As used herein, the term “or” is not an exclusive term, e.g., “A or B” would include A, B, or A and B.

As used herein, the term “or” is not an exclusive term, e.g., “A or B” would include A, B, or A and B.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example, a phenyl group, a naphthyl group, and the like, two or more hydrocarbon aromatic moieties may be linked by a sigma bond and may be, for example, a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied. A hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied. Electrons formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to example embodiments is described.

The compound for the organic optoelectronic device according to example embodiments is represented by Chemical Formula 1.

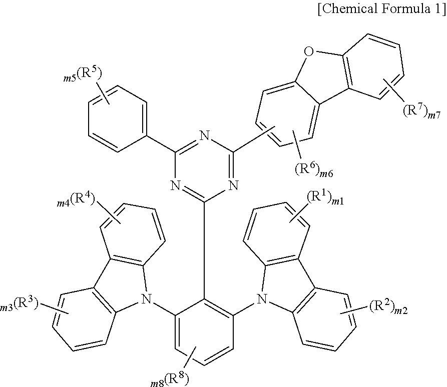

In Chemical Formula 1, R¹ to R⁷ may each independently be or include, e.g., hydrogen, deuterium, a cyano group, a substituted or unsubstituted C1 to C10 alkyl group, a substituted or unsubstituted C1 to C10 alkylsilyl group, or a substituted or unsubstituted C6 to C12 aryl group.

R⁸ may be or include, e.g., hydrogen, deuterium, a substituted or unsubstituted C1 to C10 alkyl group, or a substituted or unsubstituted C6 to C12 aryl group.

m1 to m4 and m7 may each independently be, e.g., an of integer of 1 to 4.

m5 may be, e.g., an integer of 1 to 5.

m6 and m8 may each independently be, e.g., an integer of 1 to 3.

The compound represented by Chemical Formula 1 may help realize high efficiency and long life-span by substituting phenylene linked to triazine with carbazole at both ortho-positions to have steric hindrance, thereby inducing a small TeV, and preventing a relaxation route of excitons due to steric hindrance. In addition, by substituting the triazine with dibenzothiophene to have fast electron mobility, the device may be operated quickly, and the stability for electrons may be greatly increased to realize improved life-span characteristics while maintaining high efficiency.

In particular, by substituting carbazole at the ortho-position, a low TeV may be secured, and thus, the effect of suppressing thermal decomposition during the device fabrication stage may be achieved.

In an implementation, m1 may be, e.g., 2, 3, or 4, and each R¹ may be the same or different from each other.

In an implementation, m2 may be, e.g., 2, 3, or 4, and each R² may be the same or different from each other.

In an implementation, m3 may be, e.g., 2, 3, or 4, and each R³ may be the same or different from each other.

In an implementation, m4 may be, e.g., 2, 3, or 4, and each R⁴ may be the same or different from each other.

In an implementation, m5 may be, e.g., 2, 3, 4, or 5, and each R⁵ may be the same or different from each other.

In an implementation, m6 may be, e.g., 2 or 3, and each R⁶ may be the same or different from each other.

In an implementation, m7 may be, e.g., 2, 3, or 4, and each R⁷ may be the same or different from each other.

In an implementation, m8 may be, e.g., 2 or 3, and each R⁸ may be the same or different from each other.

In an implementation, Chemical Formula 1 may be, e.g., represented by one of Chemical Formula 1-1 to Chemical Formula 1-4.

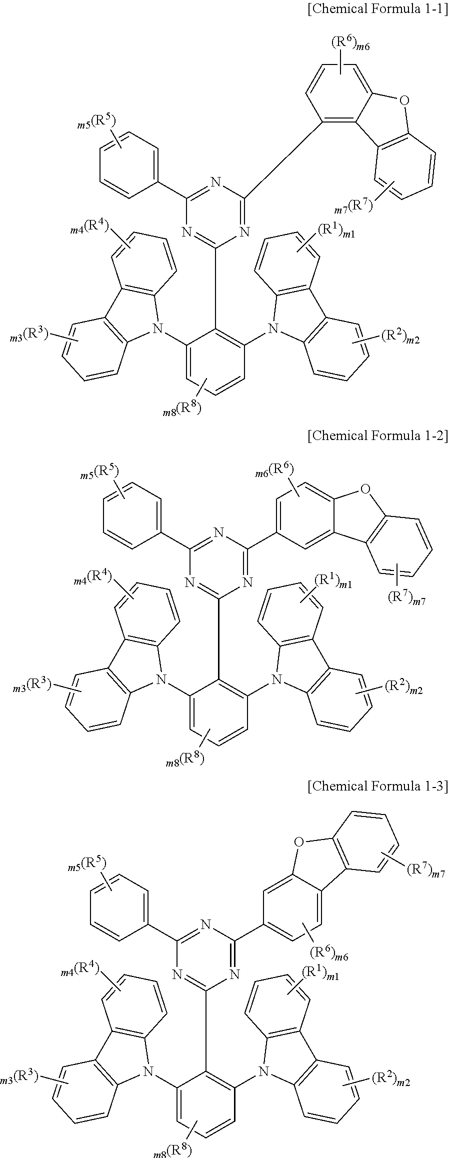

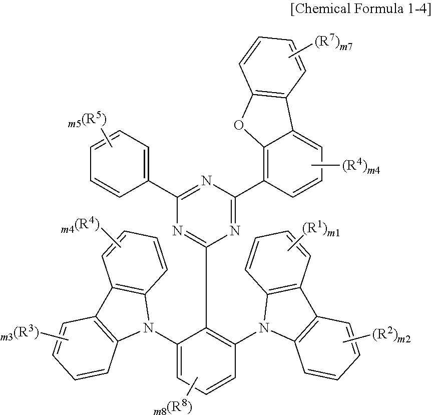

In Chemical Formula 1-1 to Chemical Formula 1-4, R¹ to R⁸ and m1 to m8 are defined the same as those described above.

In an implementation, R¹ to R⁷ may each independently be, e.g., hydrogen, deuterium, a cyano group, a substituted or unsubstituted tert-butyl group, a substituted or unsubstituted trimethylsilyl group, or a substituted or unsubstituted phenyl group.

In an implementation, R¹ may be, e.g., hydrogen, deuterium, or a substituted or unsubstituted phenyl group.

In an implementation, the compound represented by Chemical Formula 1 may be, e.g., a compound of Group 1.

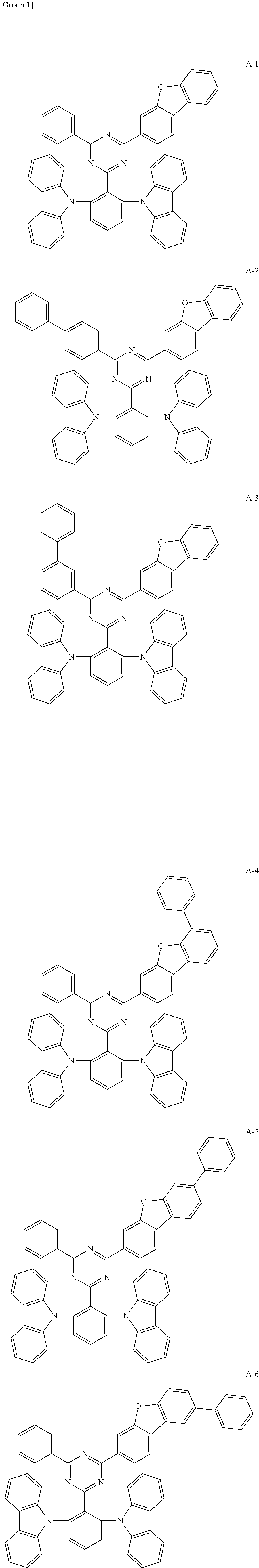

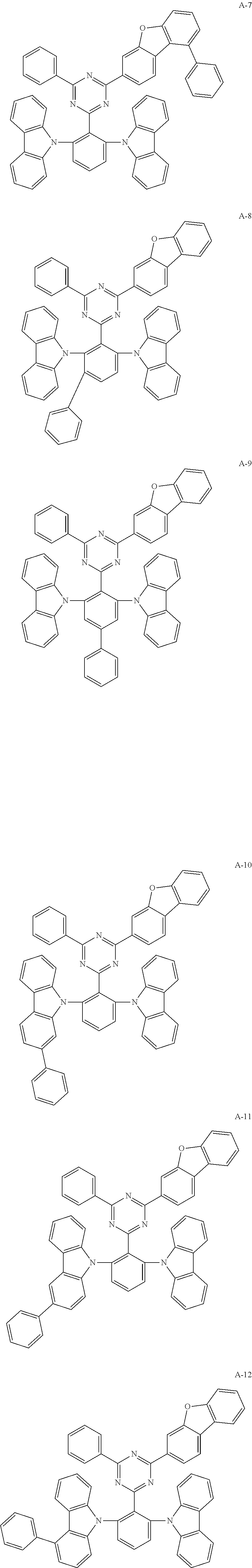

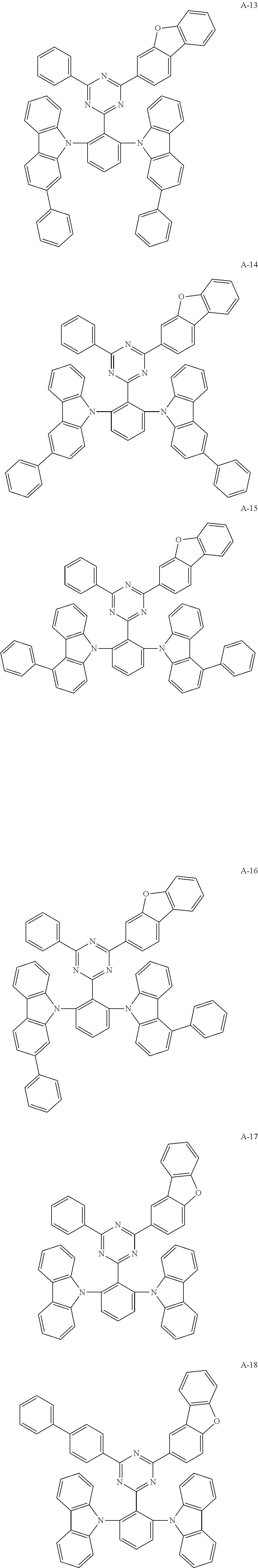

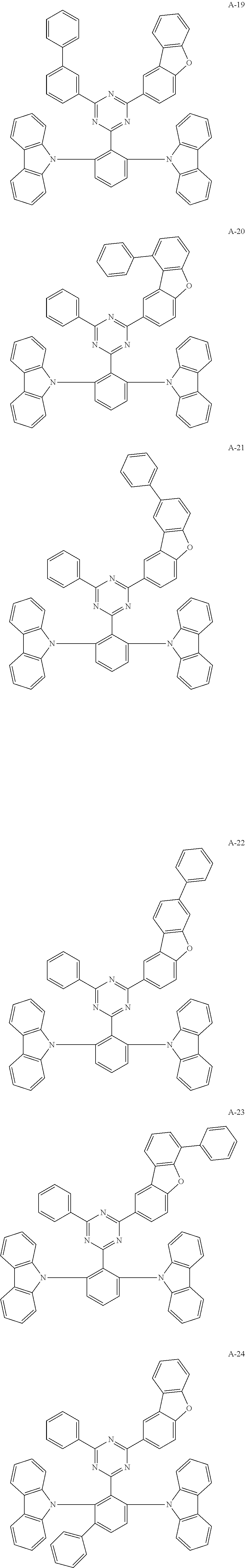

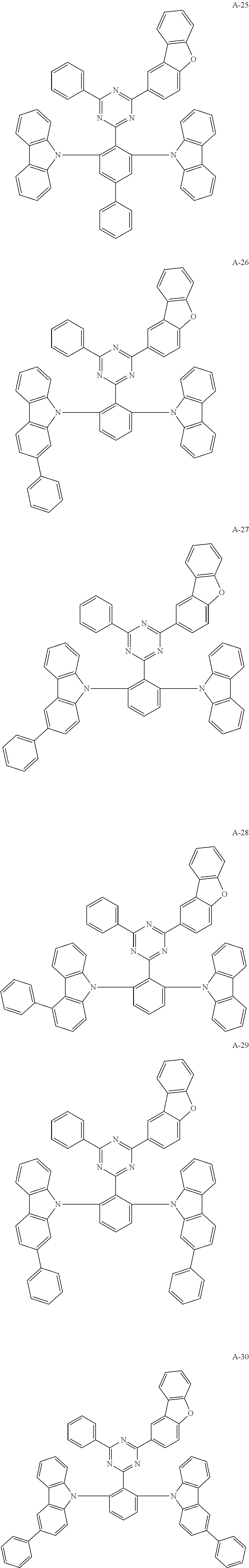

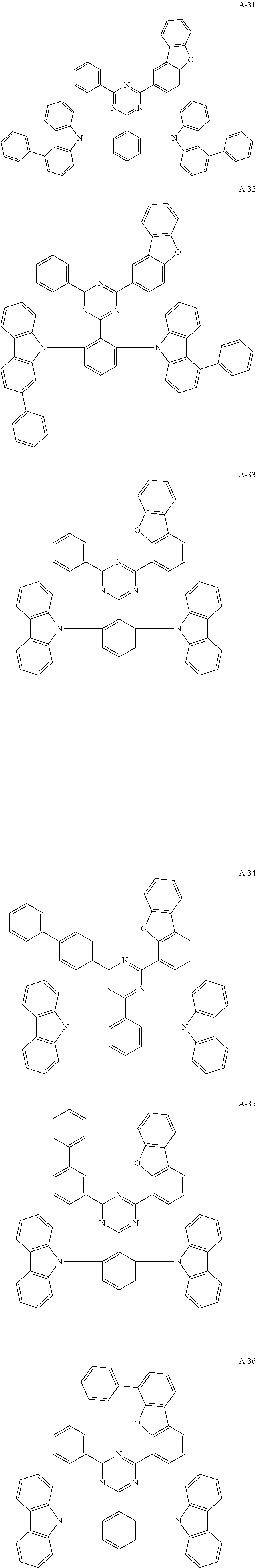

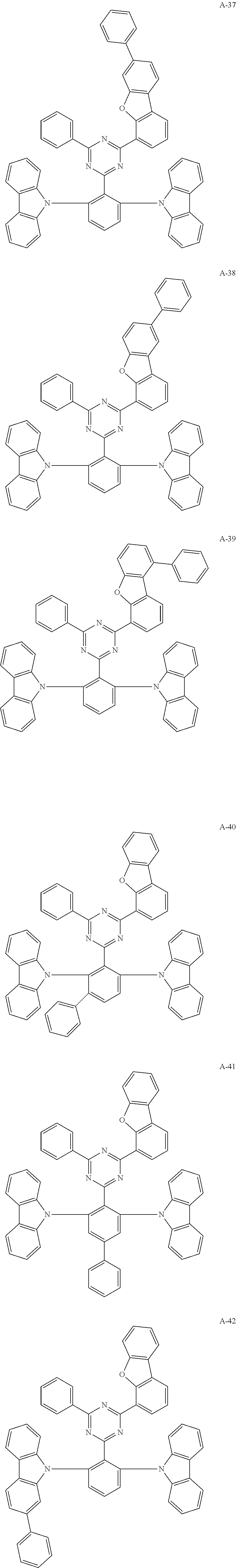

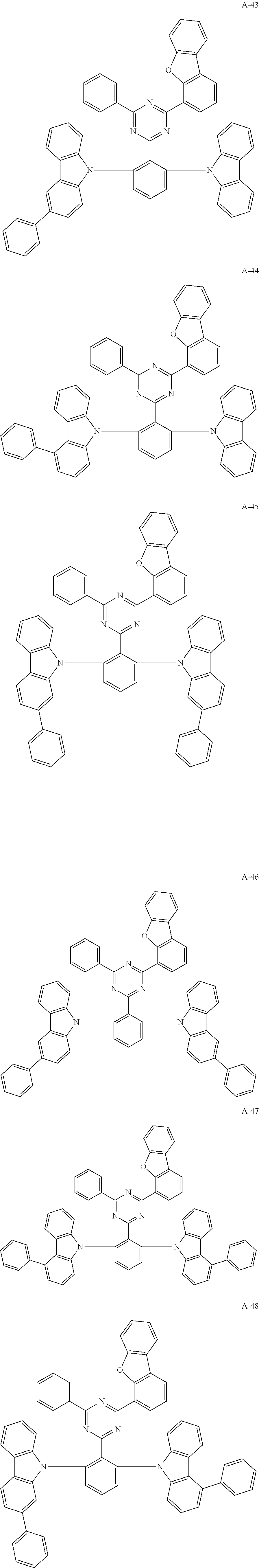

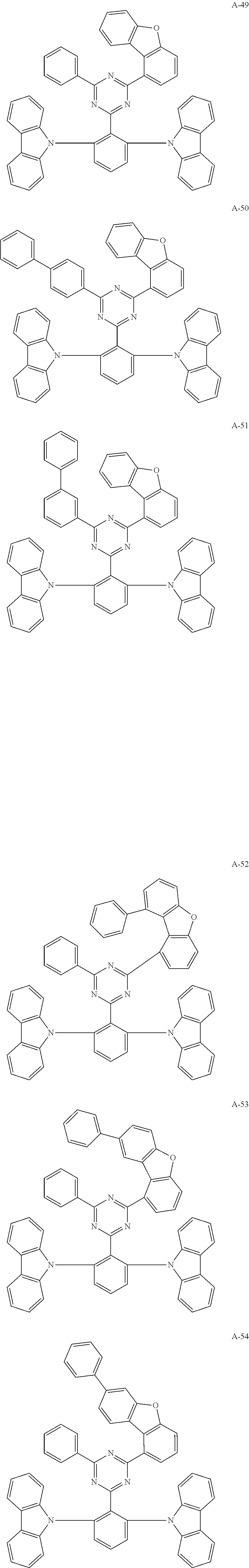

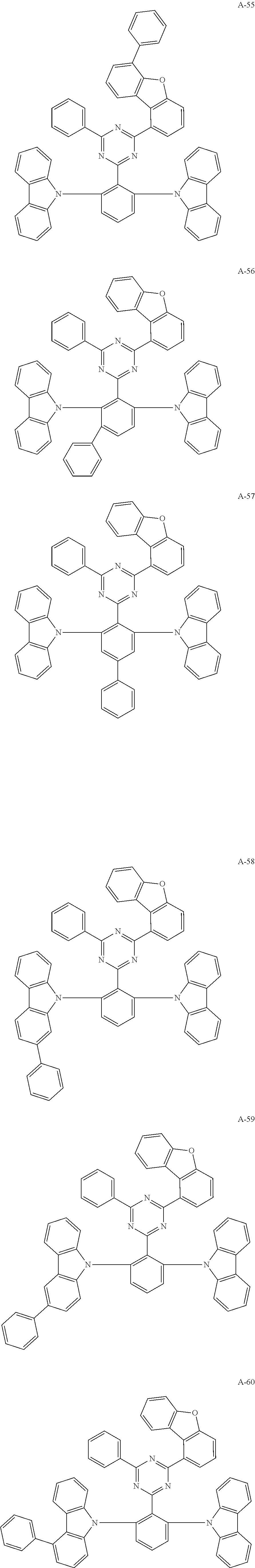

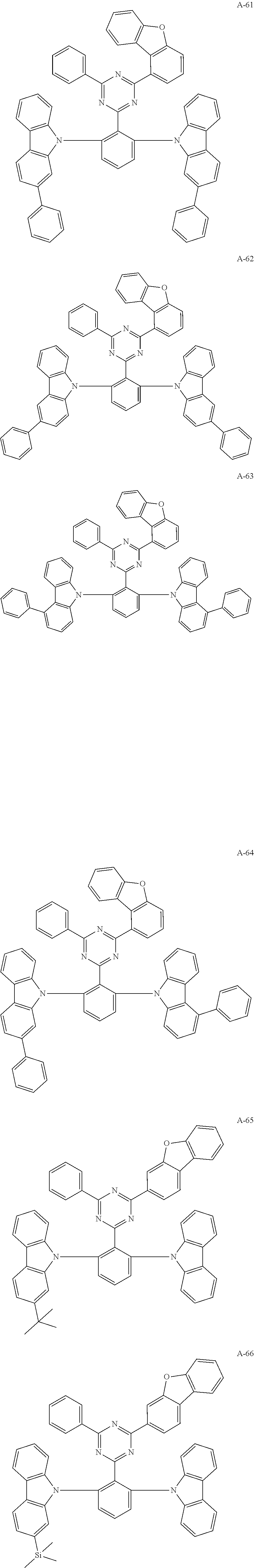

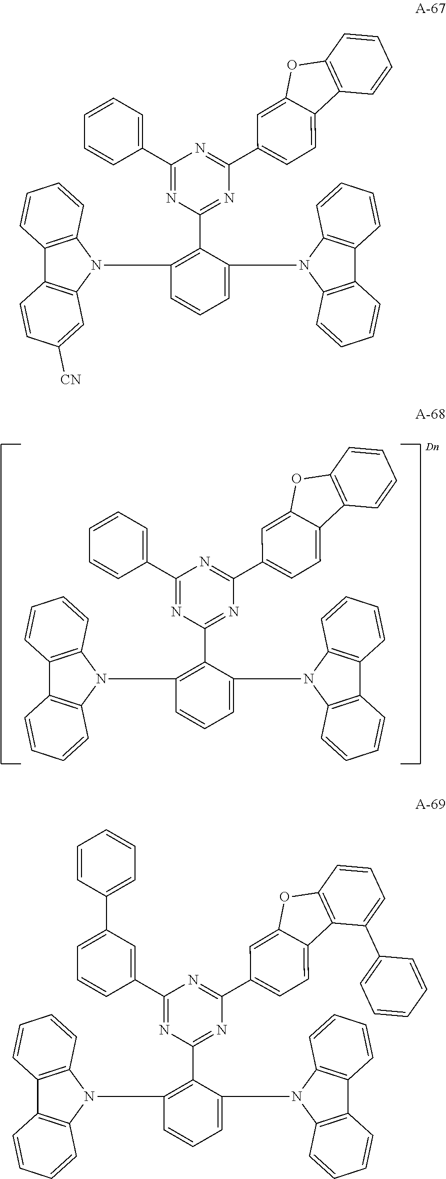

Dn represents the number of deuterium substitutions and is an integer between 1 and 30.

Depending on the value of Dn, Compound A-68 may be, e.g., represented by one of Compounds A-70 to A-72.

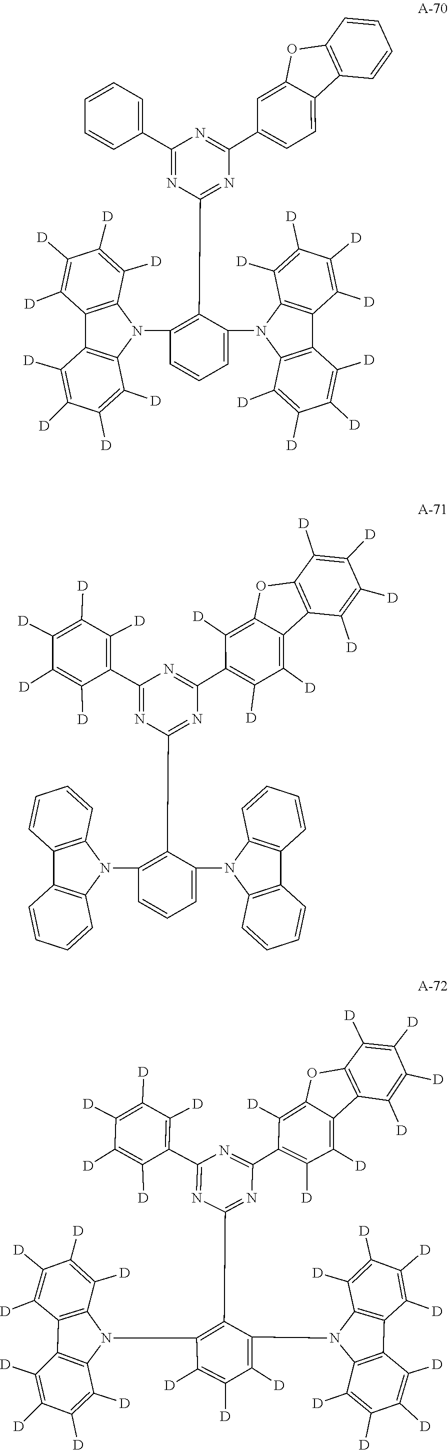

In Compound A-70, Dn is 16, in Compound A-71, Dn is 12, and in Compound A-72, Dn is 30.

According to some example embodiments, a composition for an organic optoelectronic device includes, e.g., a first compound and a second compound, wherein the first compound may be the aforementioned compound for the organic optoelectronic device, and the second compound may be a compound represented by Chemical Formula 2, a combination of Chemical Formula 3 and Chemical Formula 4, or Chemical Formula 5.

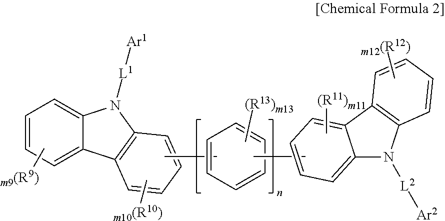

In Chemical Formula 2, R⁹ to R¹³ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar^{l} and Ar² may each independently be or include, e.g., a substituted or unsubstituted C6 to C20 aryl group or a substituted or unsubstituted C2 to C30 heterocyclic group.

L¹ and L² may each independently be or include, e.g., a single bond or a substituted or unsubstituted C6 to C20 arylene group.

m9, m12, and m13 may each independently be, e.g., an integer of 1 to 4.

m10 and m11 may each independently be, e.g., an integer of 1 to 3.

n may be, e.g., an integer 0 to 2.

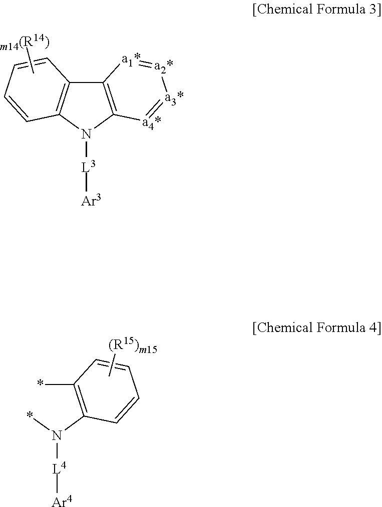

In Chemical Formula 3 and Chemical Formula 4, two adjacent ones of a₁* to a₄* of Chemical Formula 3 may be linking carbons linked at * of Chemical Formula 4, the remaining two of a₁* to a₄* of Chemical Formula 3 not linked at * of Chemical Formula 4 may be, e.g., C-L^{a}-R^{a}.

L^{a}, L³, and L⁴ may each independently be or include, e.g., a single bond or a substituted or unsubstituted C6 to C20 arylene group.

R^{a}, R¹⁴, and R¹⁵ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be or include, e.g., a substituted or unsubstituted C6 to C20 aryl group or a substituted or unsubstituted C2 to C30 heterocyclic group.

m14 and m15 may each independently be, e.g., an integer of 1 to 4.

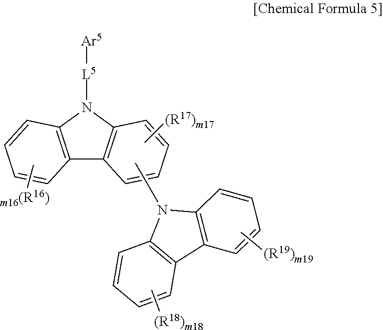

In Chemical Formula 5, L⁵ may be or include, e.g., a single bond or a substituted or unsubstituted C6 to C20 arylene group.

R¹⁶ to R¹⁹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁵ may be or include, e.g., a substituted or unsubstituted C6 to C20 aryl group or a substituted or unsubstituted C2 to C30 heterocyclic group.

m16, m18, and m19 may each independently be, e.g., an integer of 1 to 4.

m17 may be, e.g., an integer of 1 to 3.

The second compound may be used in a light emitting layer together with the first compound to improve luminous efficiency and life-span characteristics by increasing charge mobility and enhancing stability.

In an implementation, m9 may be, e.g., 2, 3, or 4, and each R⁹ may be the same or different from each other.

In an implementation, m10 may be, e.g., 2 or 3, and each R¹⁰ may be the same or different from each other.

In an implementation, m11 may be, e.g., 2 or 3, and each R¹¹ may be the same or different from each other.

In an implementation, m12 may be, e.g., 2, 3, or 4, and each R¹² may be the same or different from each other.

In an implementation, m13 may be, e.g., 2, 3, or 4, and each R¹³ may be the same or different from each other.

In an implementation, m14 may be, e.g., 2, 3, or 4, and each R¹⁴ may be the same or different from each other.

In an implementation, m15 may be, e.g., 2, 3, or 4, and each R¹⁵ may be the same or different from each other.

In an implementation, m16 may be, e.g., 2, 3, or 4, and each R¹⁶ may be the same or different from each other,

In an implementation, m17 may be, e.g., 2 or 3, and each R¹⁷ may be the same or different from each other.

In an implementation, m18 may be, e.g., 2, 3, or 4, and each R¹⁸ may be the same or different from each other, and

In an implementation, m19 may be, e.g., 2, 3, or 4, and each R¹⁹ may be the same or different from each other.

In an implementation, in Chemical Formula 2, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, L¹ and L² may each independently be, e.g., a single bond, a substituted or unsubstituted phenylene group, or a substituted or unsubstituted biphenylene group.

In an implementation, R⁹ to R¹³ may each independently be, e.g., hydrogen, deuterium, or a substituted or unsubstituted C6 to C12 aryl group.

n may be, e.g., 0 or 1.

In an implementation, “substituted” in Chemical Formula 2 may mean, e.g. that at least one hydrogen is substituted with deuterium, a C1 to C4 alkyl group, a C6 to C18 aryl group, or a C2 to C30 heteroaryl group.

In an implementation, in Chemical Formula 2, Ar^{l} and Ar² may each independently be, e.g., a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, Chemical Formula 2 may be represented by, e.g., one of Chemical Formula 2-1 to Chemical Formula 2-15.

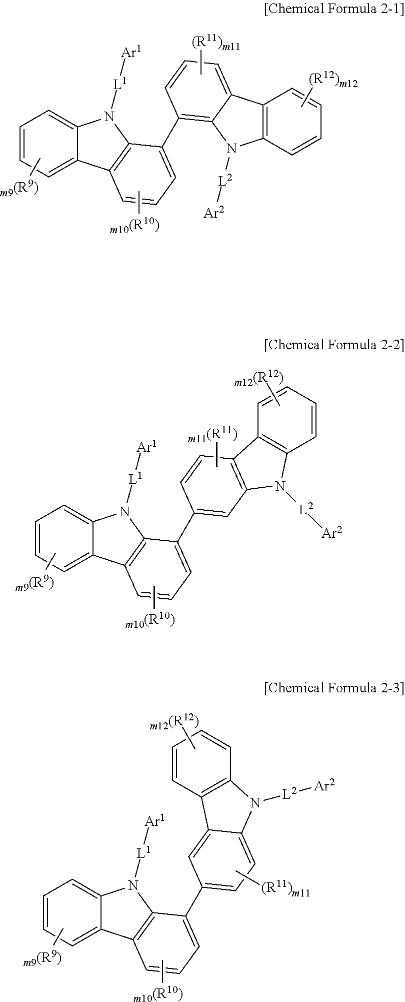

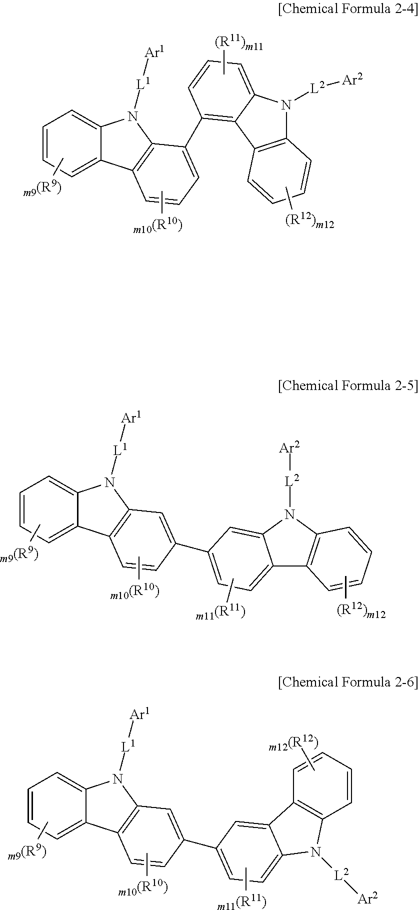

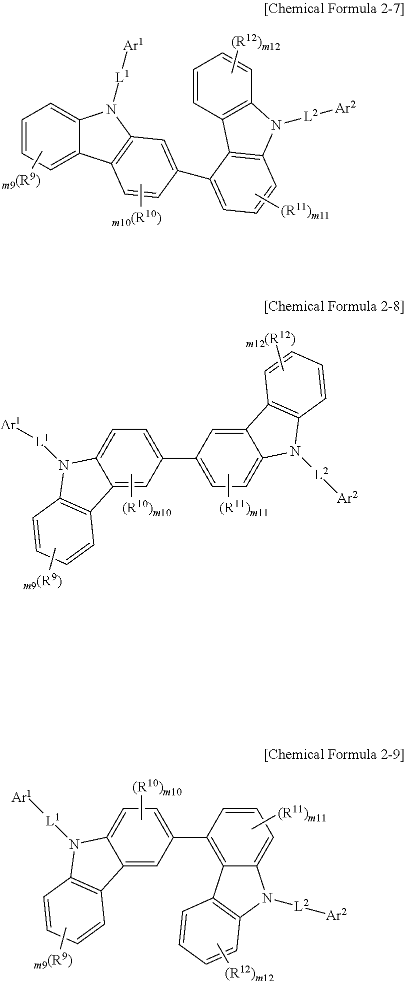

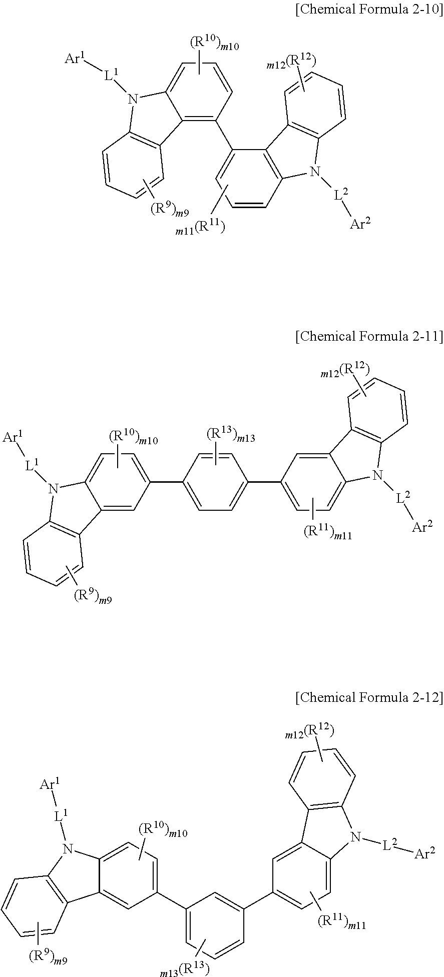

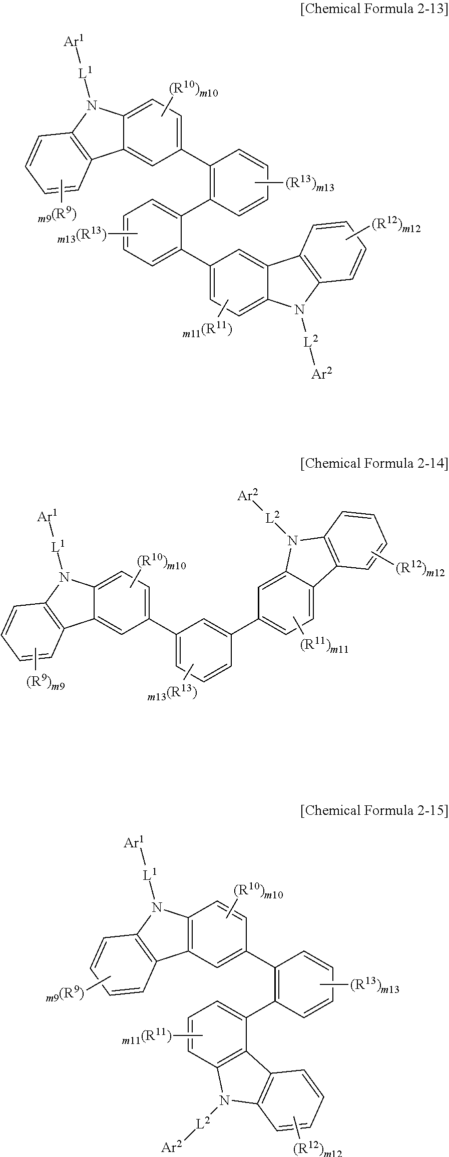

In Chemical Formula 2-1 to Chemical Formula 2-15, R⁹ to R¹³ may each independently be, e.g., hydrogen, deuterium, or a substituted or unsubstituted C6 to C12 aryl group, and moieties *-L¹-Ar¹ and *-L²-Ar² may each independently be, e.g., a moiety of Group I.

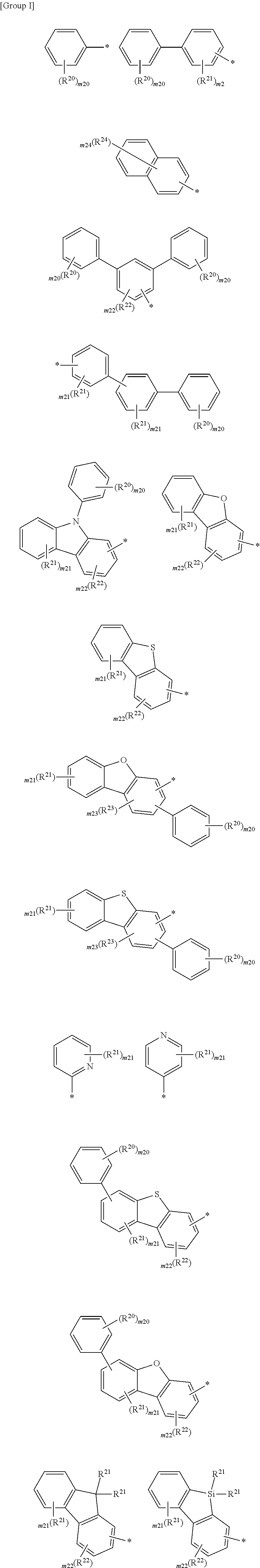

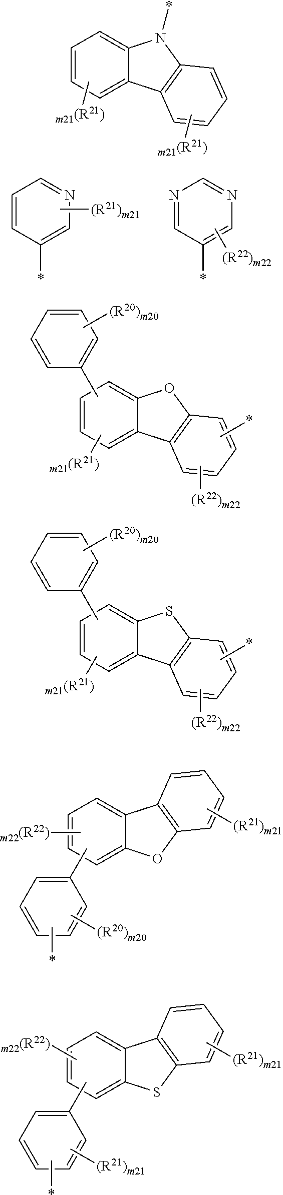

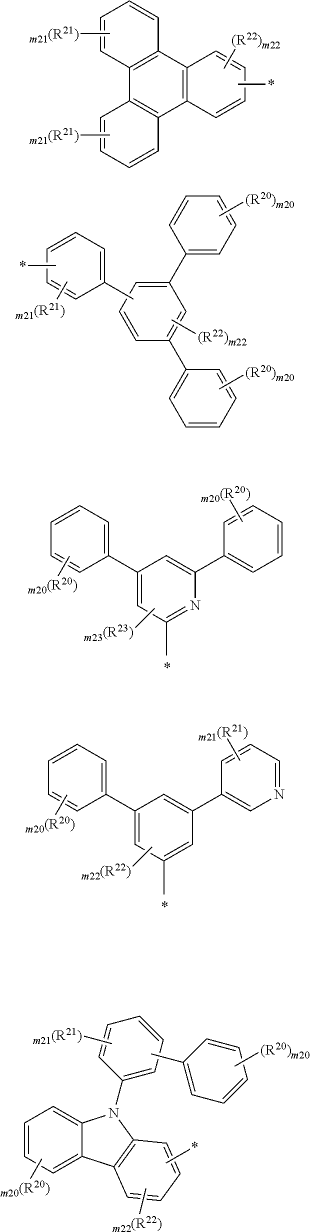

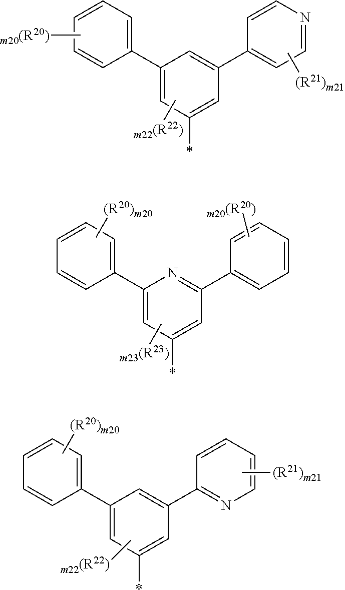

In Group I, R²⁰ to R²⁴ may each independently be, e.g., hydrogen, deuterium, a cyano group, a substituted or unsubstituted C1 to C10 alkyl group, or a substituted or unsubstituted C6 to C12 aryl group.

m20 may be, e.g., an integer of 1 to 5.

m21 may be, e.g., an integer of 1 to 4.

m22 may be, e.g., an integer of 1 to 3.

m23 may be, e.g., an integer of 1 or 2.

m24 may be, e.g., an integer of 1 to 7.

* is a linking point.

In an implementation, m20 may be, e.g., 2, 3, 4, or 5, each of R²⁰ may be the same or different from each other.

In an implementation, m21 may be, e.g., 2, 3, or 4, each of R²¹ may be the same or different from each other.

In an implementation, m22 may be, e.g., 2 or 3, and R²² may be the same or different from each other.

In an implementation, m23 may be, e.g., 2 or 3, and each R²³ may be the same or different from each other.

In an implementation, m24 may be, e.g., 2, 3, 4, 5, 6, or 7, and each R²⁴ may be the same or different from each other.

The combination of Chemical Formula 3 and Chemical Formula 4 may be, e.g., represented by one of Chemical Formula 3A, Chemical Formula 3B, Chemical Formula 3C, Chemical Formula 3D, and Chemical Formula 3E.

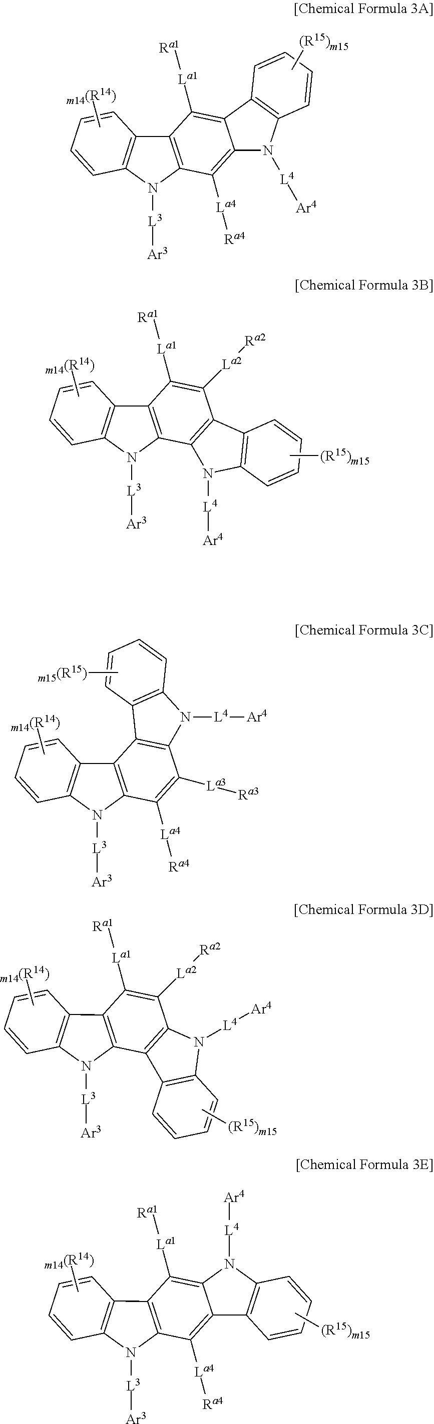

In Chemical Formula 3A to Chemical Formula 3E, L³, L⁴, Ar³, Ar⁴, R¹⁴, R¹⁵, m14 and m15 are the same as those described above,

L^{a1} to L^{a4} may be defined the same as L³ and L⁴ described above.

R^{a1} to R^{a4} may be defined the same as R¹⁴ and R¹⁵ described above.

In an implementation, in Chemical Formula 3 and Chemical Formula 4, Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R^{a1} to R^{a4}, R¹⁴ and R¹⁵ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3 and Chemical Formula 4, moieties *-L³-Ar³ and *-L⁴-Ar⁴ may each be, e.g., a moiety of Group I.

In an implementation, R^{a1} to R^{a4}, R¹⁴, and R¹⁵ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, R¹⁴, and R¹⁵ may each independently be, e.g., hydrogen, deuterium, a cyano group, or a substituted or unsubstituted phenyl group.

In an implementation, R^{a1} to R^{a4}, R¹⁴, and R¹⁵ may each independently be, e.g., hydrogen, deuterium, or a substituted or unsubstituted phenyl group.

Chemical Formula 5 may be, e.g., represented by one of Chemical Formula 5-1 to Chemical Formula 5-4.

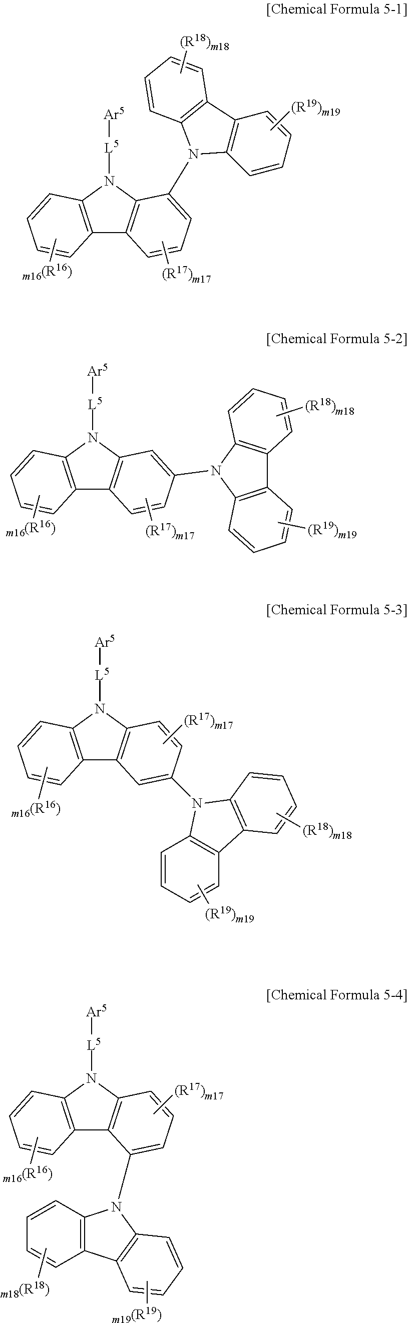

In Chemical Formula 5-1 to Chemical Formula 5-4, L⁵, Ar⁵, R¹⁶ to R¹⁸, and m16 to m18 may be defined the same as those described above.

In an implementation, in Chemical Formula 5, Ar⁵ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R¹⁶ to R¹⁸ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 5, moiety *-L⁵-Ar⁵ may be, e.g., a moiety of Group I.

In an implementation, R¹⁶ to R¹⁹ may each independently be, e.g., hydrogen, deuterium, a cyano group, or a substituted or unsubstituted phenyl group.

Chemical Formula 2 may be, e.g., represented by Chemical Formula 2-8, and in Chemical Formula 2-8, Ar¹ and Ar² may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L¹ and L² may each independently be, e.g., a single bond or a substituted or unsubstituted C6 to C20 arylene group, R⁹ to R¹² may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 2-8, R⁹ to R¹² may each independently be, e.g., hydrogen, deuterium, or a substituted or unsubstituted C6 to C12 aryl group, and moieties *-L¹-Ar^{l} and *-L²-Ar² may each independently be, e.g., a moiety of Group I.

In an implementation, the second compound may be, e.g., represented by Chemical Formula 3C, and in Chemical Formula 3C, L^{a3} and L^{a4} may each be, e.g., a single bond, L³ and L⁴ may each independently be, e.g., a single bond or a substituted or unsubstituted C6 to C12 arylene group, R¹³, R¹⁴, R^{a3}, and R^{a4} may each be, e.g., hydrogen, deuterium, or a phenyl group, and Ar³ and Ar⁴ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 4C, L^{a3} and L^{a4} may be, e.g., single bonds, R¹³, R¹⁴, R³, and R^{a4} may each independently be, e.g., hydrogen, deuterium, or a C6 to C12 aryl group, and moieties *-L³-Ar³ and *-L⁴-Ar⁴ may each independently be, e.g., a moiety of Group I.

In an implementation, the second compound for an optoelectronic device may be, e.g., a compound of Group 2.

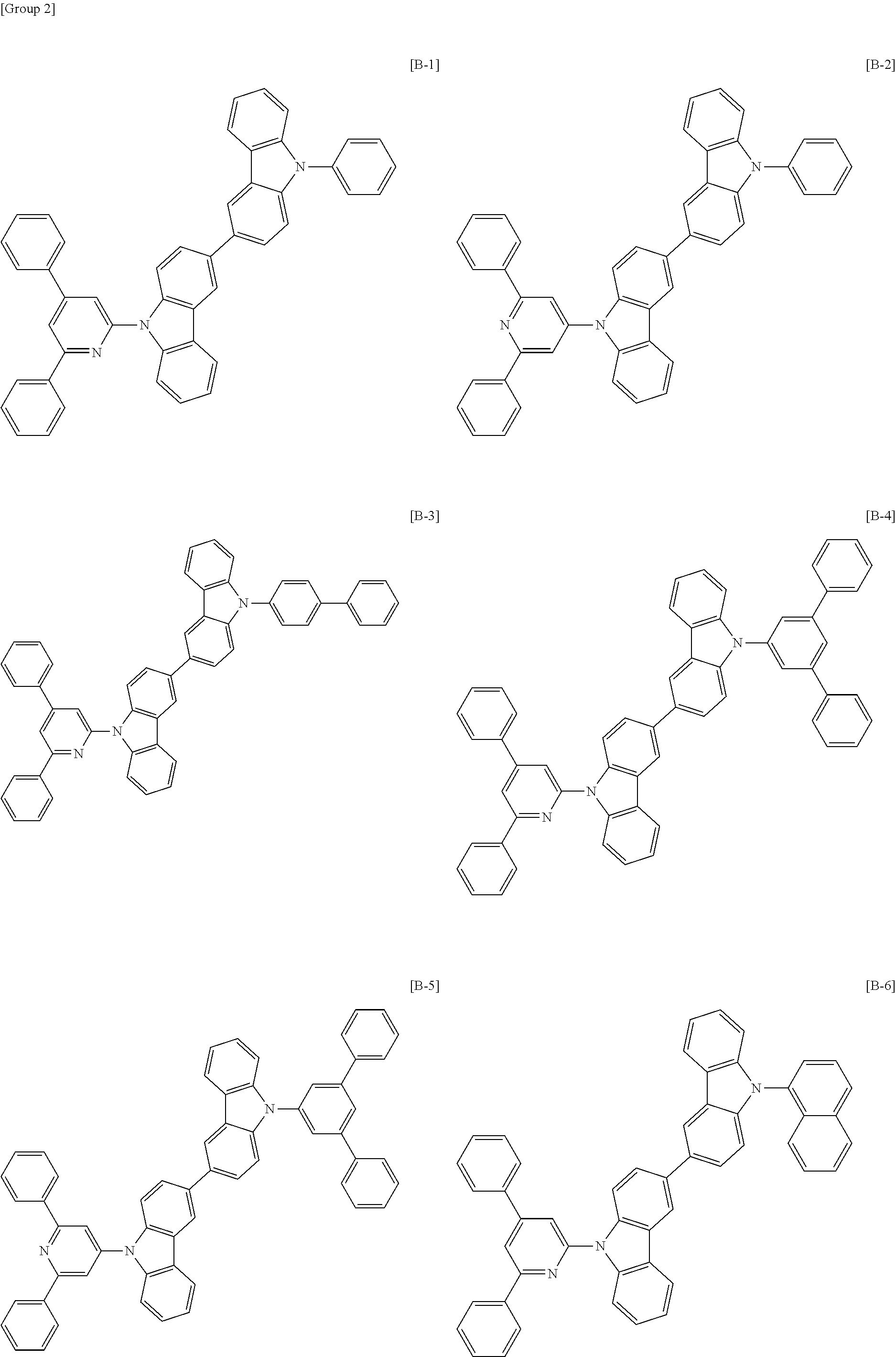

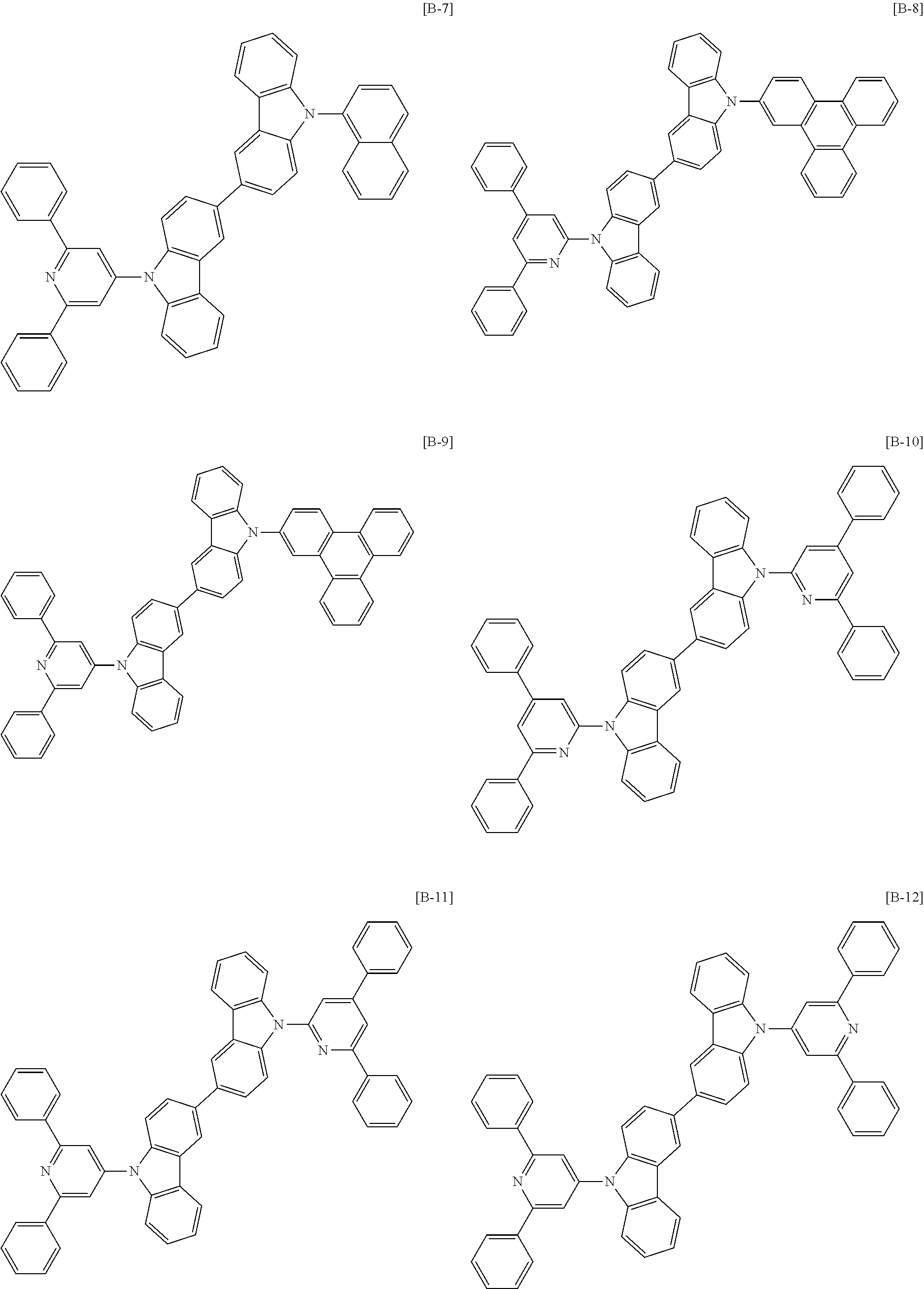

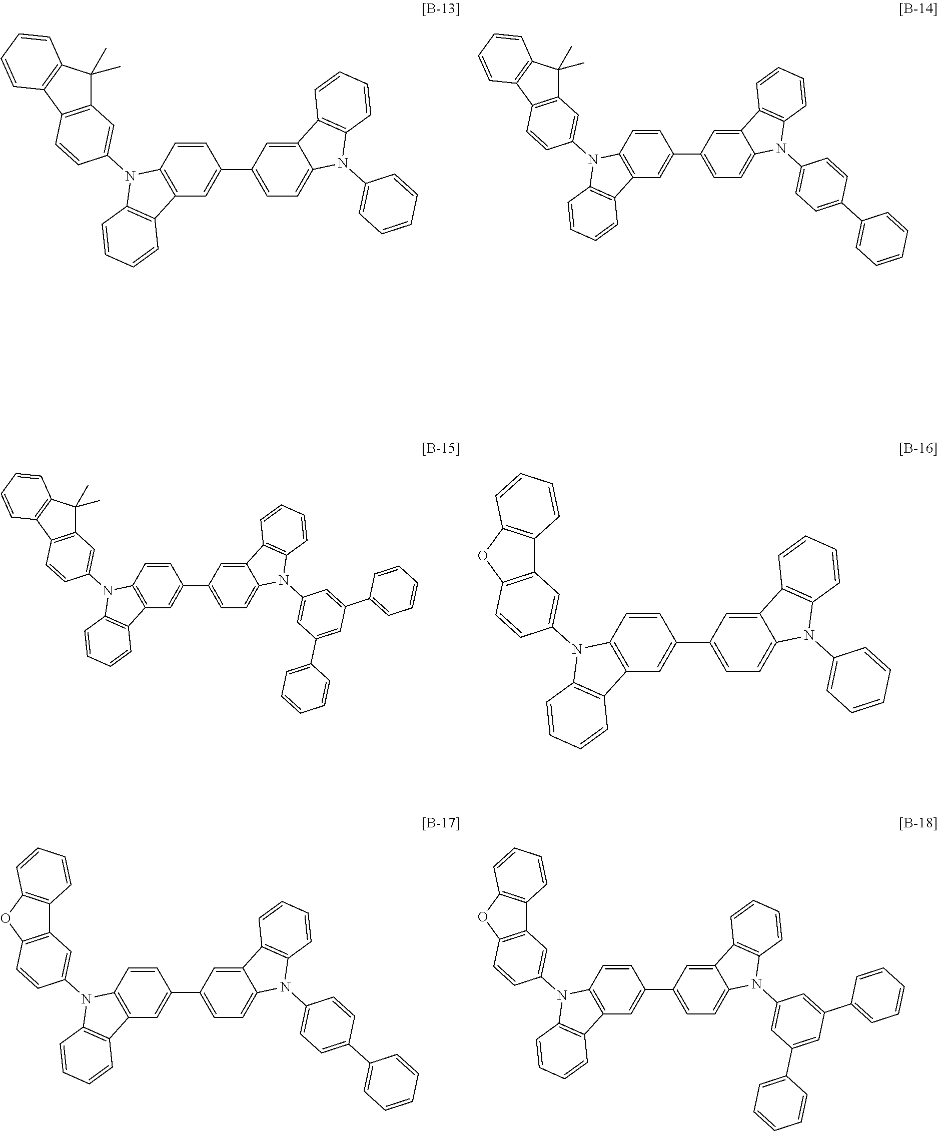

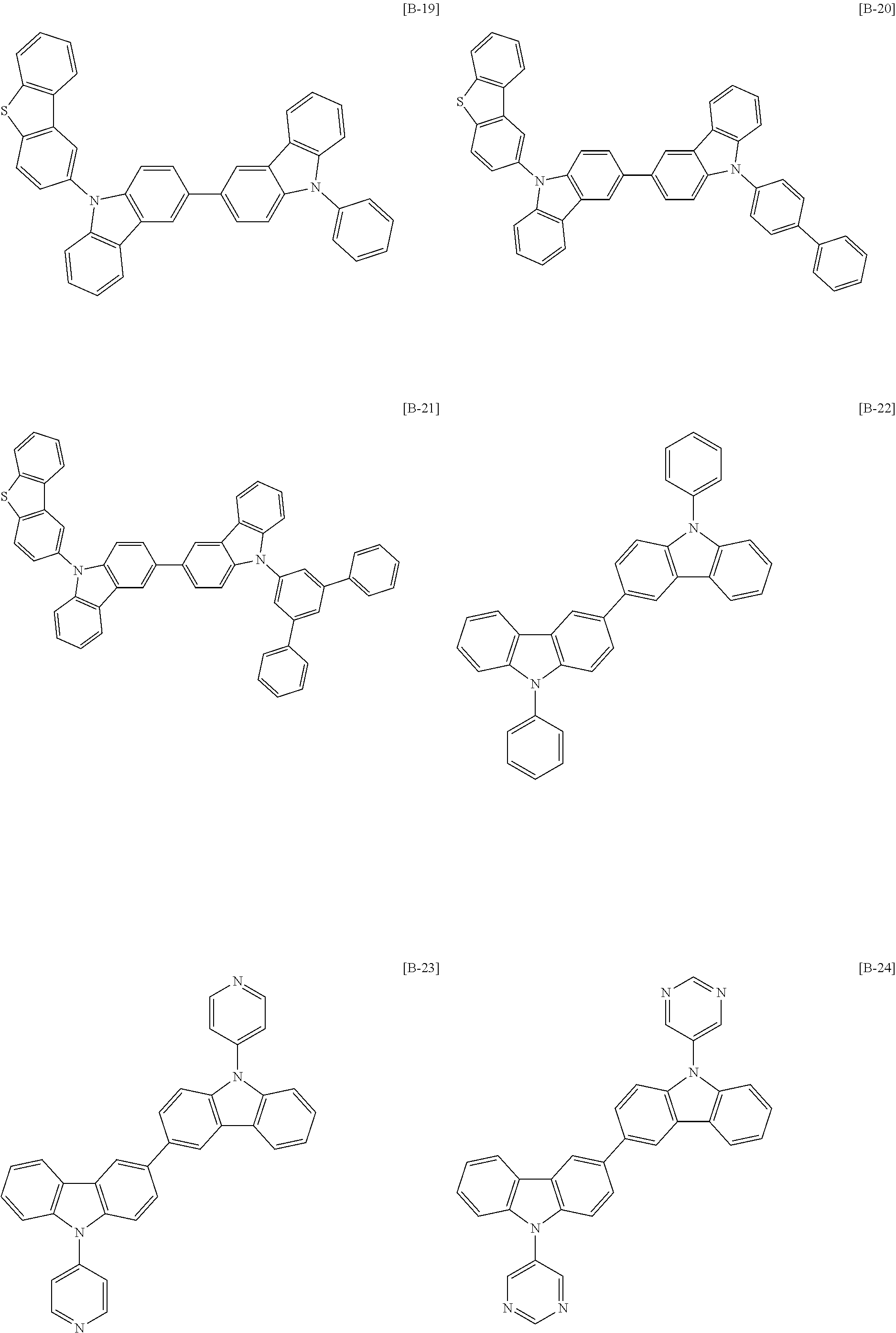 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1]

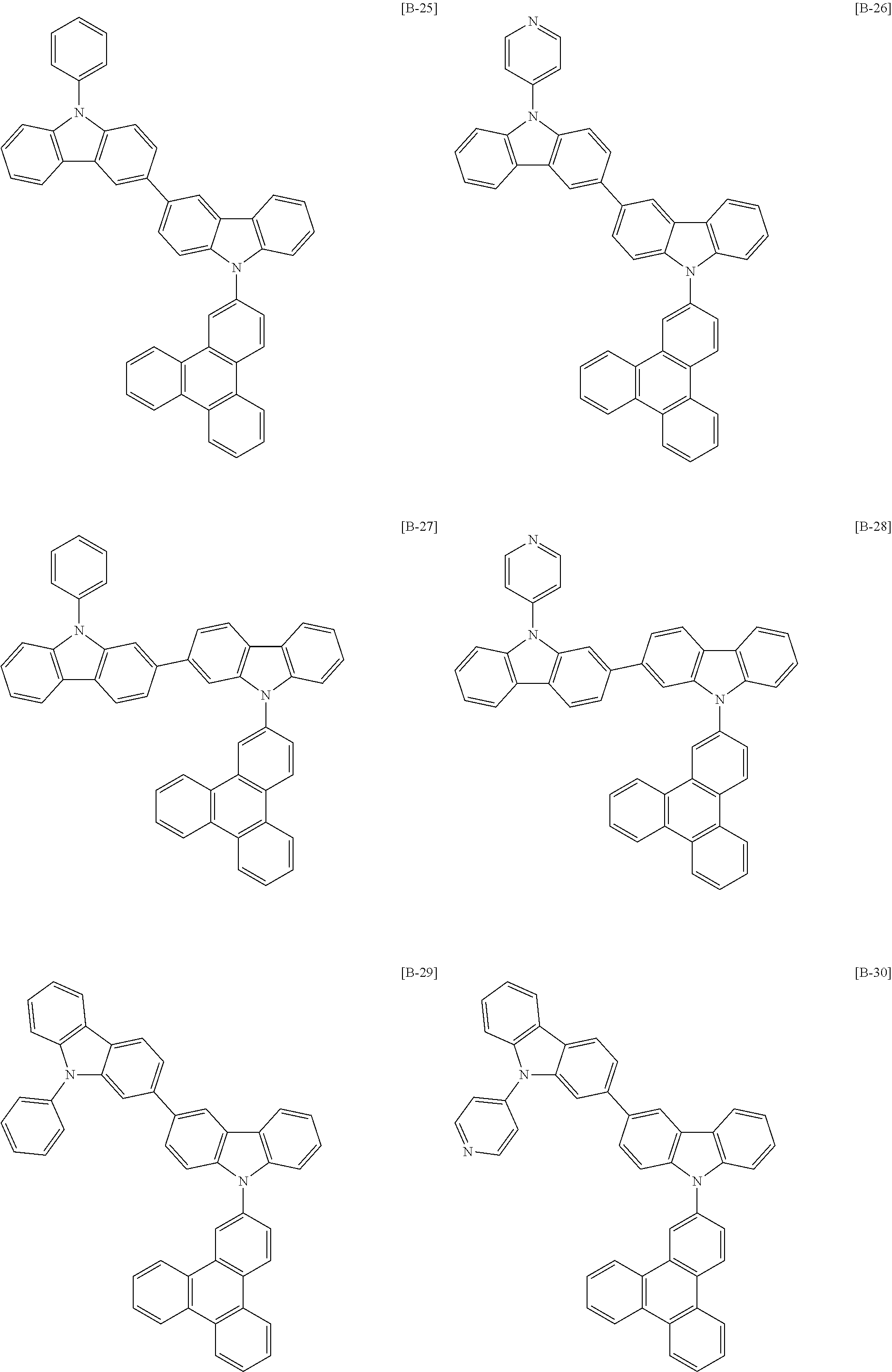

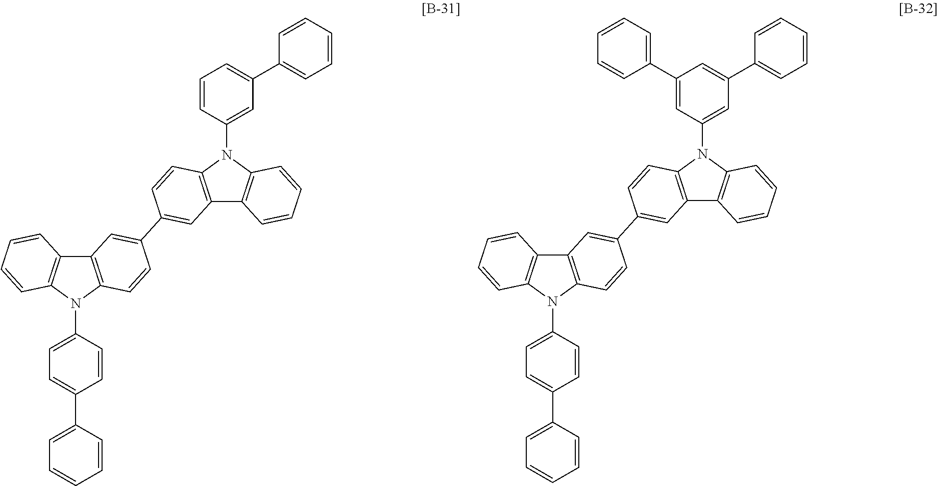 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

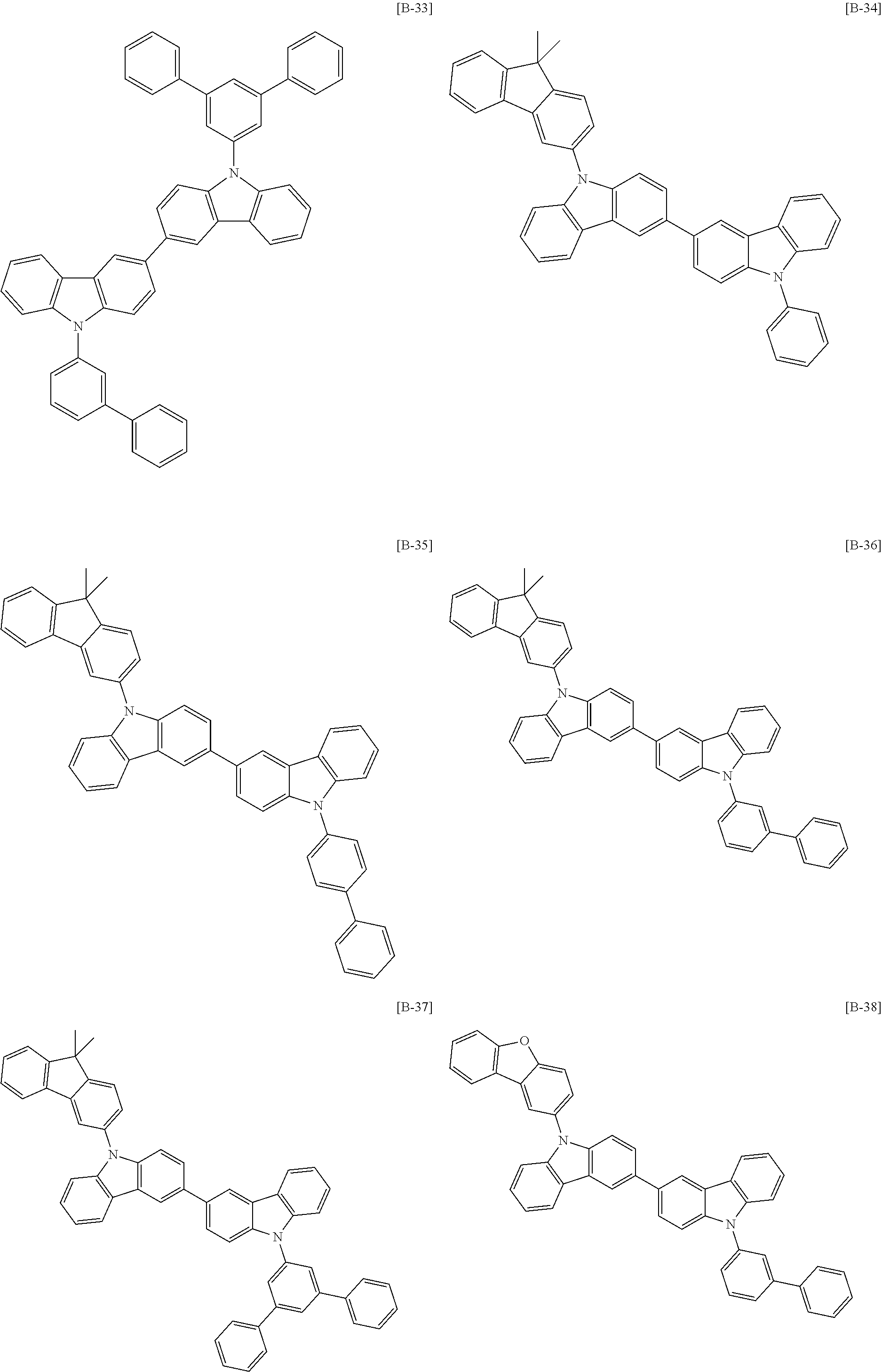

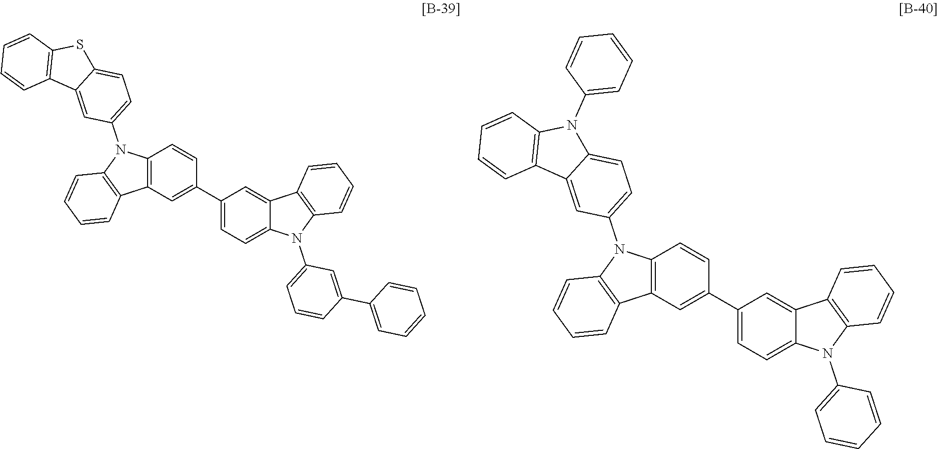 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

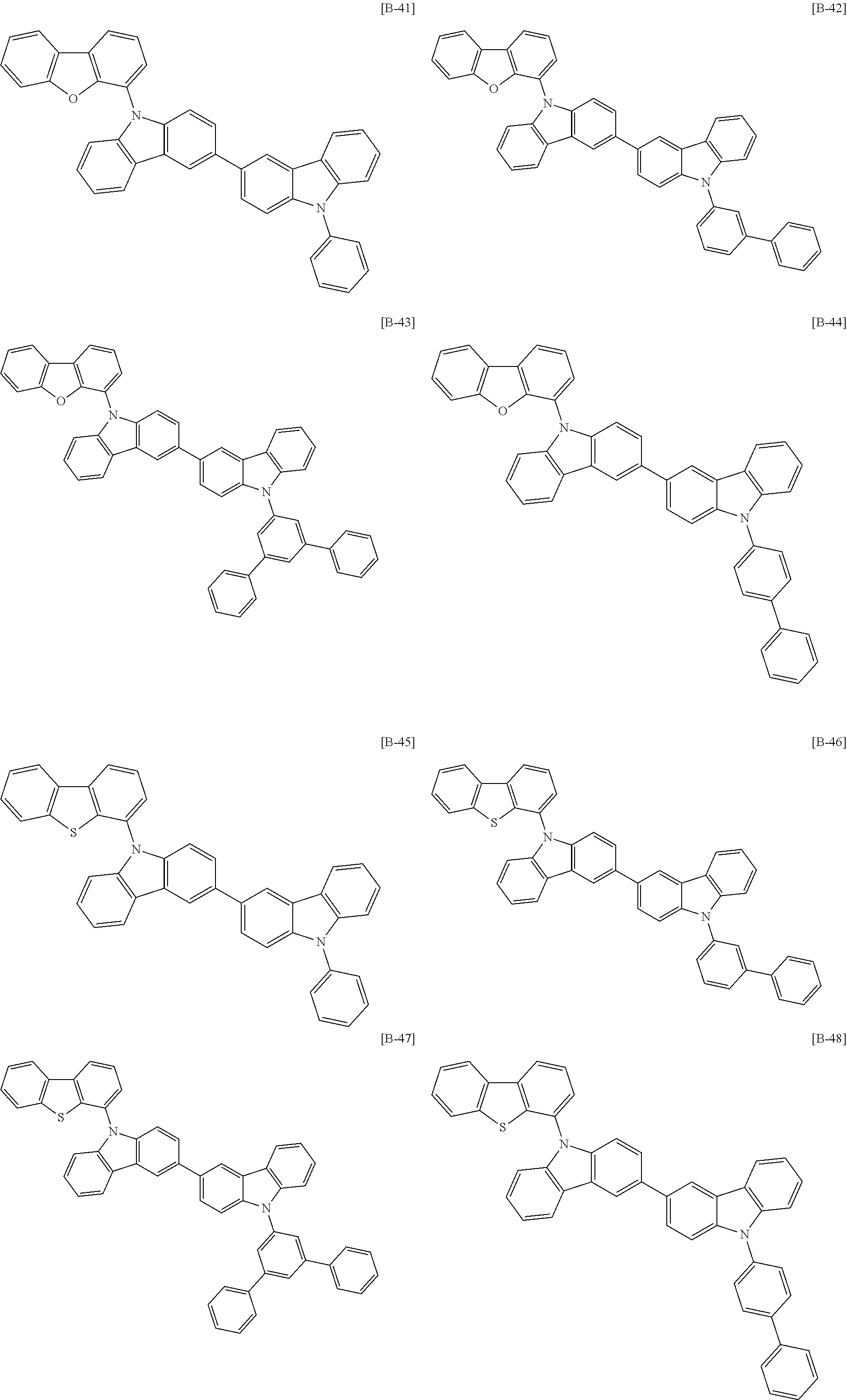

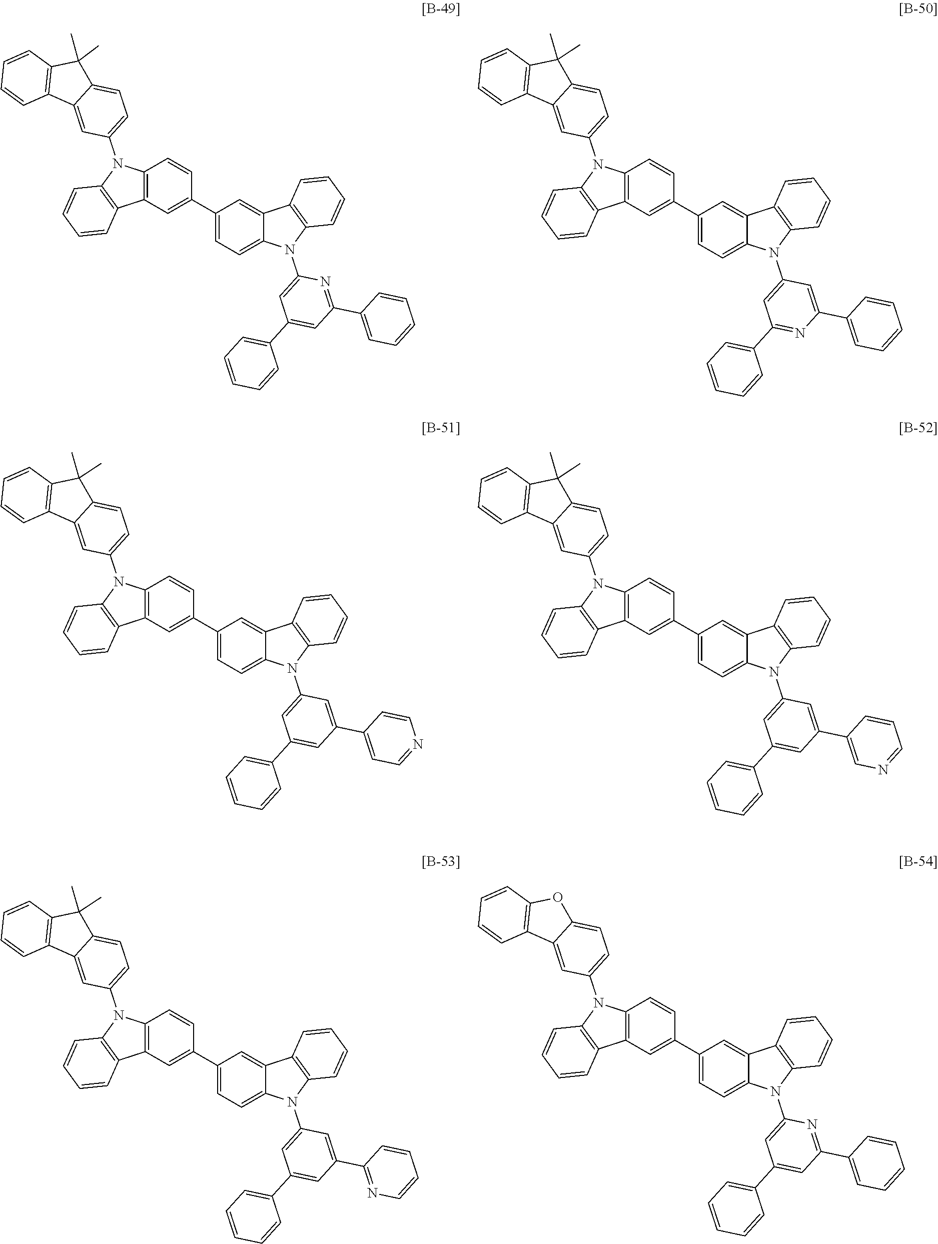

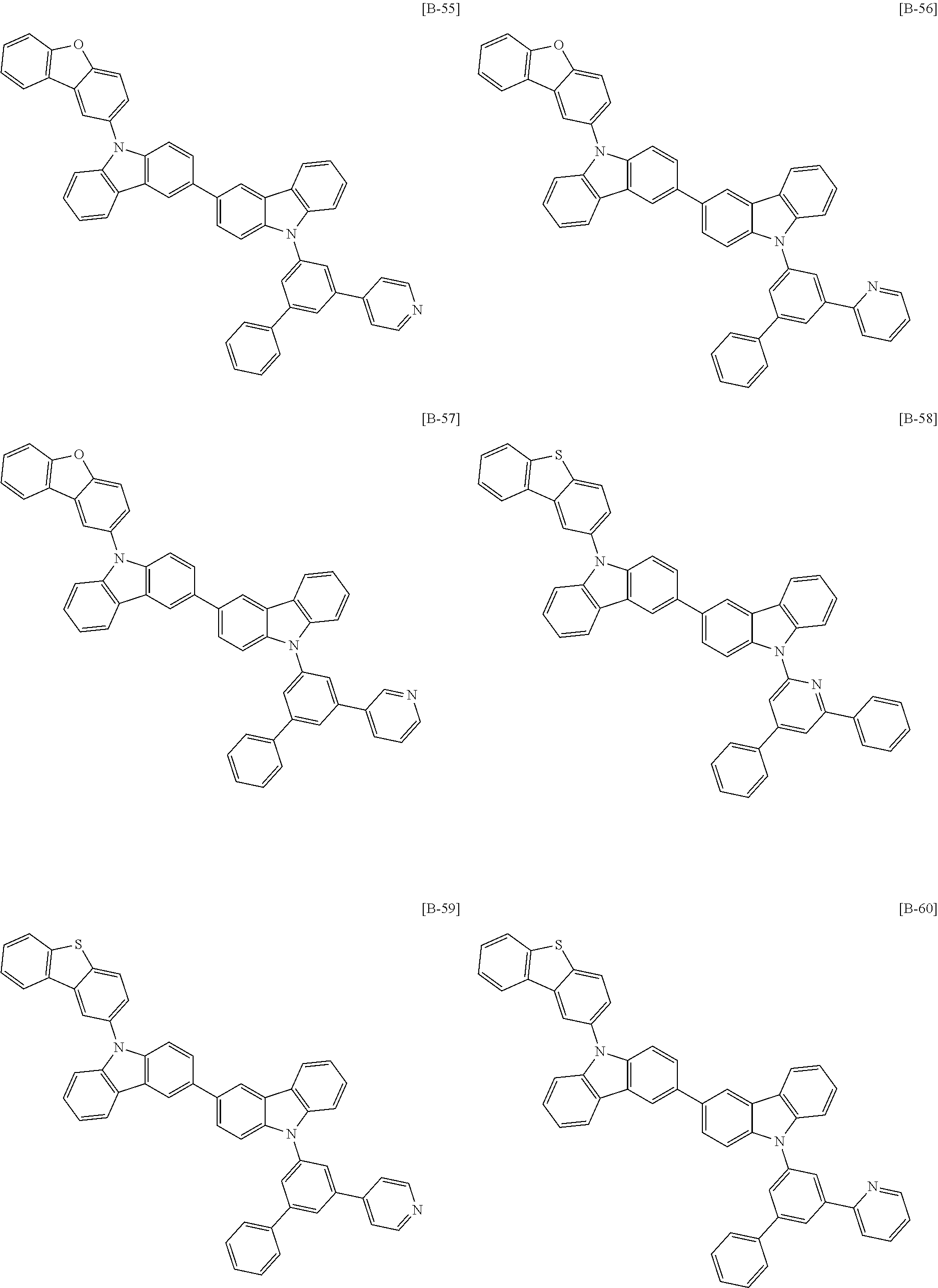

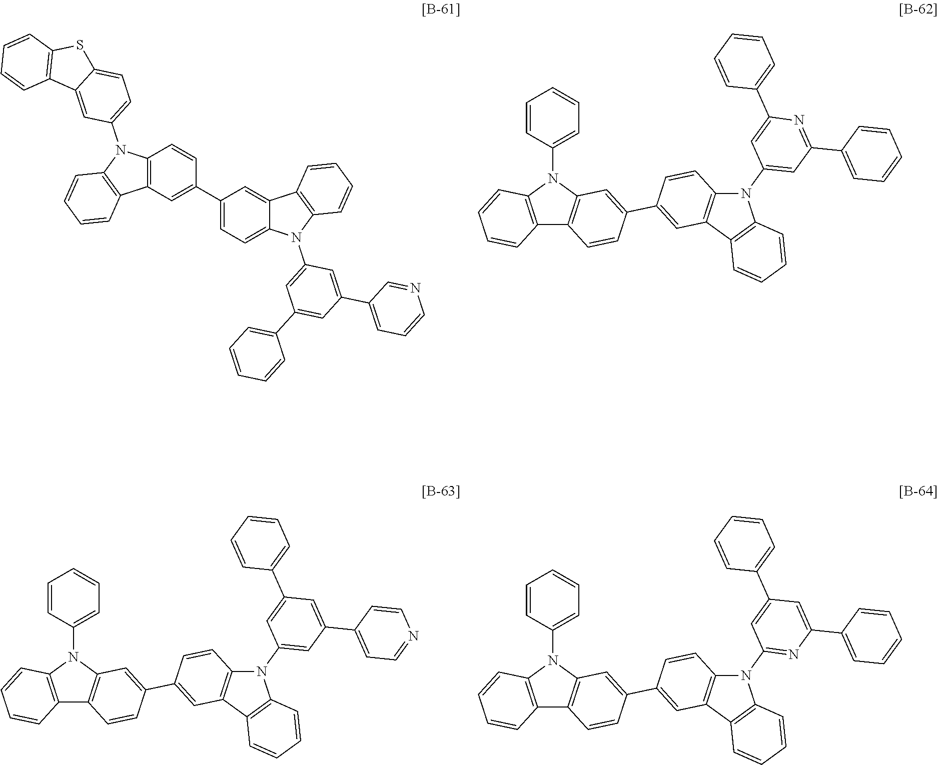 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1]

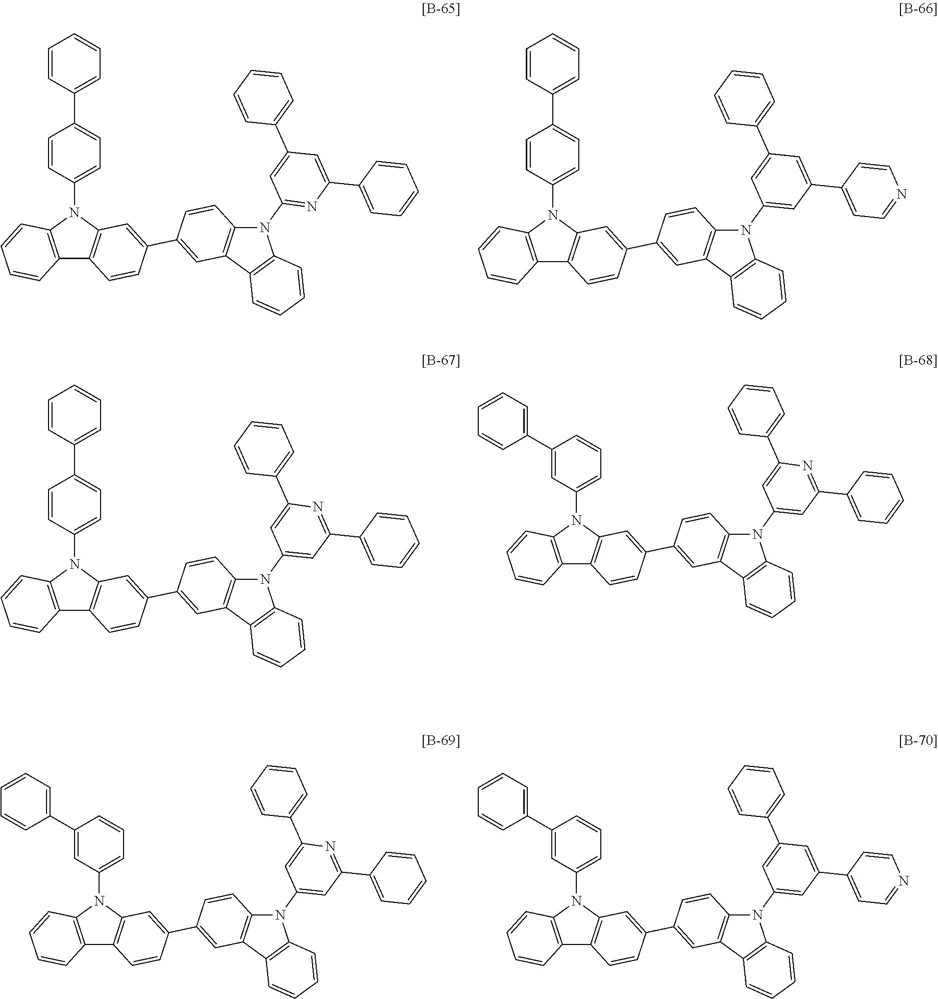

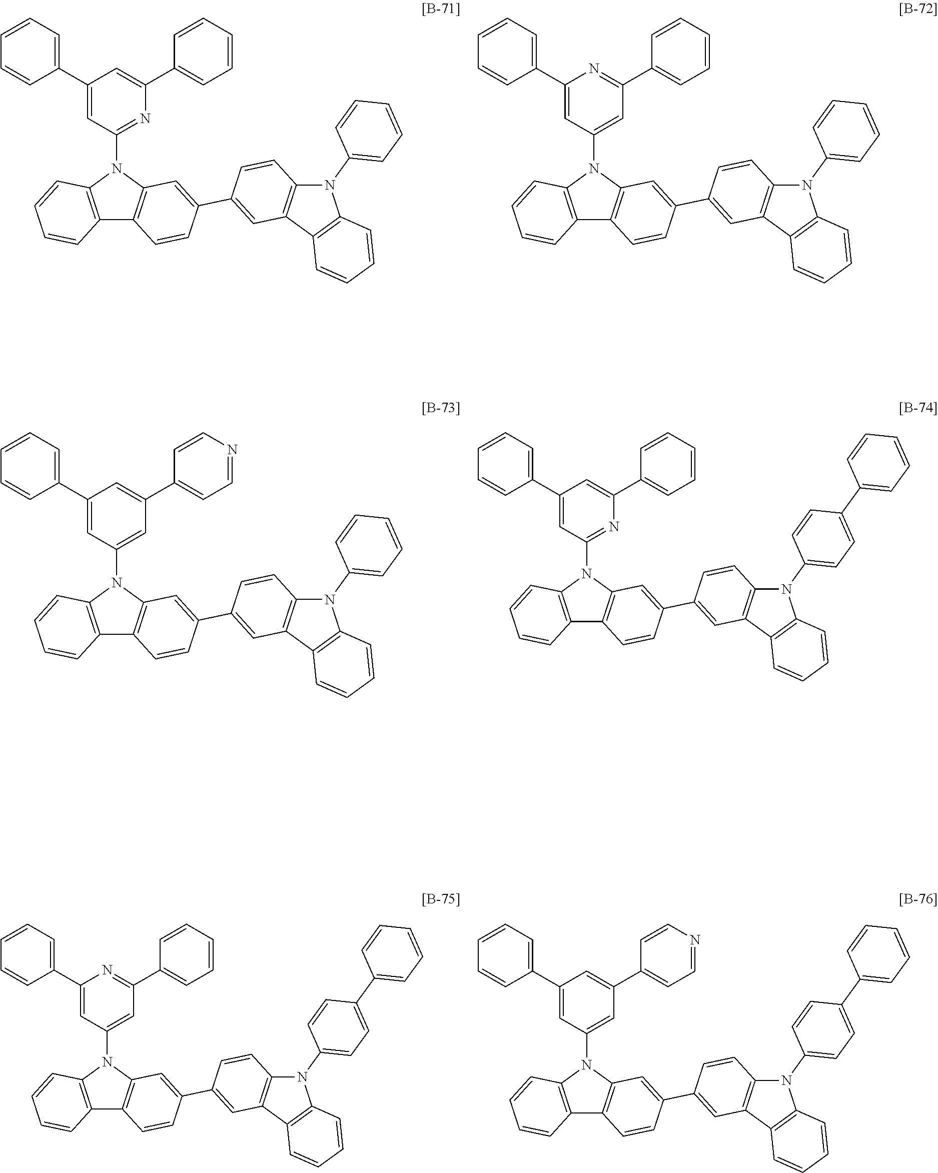

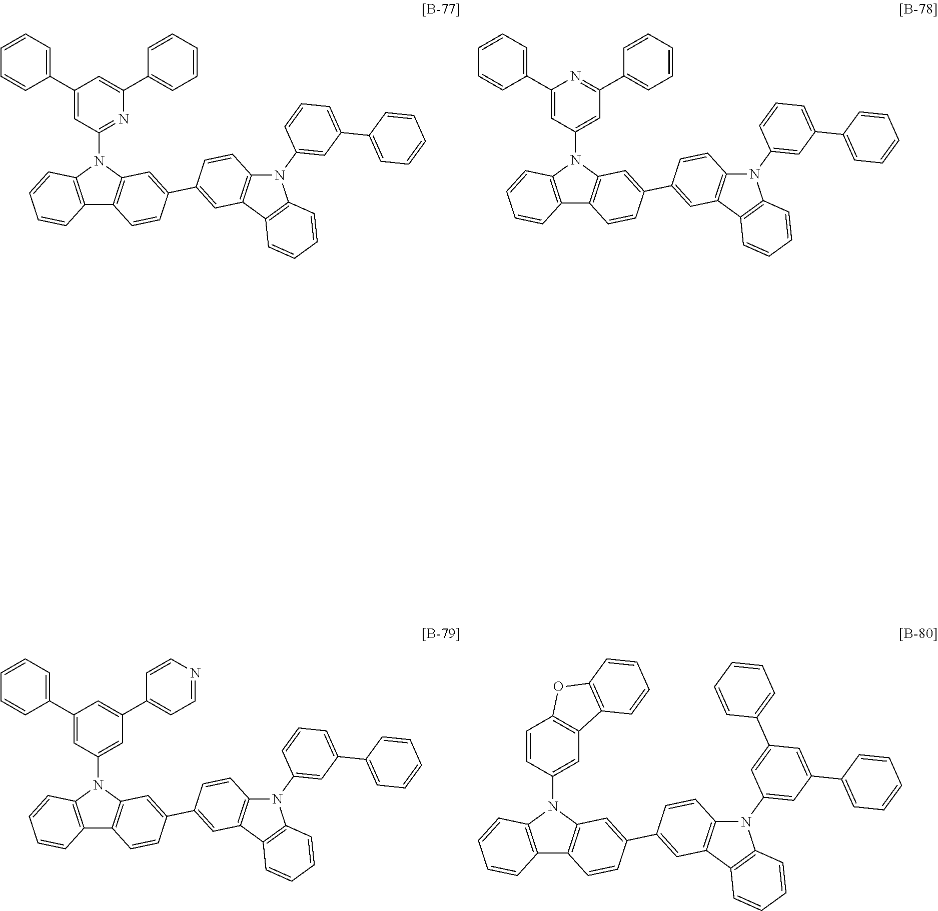 [SMILES: c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

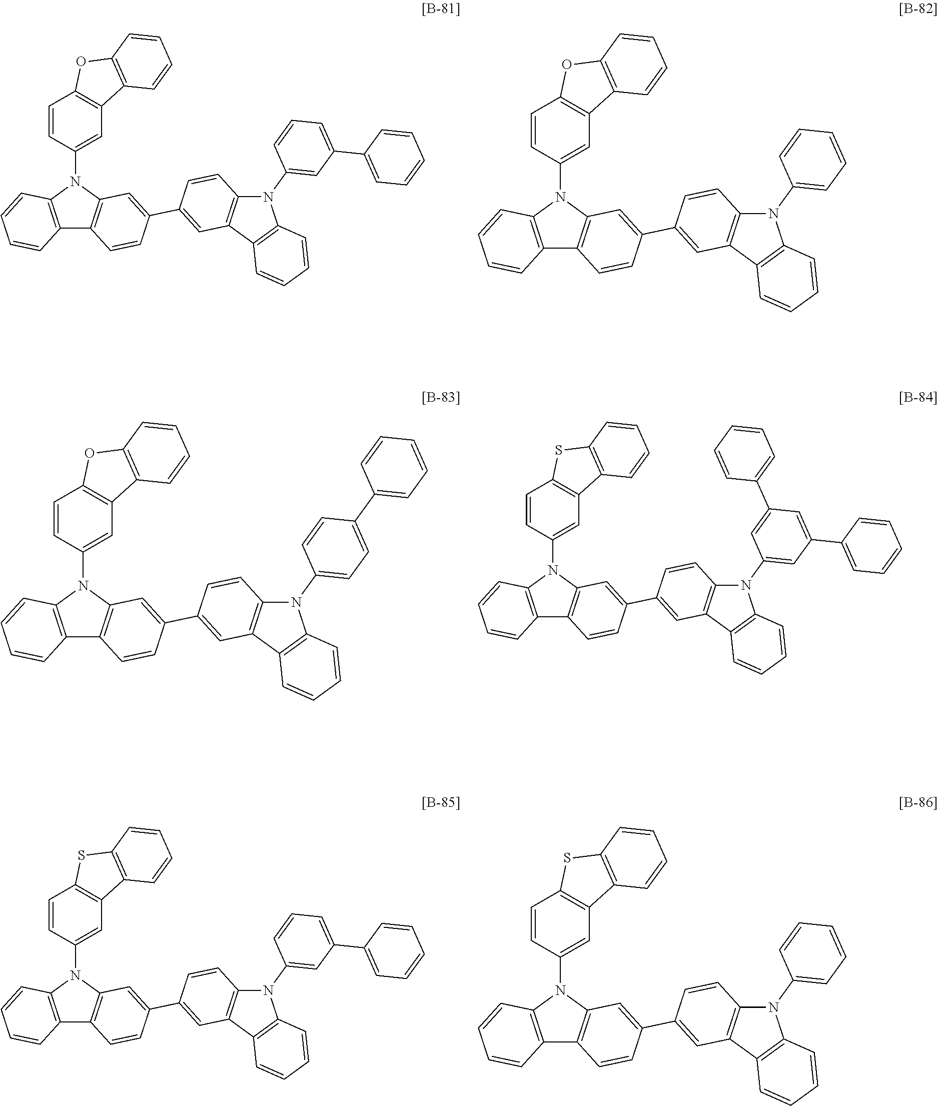

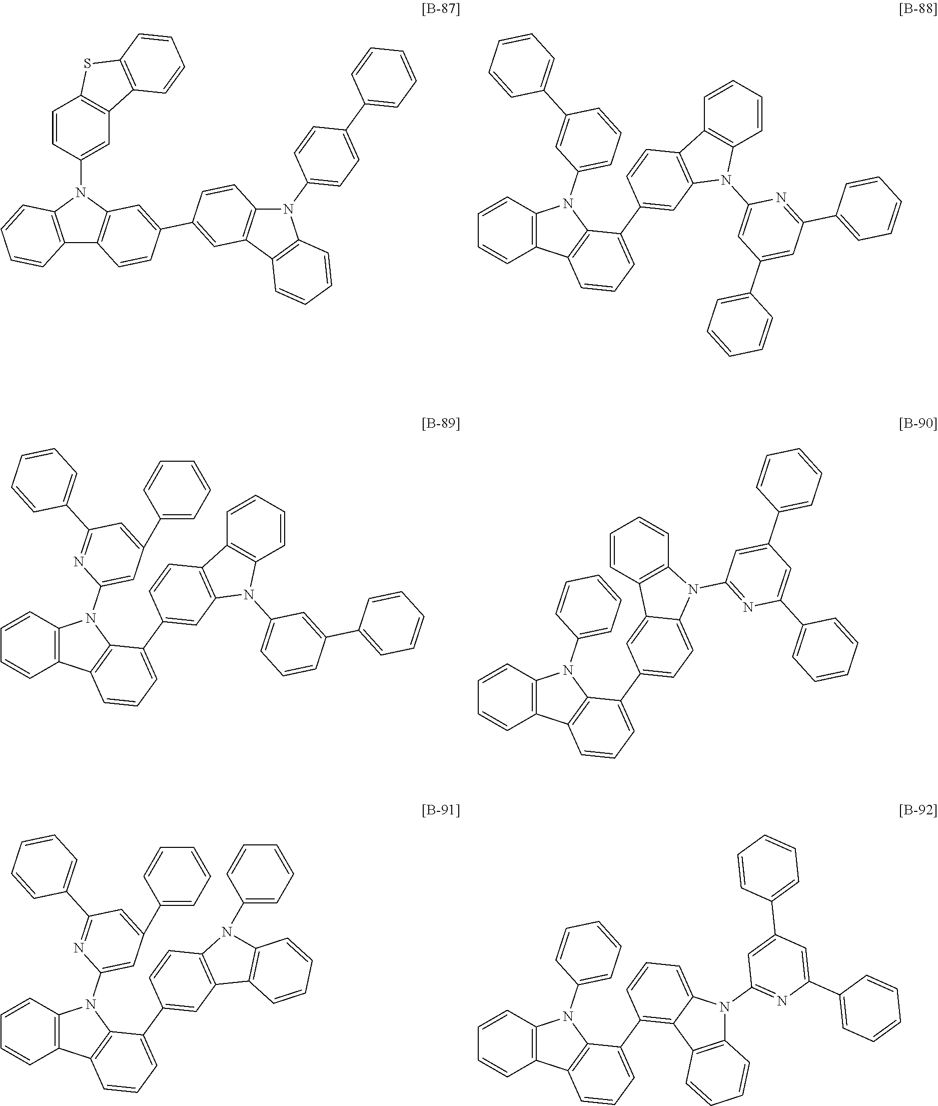

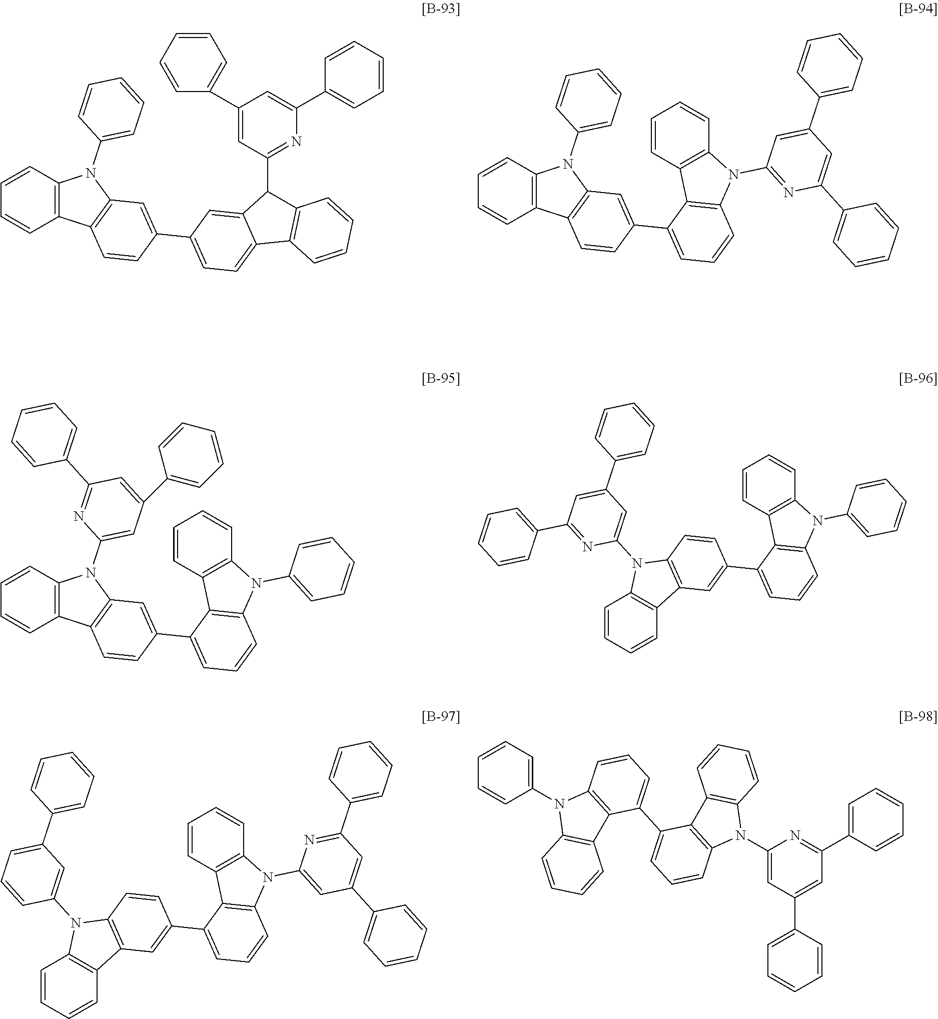

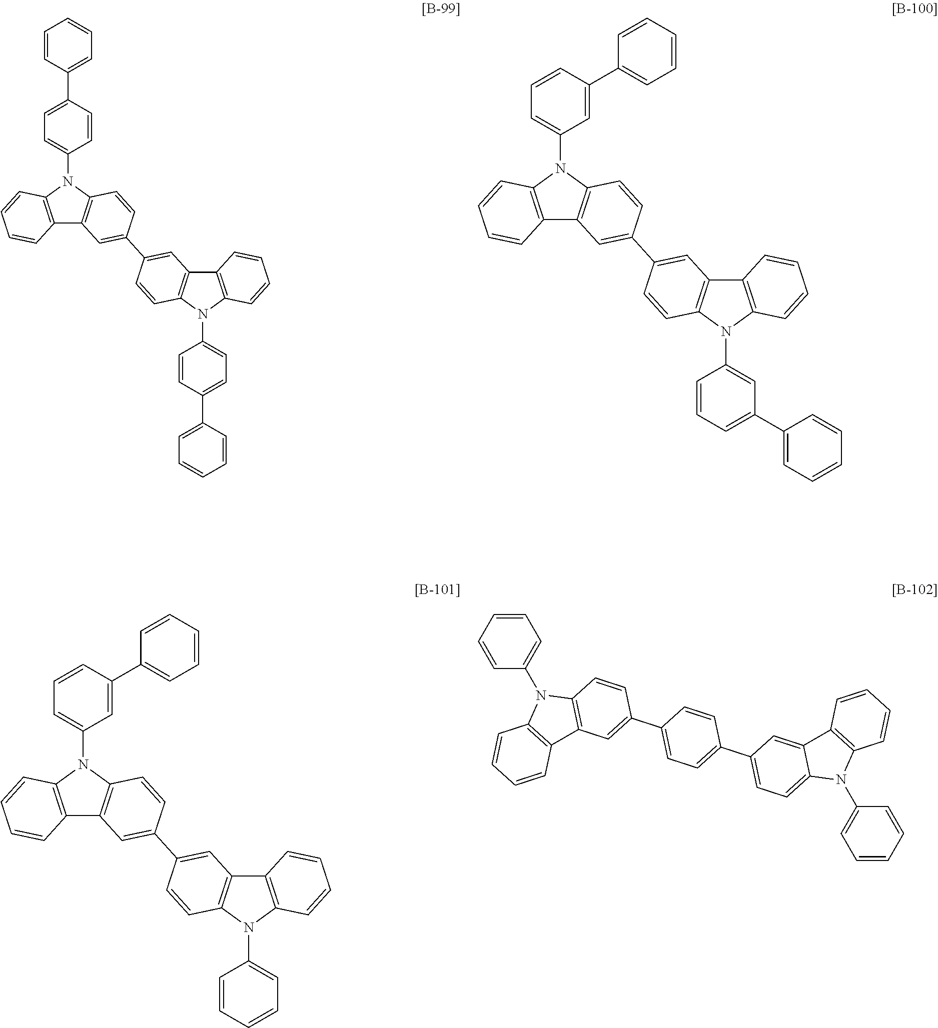 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1]

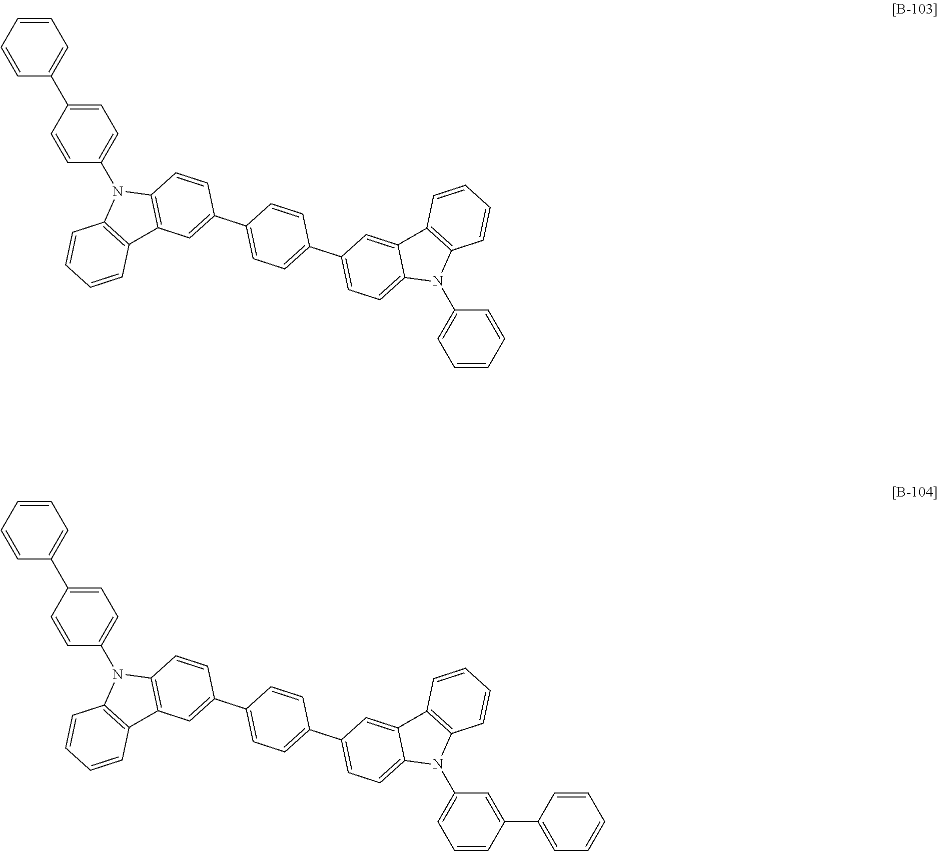 [SMILES: c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

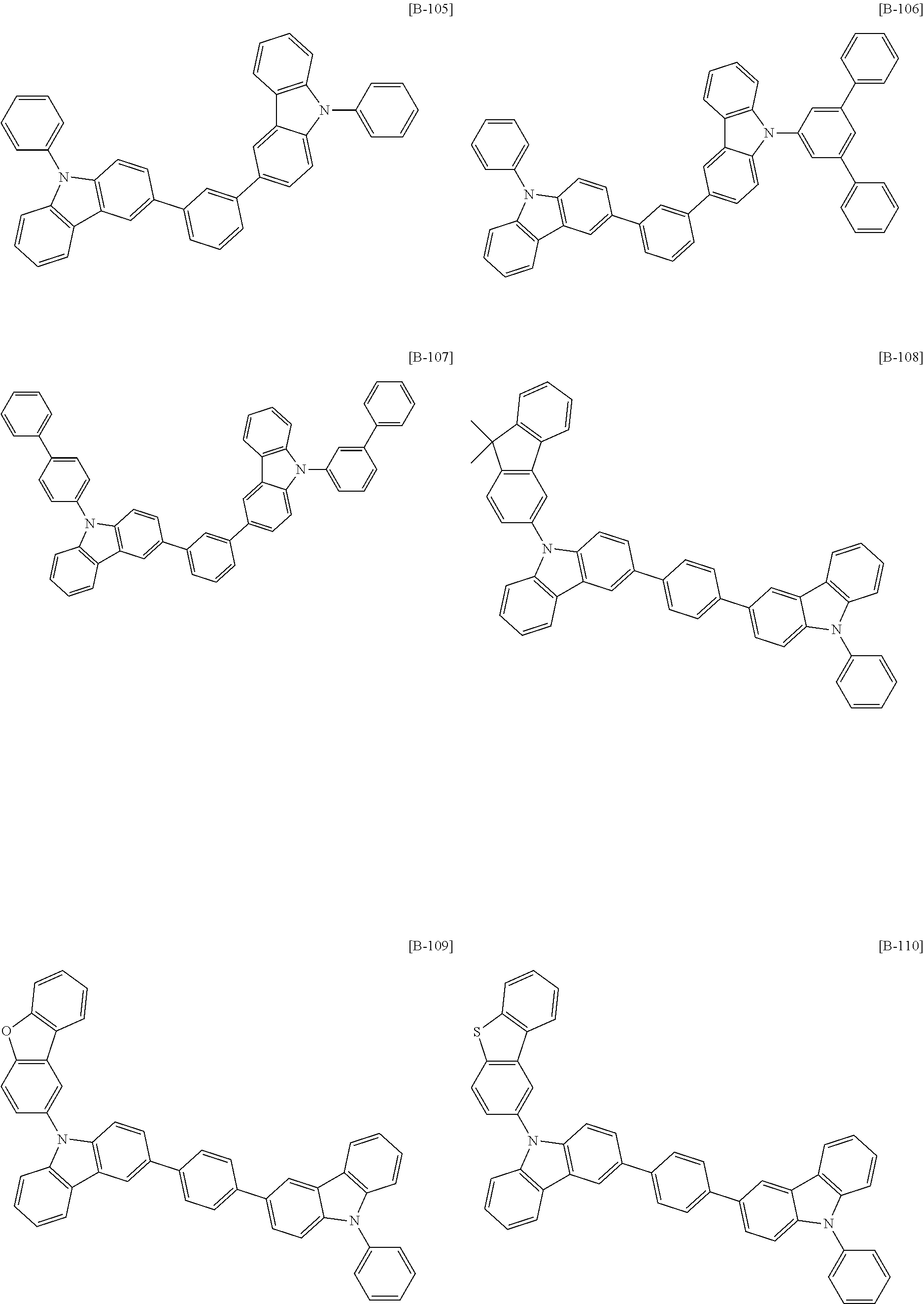

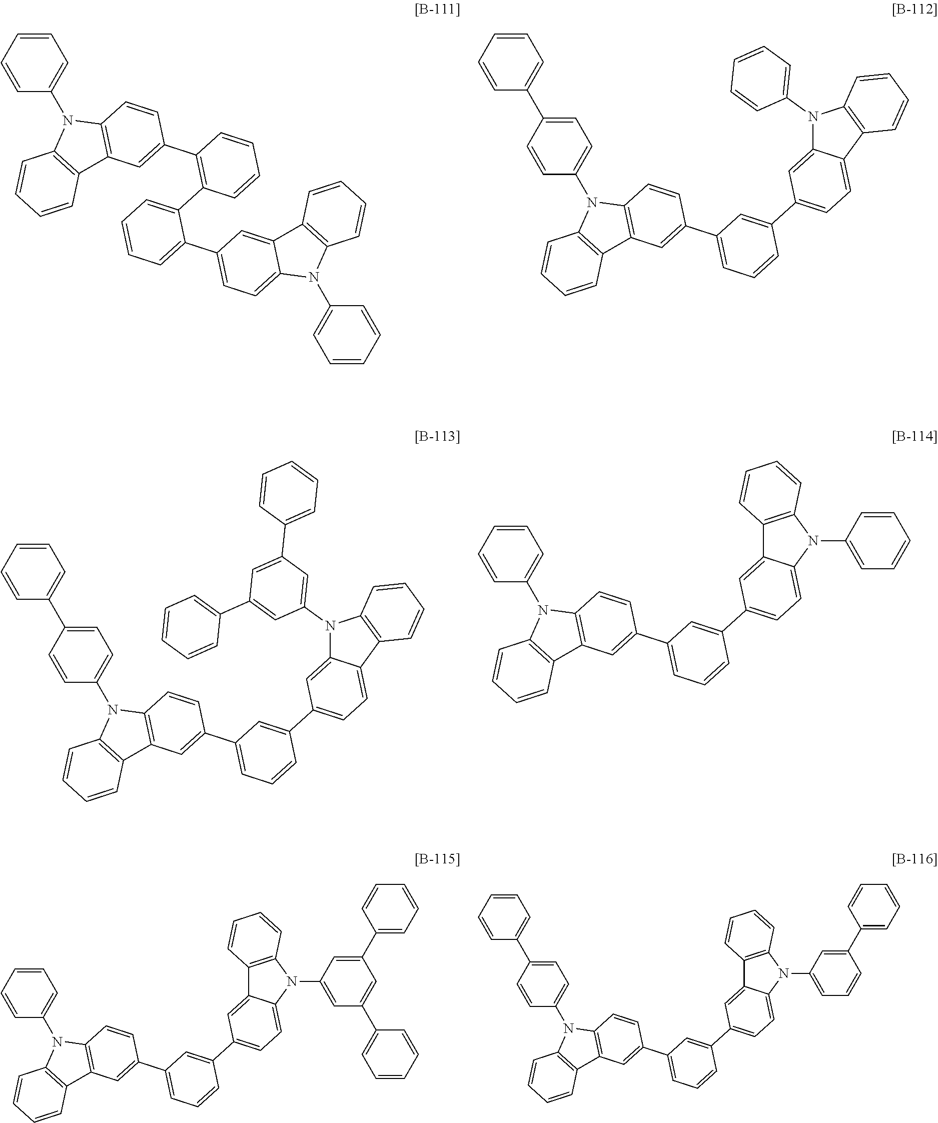

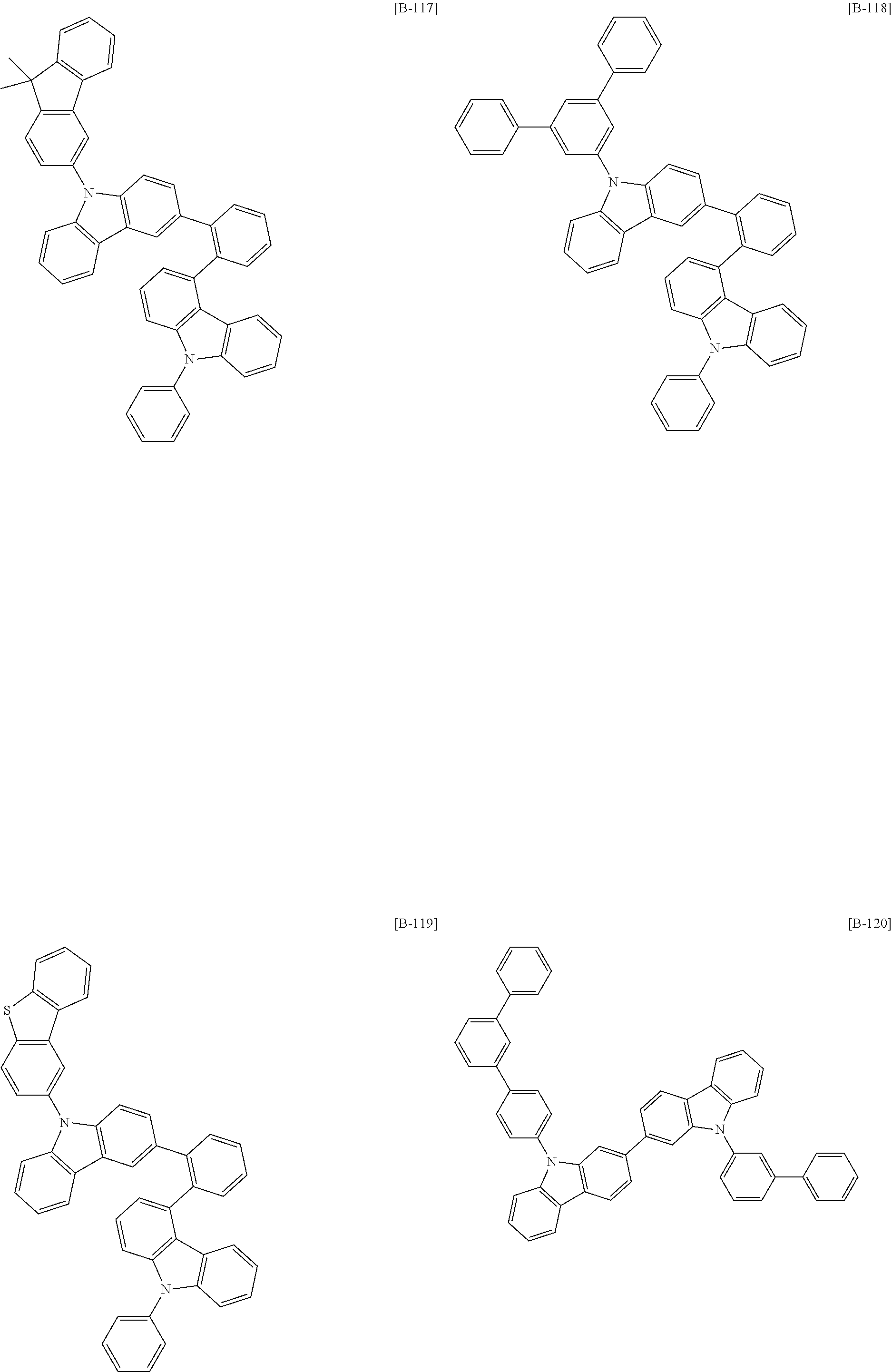 [SMILES: CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

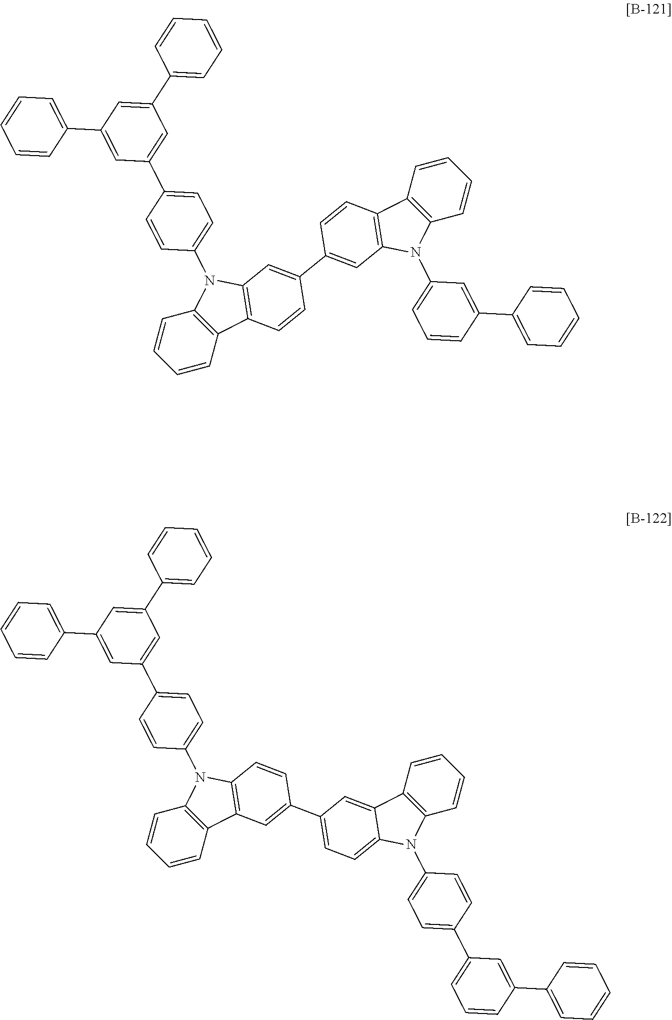 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

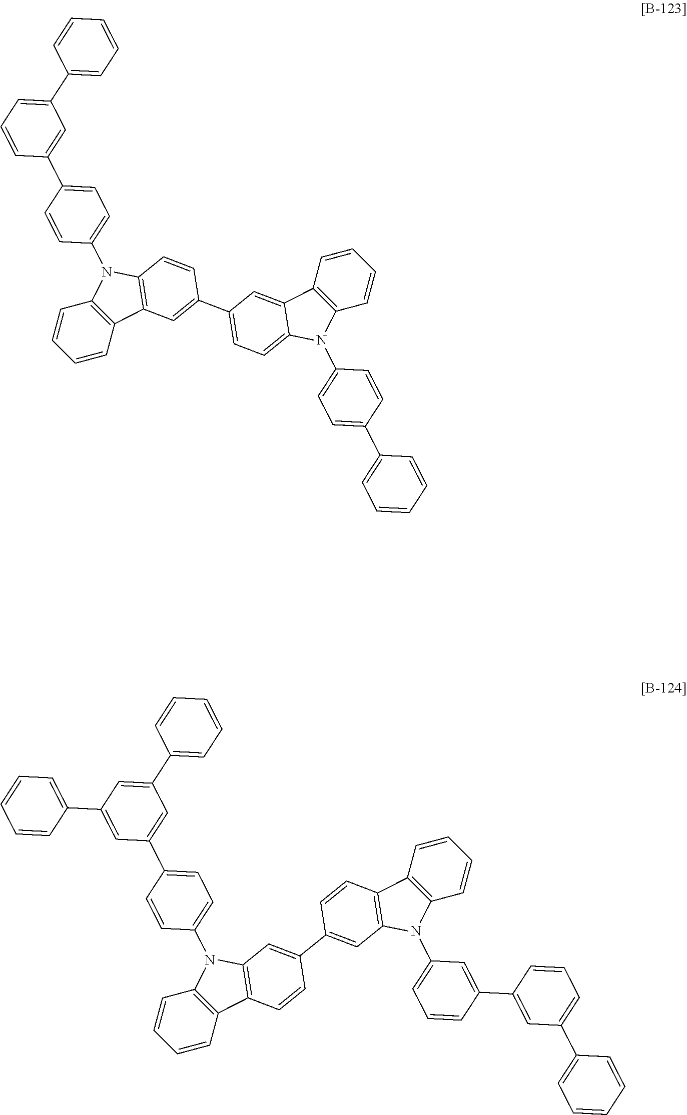 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

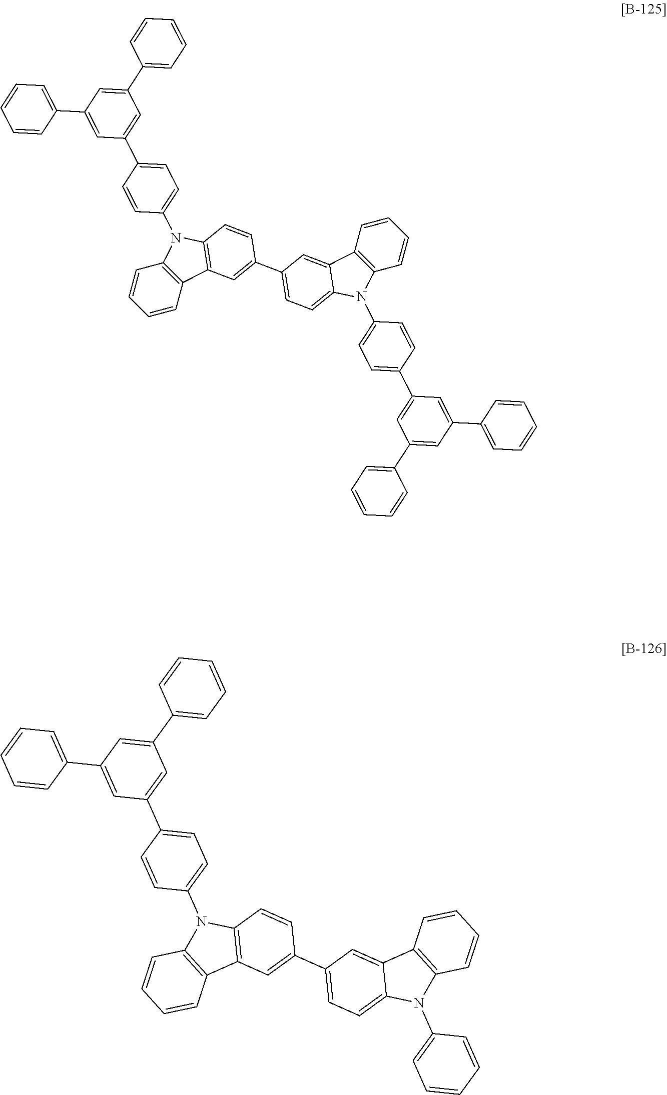 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

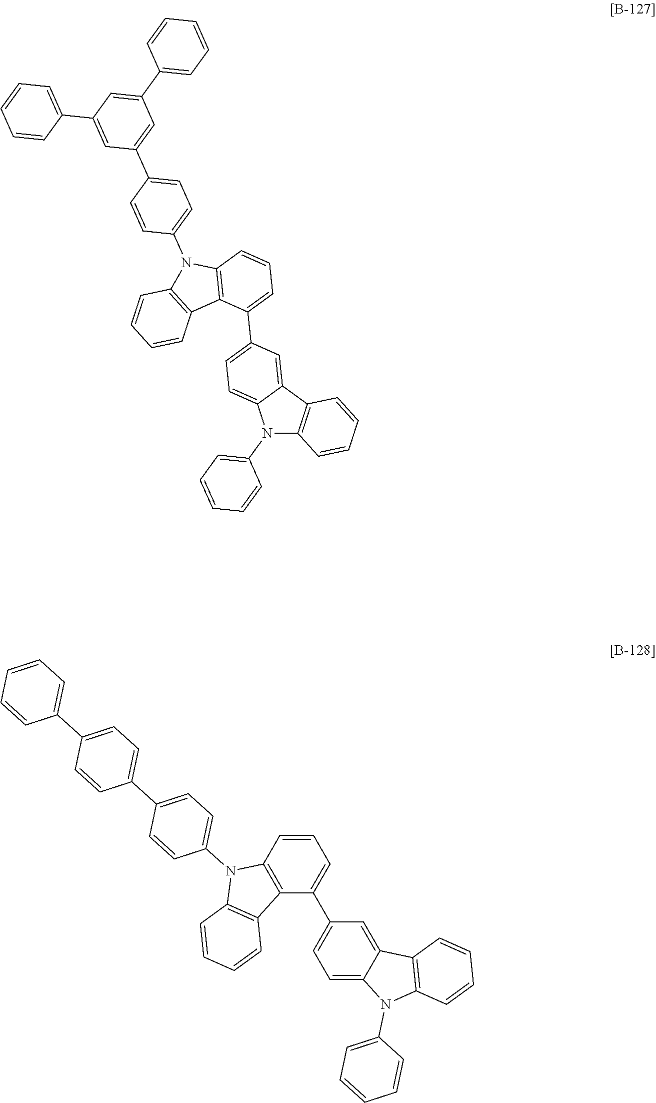 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

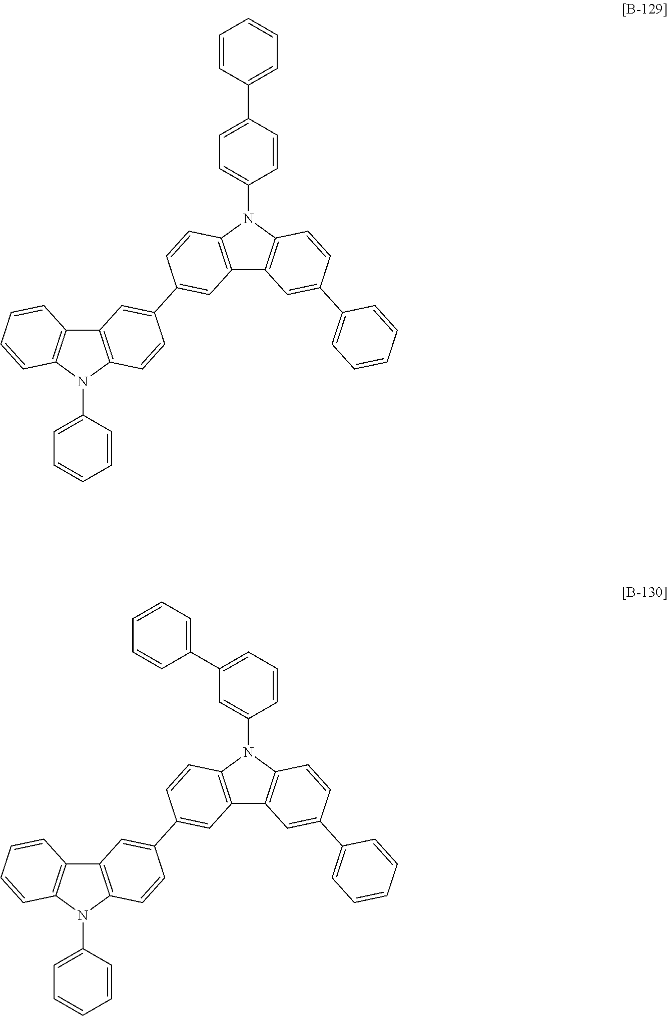 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

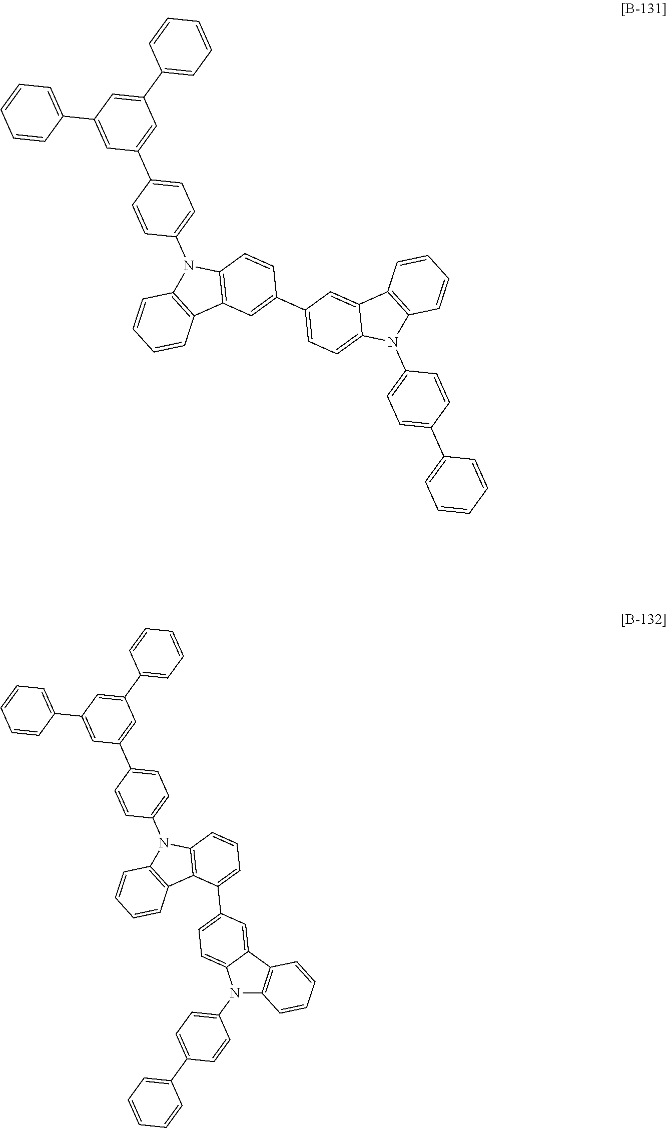 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1]

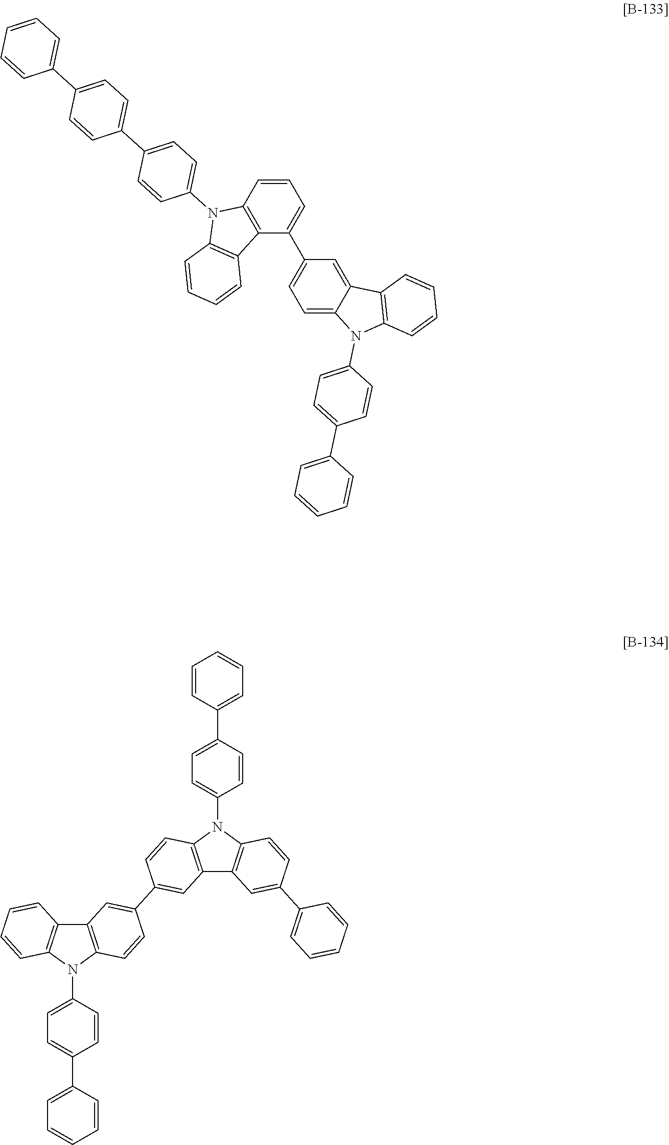 [SMILES: c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

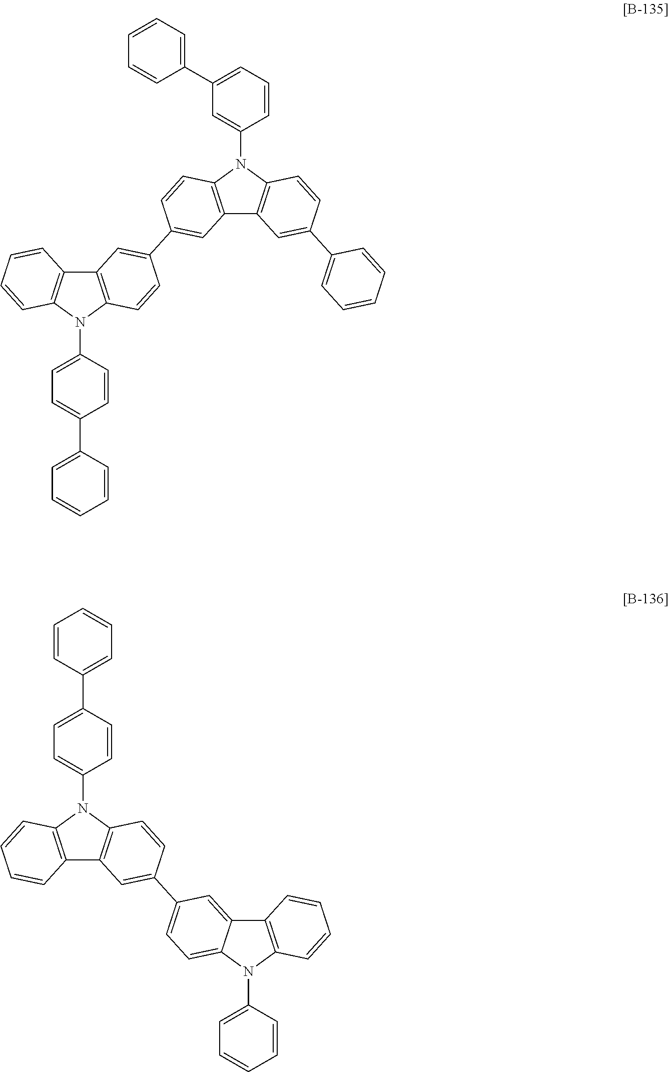 [SMILES: c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1]

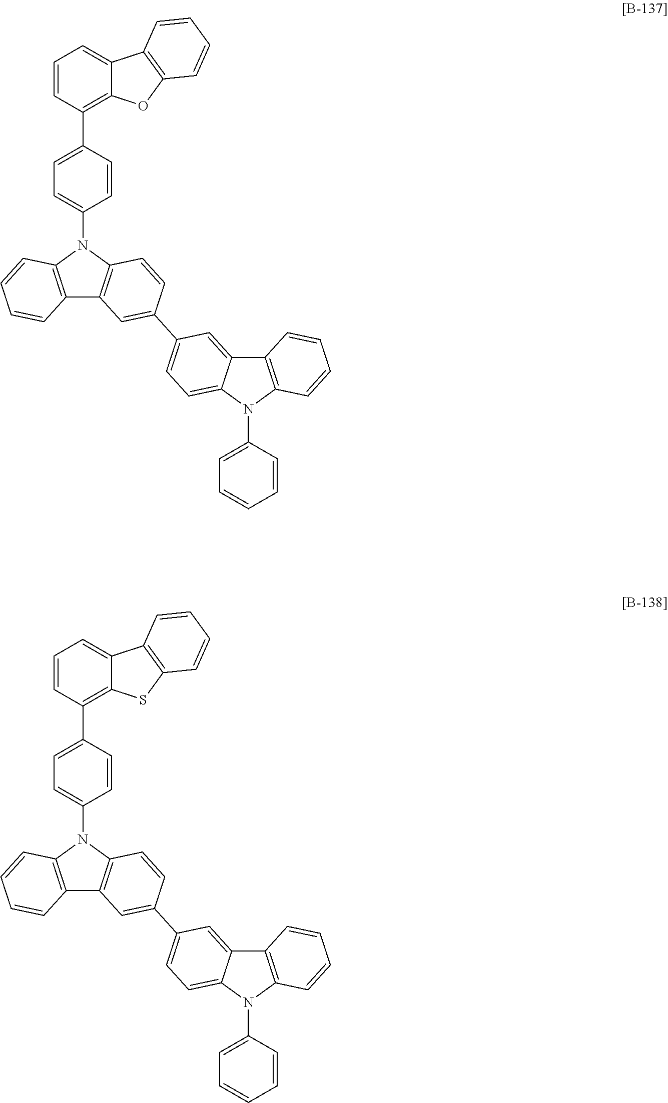 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

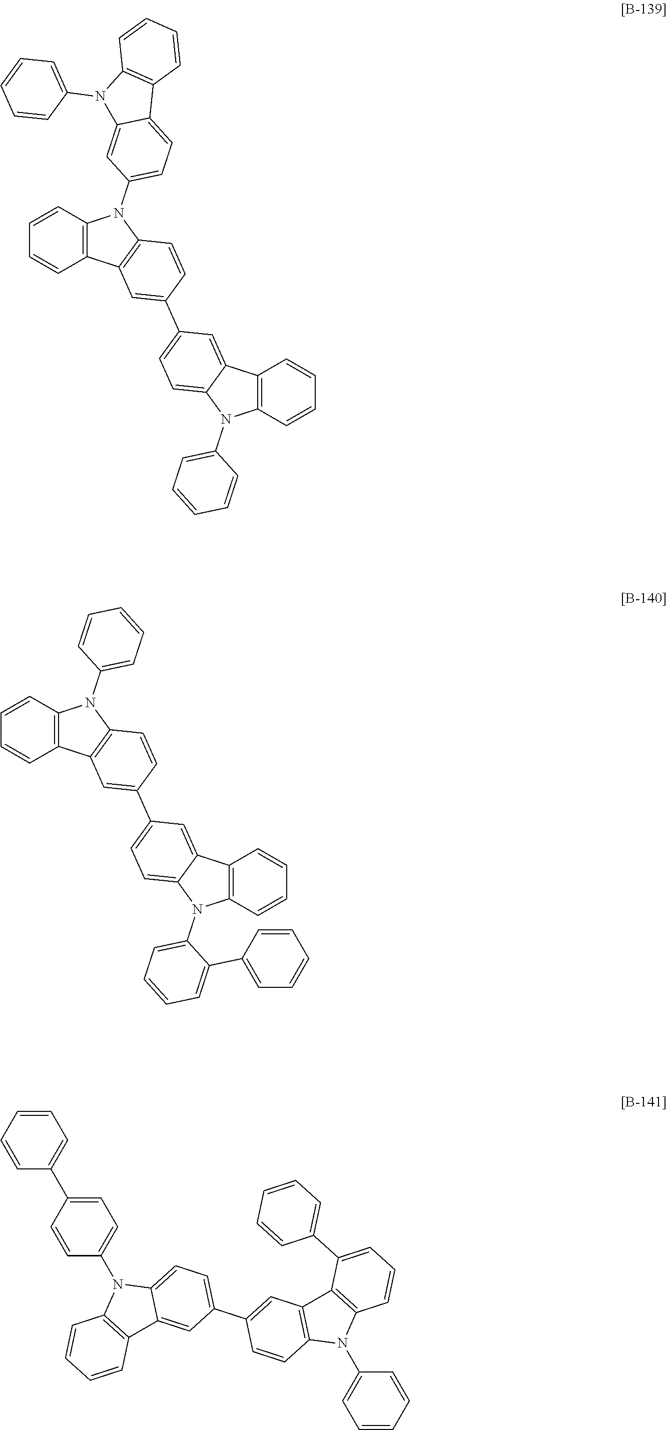 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1]

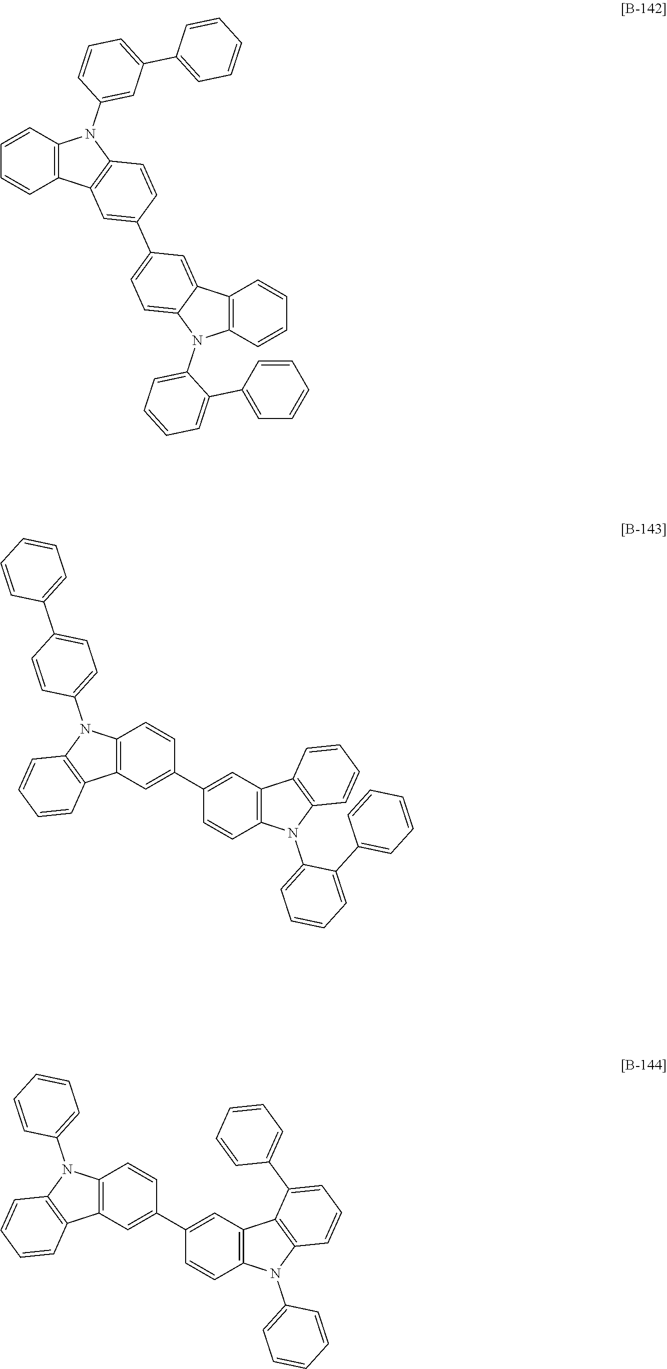 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc3c2c2cc(-c4ccc5c(c4)c4ccccc4n5-c4ccccc4)ccc2n3-c2ccccc2)cc1]

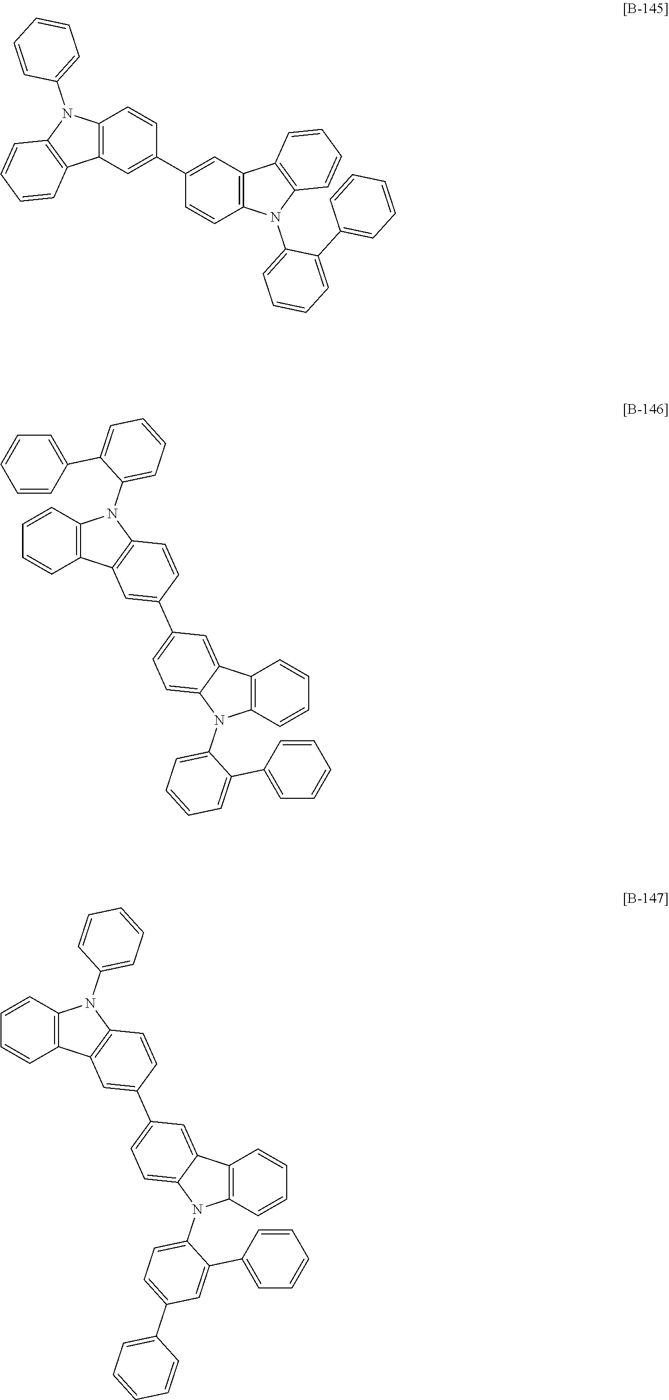 [SMILES: c1ccc(-c2ccccc2-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1]

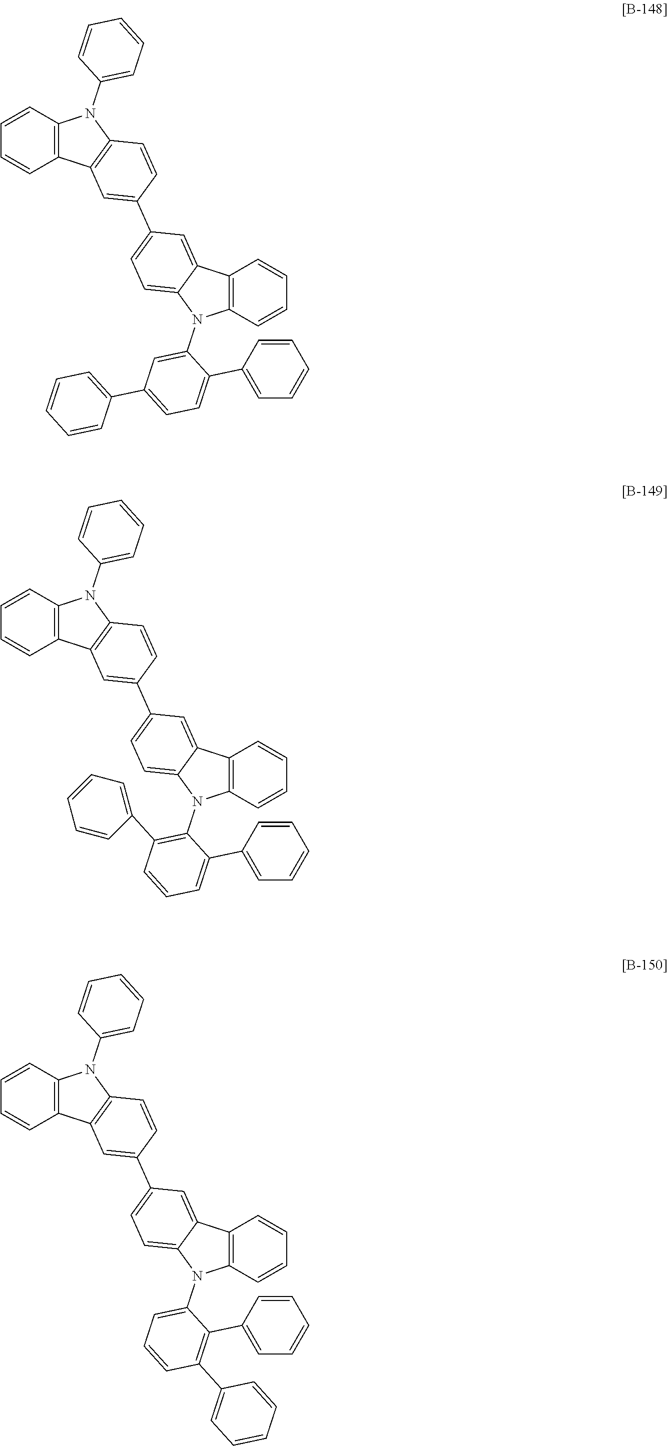 [SMILES: c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2-c2ccccc2)cc1]

Examples of Compound B-1 to Compound B-150 listed in Group 2 in which at least one hydrogen is replaced with deuterium are shown in Compound B-151 to Compound B-195, exemplified below.

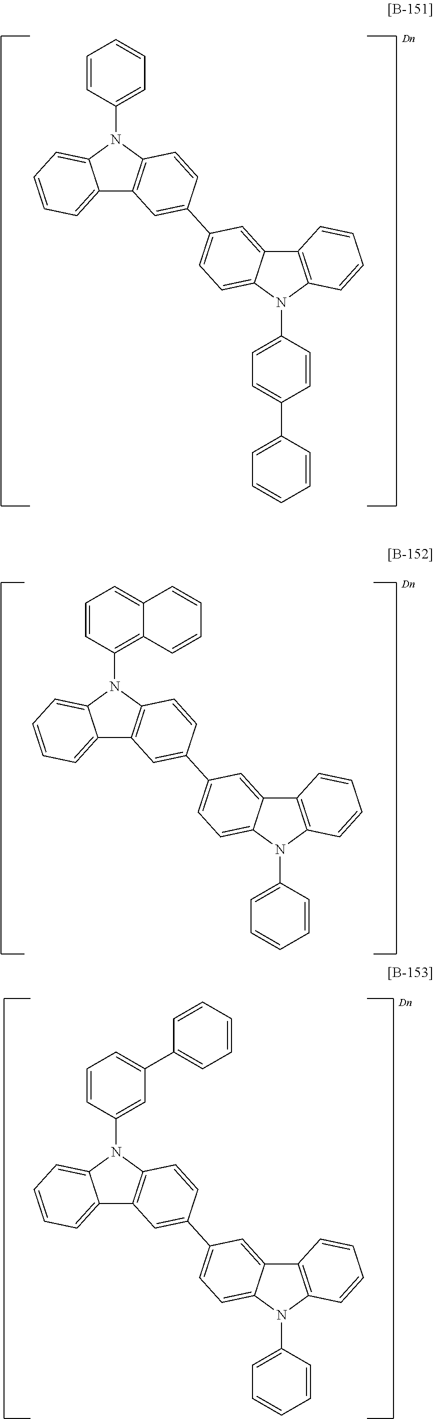

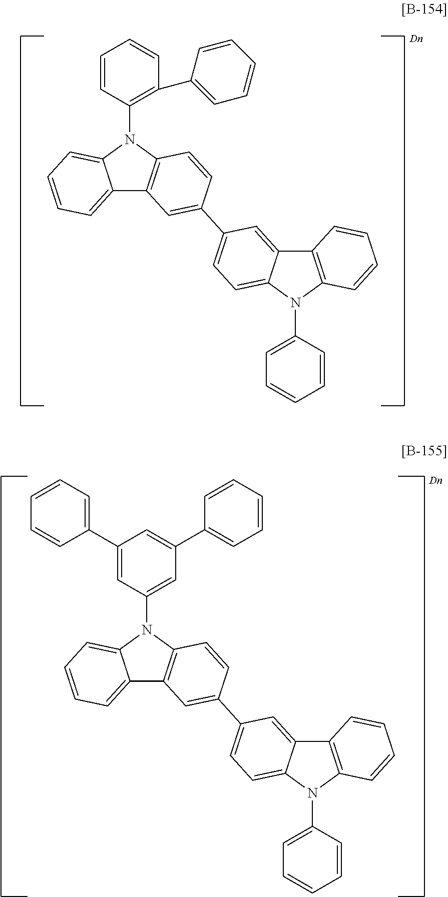

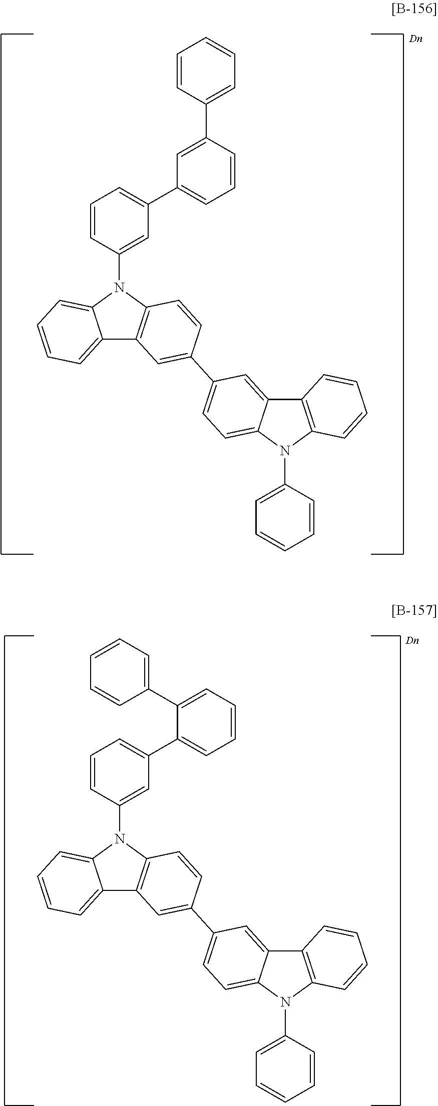

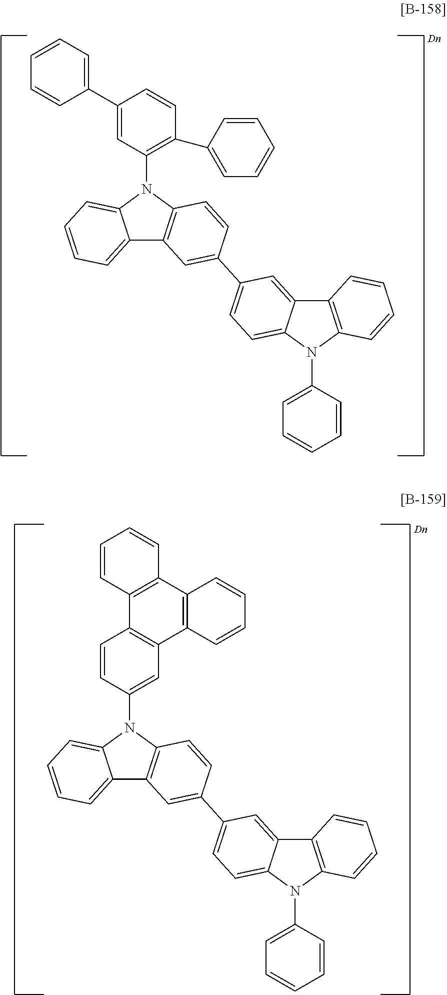

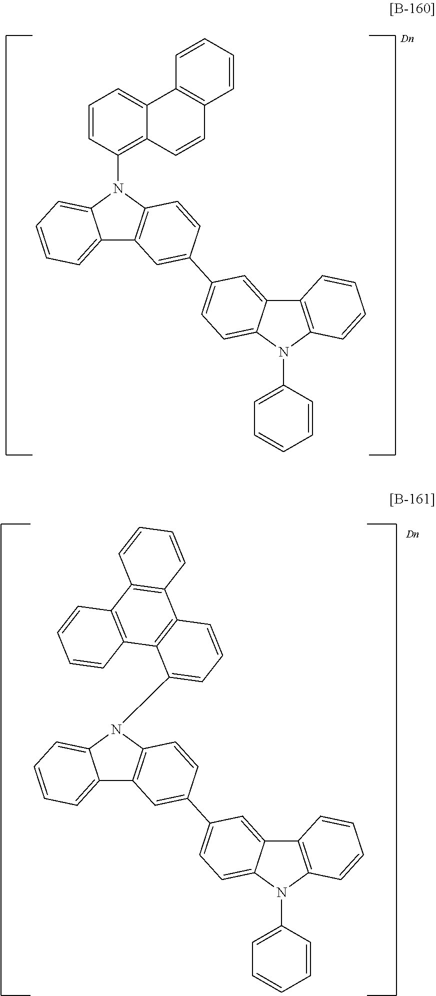

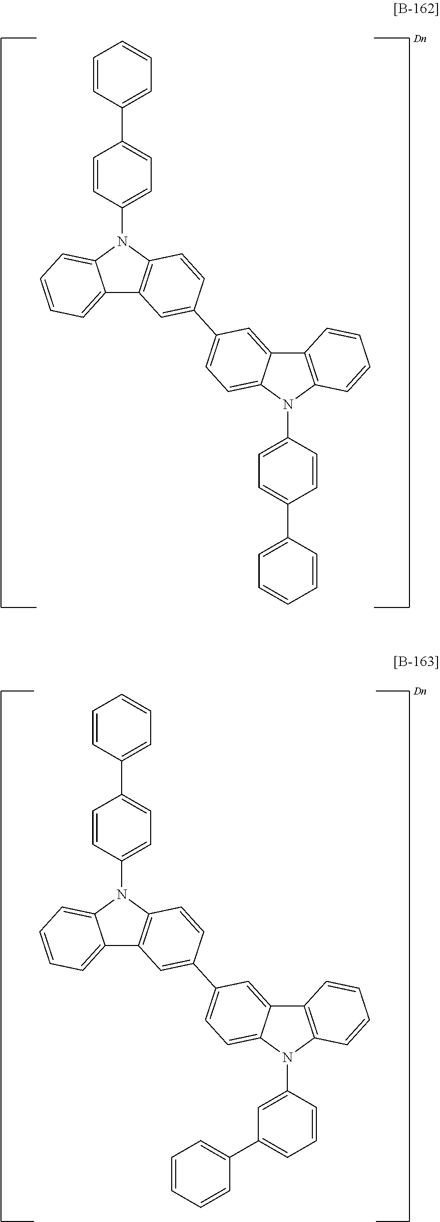

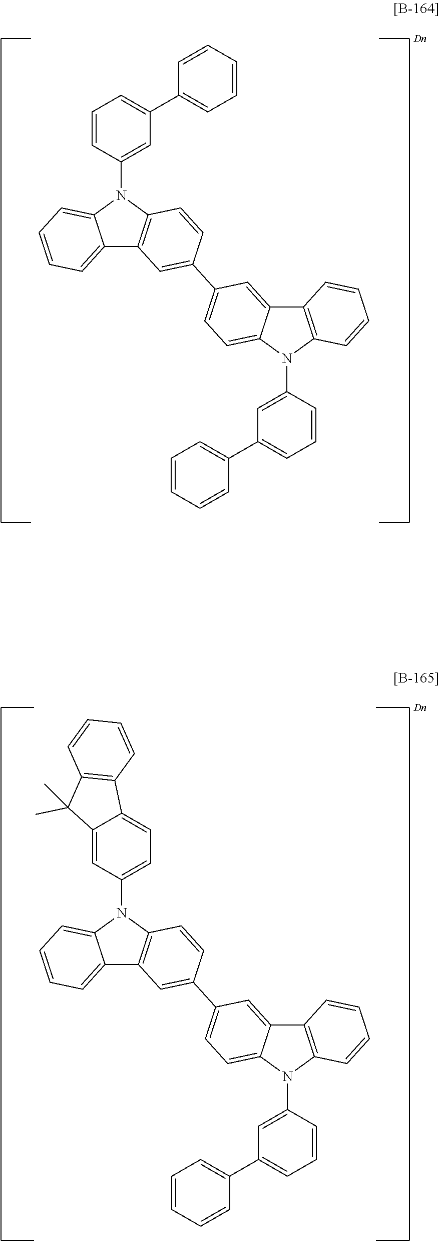

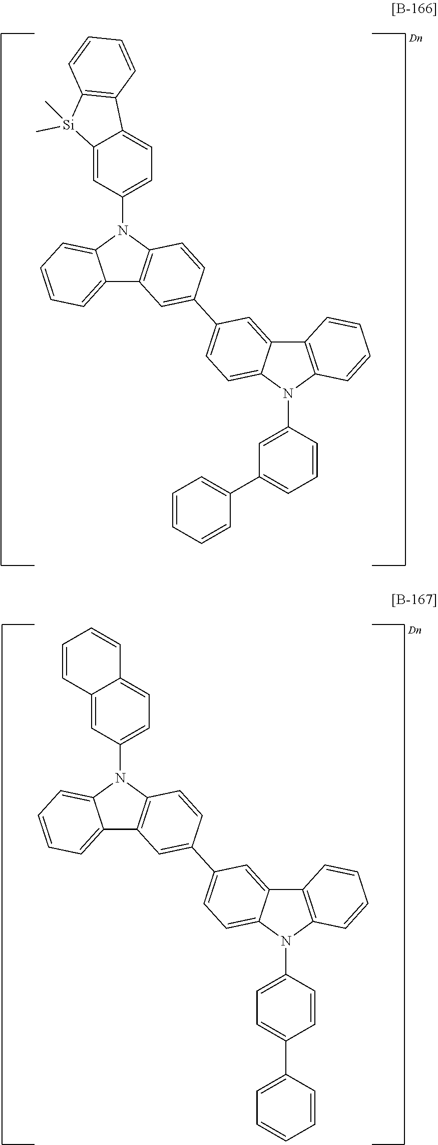

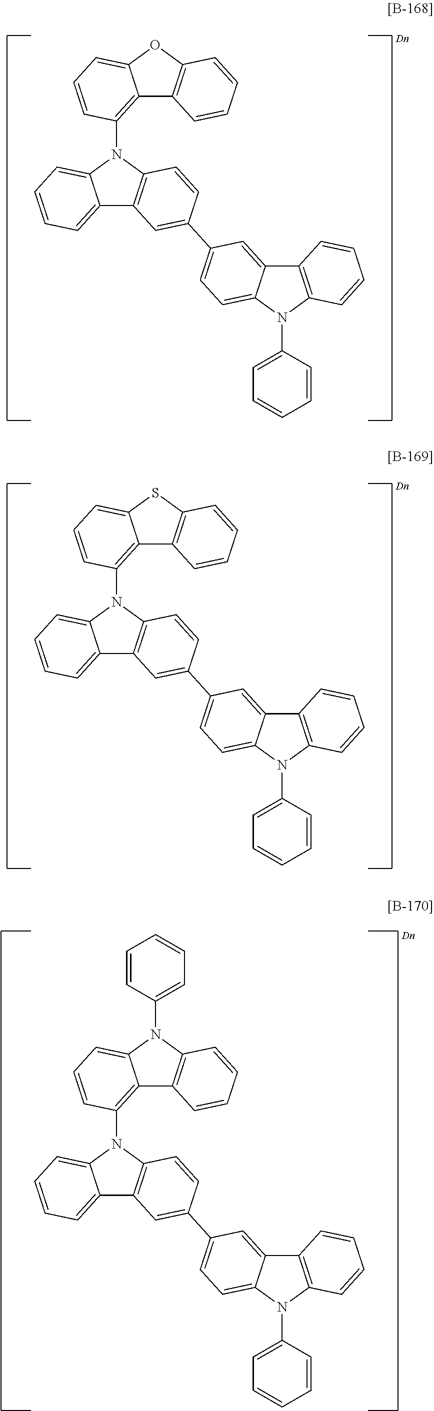

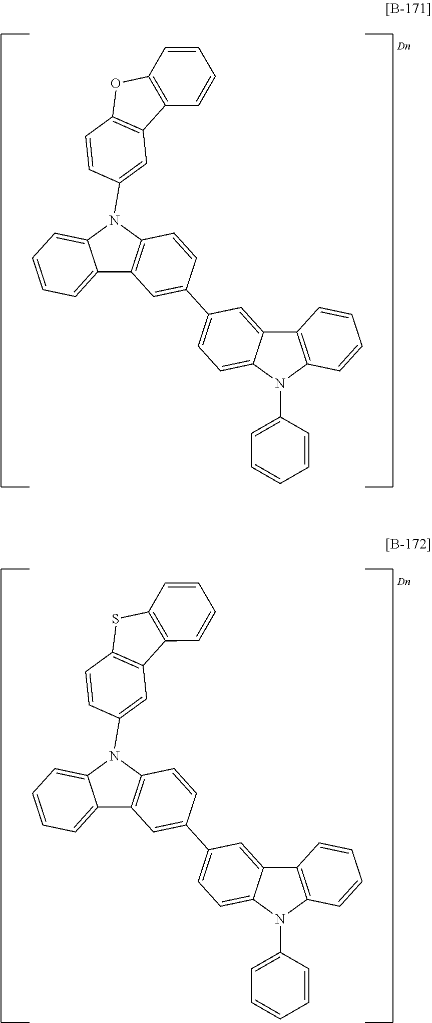

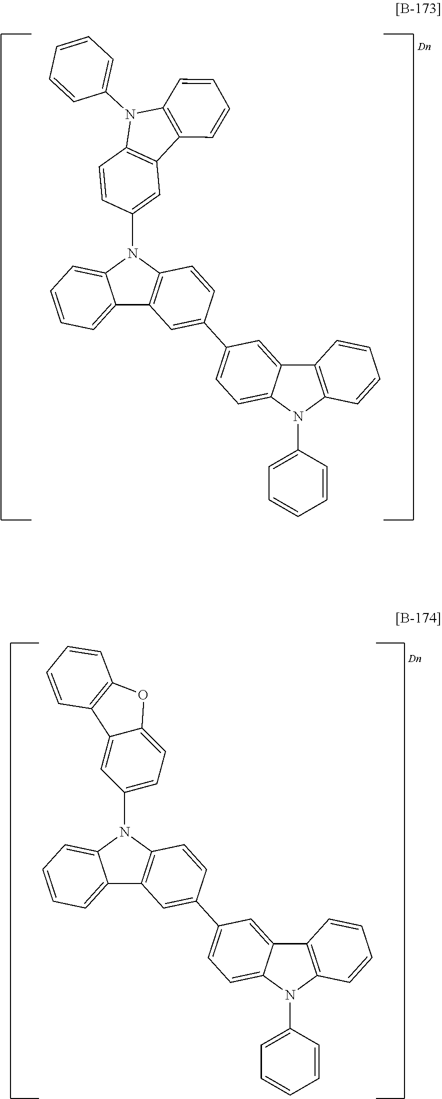

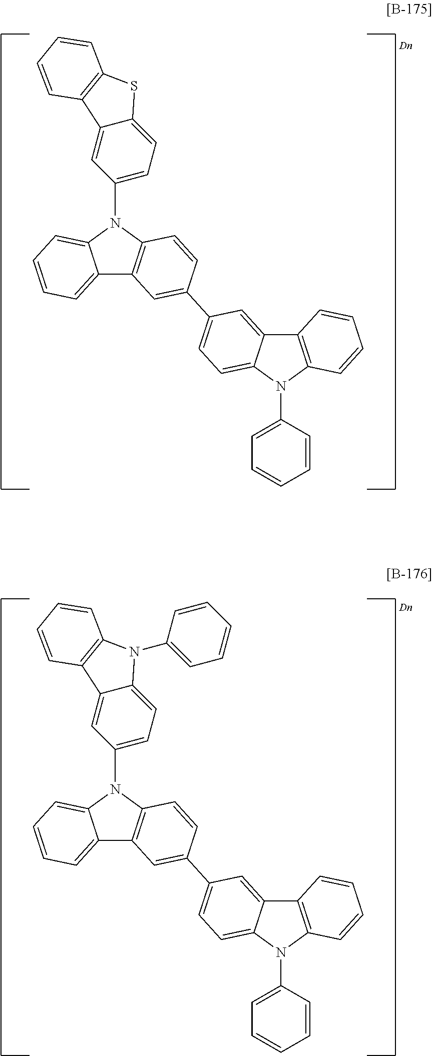

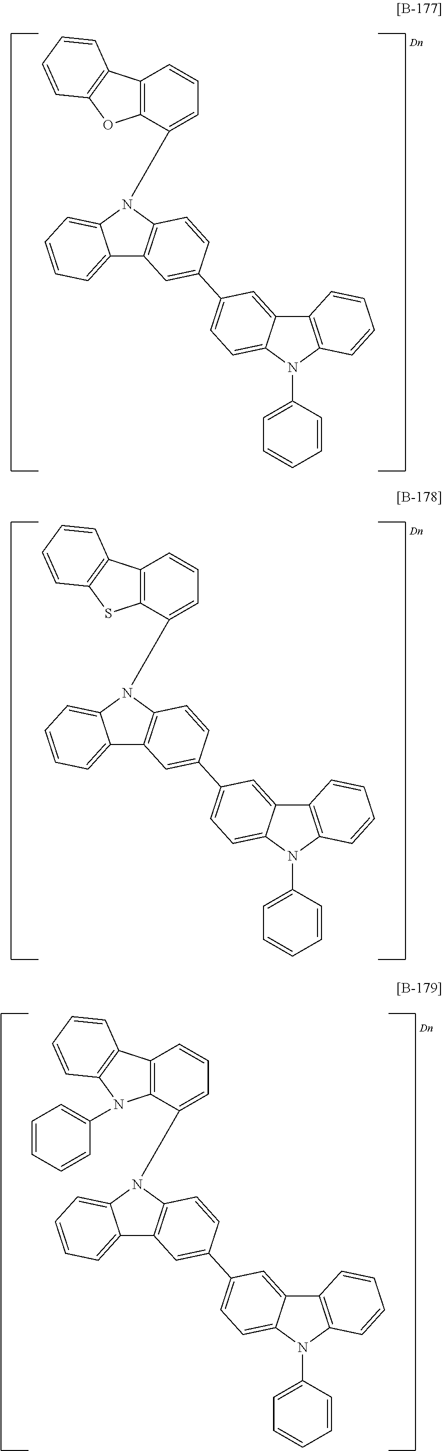

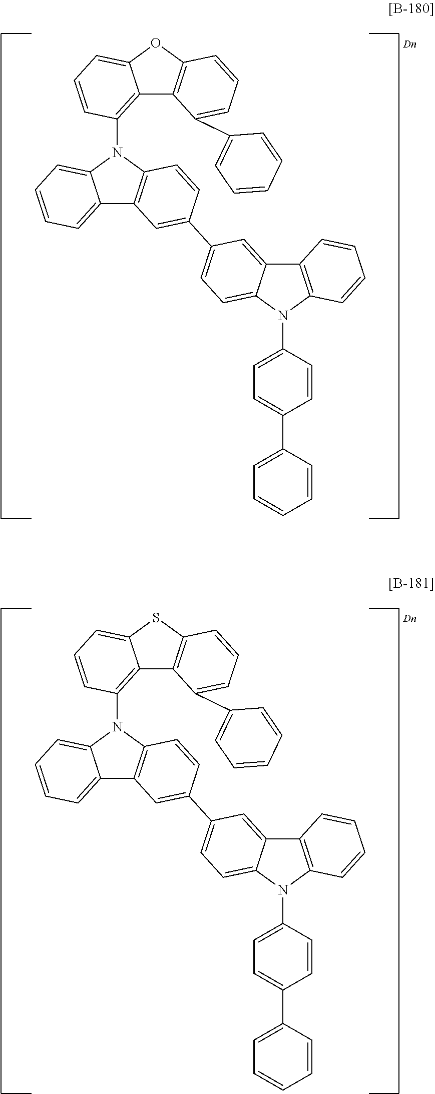

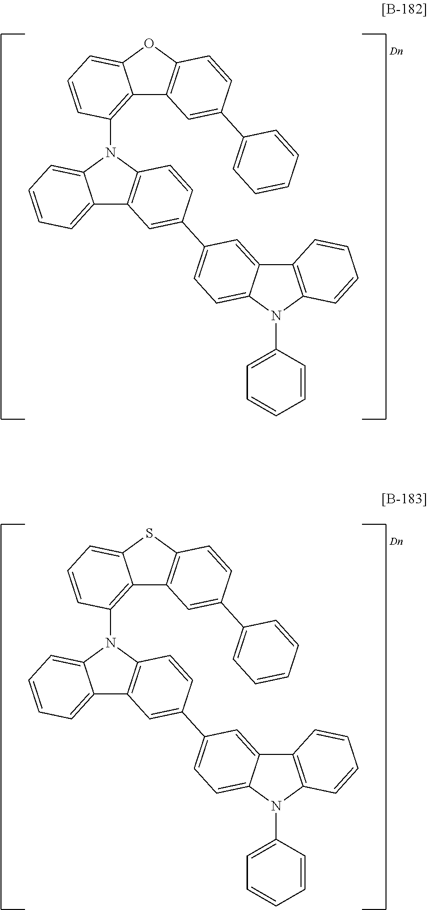

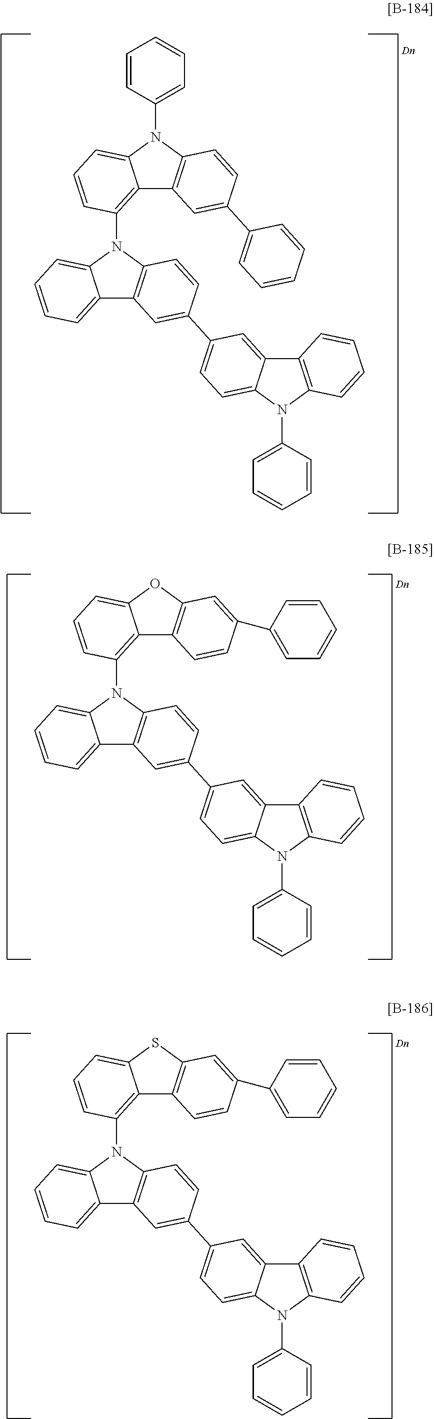

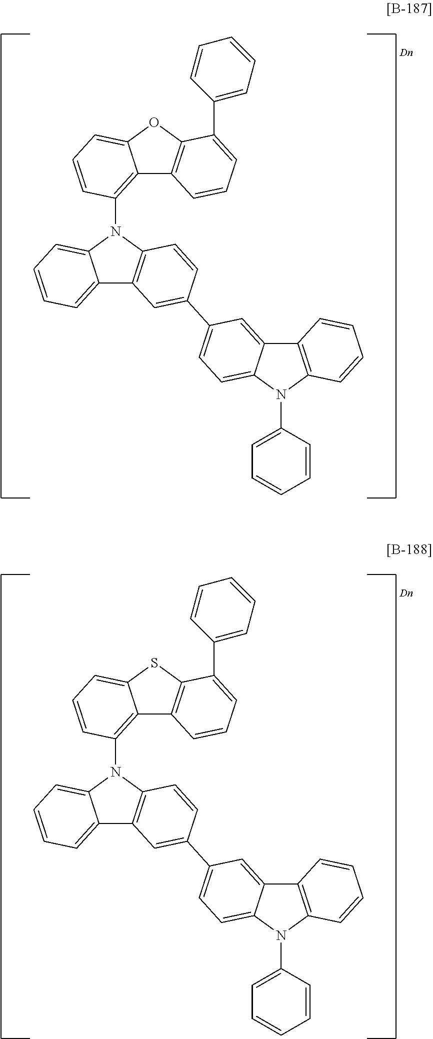

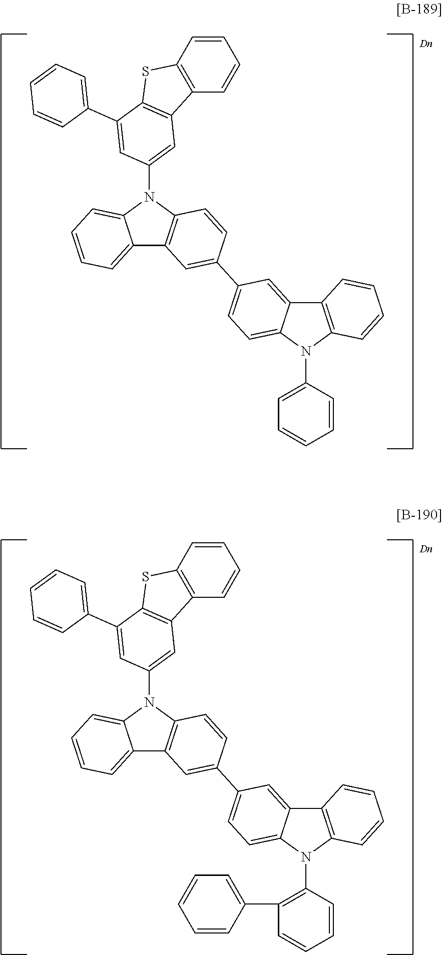

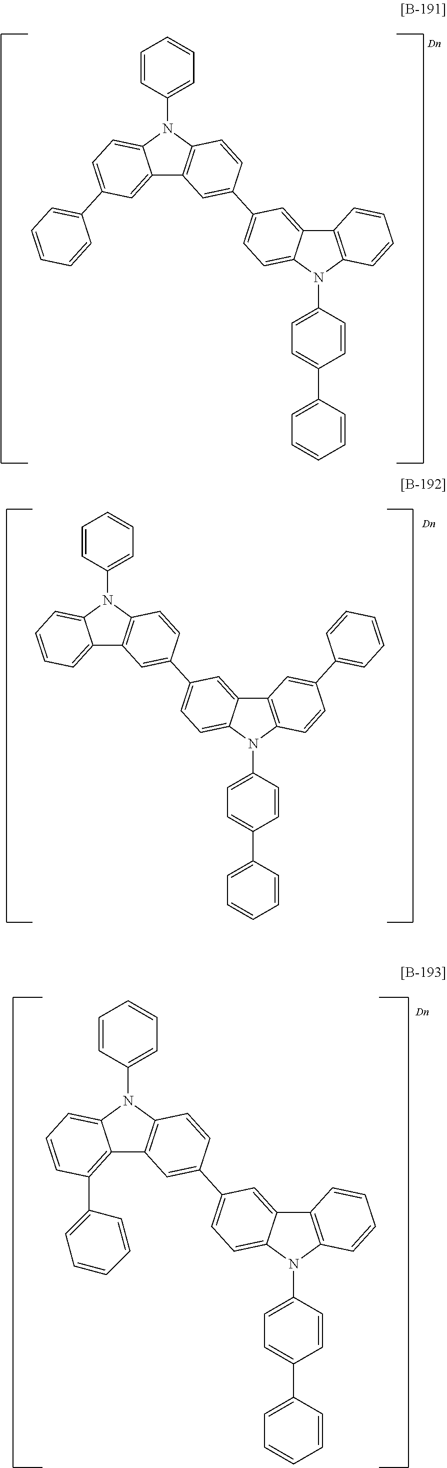

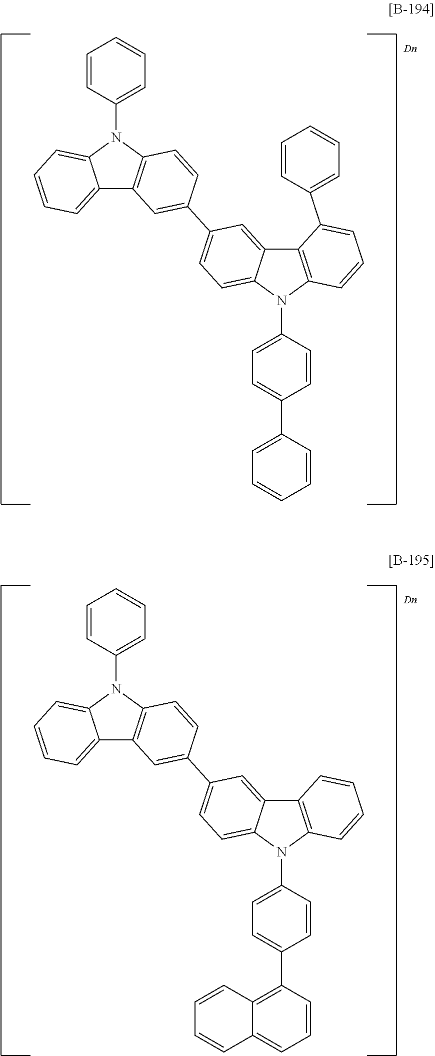

(Dn refers to the number of deuterium atoms substituted and indicates a structure in which one or more deuterium atoms are substituted)

The position of deuterium substitution and the substitution degree of deuterium substitution may include all changeable ranges within Compound B-1 to Compound B-195.

Specific structures of Compound B-151 to Compound B-195 are presented in Compound B-196 to B-234 below.

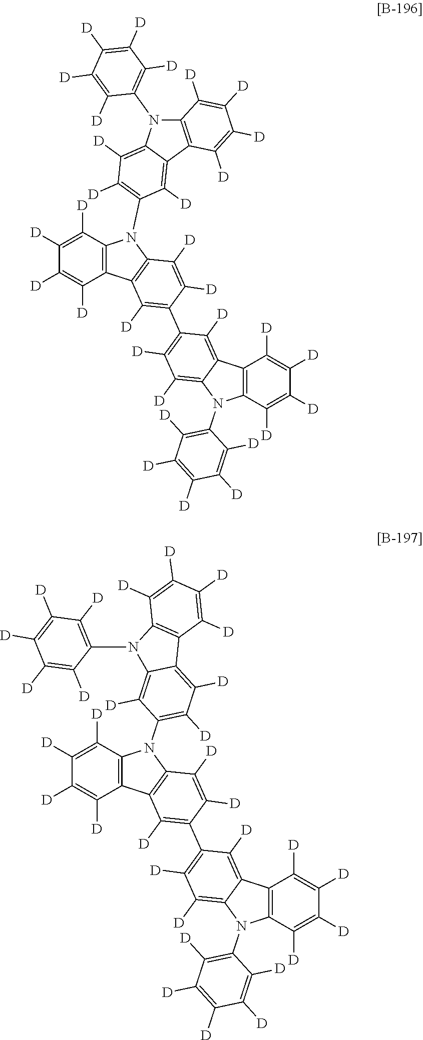

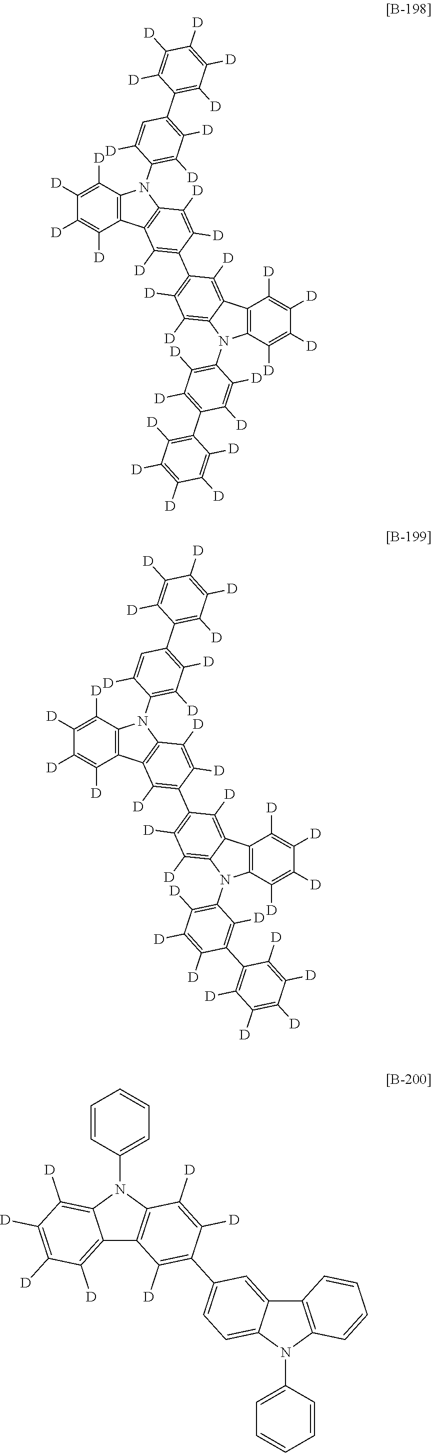

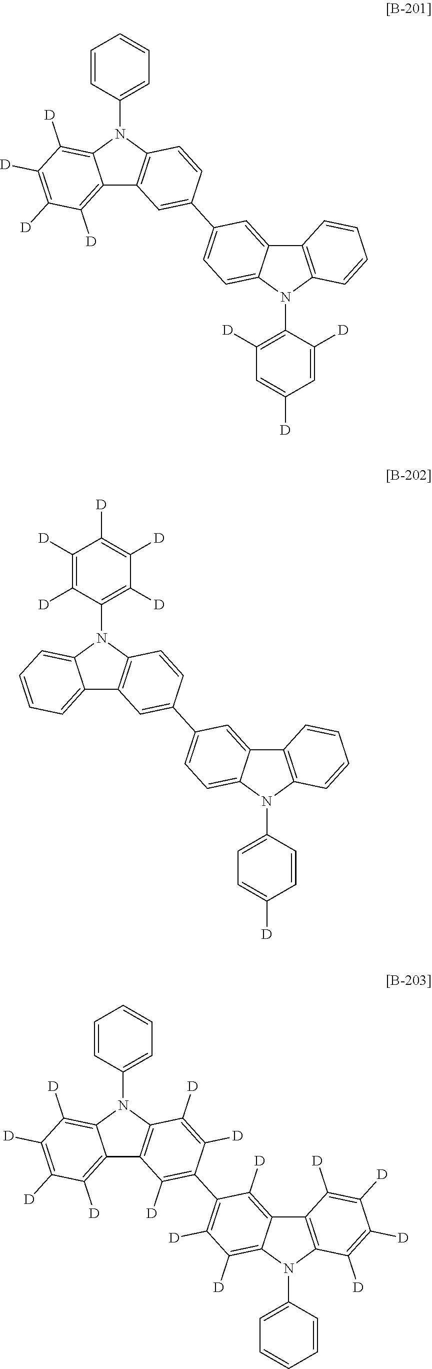 [SMILES: [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

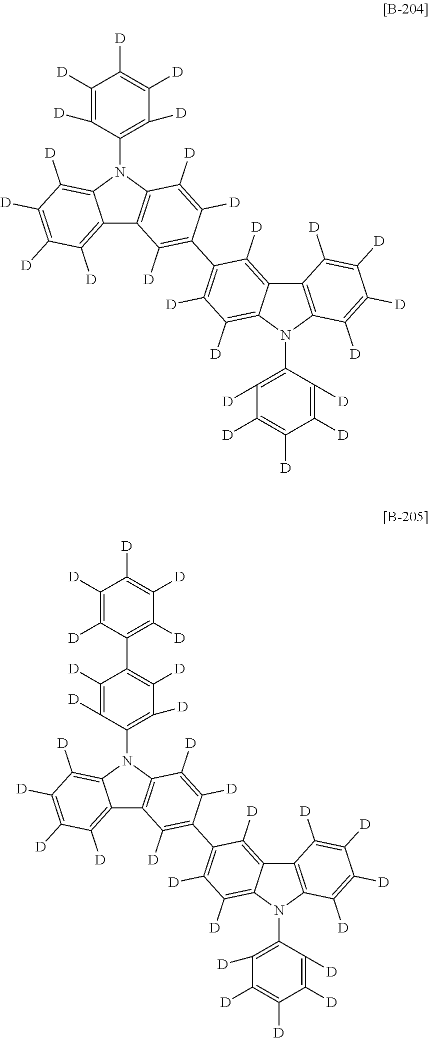 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

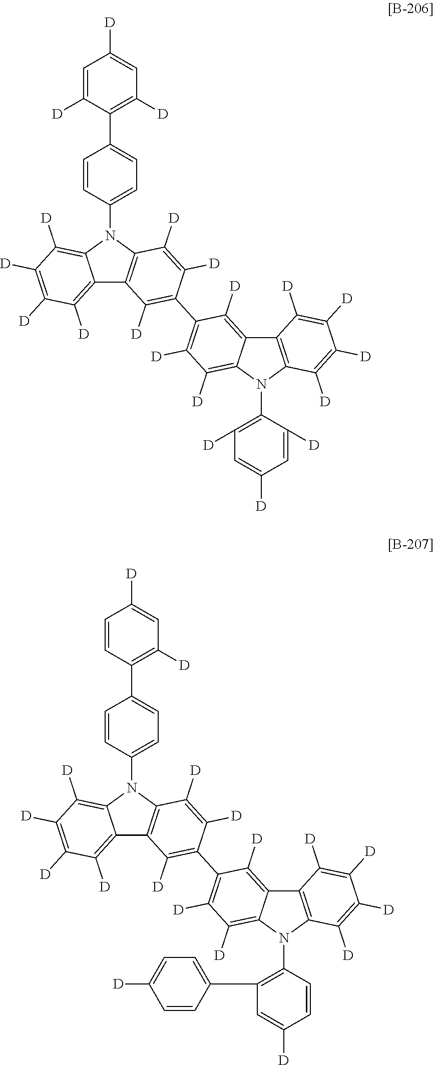 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1 ; [2H]c1ccc(-c2cc([2H])ccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

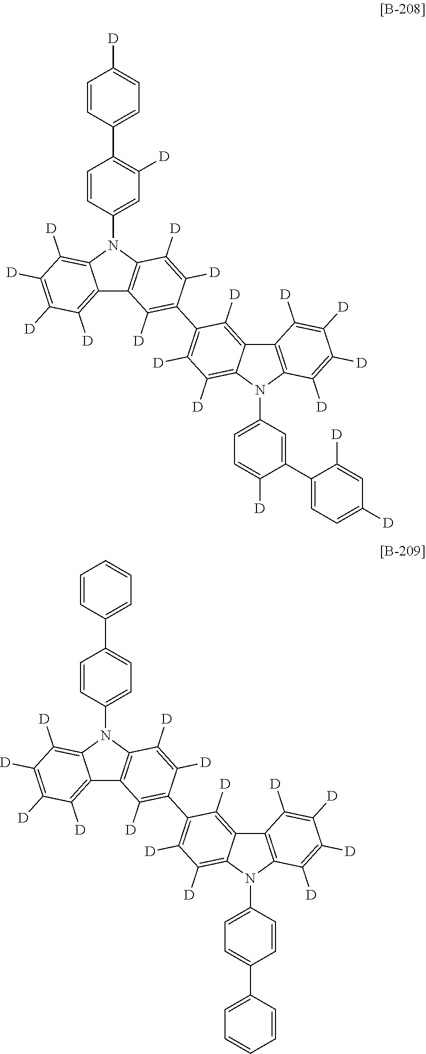 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

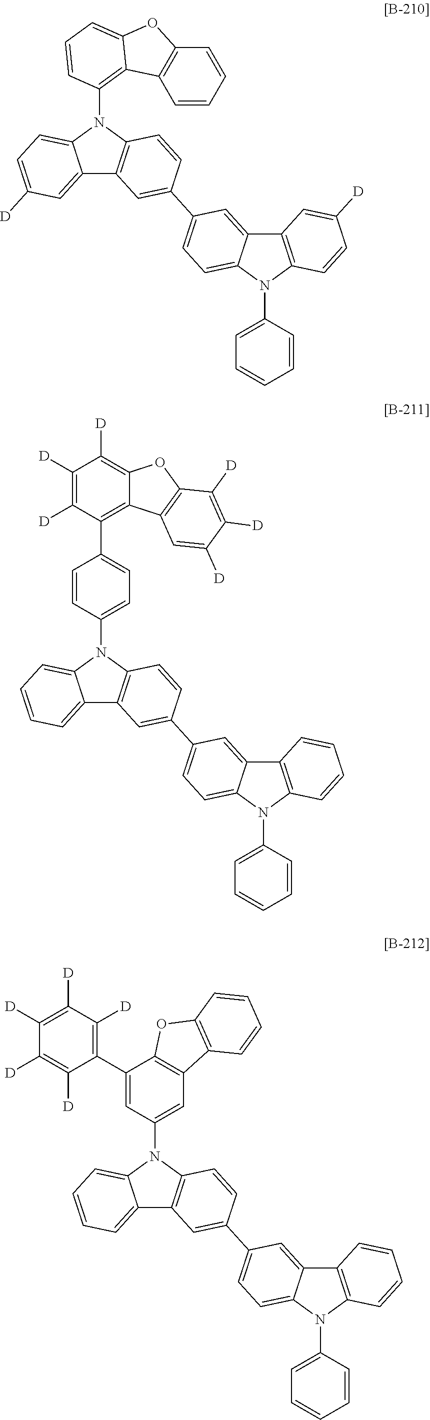 [SMILES: [2H]c1ccc2c(c1)c1cc(-c3ccc4c(c3)c3cc([2H])ccc3n4-c3cccc4oc5ccccc5c34)ccc1n2-c1ccccc1 ; [2H]c1cc2c(oc3c([2H])c([2H])c([2H])c(-c4ccc(-n5c6ccccc6c6cc(-c7ccc8c(c7)c7ccccc7n8-c7ccccc7)ccc65)cc4)c32)c([2H])c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

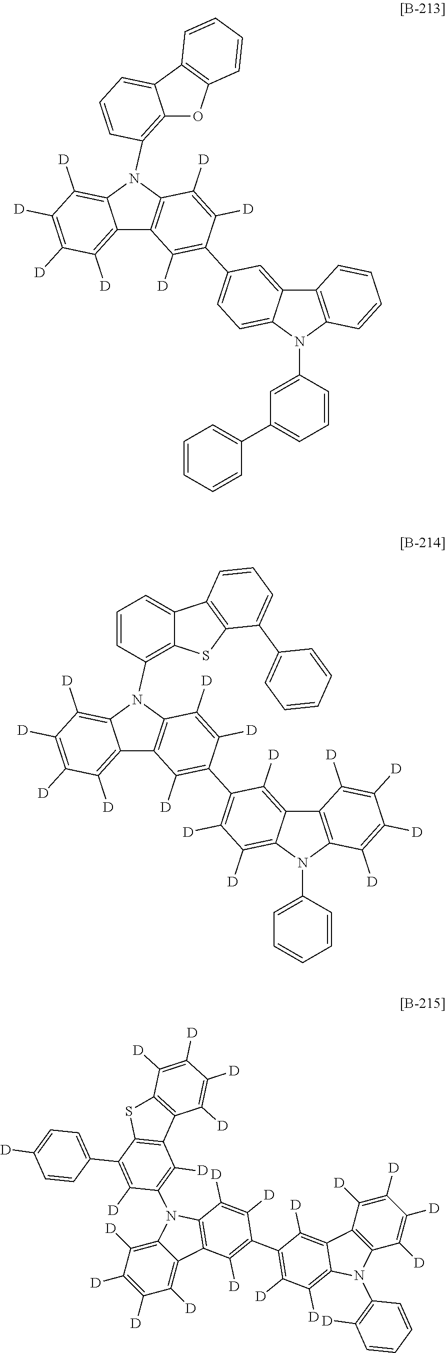

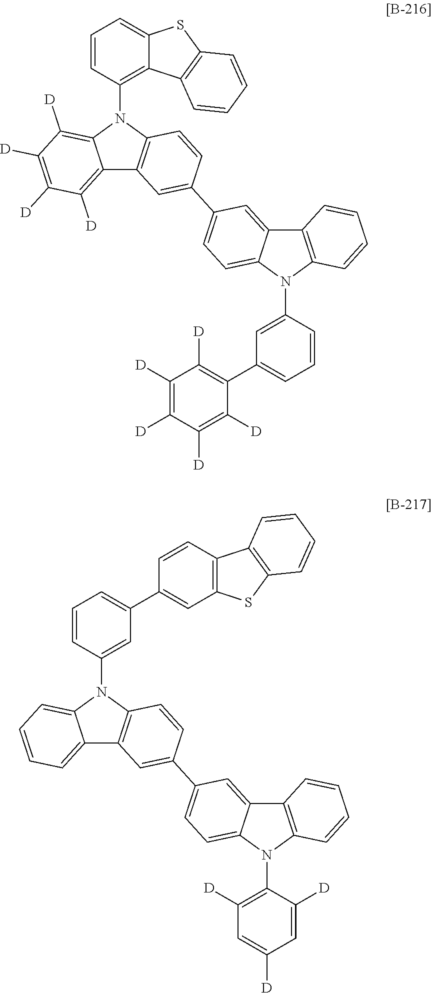 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

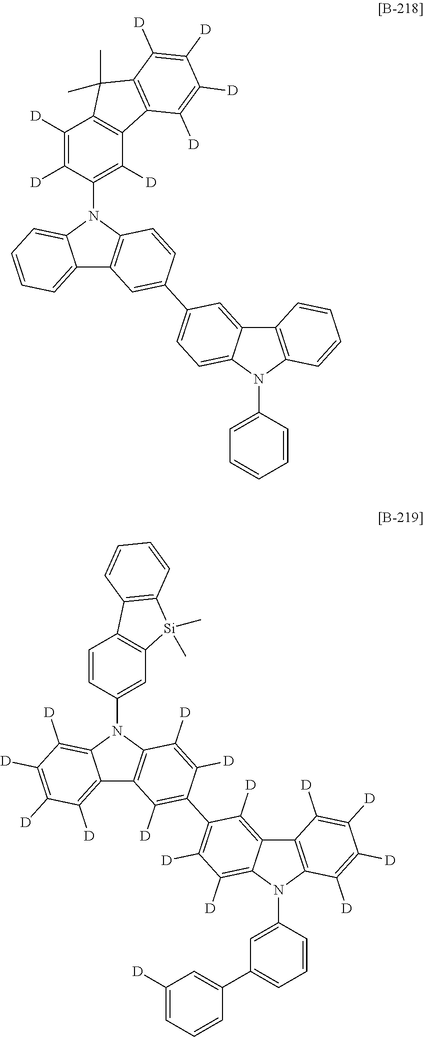 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

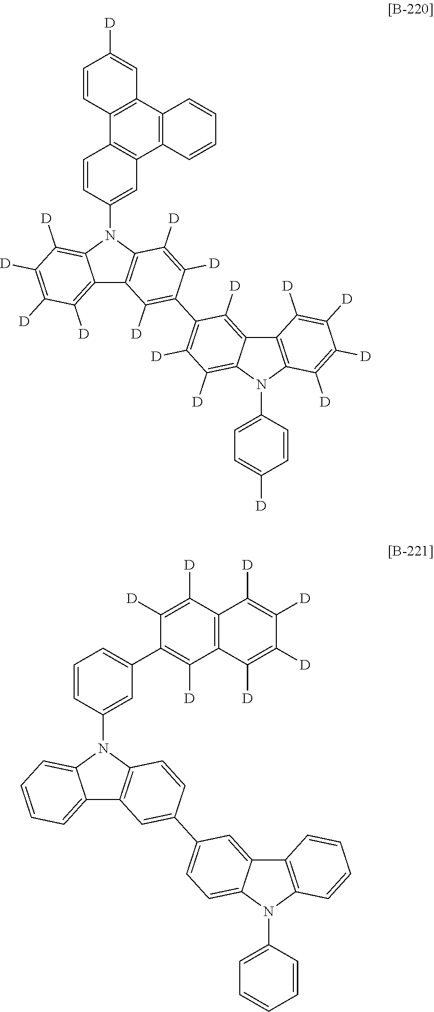 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H]]

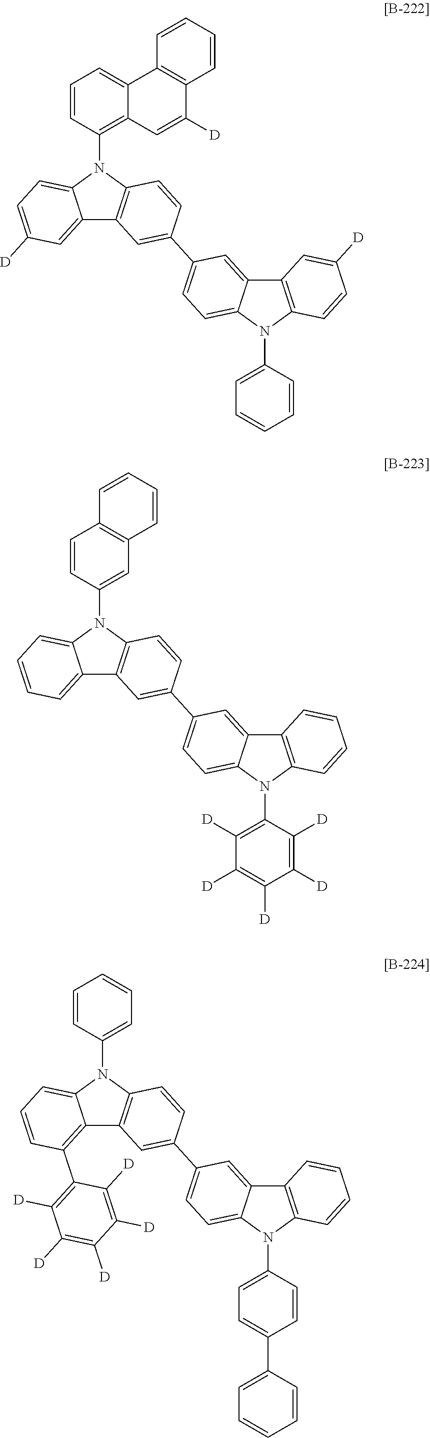 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H] ; [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H]]

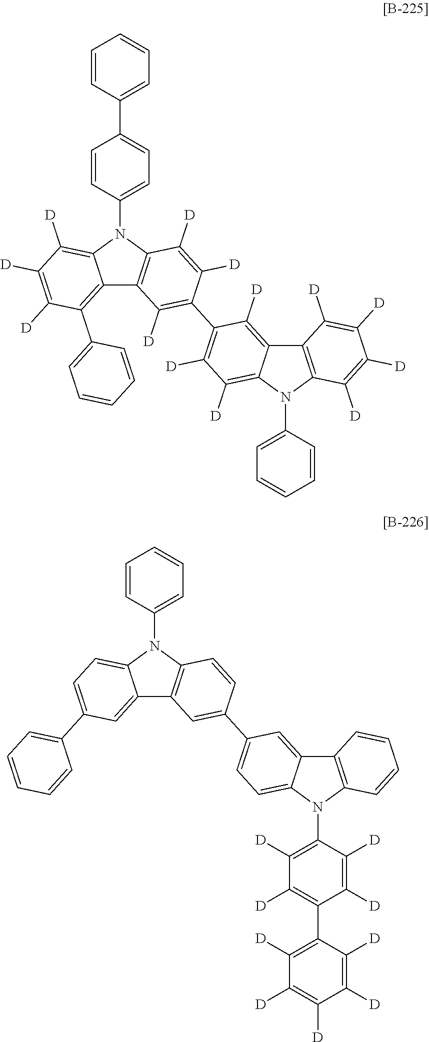 [SMILES: [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1 ; [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

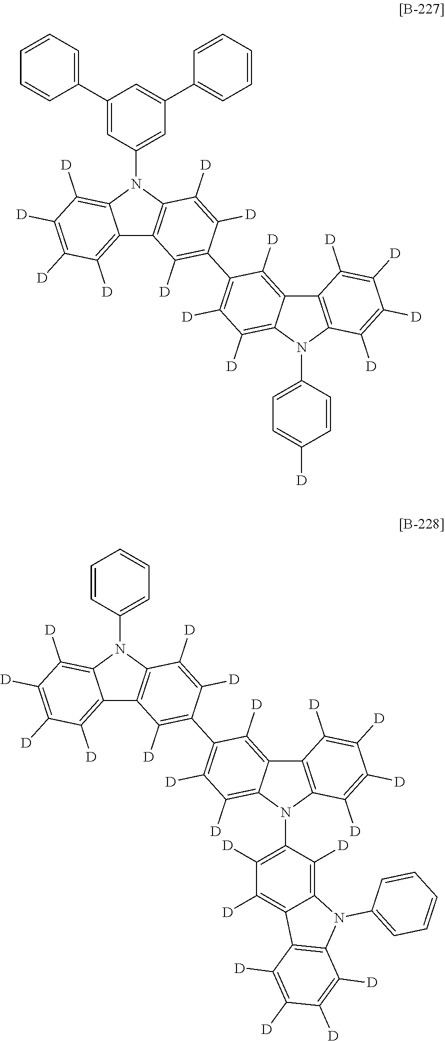 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

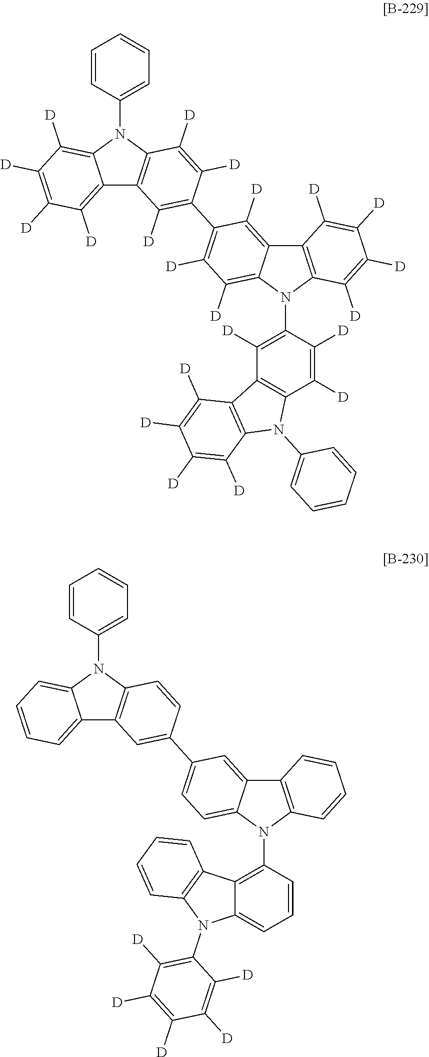 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H]]

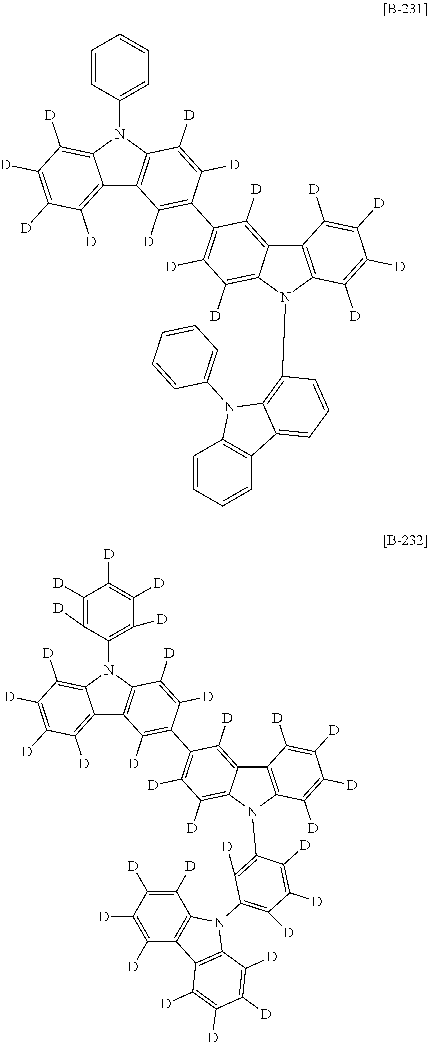 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H]]

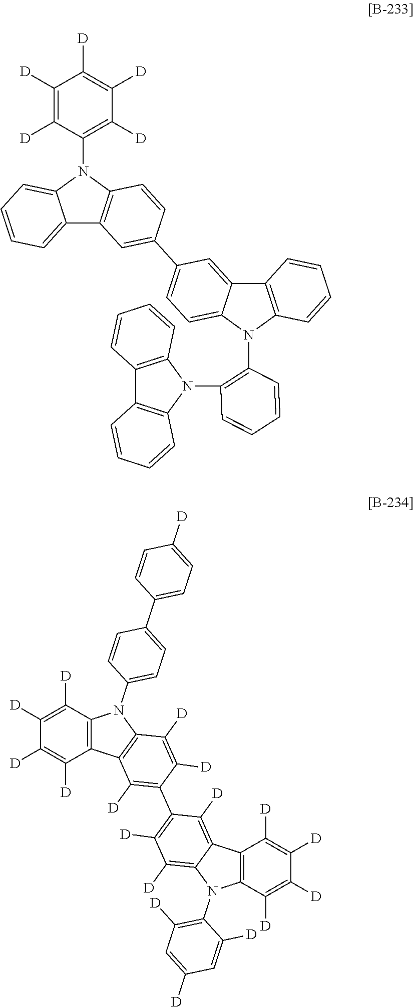 [SMILES: [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

In an implementation, the second compound for the organic optoelectronic device may be, e.g., a compound of Group 3.

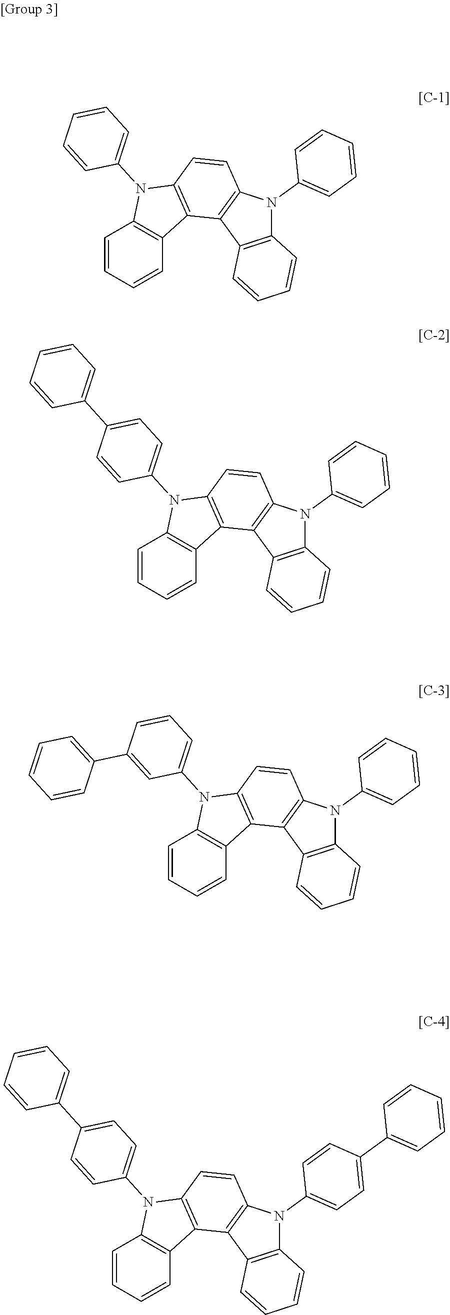 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

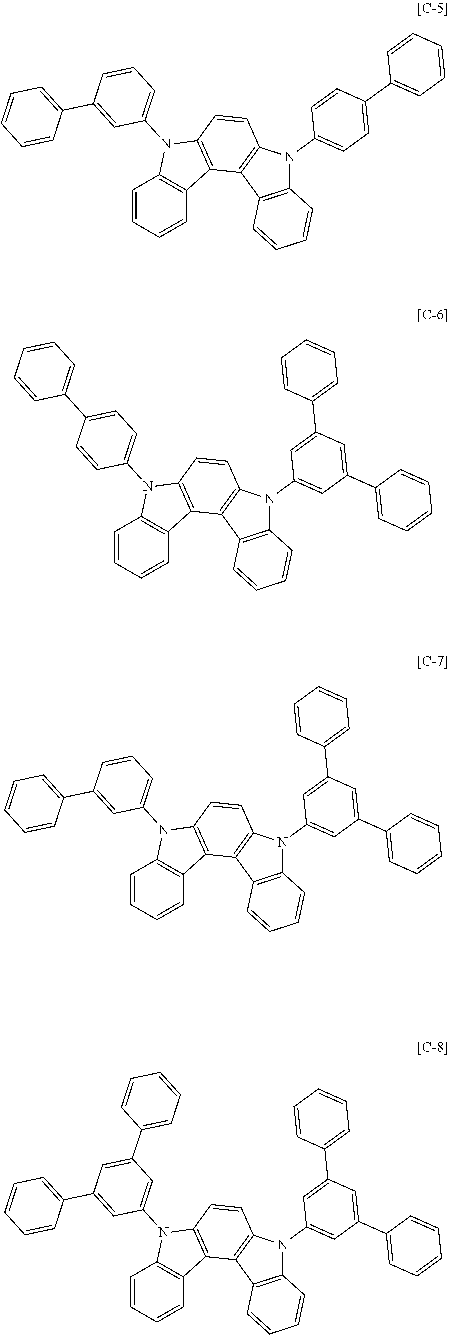 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

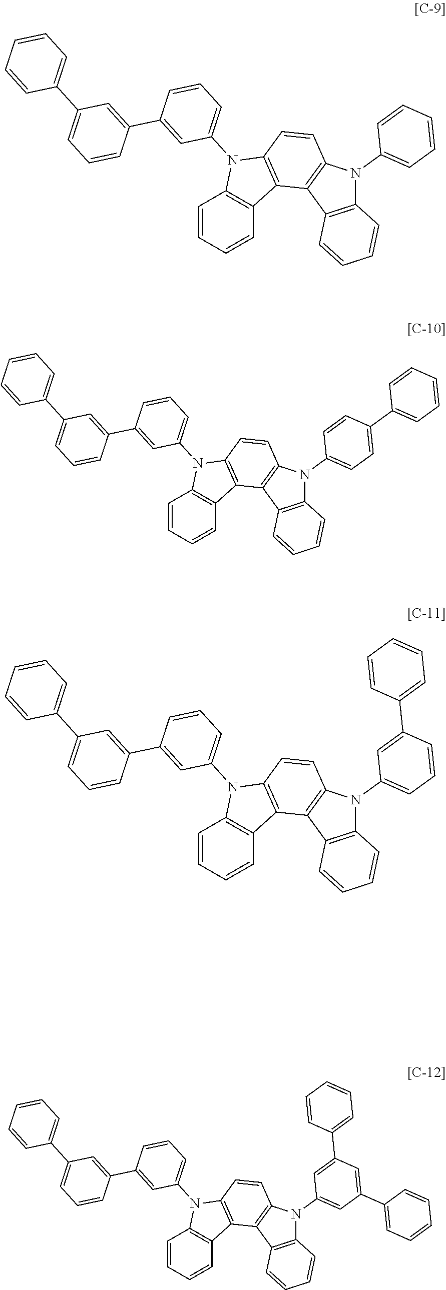 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

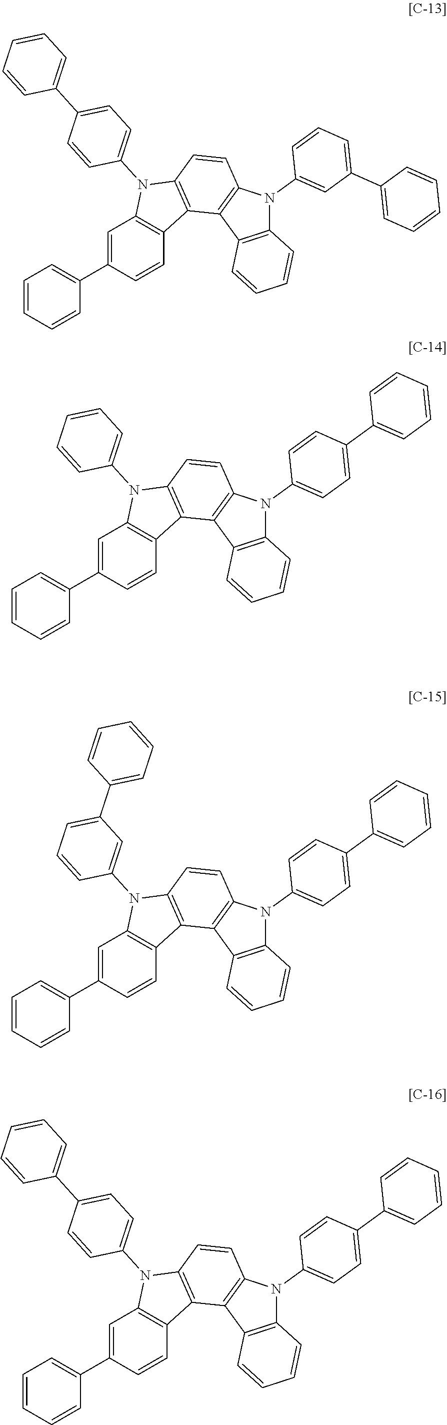 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

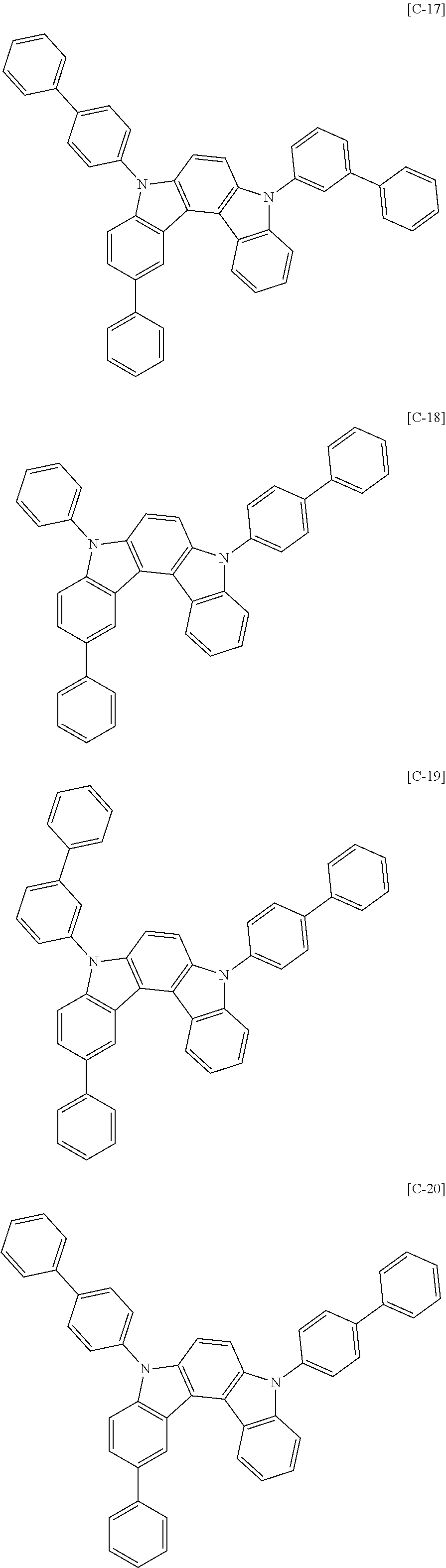

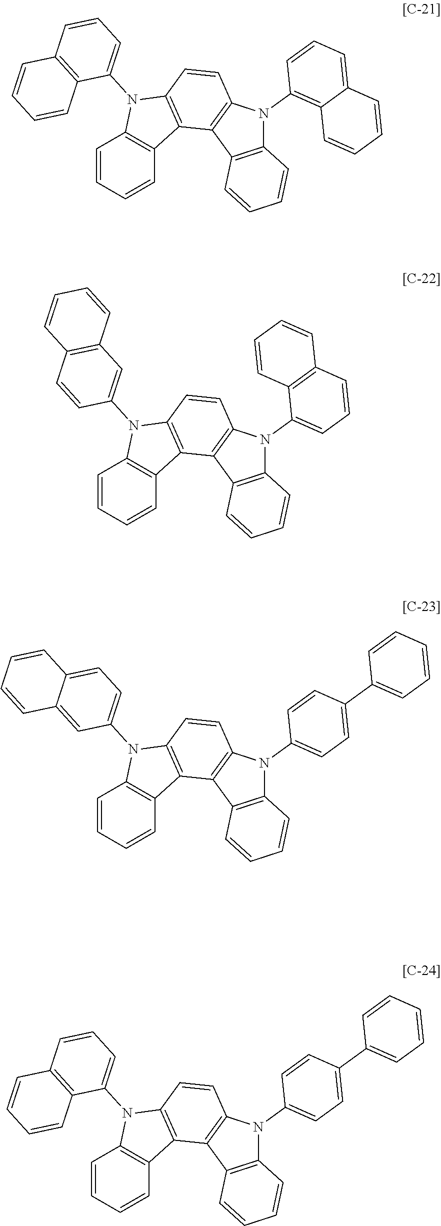 [SMILES: c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6cccc7ccccc67)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

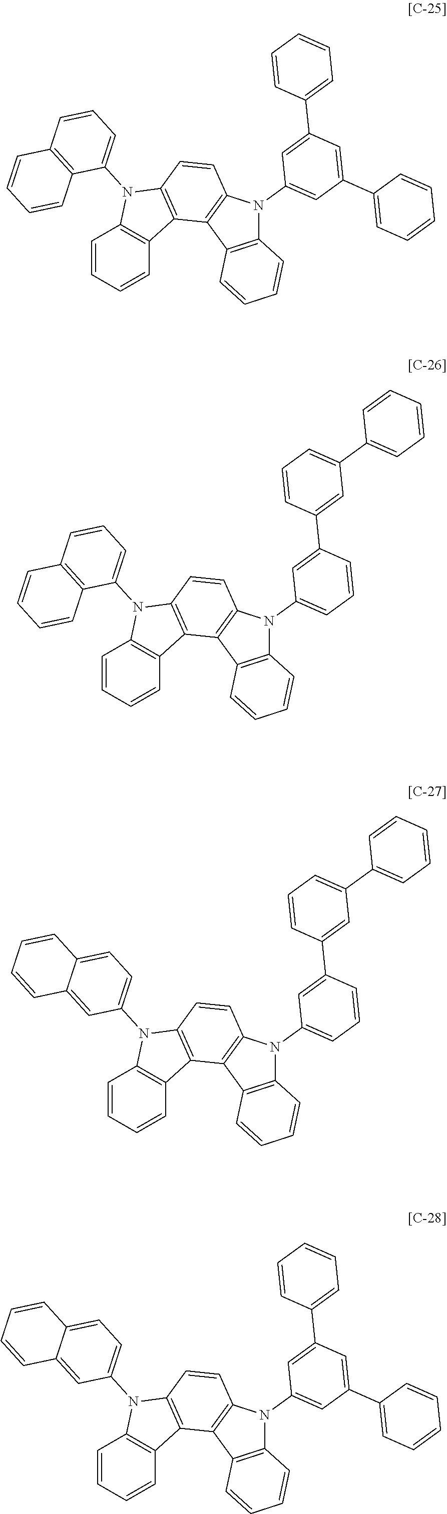 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1 ; c1ccc(-c2cc(-c3ccccc3)cc(-n3c4ccccc4c4c5c6ccccc6n(-c6ccc7ccccc7c6)c5ccc43)c2)cc1]

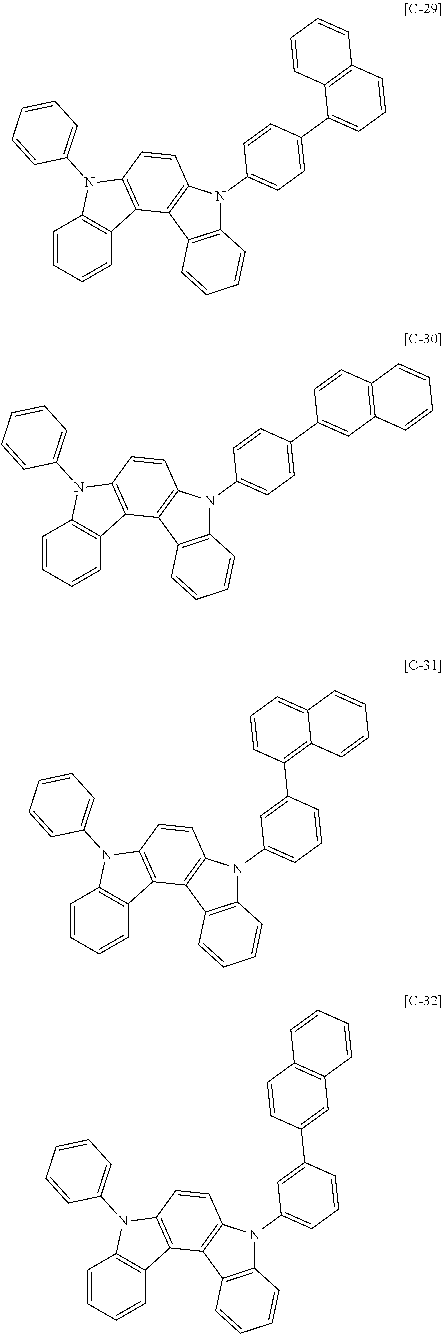 [SMILES: c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1]

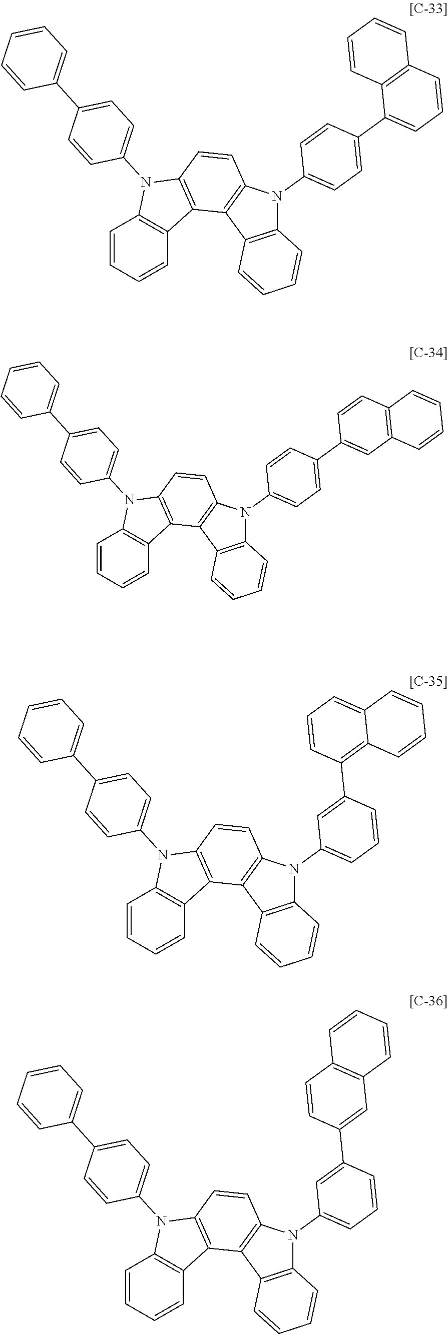 [SMILES: c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

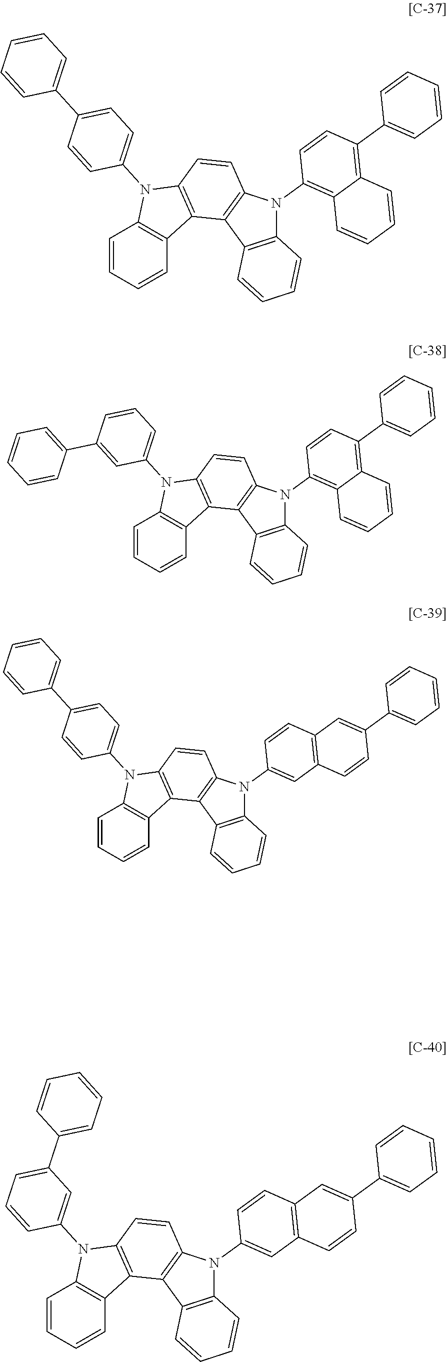 [SMILES: c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1]

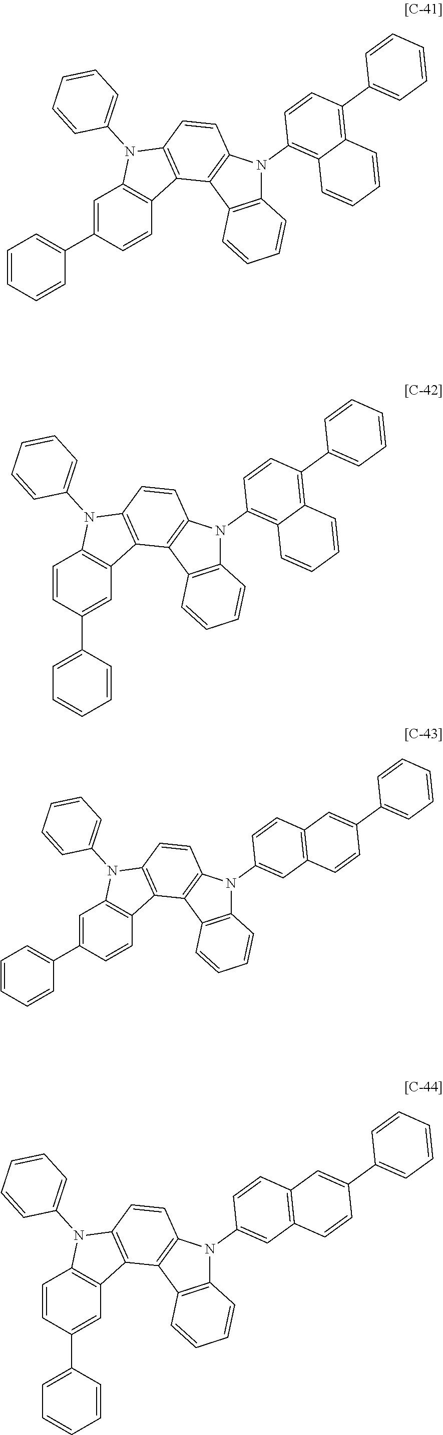 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

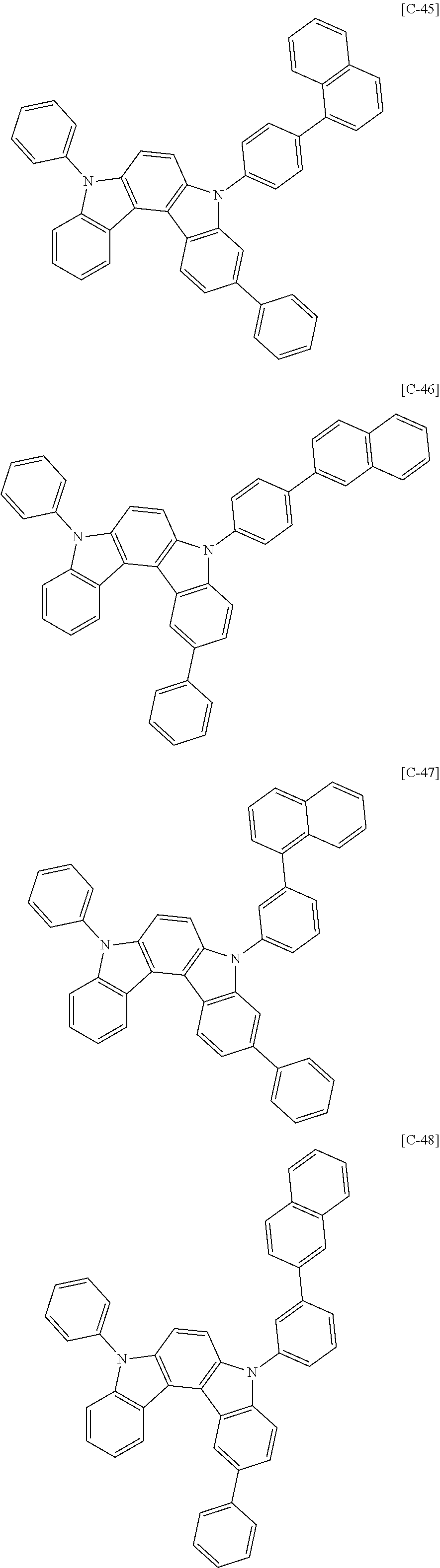

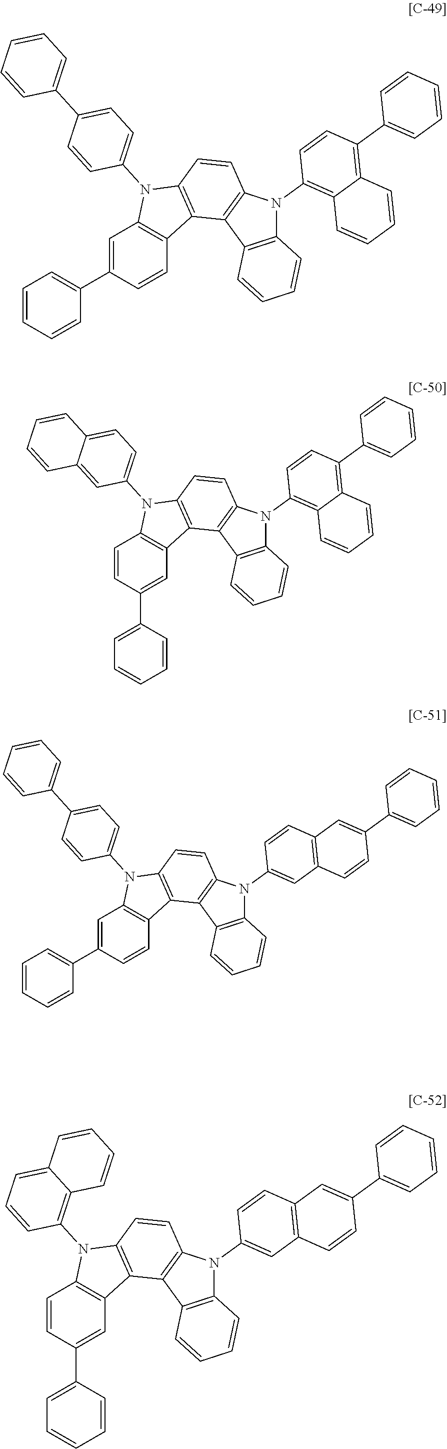 [SMILES: c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1]

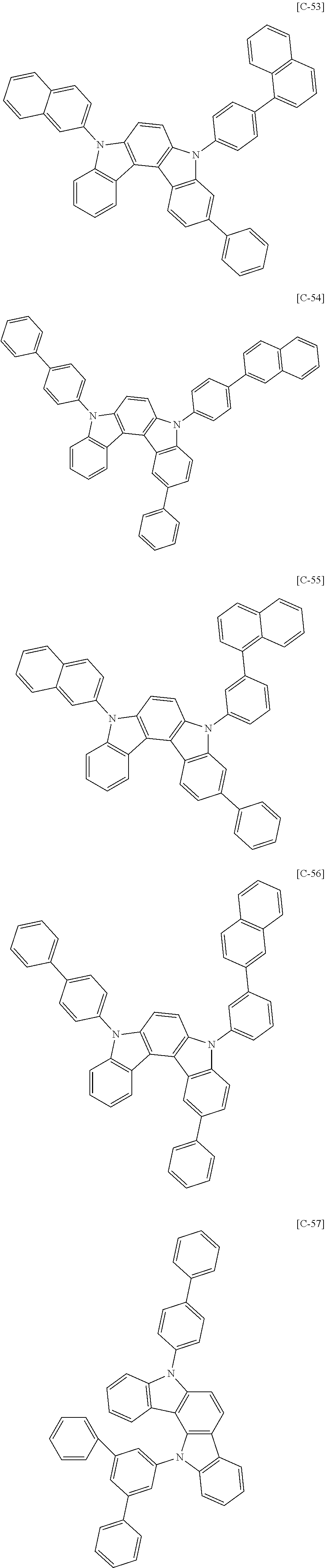

Examples of Compound C-1 to Compound C-57 in which at least one hydrogen is replaced with deuterium are shown in Compound C-58 to Compound C-72, exemplified below.

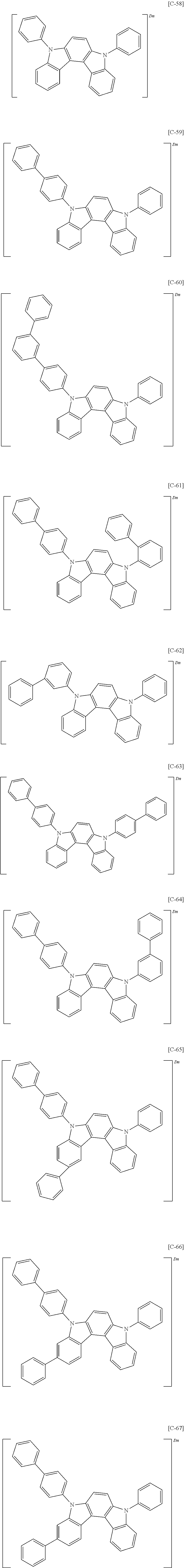

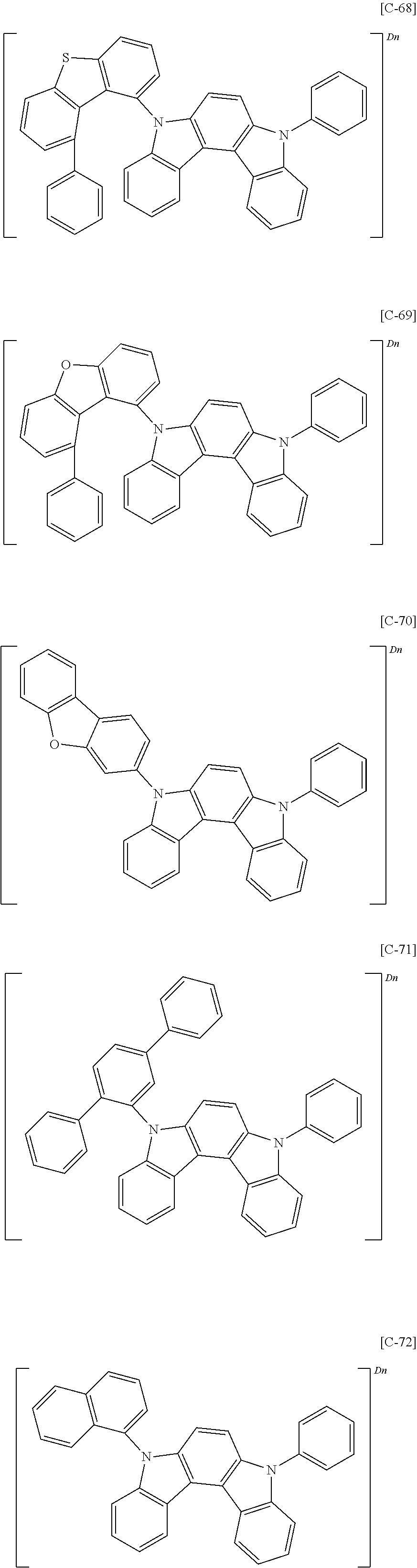

(Dn refers to the number of deuterium atoms substituted and indicates a structure in which one or more deuterium atoms are substituted)

The position of deuterium substitution and the substitution degree of deuterium substitution may include all changeable ranges within the range of Compound C-1 to Compound C-72.

Specific structures of Compounds C-58 to C-72 are presented in Compound C-73 to Compound C-102 below.

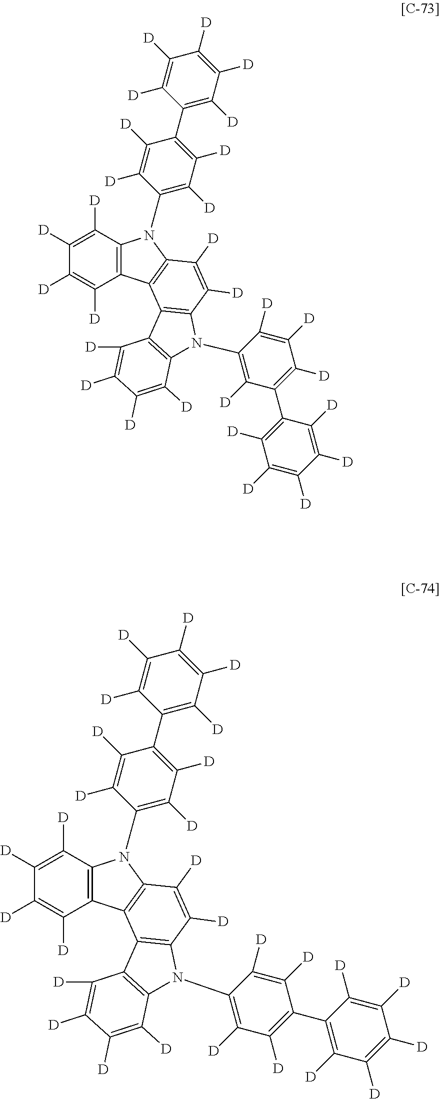 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

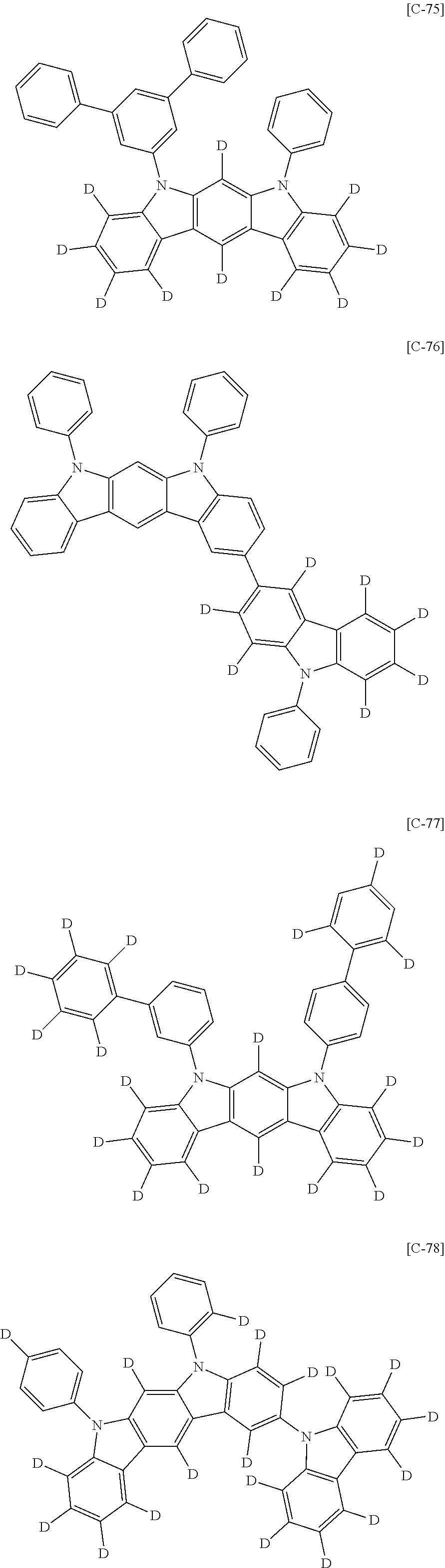

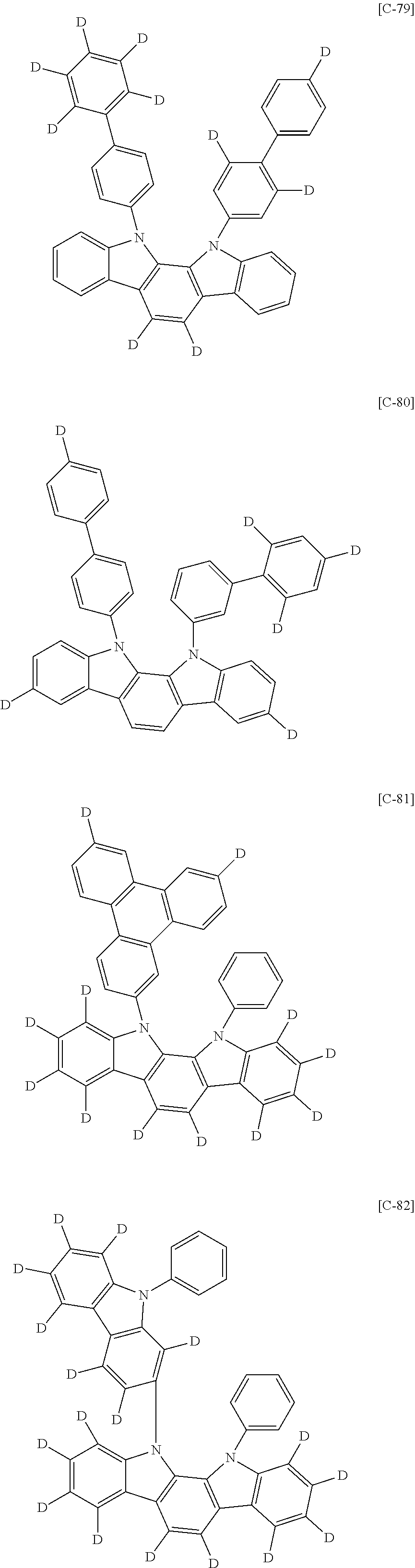

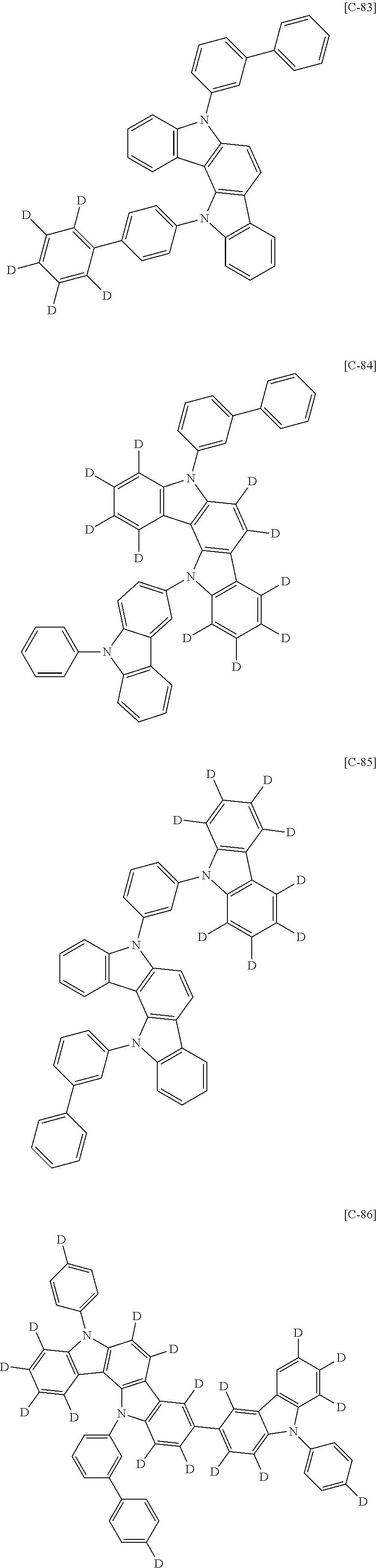

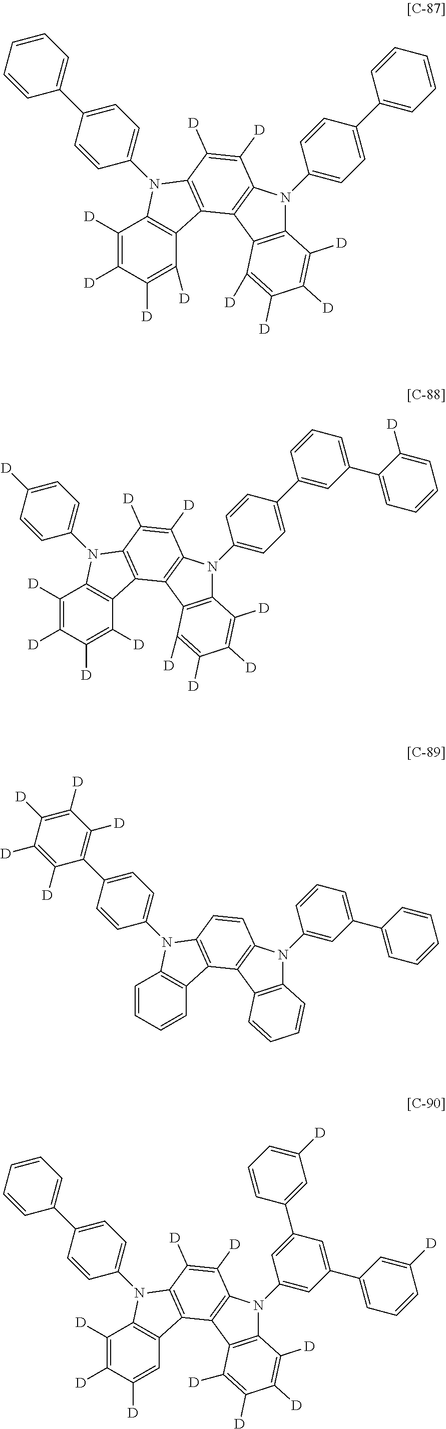

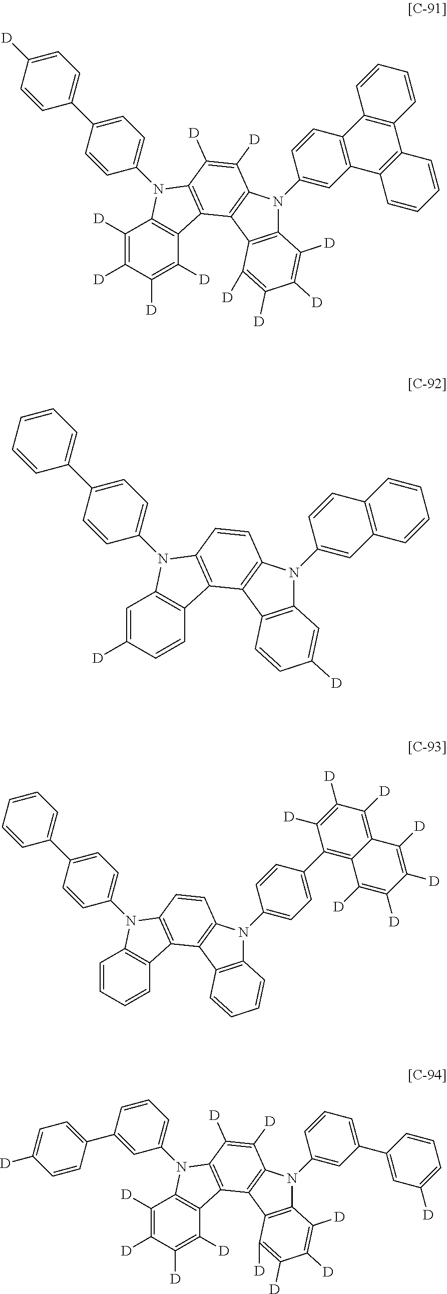 [SMILES: [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1 ; [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H] ; [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1]

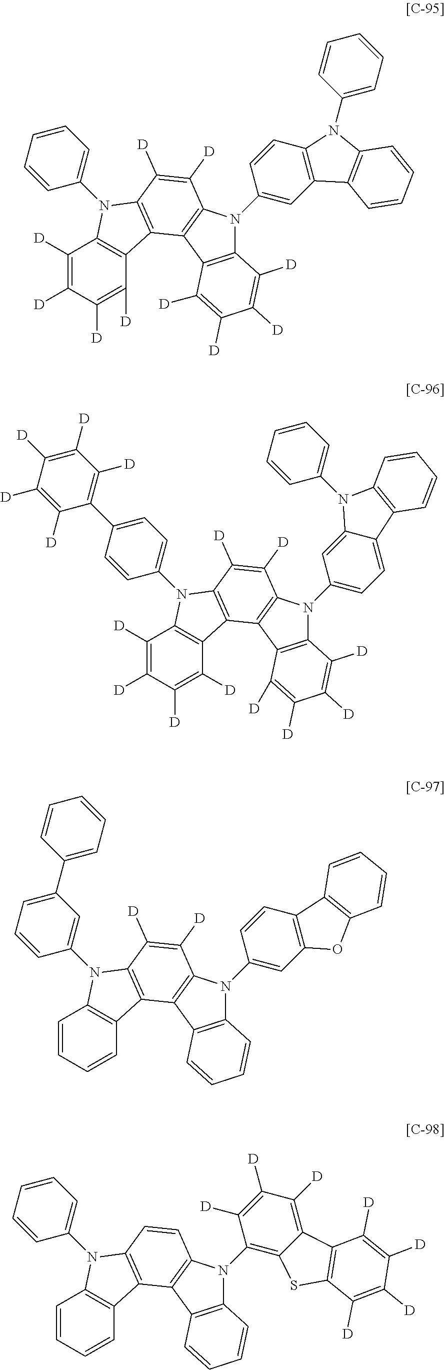 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1 ; [2H]c1c([2H])c([2H])c(-c2ccc(-n3c4c([2H])c([2H])c([2H])c([2H])c4c4c5c6c([2H])c([2H])c([2H])c([2H])c6n(-c6ccc7c8ccccc8n(-c8ccccc8)c7c6)c5c([2H])c([2H])c43)cc2)c([2H])c1[2H] ; [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

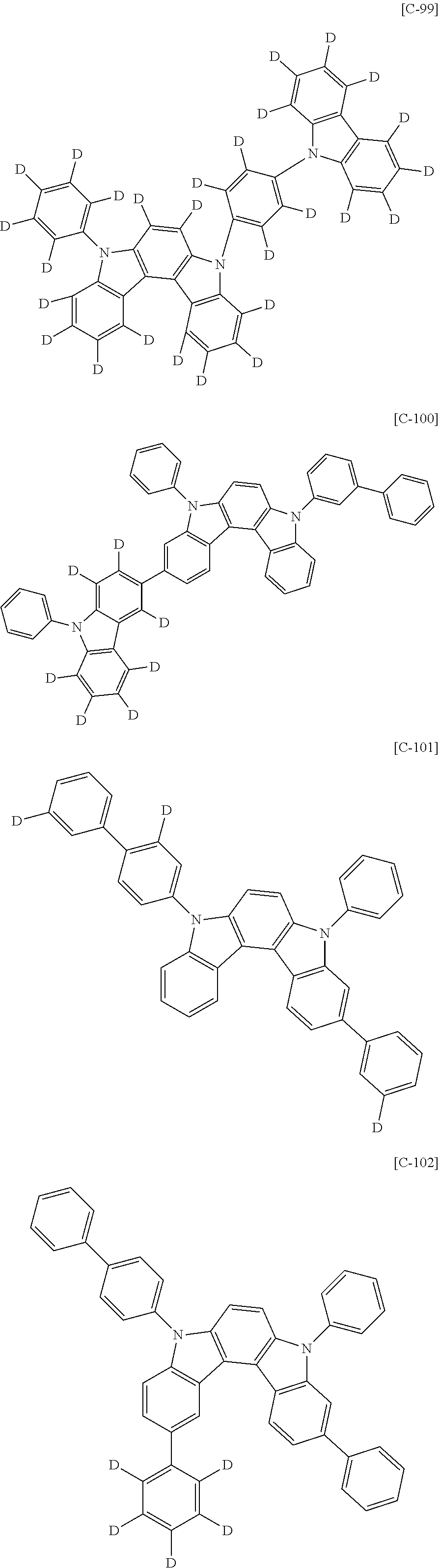

In an implementation, the second compound for the organic optoelectronic device may be, e.g., a compound of Group 4.

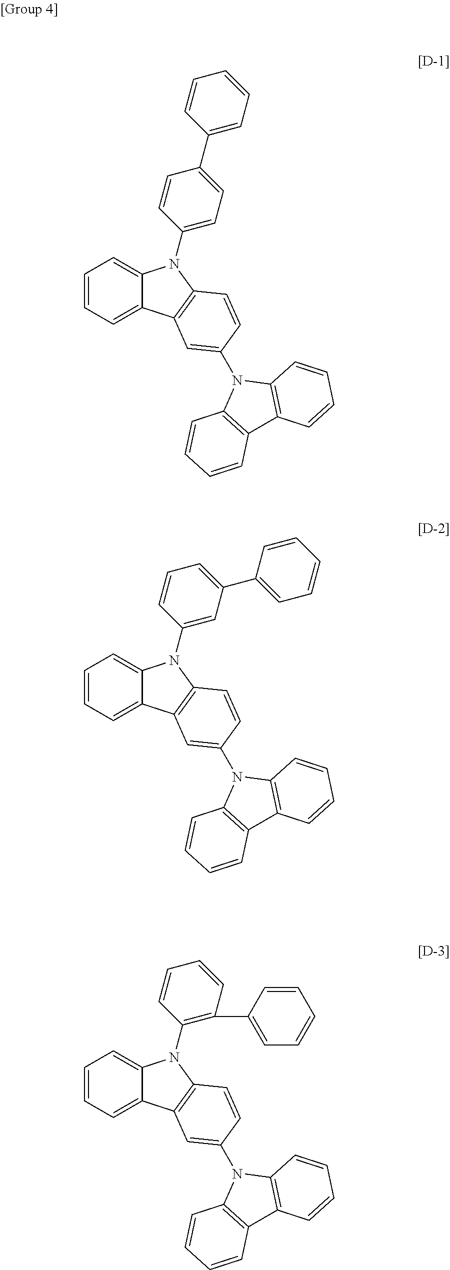 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1]

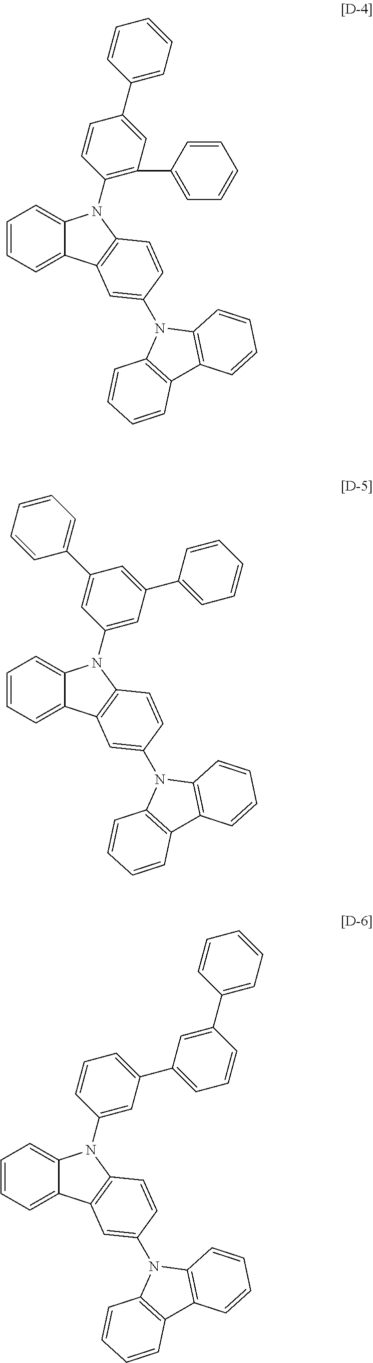 [SMILES: c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1 ; c1ccc(-c2cccc(-c3cccc(-n4c5ccccc5c5cc(-n6c7ccccc7c7ccccc76)ccc54)c3)c2)cc1]

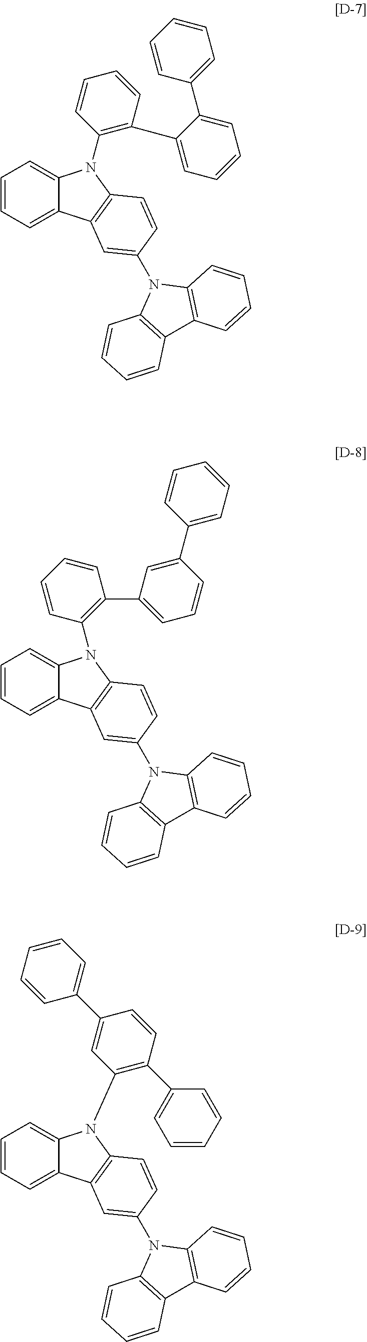 [SMILES: c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1]

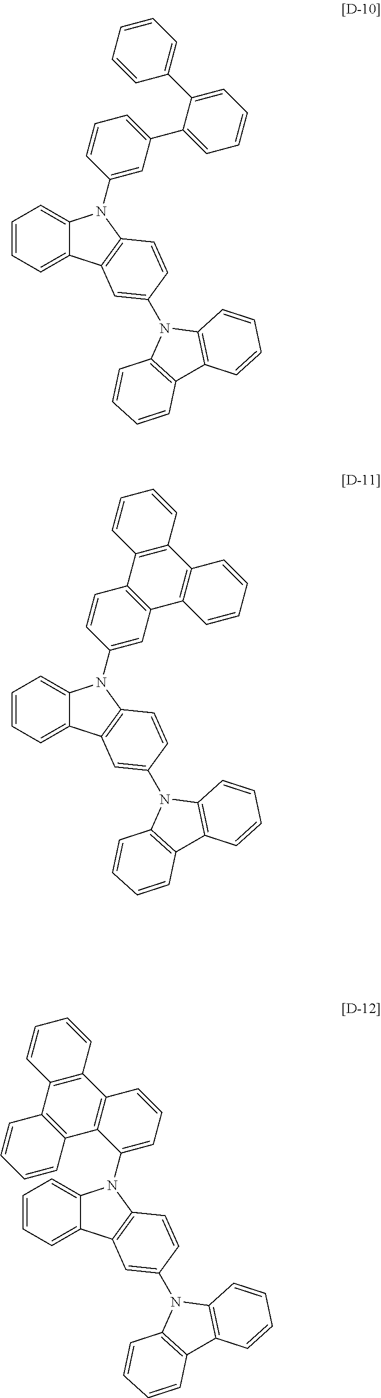 [SMILES: c1ccc(-c2ccccc2-c2cccc(-n3c4ccccc4c4cc(-n5c6ccccc6c6ccccc65)ccc43)c2)cc1 ; c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21]

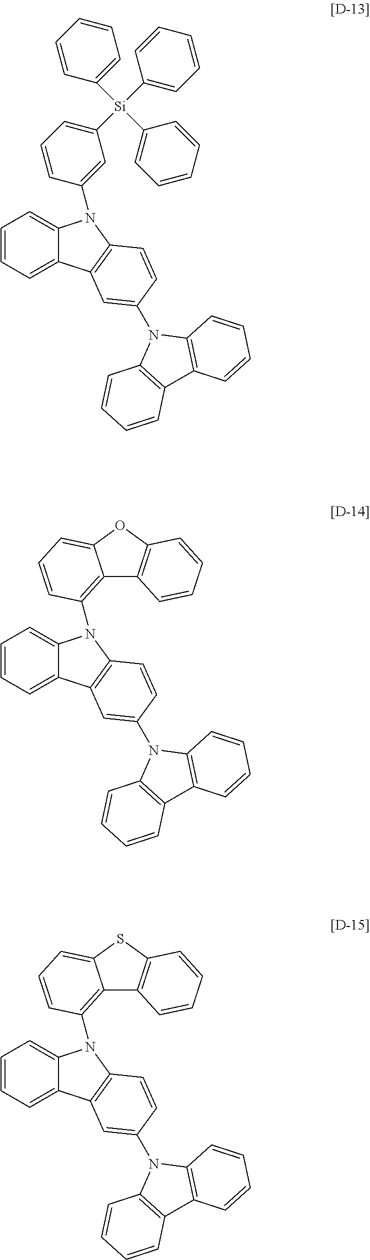 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

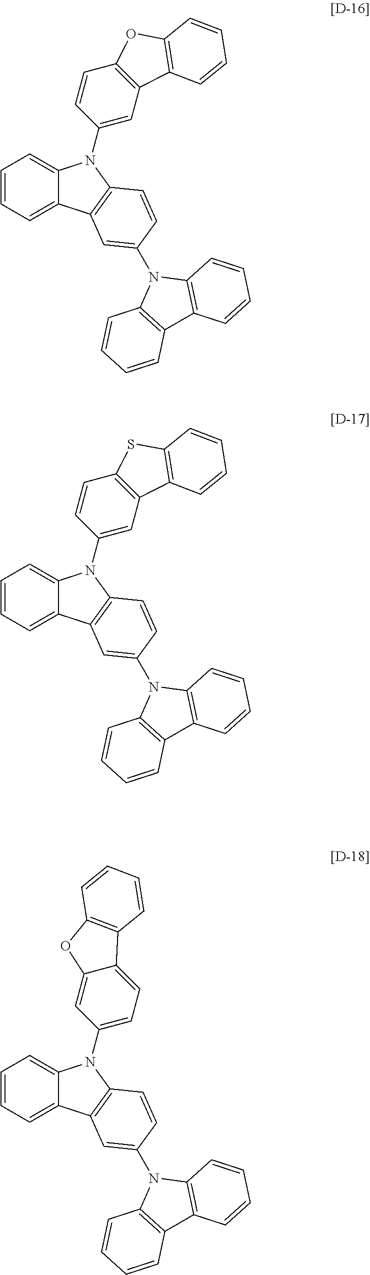 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12]

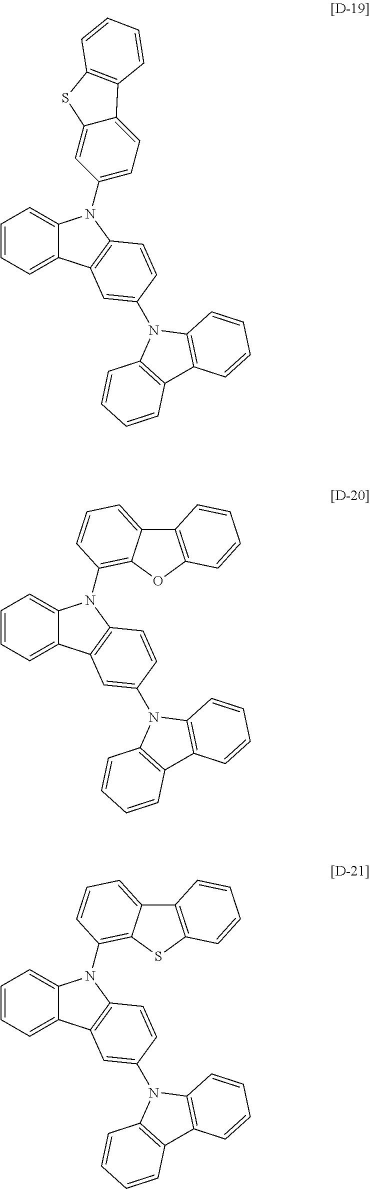 [SMILES: c1ccc2c(c1)sc1cc(-n3c4ccccc4c4cc(-n5c6ccccc6c6ccccc65)ccc43)ccc12 ; c1ccc2c(c1)oc1c(-n3c4ccccc4c4cc(-n5c6ccccc6c6ccccc65)ccc43)cccc12 ; c1ccc2c(c1)sc1c(-n3c4ccccc4c4cc(-n5c6ccccc6c6ccccc65)ccc43)cccc12]

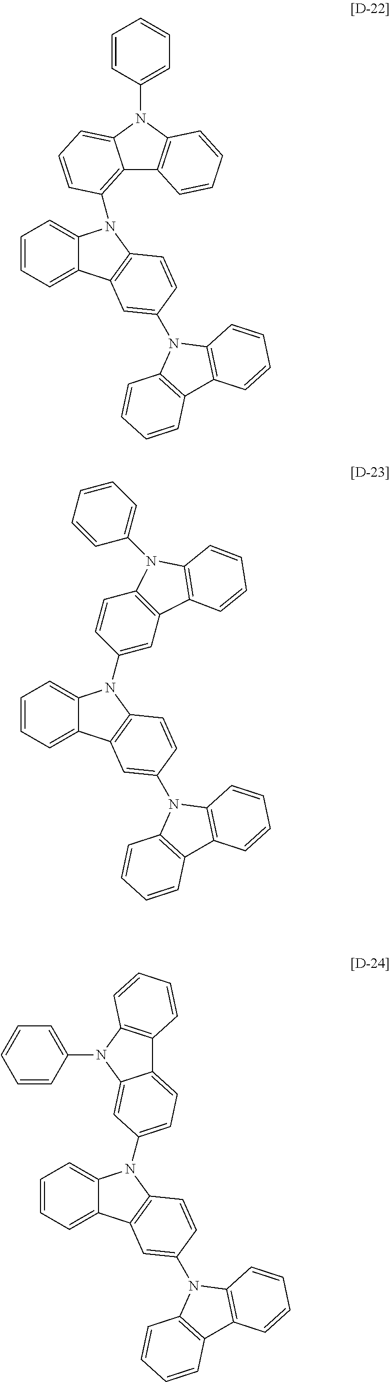 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1]

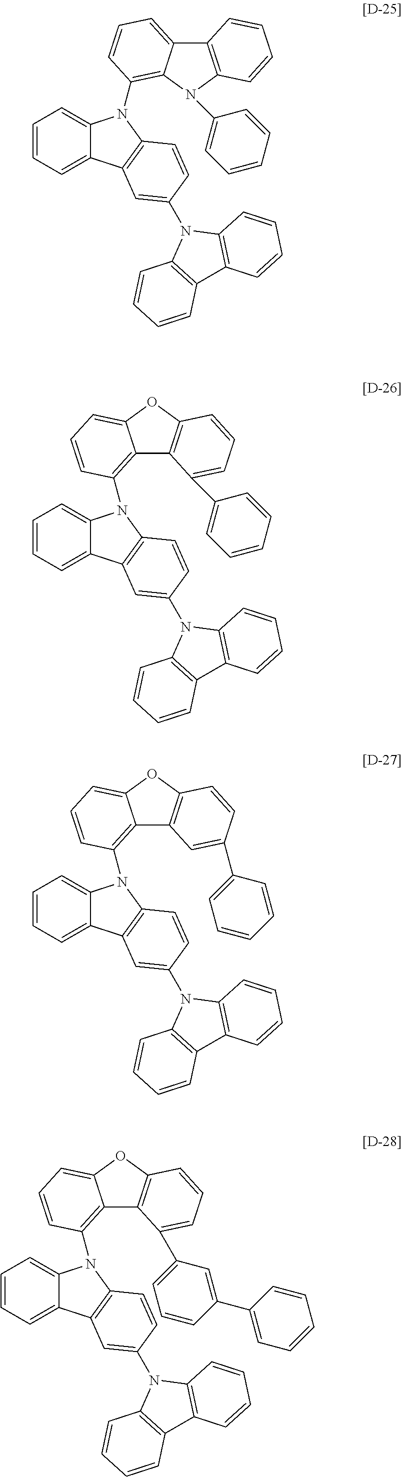 [SMILES: c1ccc(-n2c3ccccc3c3cccc(-n4c5ccccc5c5cc(-n6c7ccccc7c7ccccc76)ccc54)c32)cc1 ; c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1 ; c1ccc(-c2cccc(-c3cccc4oc5cccc(-n6c7ccccc7c7cc(-n8c9ccccc9c9ccccc98)ccc76)c5c34)c2)cc1]

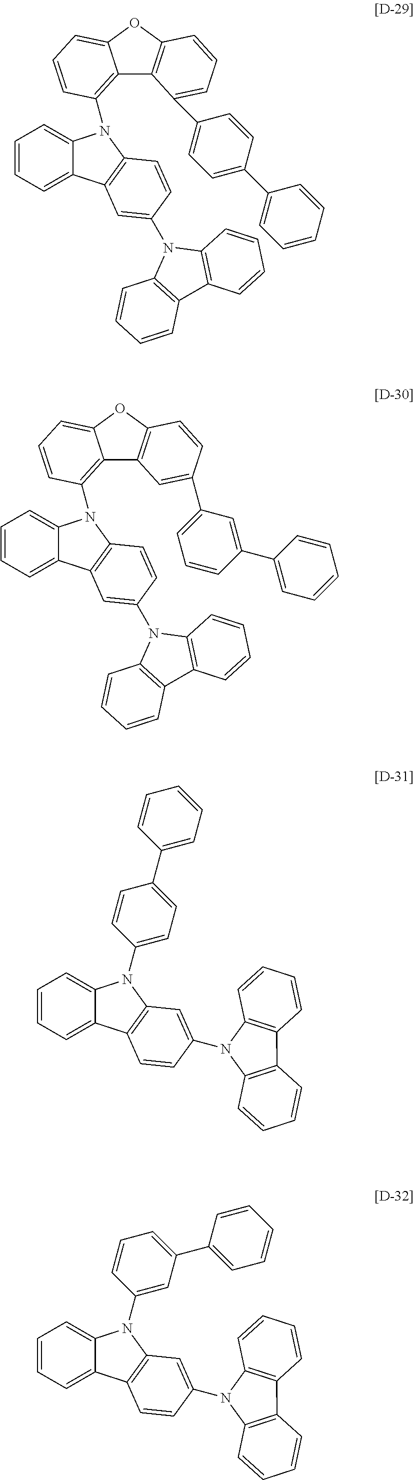 [SMILES: c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1 ; c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1]

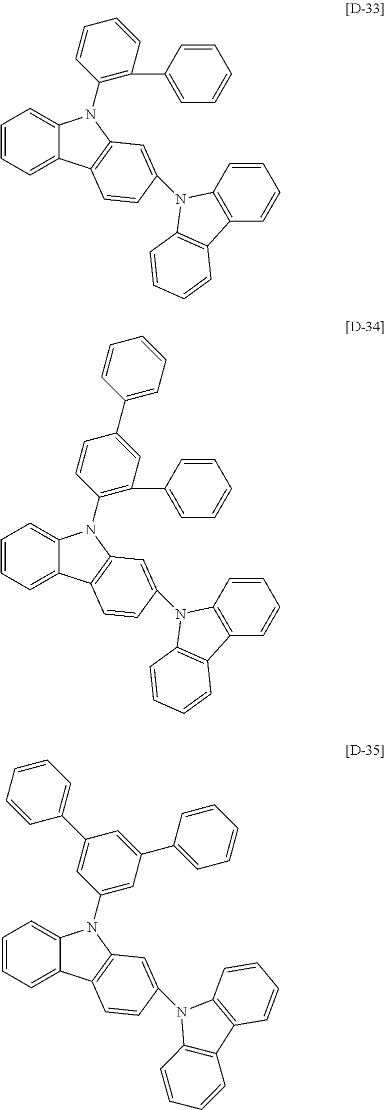 [SMILES: c1ccc(-c2ccccc2-n2c3ccccc3c3ccc(-n4c5ccccc5c5ccccc54)cc32)cc1 ; c1ccc(-c2ccc(-n3c4ccccc4c4ccc(-n5c6ccccc6c6ccccc65)cc43)c(-c3ccccc3)c2)cc1 ; c1ccc(-c2cc(-c3ccccc3)cc(-n3c4ccccc4c4ccc(-n5c6ccccc6c6ccccc65)cc43)c2)cc1]

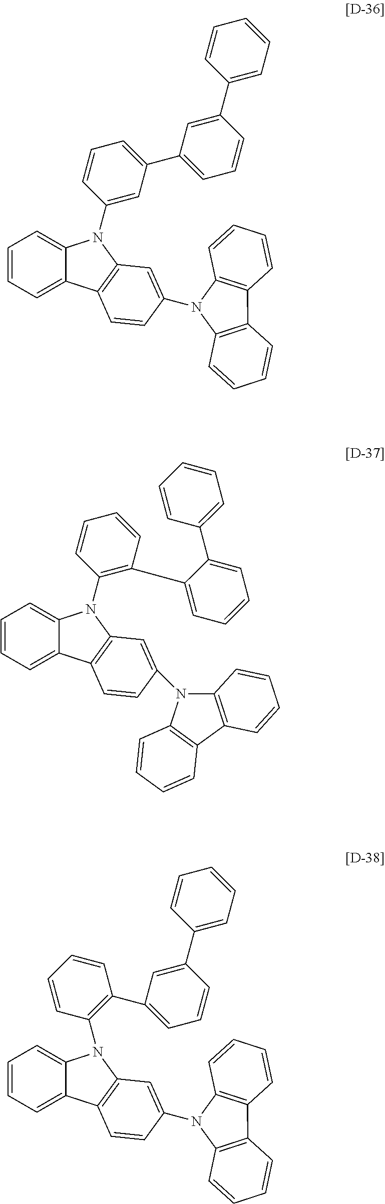 [SMILES: c1ccc(-c2cccc(-c3cccc(-n4c5ccccc5c5ccc(-n6c7ccccc7c7ccccc76)cc54)c3)c2)cc1 ; c1ccc(-c2ccccc2-c2ccccc2-n2c3ccccc3c3ccc(-n4c5ccccc5c5ccccc54)cc32)cc1 ; c1ccc(-c2cccc(-c3ccccc3-n3c4ccccc4c4ccc(-n5c6ccccc6c6ccccc65)cc43)c2)cc1]

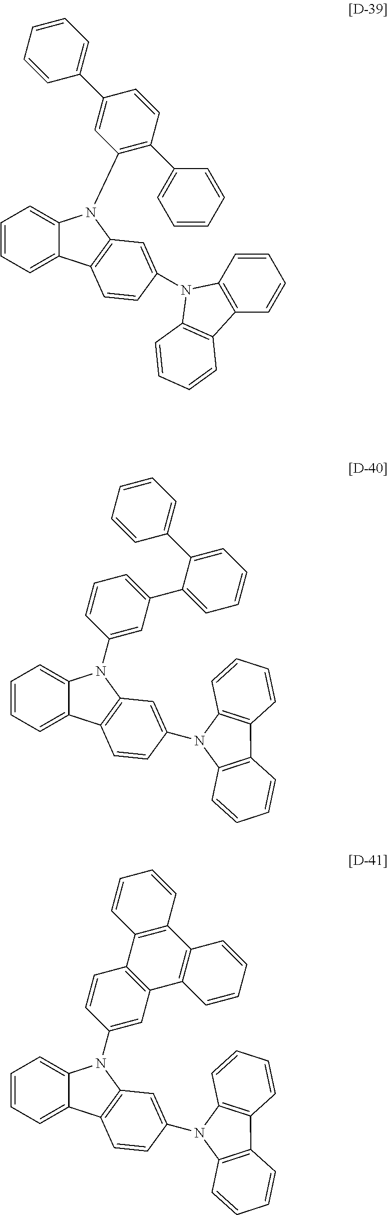 [SMILES: c1ccc(-c2ccc(-c3ccccc3)c(-n3c4ccccc4c4ccc(-n5c6ccccc6c6ccccc65)cc43)c2)cc1 ; c1ccc(-c2ccccc2-c2cccc(-n3c4ccccc4c4ccc(-n5c6ccccc6c6ccccc65)cc43)c2)cc1 ; c1ccc2c(c1)c1ccccc1c1cc(-n3c4ccccc4c4ccc(-n5c6ccccc6c6ccccc65)cc43)ccc21]

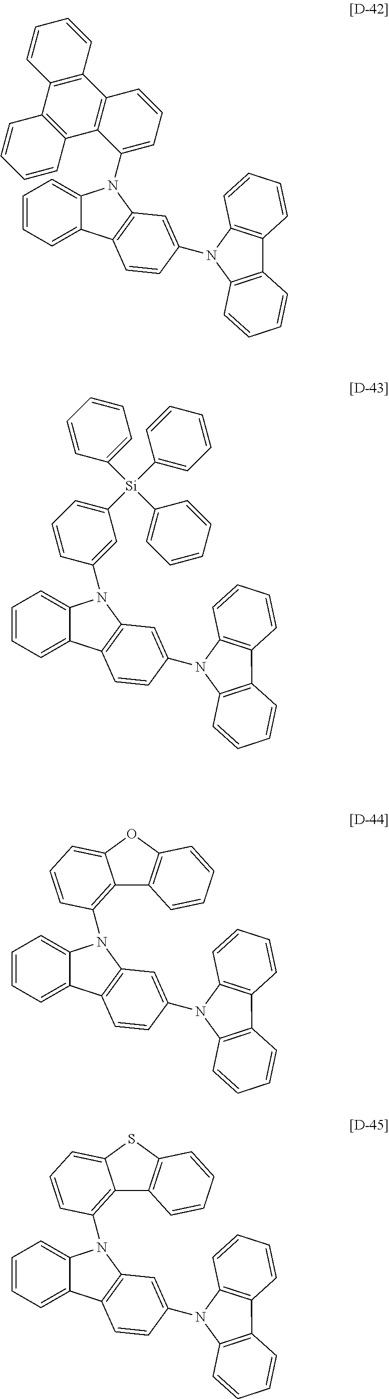 [SMILES: c1ccc2c(c1)c1ccccc1c1c(-n3c4ccccc4c4ccc(-n5c6ccccc6c6ccccc65)cc43)cccc21 ; c1ccc([Si](c2ccccc2)(c2ccccc2)c2cccc(-n3c4ccccc4c4ccc(-n5c6ccccc6c6ccccc65)cc43)c2)cc1 ; c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12]

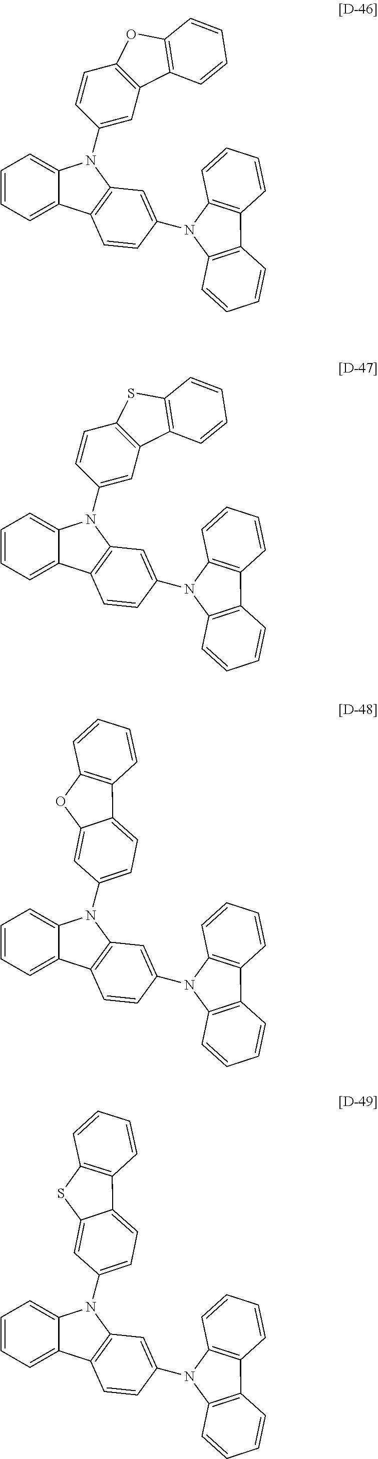 [SMILES: c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12 ; c1ccc2c(c1)oc1cc(-n3c4ccccc4c4ccc(-n5c6ccccc6c6ccccc65)cc43)ccc12 ; c1ccc2c(c1)sc1cc(-n3c4ccccc4c4ccc(-n5c6ccccc6c6ccccc65)cc43)ccc12]

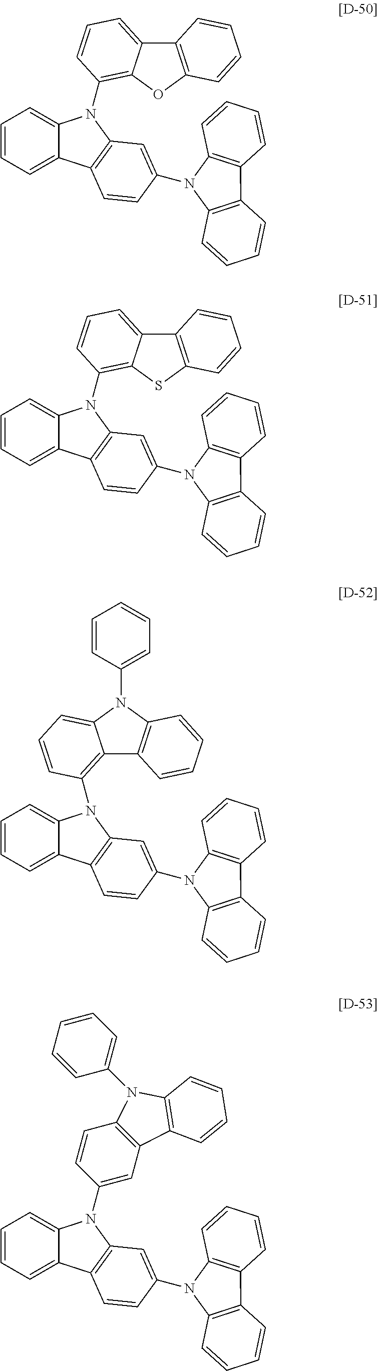 [SMILES: c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12 ; c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1]

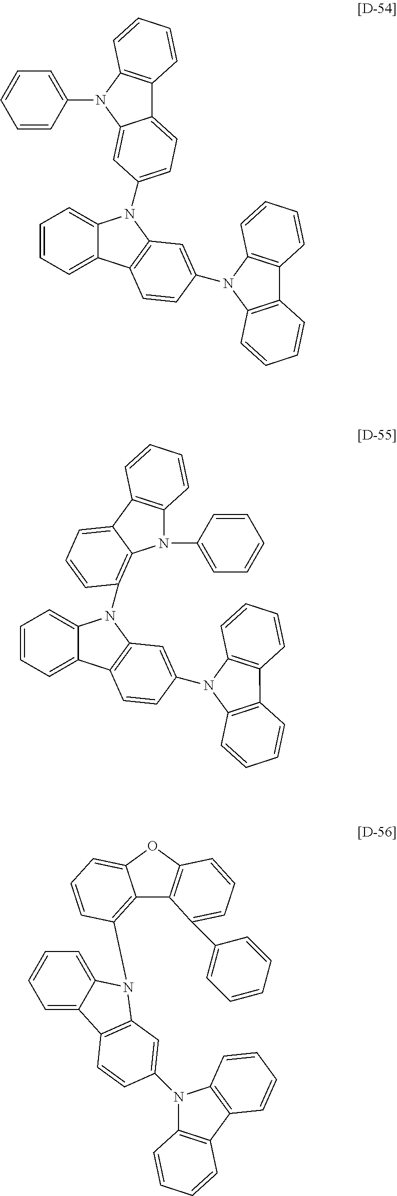 [SMILES: c1ccc(-n2c3ccccc3c3ccc(-n4c5ccccc5c5ccc(-n6c7ccccc7c7ccccc76)cc54)cc32)cc1 ; c1ccc(-n2c3ccccc3c3cccc(-n4c5ccccc5c5ccc(-n6c7ccccc7c7ccccc76)cc54)c32)cc1 ; c1ccc(-c2cccc3oc4cccc(-n5c6ccccc6c6ccc(-n7c8ccccc8c8ccccc87)cc65)c4c23)cc1]

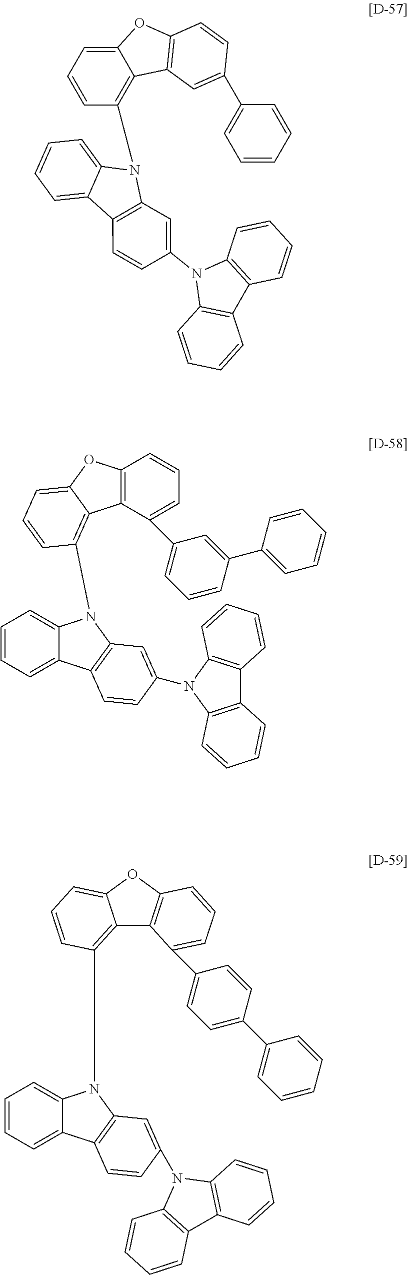 [SMILES: c1ccc(-c2ccc3oc4cccc(-n5c6ccccc6c6ccc(-n7c8ccccc8c8ccccc87)cc65)c4c3c2)cc1 ; c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1]

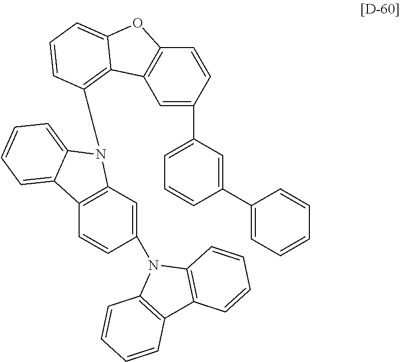 [SMILES: c1ccc(-c2cccc(-c3ccc4oc5cccc(-n6c7ccccc7c7ccc(-n8c9ccccc9c9ccccc98)cc76)c5c4c3)c2)cc1]

Examples of Compound D-1 to Compound D-60 listed in Group 2 in which at least one hydrogen is replaced with deuterium are shown below.

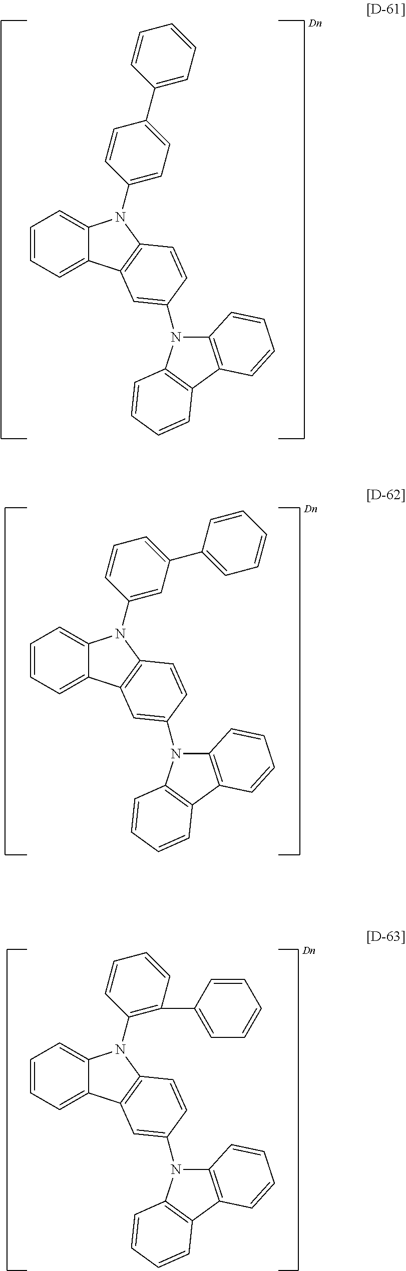

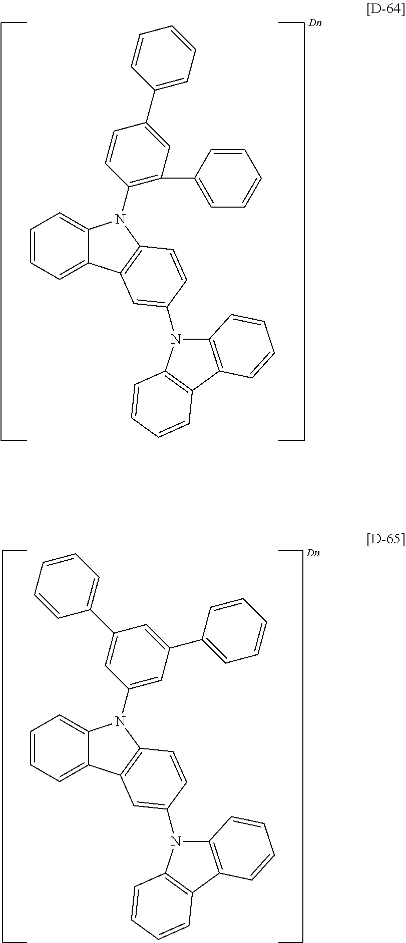

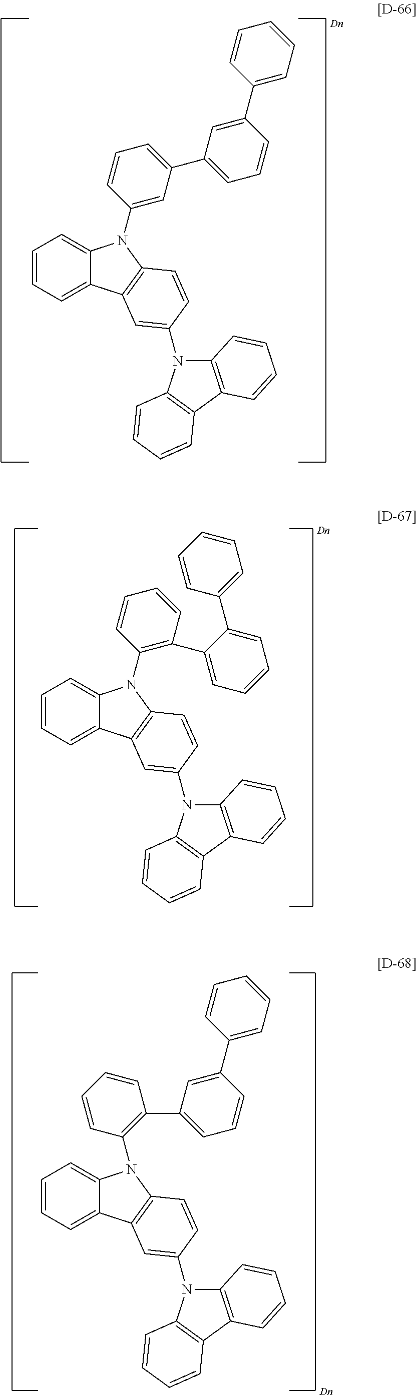

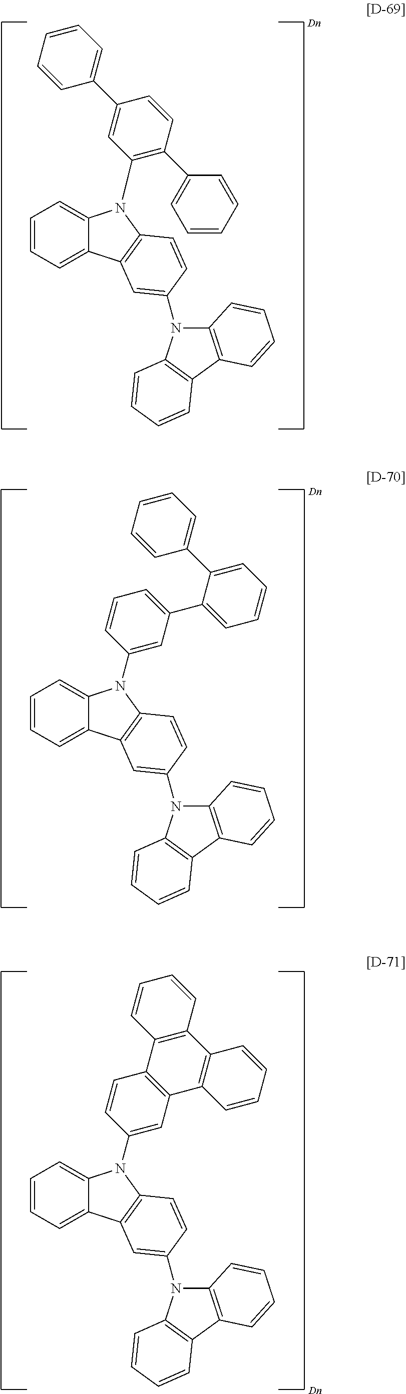

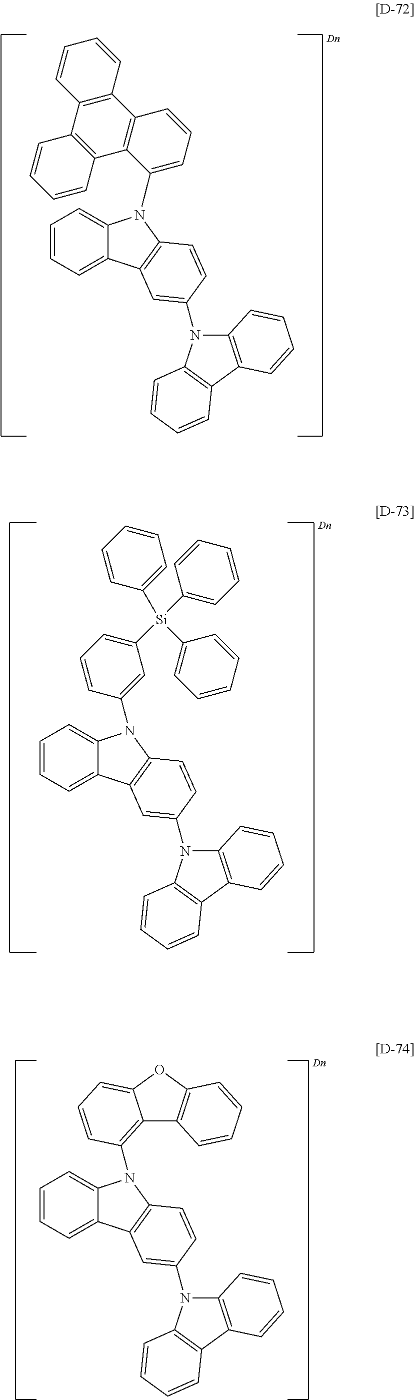

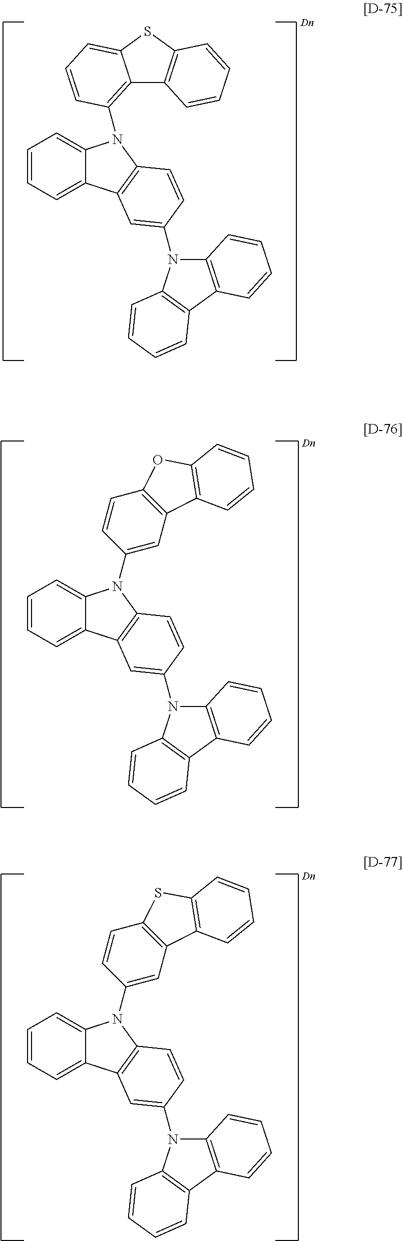

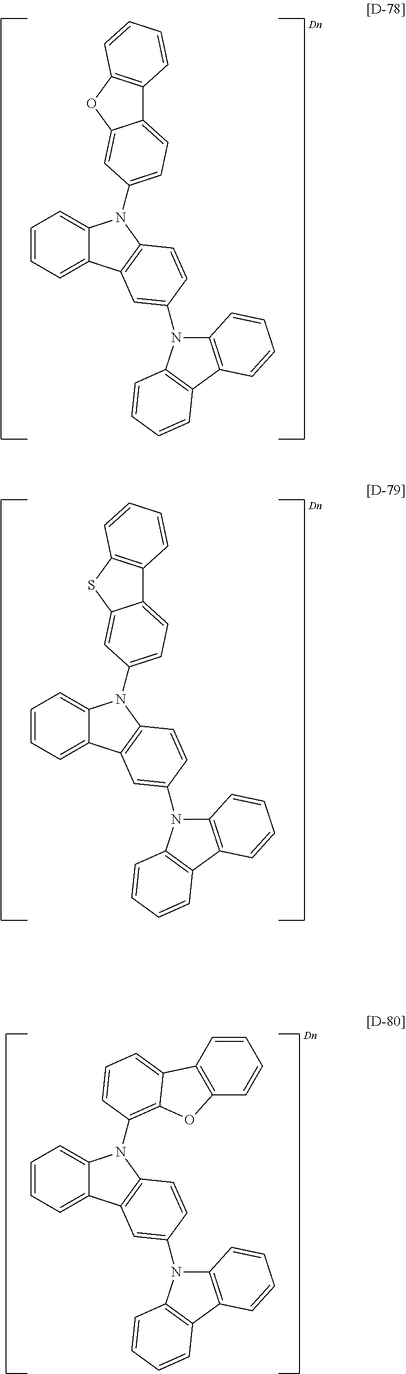

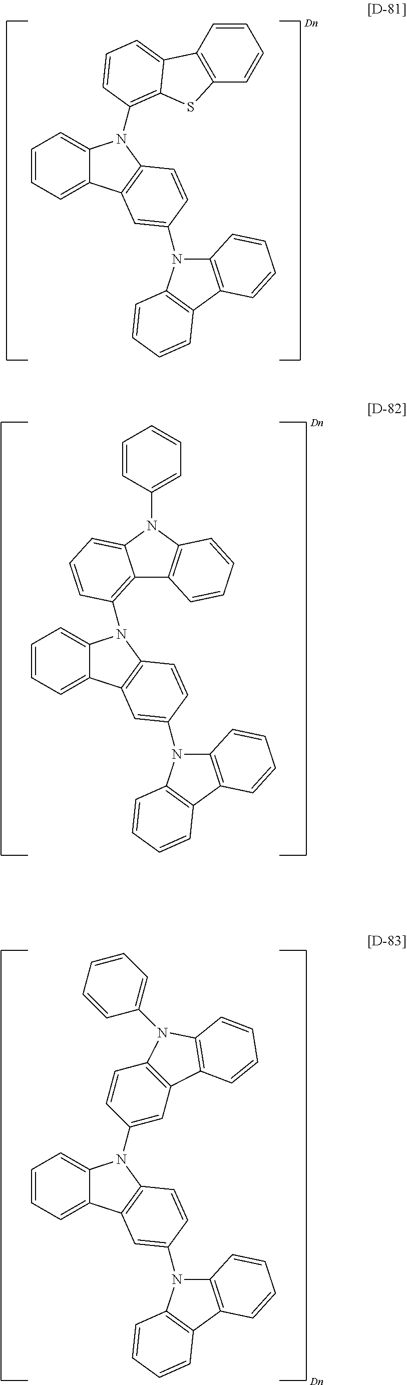

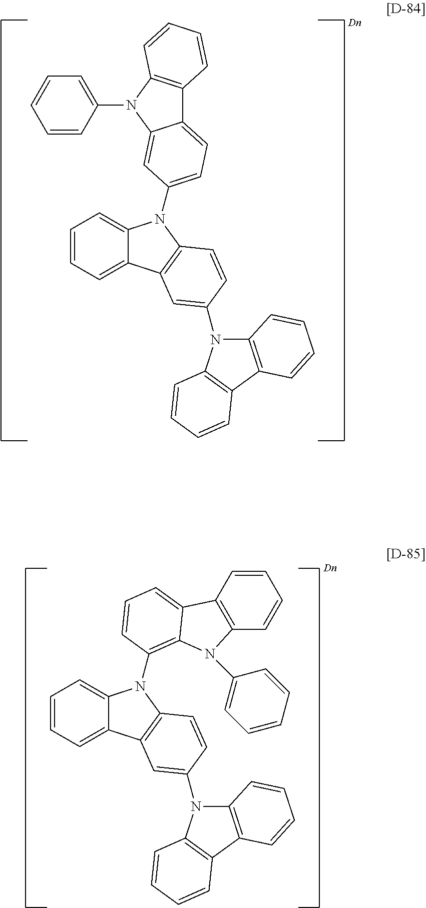

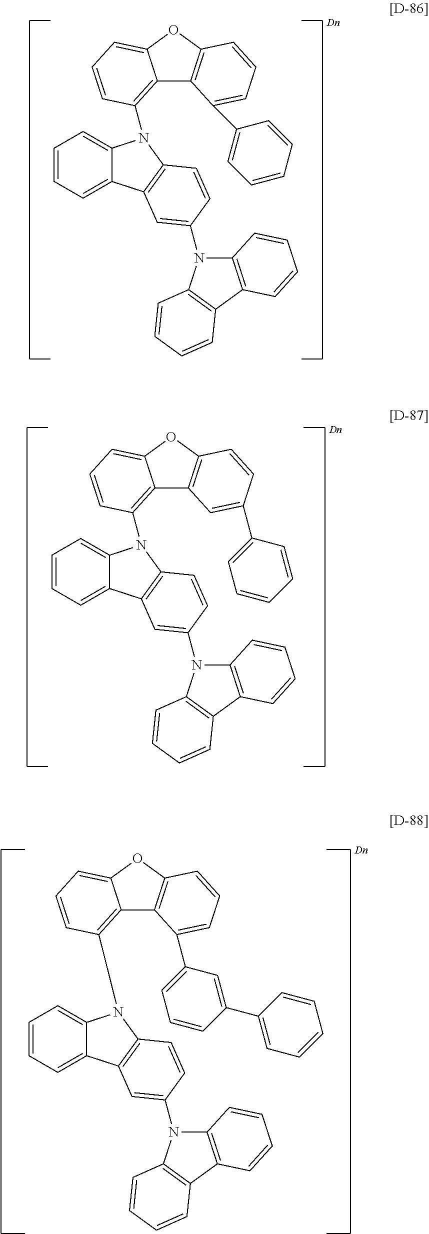

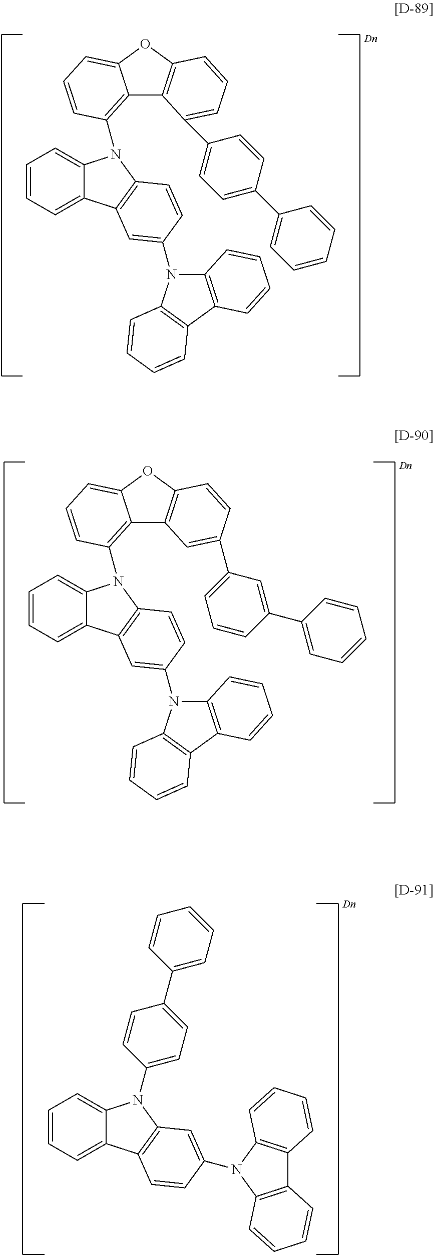

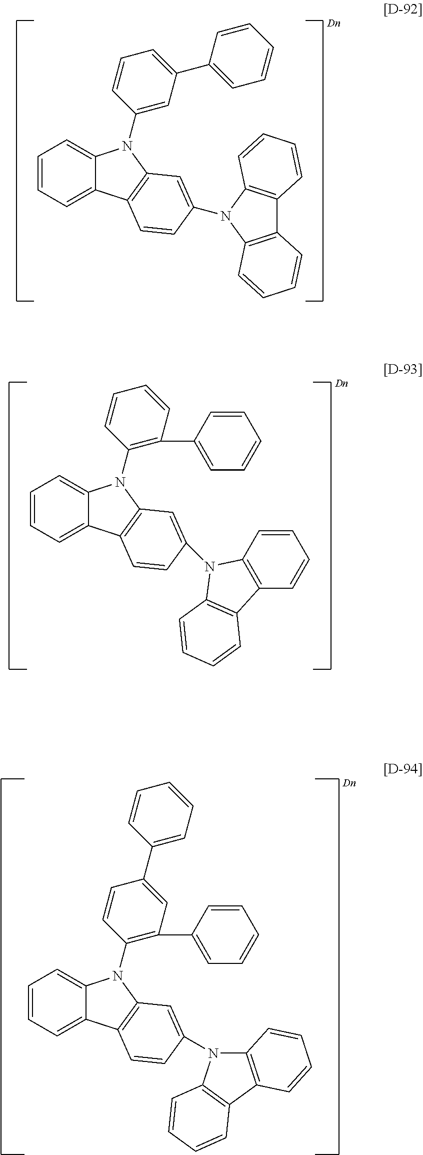

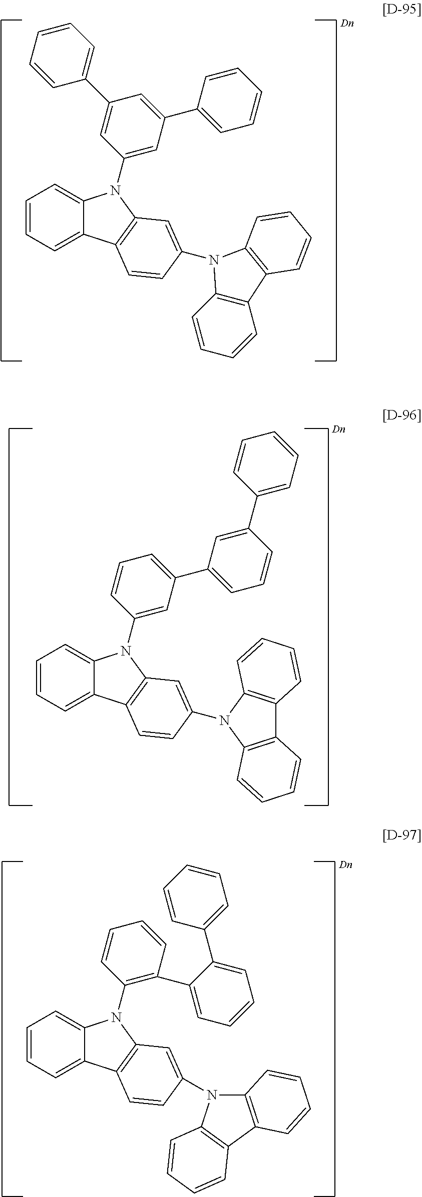

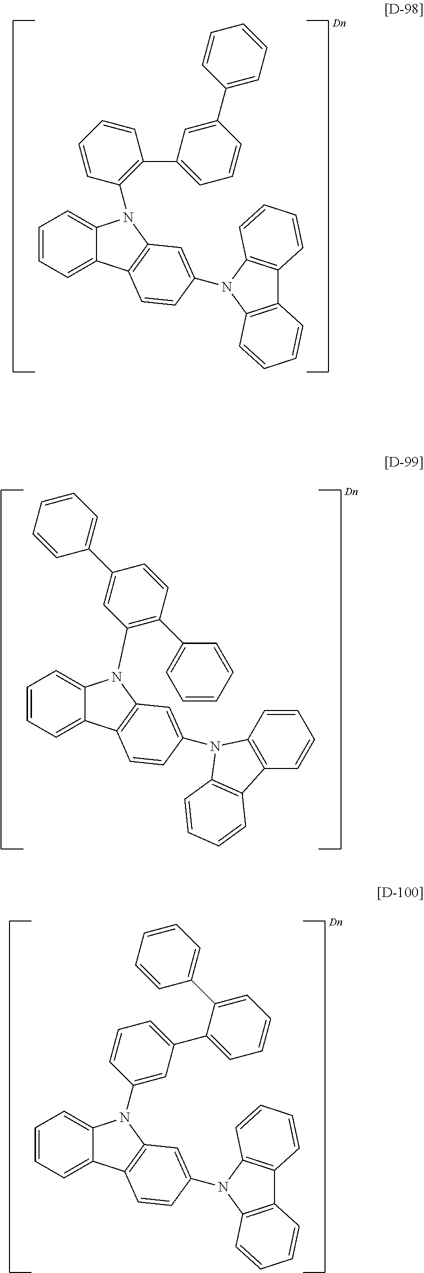

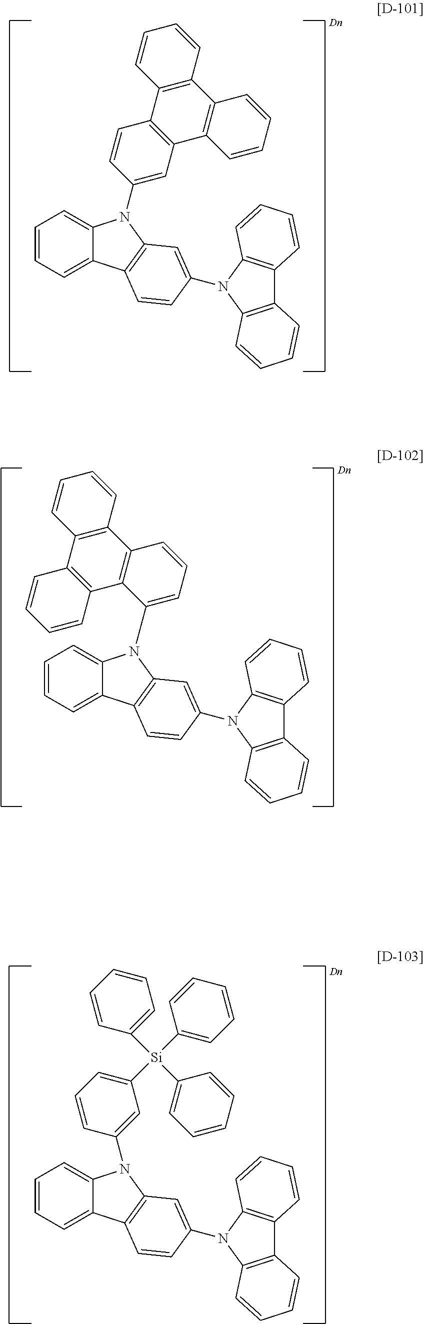

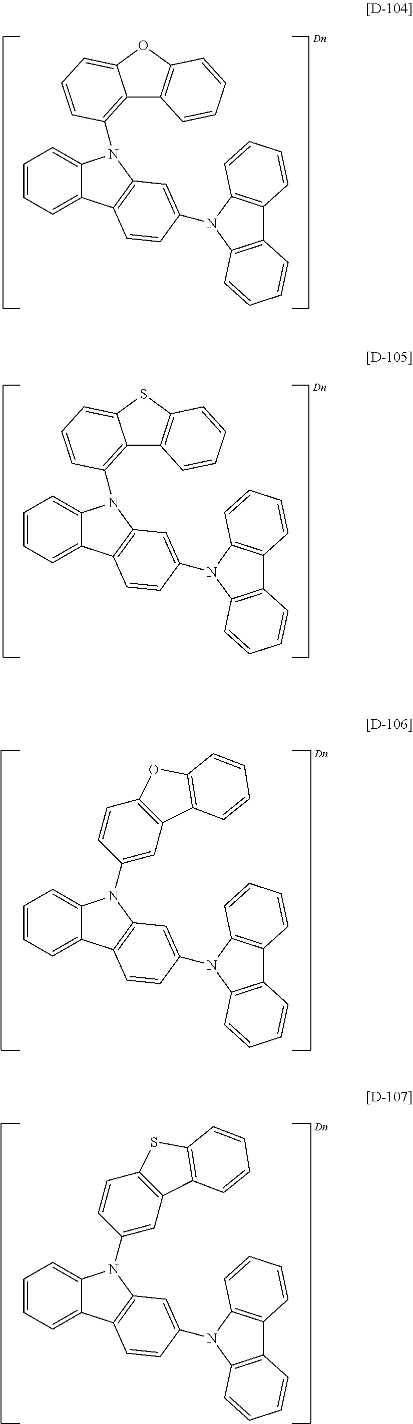

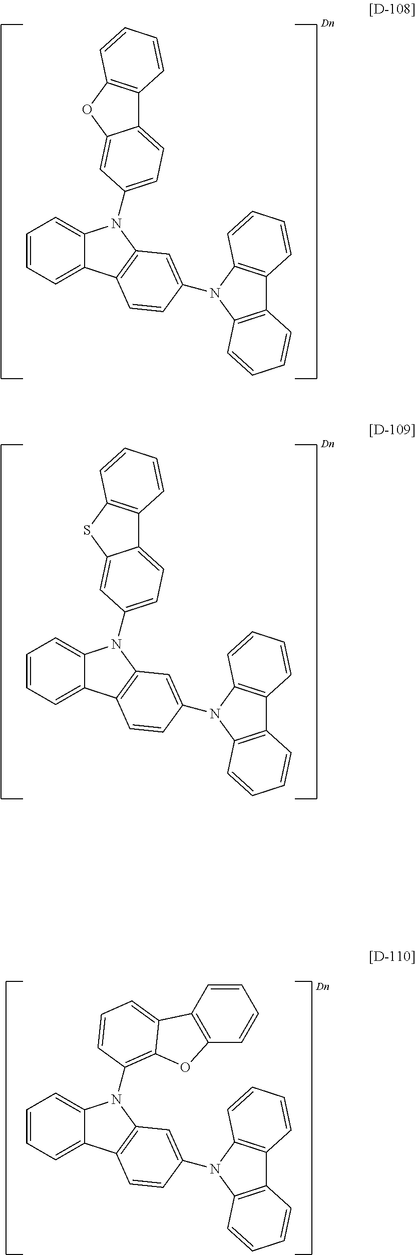

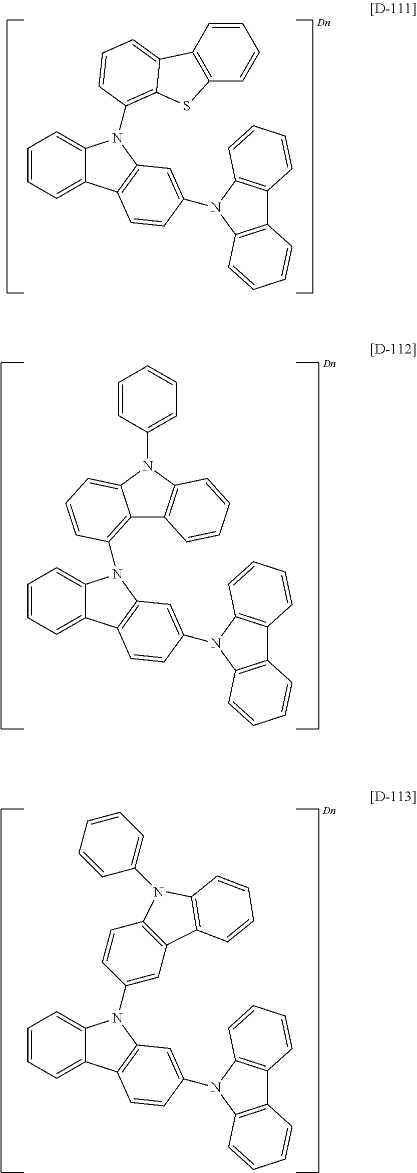

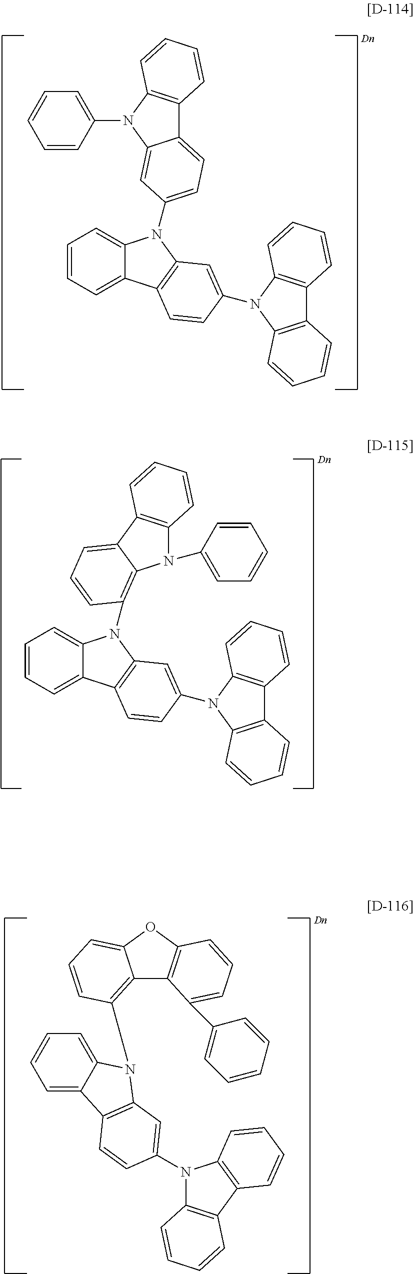

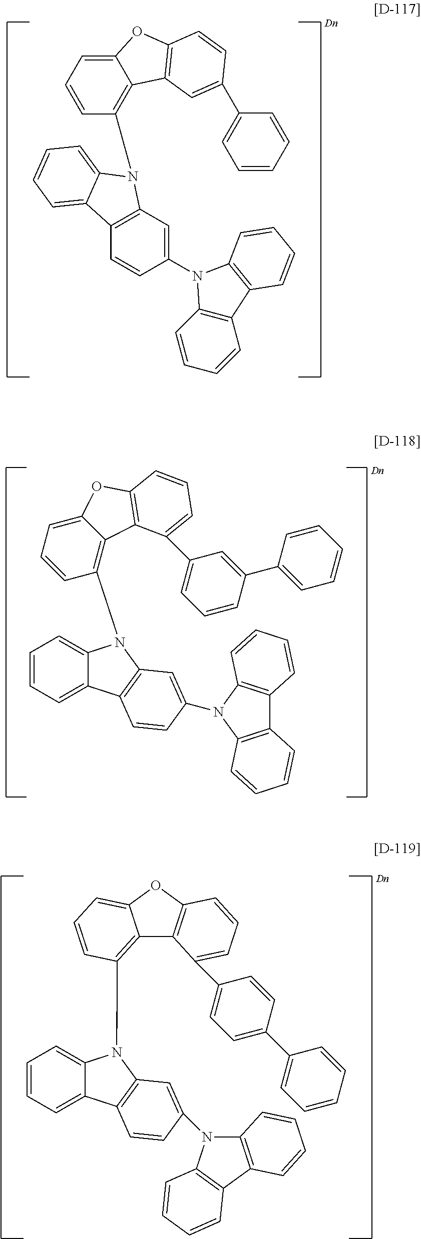

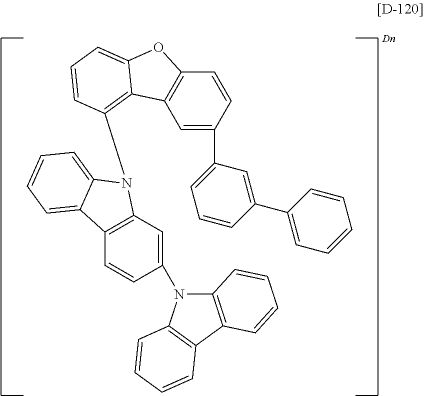

(Dn refers to the number of deuterium atoms substituted and indicates a structure in which one or more deuterium atoms are substituted)

The first compound and the second compound may be included in a weight ratio of, e.g., about 1:99 to about 99:1. Within this range, a desirable weight ratio may be adjusted using an electron transport capability of the first compound and a hole transport capability of the second compound to realize bipolar characteristics and thus help improve efficiency and life-span. Within the above range, e.g., they may be included in a weight ratio of about 10:90 to about 90:10 or about 20:80 to about 80:20, e.g., about 20:80 to about 70:30, about 20:80 to about 60:40, or about 30:70 to about 60:40. In an implementation, they may be included in a weight ratio of, e.g., about 40:60, about 50:50, or about 60:40.

Hereinafter, an organic optoelectronic device including the aforementioned compound for the organic optoelectronic device or composition for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to the FIGURE.

The FIGURE is a cross-sectional view showing an organic light emitting diode according to an embodiment.

Referring to the FIGURE, an organic light emitting diode 100 according to an embodiment may include, e.g., an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof, a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof, a multi-layer structure material such as LiF/Al, LiO₂/Al, LiF/Ca, or BaF₂/Ca.

The organic layer 105 may include, e.g., the aforementioned compound for the organic optoelectronic device or composition for the organic optoelectronic device.

The organic layer 105 may include, e.g., a light emitting layer 130, the light emitting layer 130 may include, e.g., a host and a dopant, the host may include, e.g., the aforementioned compound for the organic optoelectronic device or composition for an organic optoelectronic device, and the dopant may be, e.g., a phosphorescent dopant, e.g., a red, green or blue phosphorescent dopant, e.g., a red or green phosphorescent dopant.

The dopant is a material mixed with the compound or composition for an organic optoelectronic device in a small amount to cause light emission and may be, e.g., generally a material such as a metal complex that emits light by multiple excitation into a triplet or more. The dopant may be, e.g., an inorganic, organic, or organic-inorganic compound, and one or more types thereof may be used.

Examples of the dopant may be, e.g., a phosphorescent dopant and examples of the phosphorescent dopant may be, e.g., an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L⁶MX¹ [Chemical Formula Z]

In Chemical Formula Z, M may be, e.g., a metal, and L⁶ and X¹ may be the same or different, and may each independently be, e.g., a ligand forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁶ and X¹ may be, e.g., a bidentate ligand.

The ligands represented by L⁶ and X¹ may be, e.g., one of Chemical Formula Z-1 to Chemical Formula Z-8.

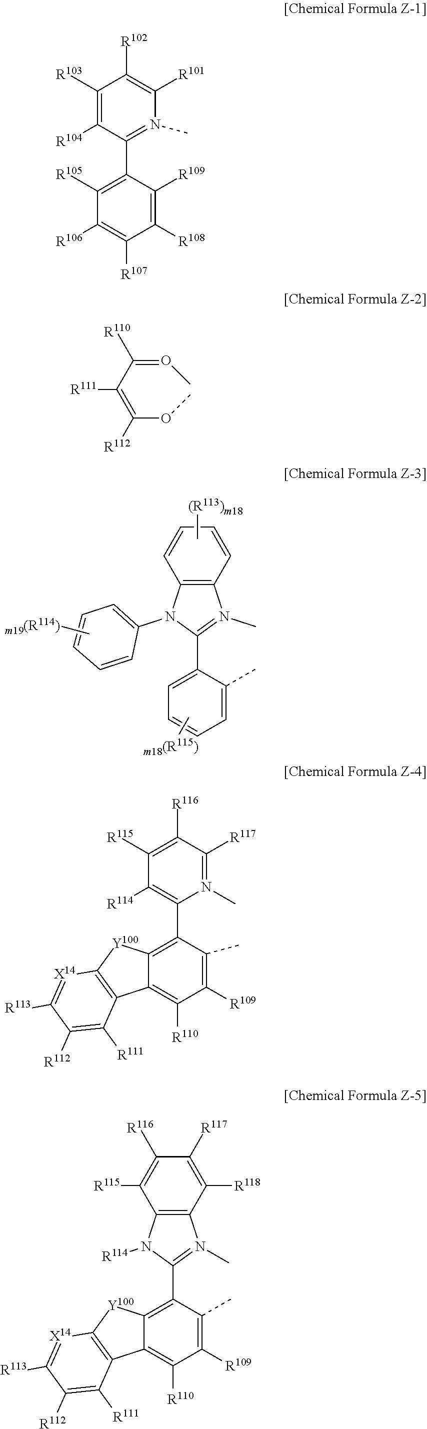

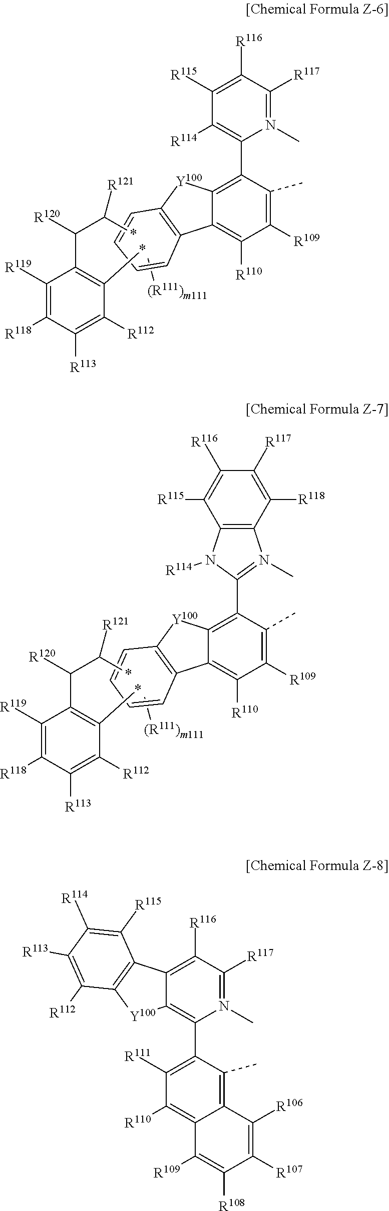

In Chemical Formula Z-1 to Chemical Formula Z-8, X¹⁴ may be, e.g., carbon or nitrogen.

Y¹⁰⁰ may be, e.g., O or S.

R¹⁰¹ to R¹²² may each independently be, e.g., hydrogen, deuterium, halogen, a substituted or unsubstituted C1 to C30 alkyl group, a substituted or unsubstituted C3 to C30 cycloalkyl group, a substituted or unsubstituted C6 to C20 aryl group, —SiR¹³³R¹³⁴R¹³⁵, or —GeR¹³³R¹³⁴R¹³⁵; or may be linked to adjacent substituents to form a substituted or unsubstituted ring, and, e.g., together with pyridine, to form a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline.

R¹³³ to R¹³⁵ may each independently be, e.g., a substituted or unsubstituted C1 to C6 alkyl group.

m18 may be, e.g., an integer of 1 to 4.

m19 may be, e.g., an integer of 1 to 5.

m111 may be, e.g., an integer of 1 or 2.

Examples of the ligands represented by L⁶ and X¹ may be or include, e.g., chemical formulas listed in Group A.

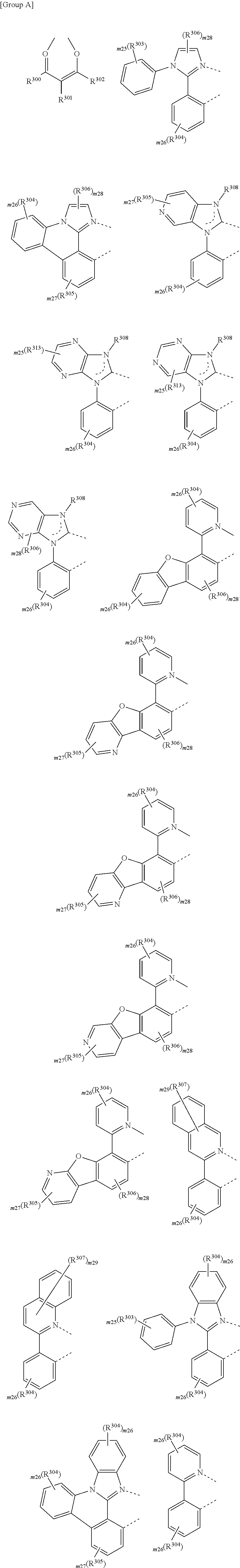

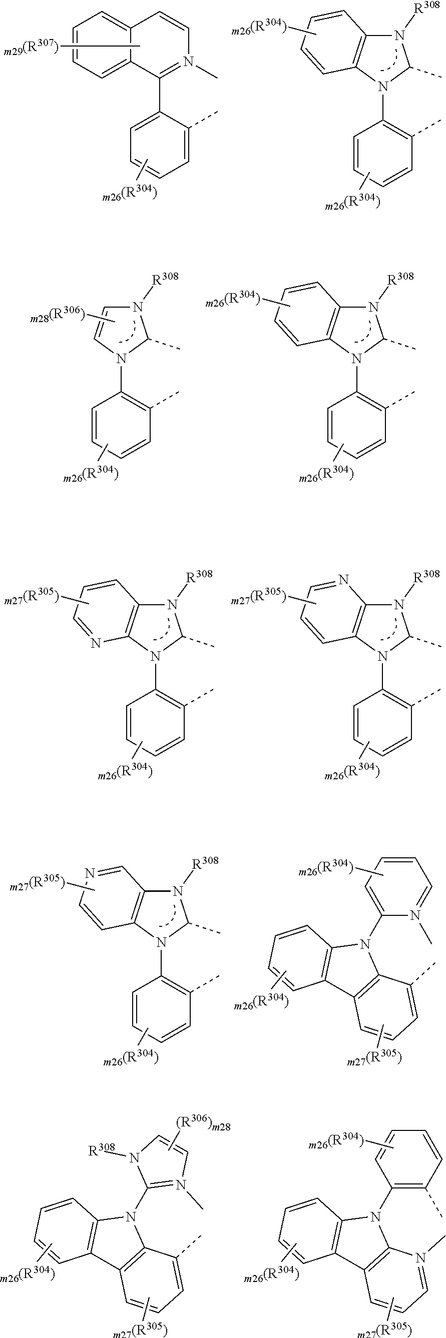

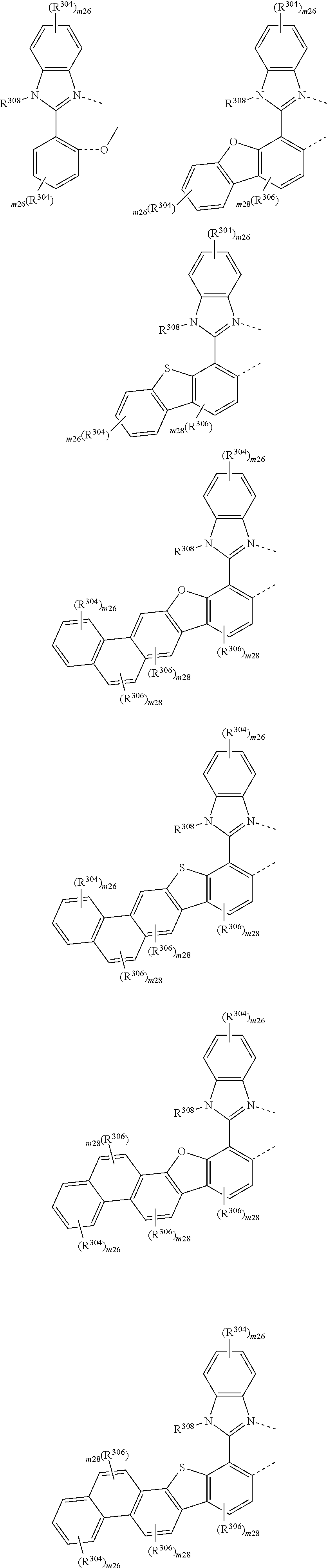

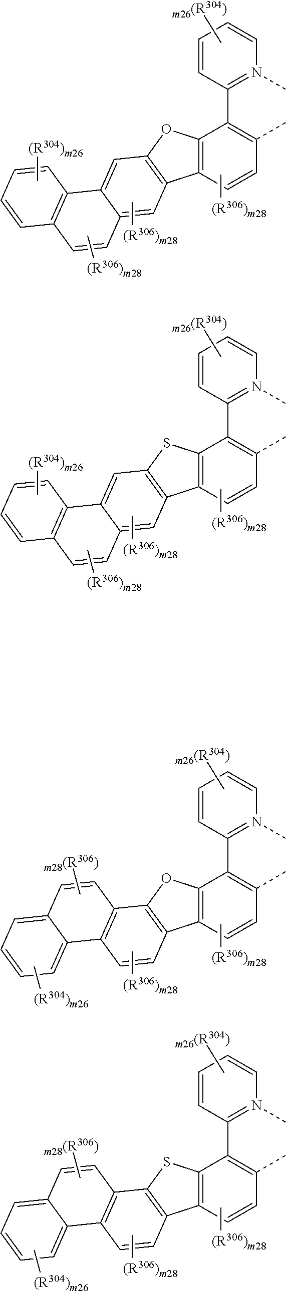

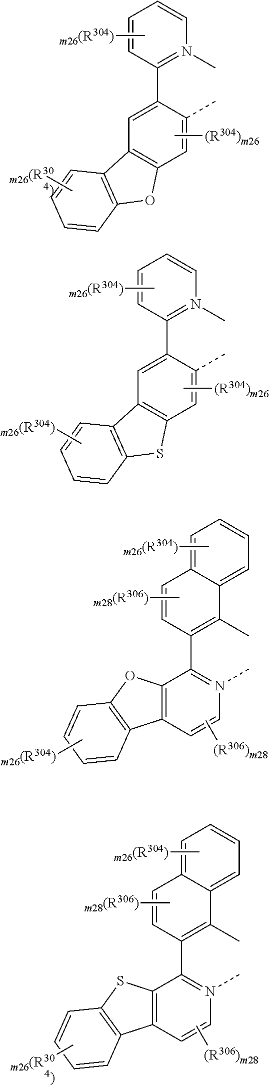

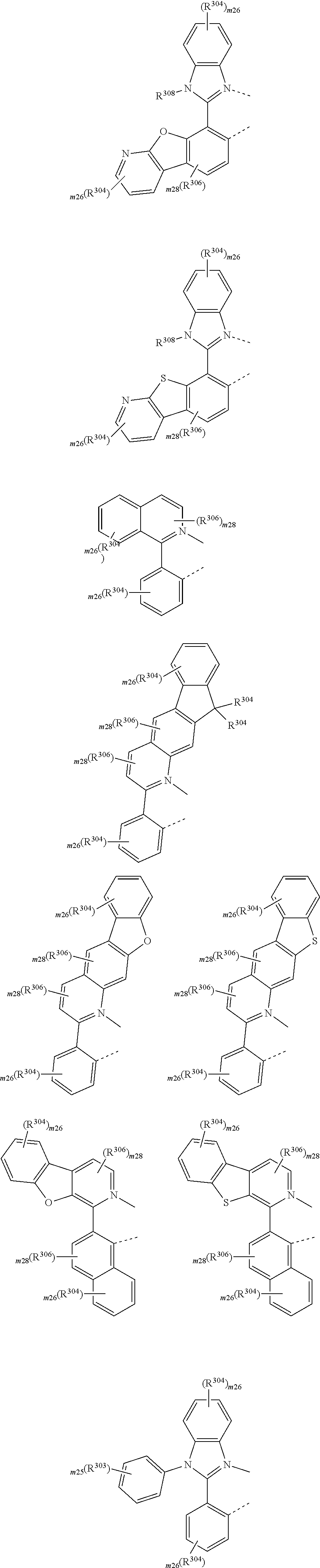

In Group A, R³⁰⁰ to R³⁰² may each independently be, e.g., hydrogen, deuterium a C1 to C30 alkyl group that is substituted or unsubstituted with a halogen, a C6 to C30 aryl group that is substituted or unsubstituted with a C1 to C30 alkyl, or a halogen.

R³⁰³ to R³⁰⁸ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group,

m25 may be, e.g., an integer of 1 to 5.

m26 may be, e.g., an integer of 1 to 4.

m27 may be, e.g., an integer of 1 to 3.

m28 may be, e.g., an integer of 1 or 2.

m29 may be, e.g., an integer of 1 to 6.

In an implementation, any one of m25 to m29 may be an integer of 2 or more, and the respective ones of R³⁰³ to R³⁰⁷ may be the same or different from each other.

The dopant according to an embodiment may be an iridium complex, and may be represented, e.g., by one of Chemical Formula 7 to Chemical Formula 9.

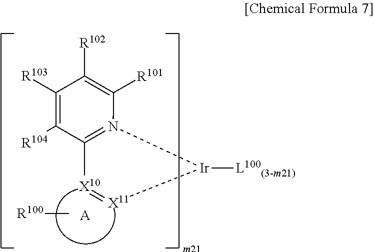

In Chemical Formula 7, ring A may be, e.g., a monocyclic ring or a polycyclic fused ring, wherein each ring among the monocyclic ring and polycyclic fused ring may be, e.g., a 5- or 6-membered carbocyclic or heterocyclic ring.

R¹⁰⁰ may represent, e.g., one to a maximum number of monovalent substituents.

In an implementation, there may be two or more of R¹⁰⁰ and each R¹⁰⁰ may be the same or different from each other.

R¹⁰¹ to R¹⁰⁴ may each independently be, e.g., hydrogen, deuterium, halogen, a substituted or unsubstituted C1 to C10 alkyl group, a substituted or unsubstituted C6 to C20 aryl group, —SiR¹¹⁴R¹¹⁵R¹¹⁶, —GeR¹¹⁴R¹¹⁵R¹¹⁶, or a combination thereof.

R¹¹⁴ to R¹¹⁶ may each independently be, e.g., a substituted or unsubstituted C1 to C6 alkyl group.

X¹⁰ and X¹¹ may each independently be, e.g., carbon or nitrogen.

L¹⁰⁰ may be, e.g., a ligand of a monovalent anion or a bidentate ligand, which coordinates to iridium through the unshared electron pair of carbon or a heteroatom.

m21 may be, e.g., an integer of 0 to 3.

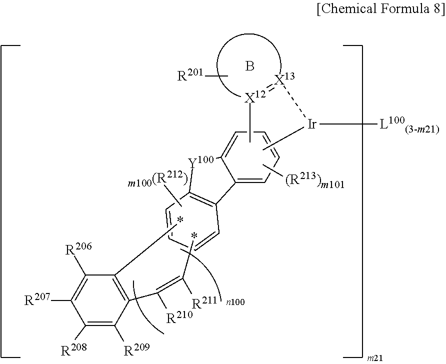

In Chemical Formula 8, ring B may be, e.g., a monocyclic ring or a polycyclic fused ring, wherein each ring among the monocyclic ring and polycyclic fused ring may be, e.g., a 5- or 6-membered carbocyclic or heterocyclic ring.

Y¹⁰⁰ may be, e.g., O or S.

R²⁰¹ may represent, e.g., one to a maximum number of monovalent substituents.

In an implementation, there may be two or more of R²⁰¹, and each R²⁰¹ may be the same or different from each other.

R²⁰⁶ to R²¹³ may each independently be, e.g., hydrogen, deuterium, halogen, a substituted or unsubstituted C1 to C10 alkyl group, a substituted or unsubstituted C6 to C20 aryl group, —SiR¹¹⁴R¹¹⁵R¹¹⁶, —GeR¹¹⁴R¹¹⁵R¹¹⁶, or a combination thereof.

R¹¹⁴ to R¹¹⁶ may each independently be, e.g., a substituted or unsubstituted C1 to C6 alkyl group.

X¹² and X¹³ may each independently be, e.g., carbon or nitrogen.

L¹⁰⁰ may be, e.g., a ligand of a monovalent anion or a bidentate ligand, which coordinates to iridium through the unshared electron pair of carbon or a heteroatom.

m21 may be an integer of 0 to 3.

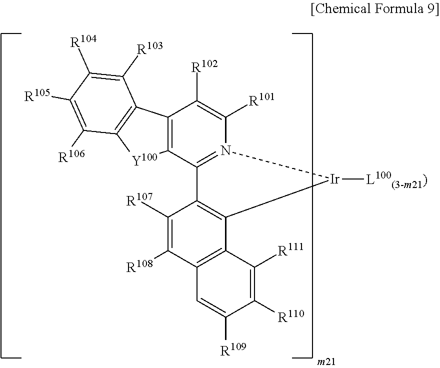

In Chemical Formula 9, Y¹⁰⁰ may be, e.g., O or S.

R¹⁰¹ to R¹¹¹ may each independently be, e.g., hydrogen, deuterium, halogen, a substituted or unsubstituted C1 to C10 alkyl group, a substituted or unsubstituted C6 to C20 aryl group, —SiR¹¹⁴R¹¹⁵R¹¹⁶, —GeR¹¹⁴R¹¹⁵R¹¹⁶, or a combination thereof.

R¹¹⁴ to R¹¹⁶ may each independently be, e.g., a substituted or unsubstituted C1 to C6 alkyl group.

L¹⁰⁰ may be, e.g., a ligand of a monovalent anion or a bidentate ligand, which coordinates to iridium through the unshared electron pair of carbon or a heteroatom.

m21 may be, e.g., an integer of 0 to 3.

In an implementation, the iridium complex may be, e.g., one of Chemical Formula 7-1 to Chemical Formula 7-6.

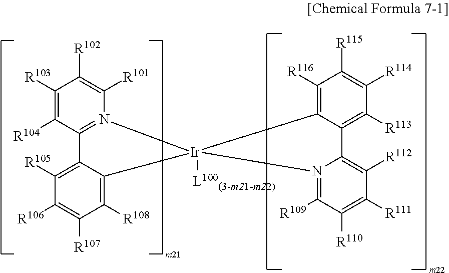

In Chemical Formula 7-1, R¹⁰¹ to R¹¹⁶ may each independently be, e.g., hydrogen, deuterium, halogen, a substituted or unsubstituted C1 to C10 alkyl group, a substituted or unsubstituted C6 to C20 aryl group, —SiR¹³²R¹³³R¹³⁴, or —GeR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be, e.g., a functional group represented by Chemical Formula V-1.

L¹⁰⁰ may be, e.g., a bidentate ligand of a monovalent anion, and may be, e.g., a ligand that coordinates to iridium through a lone pair of carbons or heteroatoms.

m21 and m22 may each independently be, e.g., an integer of 0 to 3, and m21+m22 may be, e.g., an integer of 1 to 3.

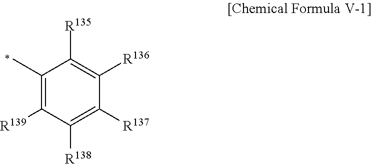

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* means a portion linked to a carbon atom.

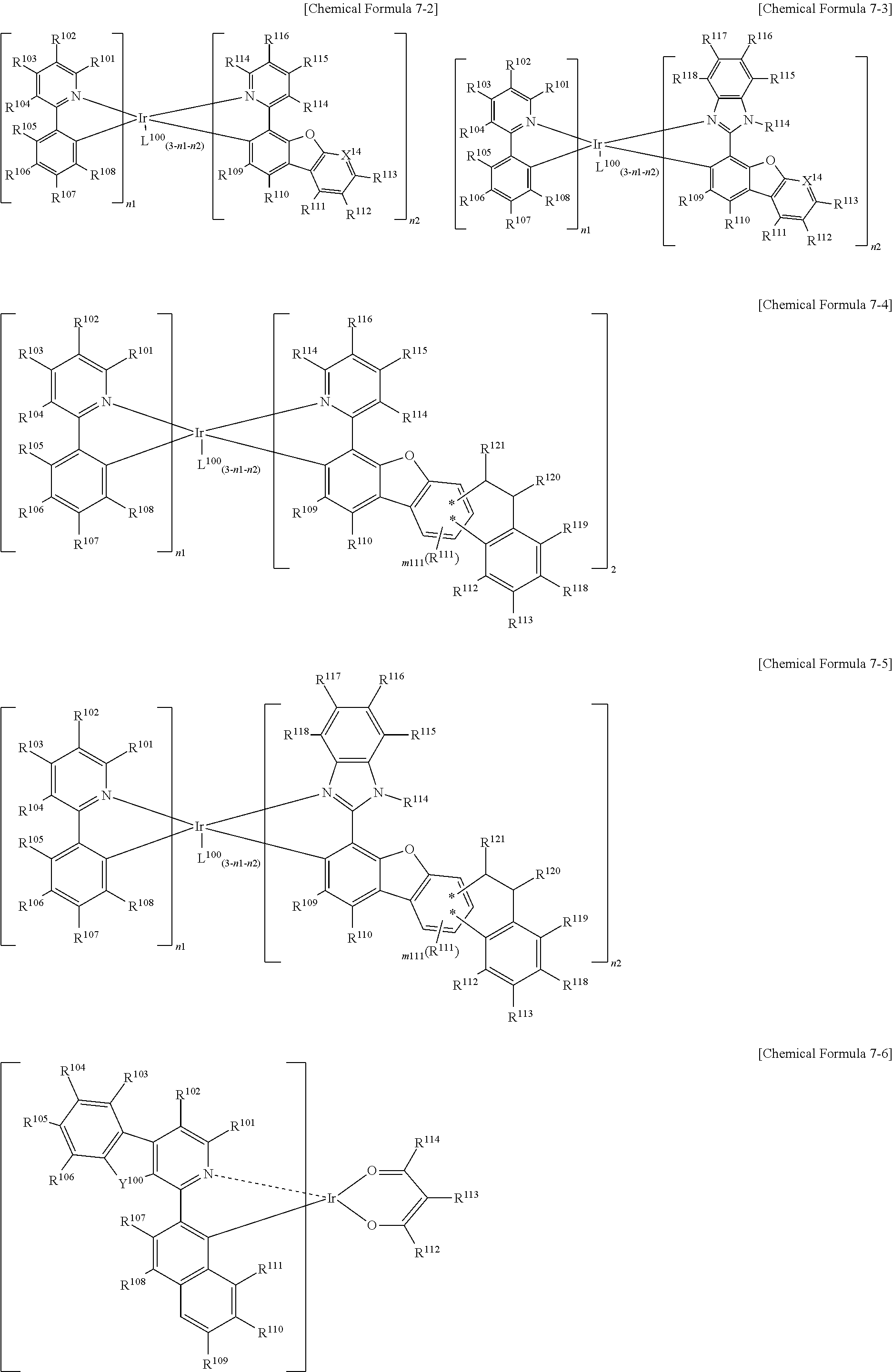

In Chemical Formula 7-2 to Chemical Formula 7-6, X¹⁴ may be, e.g., carbon or nitrogen.

Y¹⁰⁰ may be, e.g., O or S.

R¹⁰¹ to R¹²² may each independently be, e.g., hydrogen, deuterium, halogen, a substituted or unsubstituted C1 to C10 alkyl group, a substituted or unsubstituted C6 to C20 aryl group, —SiR¹³³R¹³⁴R¹³⁵, or —GeR¹³³R¹³⁴R¹³⁵.

R¹³³ to R¹³⁵ may each independently be, e.g., a substituted or unsubstituted C1 to C6 alkyl group.

L¹⁰⁰ may be, e.g., a bidentate ligand of a monovalent anion, and may be, e.g., a ligand that coordinates to iridium through a lone pair of carbons or heteroatoms.

m111 may be, e.g., an integer of 1 or 2.

n1 and n2 may each independently be, e.g., an integer of 0 to 3, and n1+n2 may be, e.g., an integer of 1 to 3.

In some example embodiments, the dopant may be a platinum complex, e.g., represented by Chemical Formula Z-10.

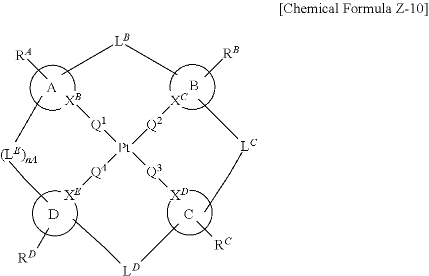

In Chemical Formula Z-10, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution or unsubstitution.

nA may be, e.g., an integer of 0 or 1.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, nA may be, e.g., 1, L^{E} may be, e.g., a direct bond, BR, NR, PR, O, S, Se, C═O, S═O, SO₂, CRR′, SiRR′, GeRR′, or a combination thereof.

In an implementation, nA may be 0 and L^{E} may not exist.

R^{A}, R^{B}, R^{C}, R^{D}, R, and R′ may each independently be, e.g., hydrogen, deuterium, a halogen, alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be, e.g., optionally linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The platinum complex may be represented, e.g., by Chemical Formula 8-1 or Chemical Formula 8-2.

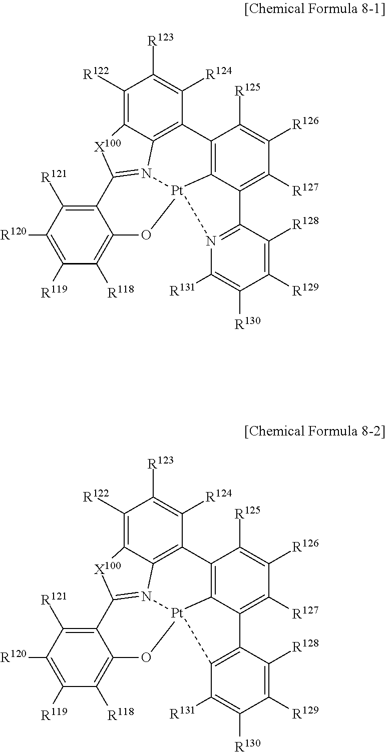

In Chemical Formula 8-1 and Chemical Formula 8-2, X¹⁰⁰ may be, e.g., O, S, or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be, e.g., —SiR¹³³R¹³⁴R¹³⁵ or a tert-butyl group.

R¹³³ to R¹³⁵ may each independently be, e.g., a substituted or unsubstituted C1 to C6 alkyl group.

The organic layer may further include, e.g., a charge transport region in addition to the light emitting layer.

The charge transport region may be, e.g., a hole transport region 140.

The hole transport region 140 may help further increase hole injection or hole mobility and help block electrons between the anode 120 and the light emitting layer 130.

Specifically, the hole transport region 140 may include, e.g., a hole transport layer between the anode 120 and the light emitting layer 130, and a hole transport auxiliary layer between the light emitting layer 130 and the hole transport layer, and, e.g., one of the compounds listed in Group B may be included in at least one layer of the hole transport layer and the hole transport auxiliary layer.

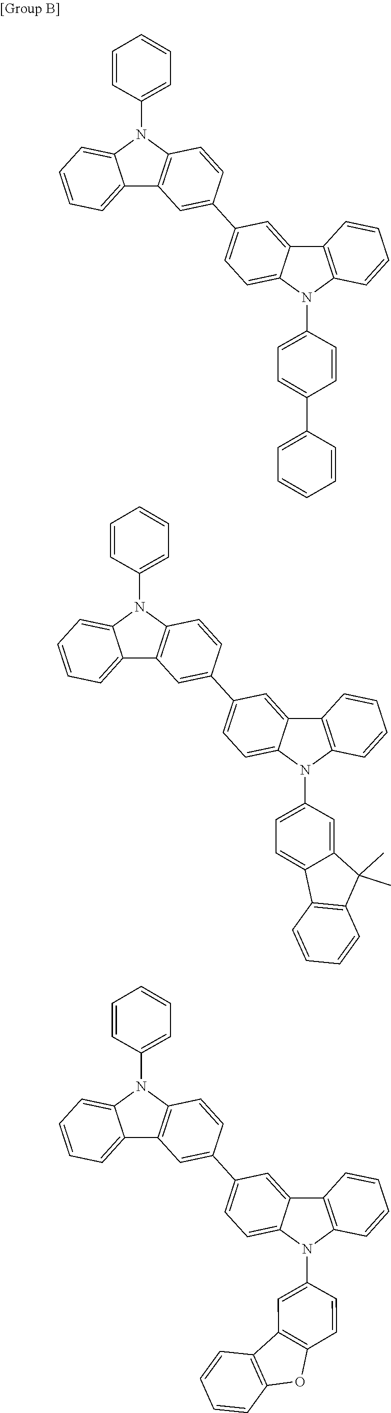 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

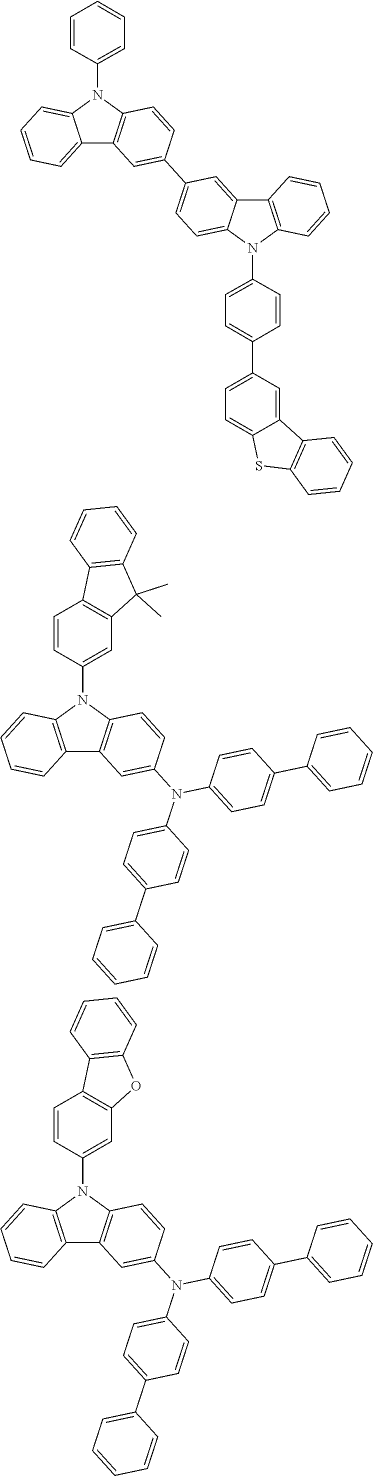

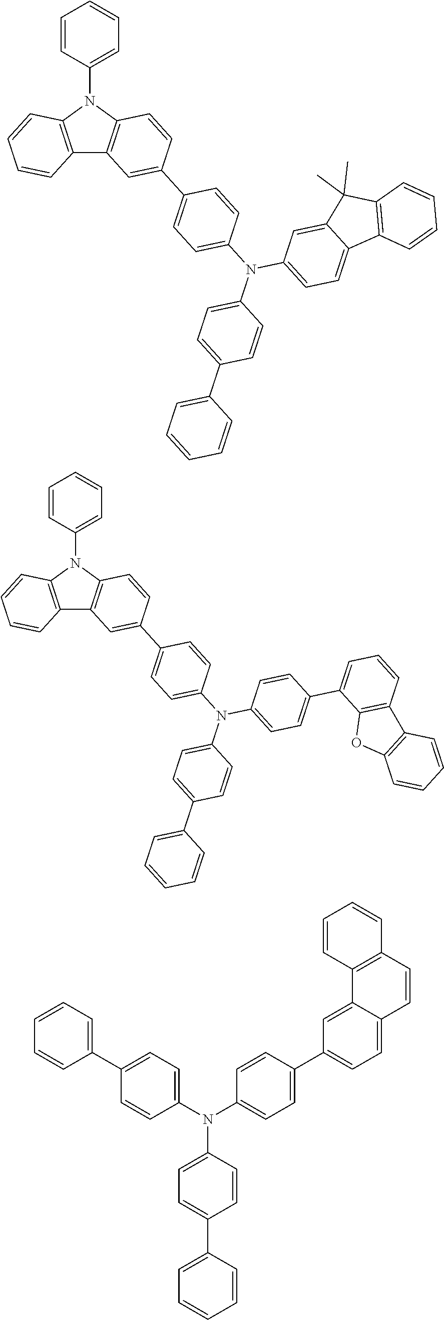 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

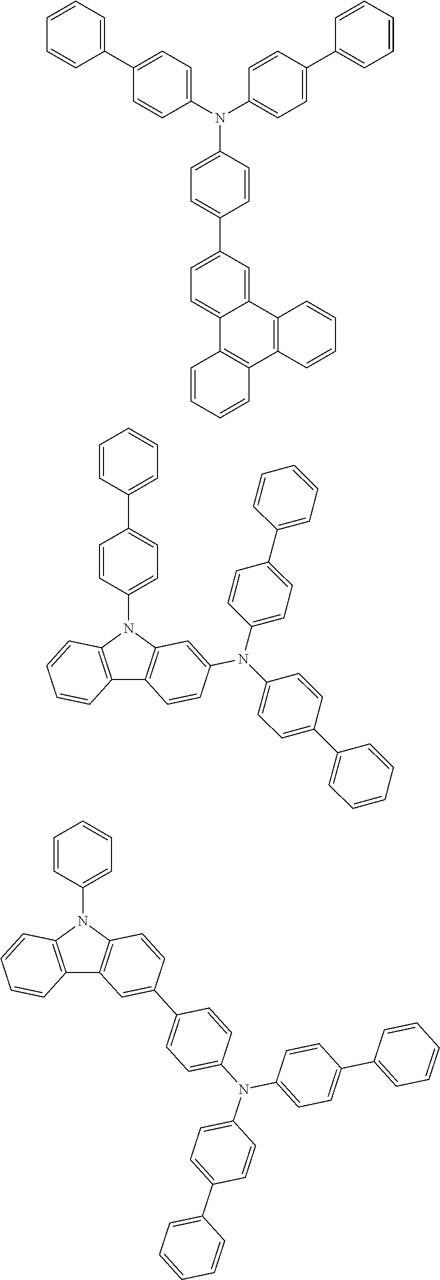 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

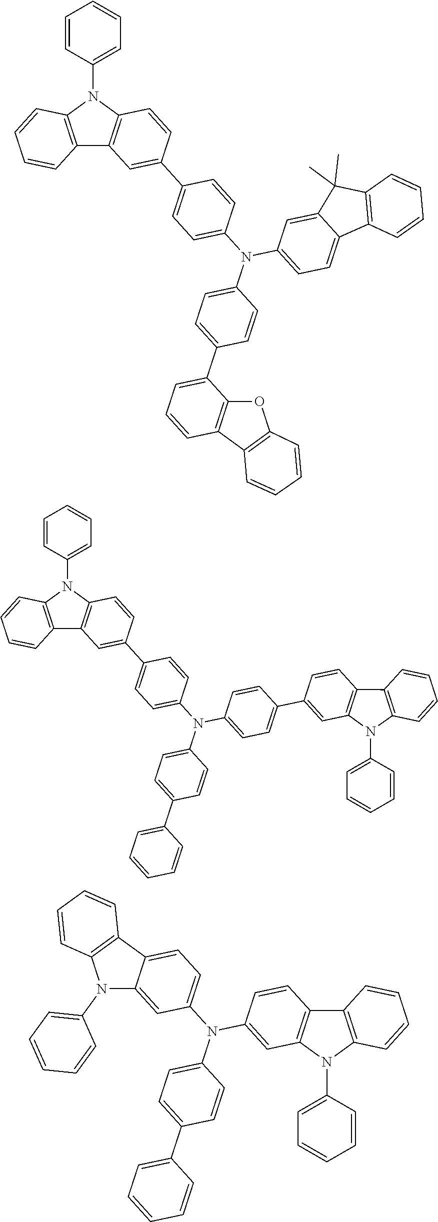

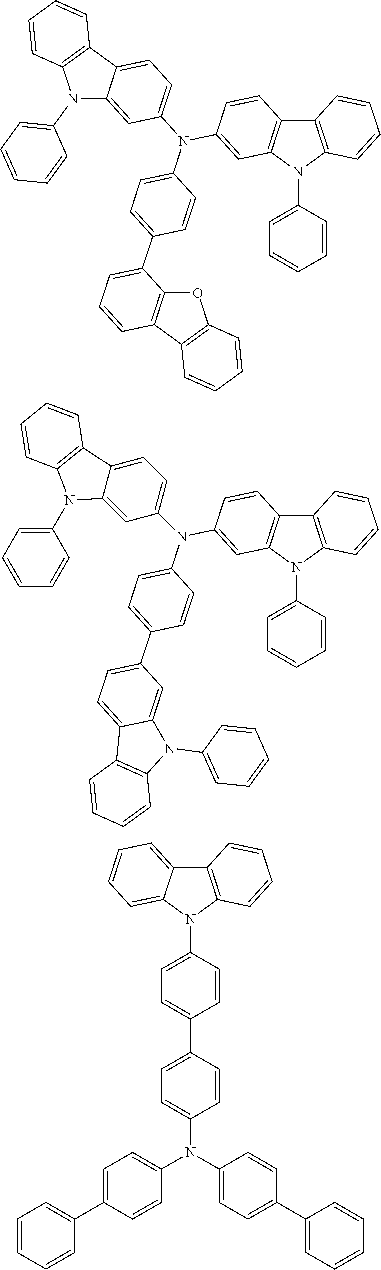

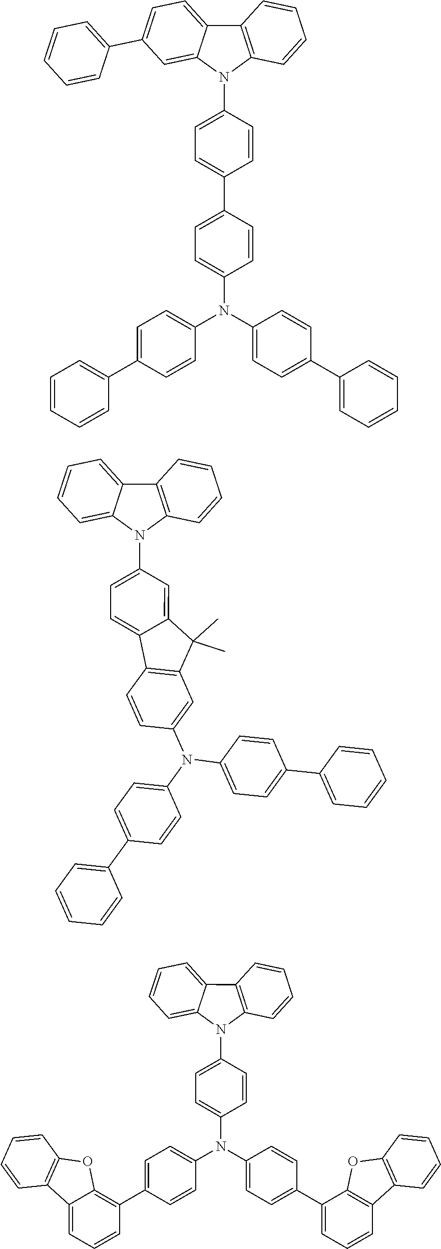

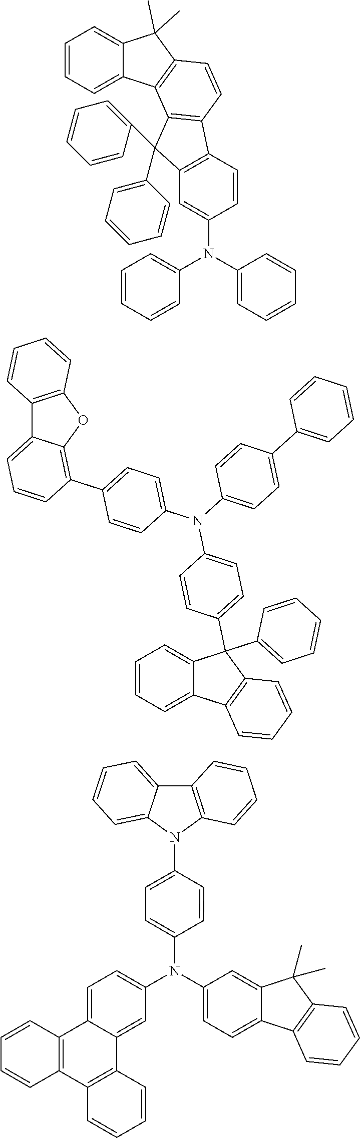

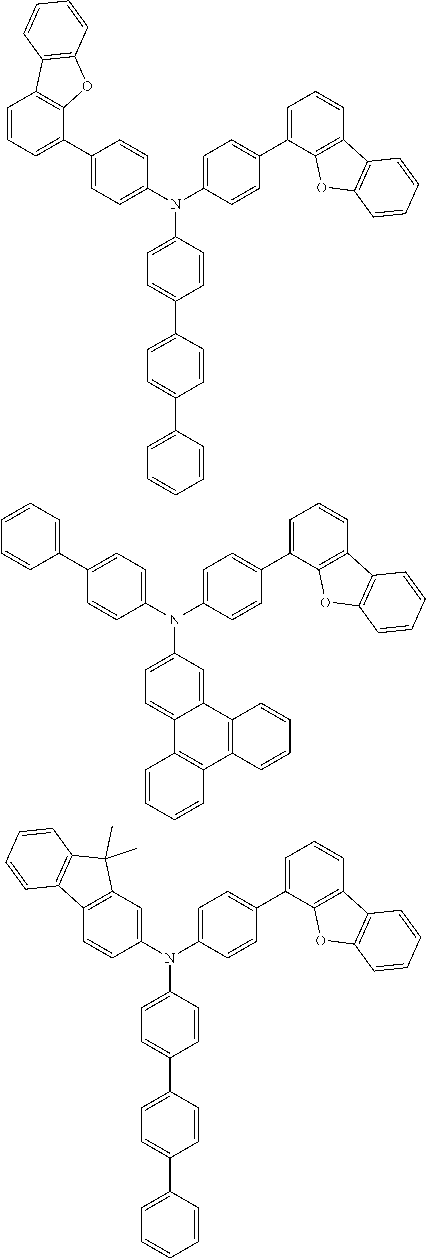

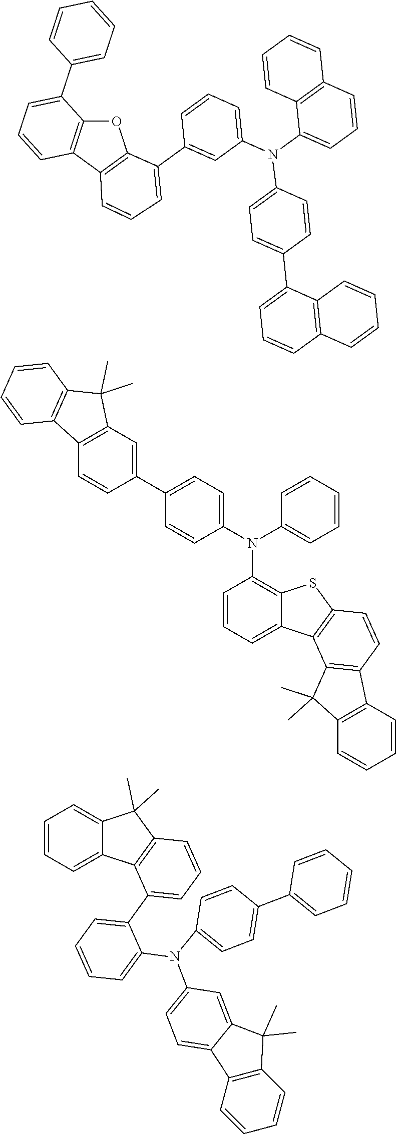 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

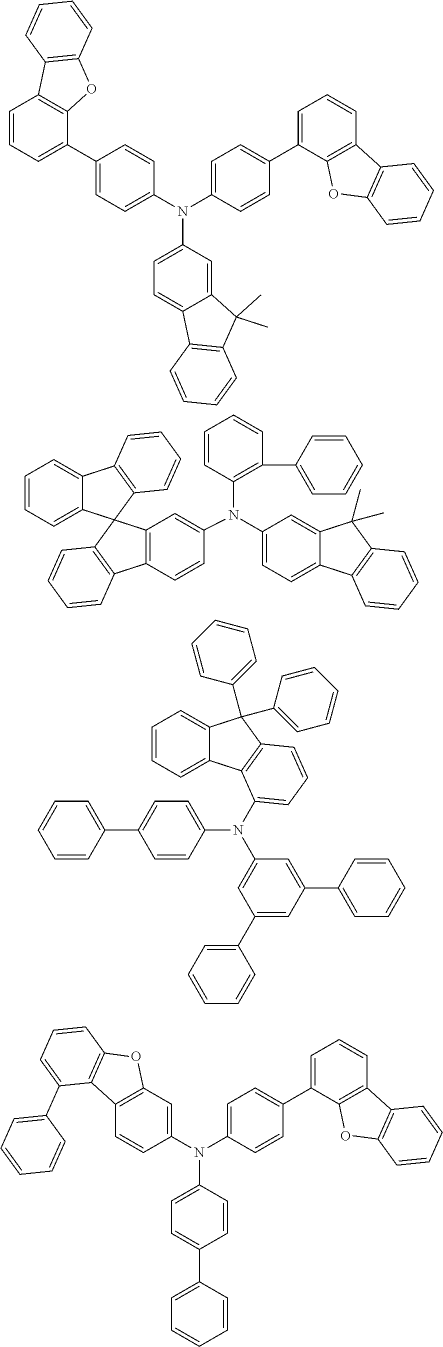

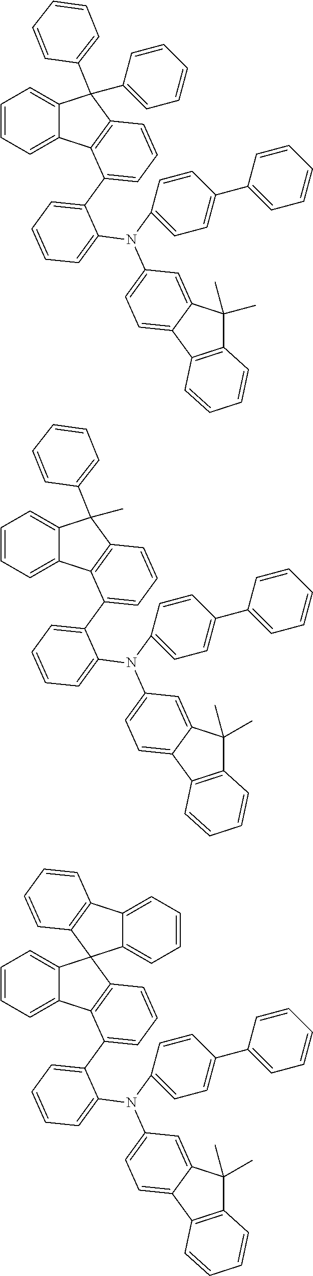 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

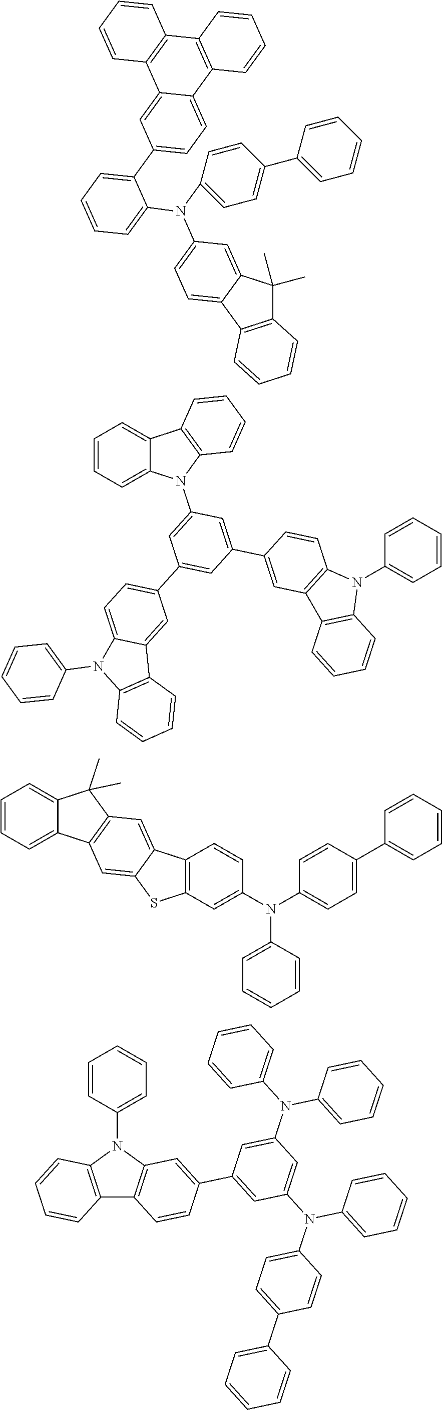

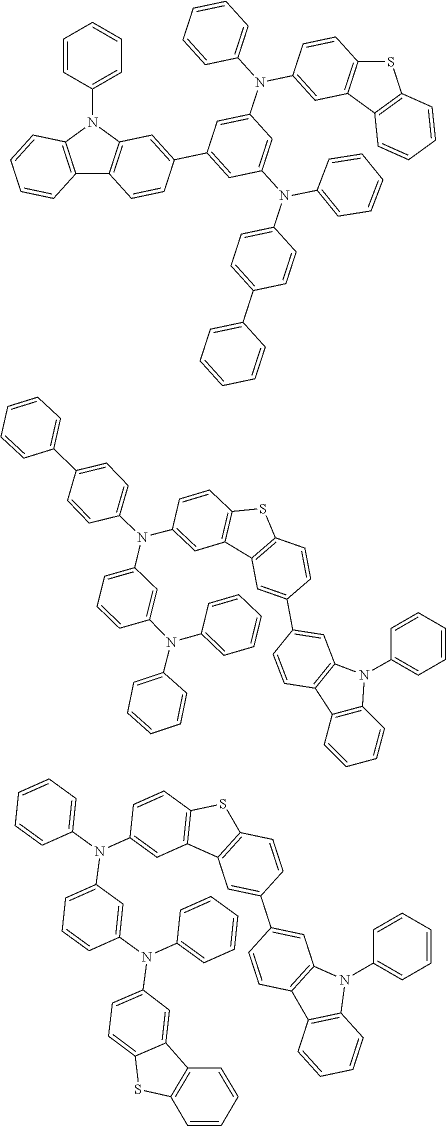 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

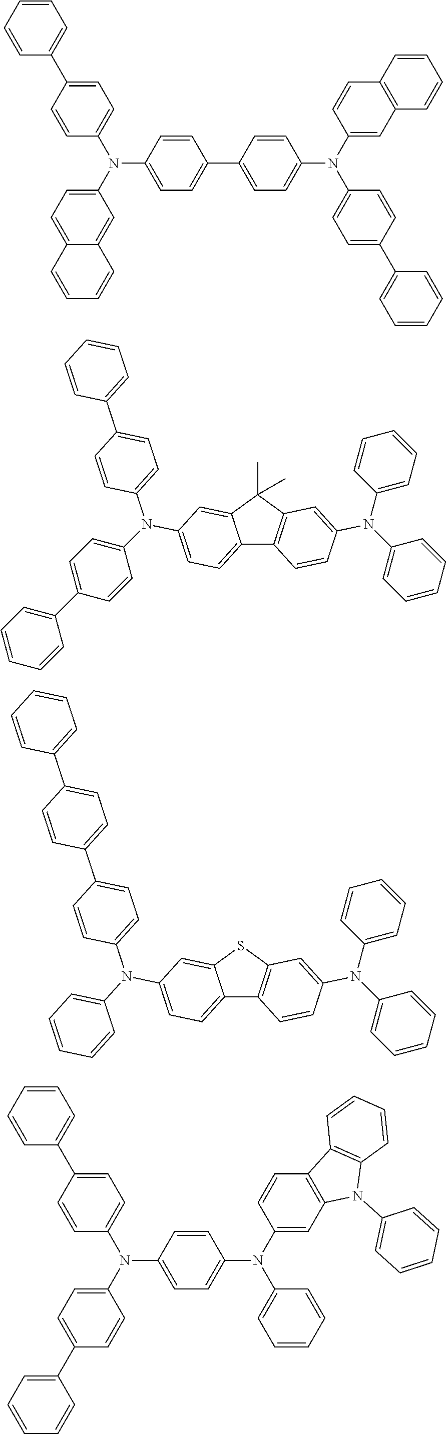

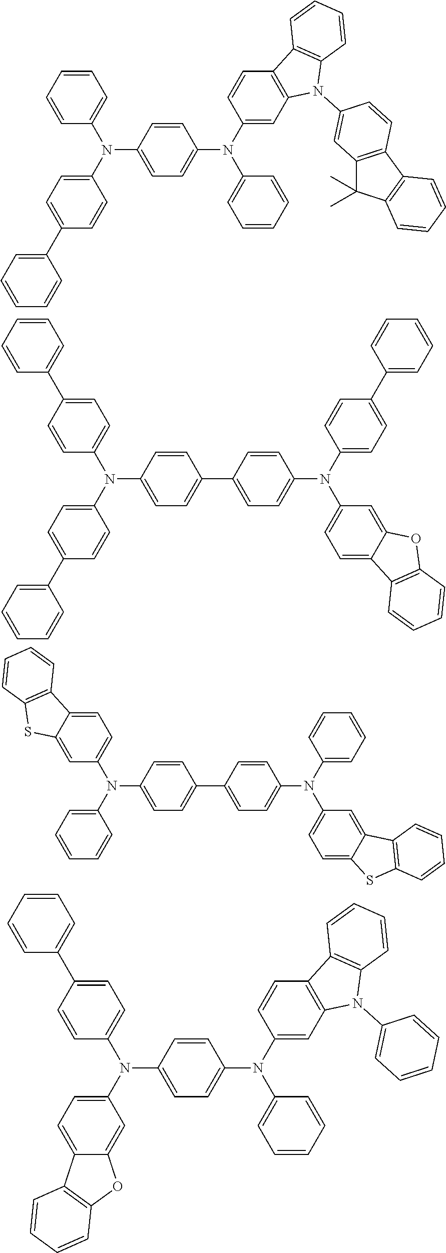

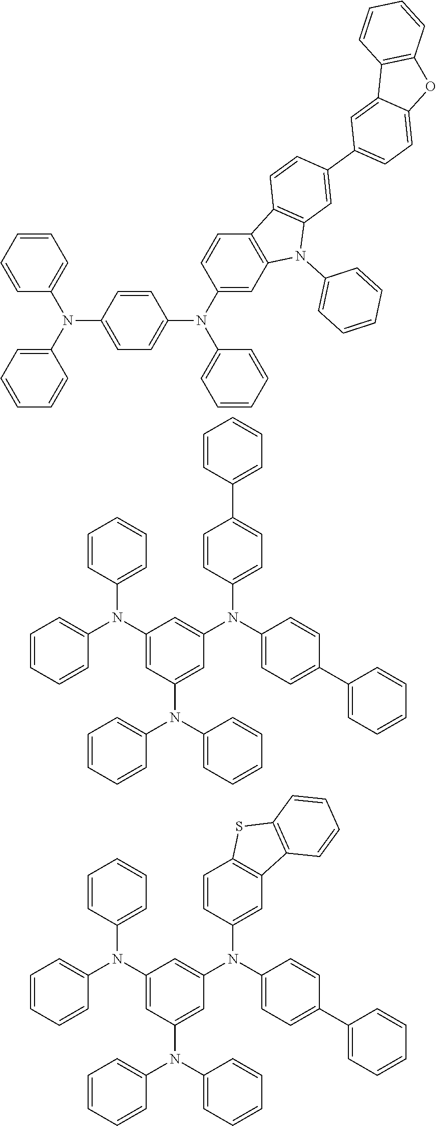

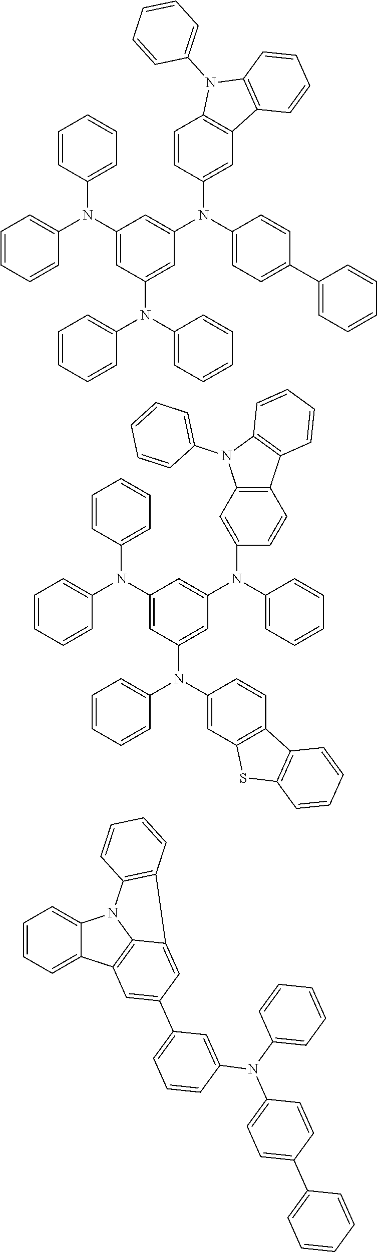

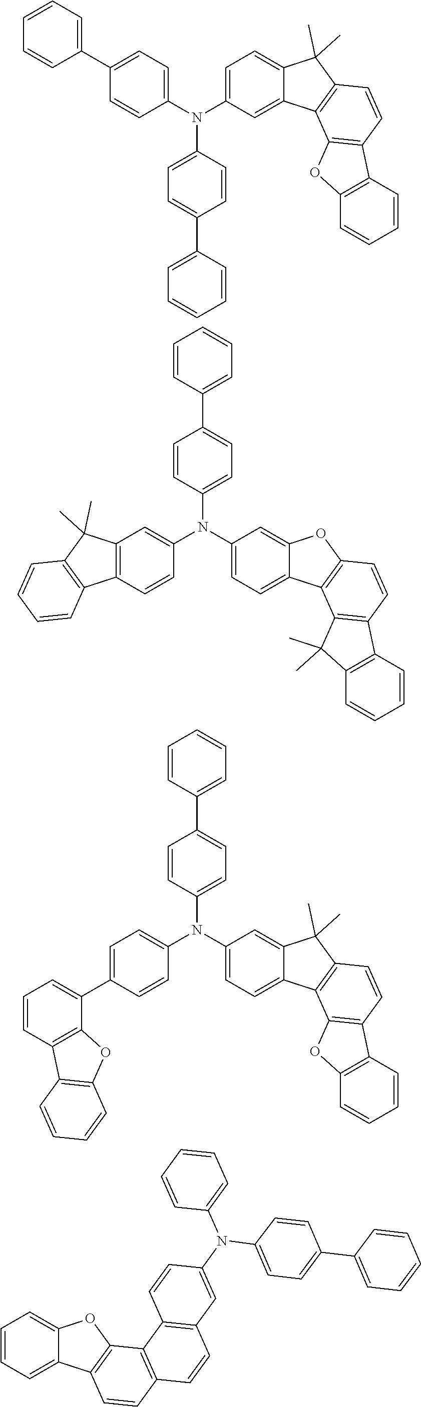

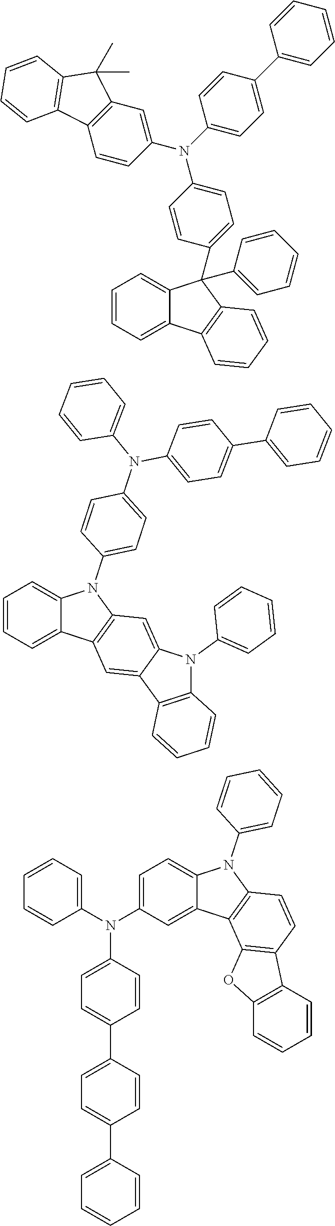

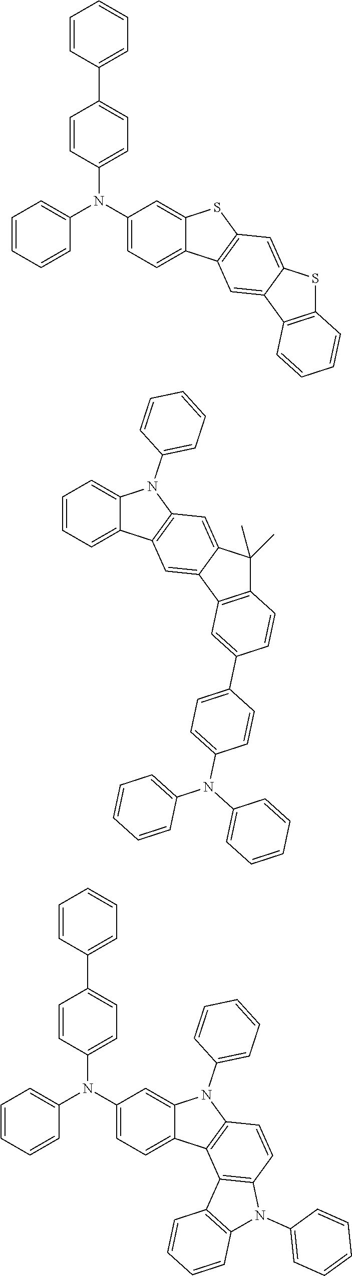 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

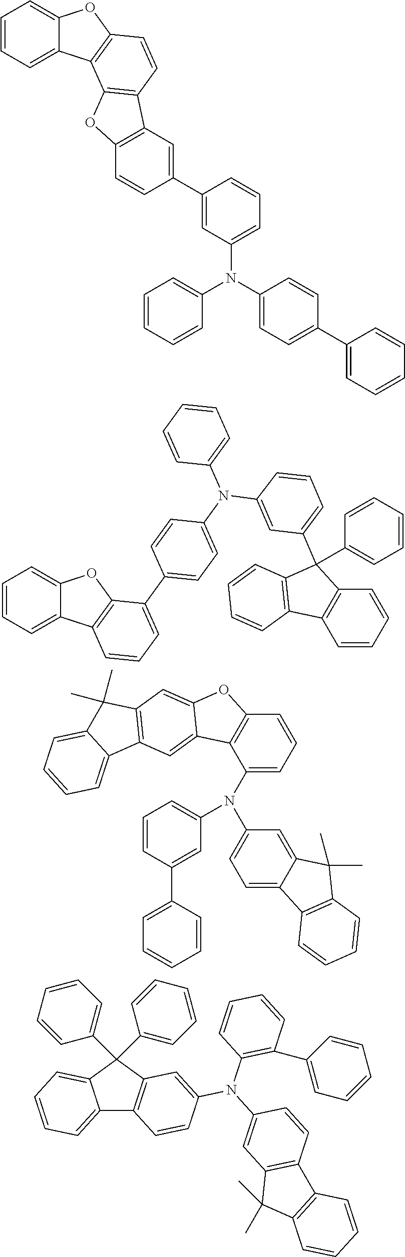

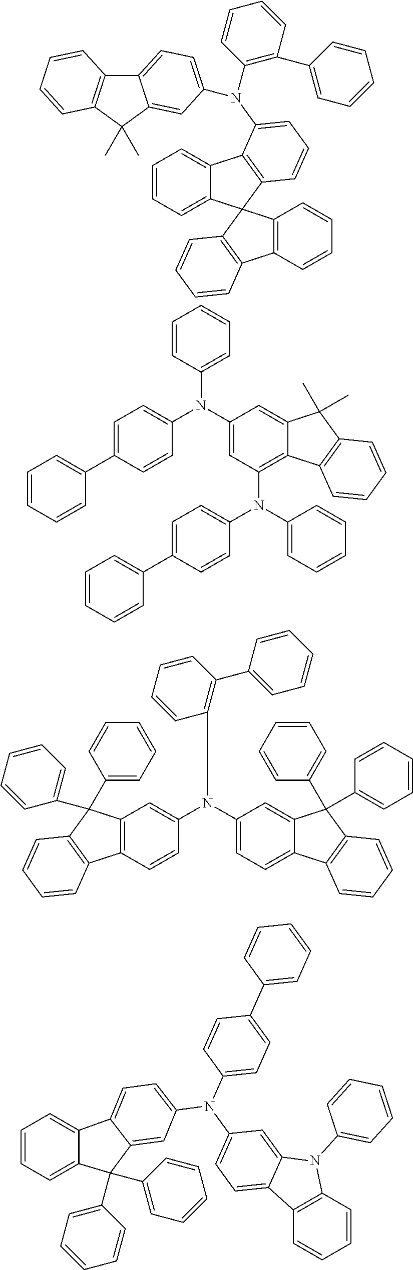

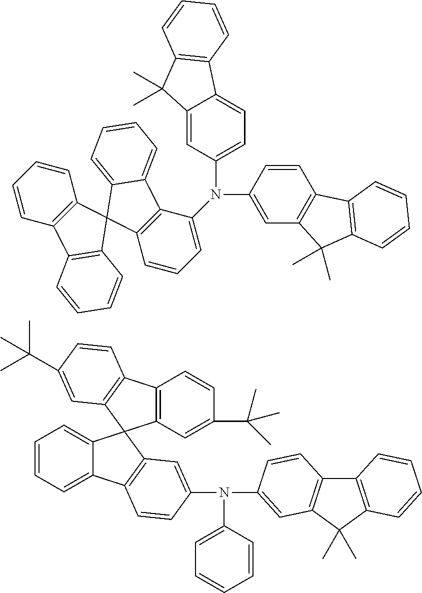

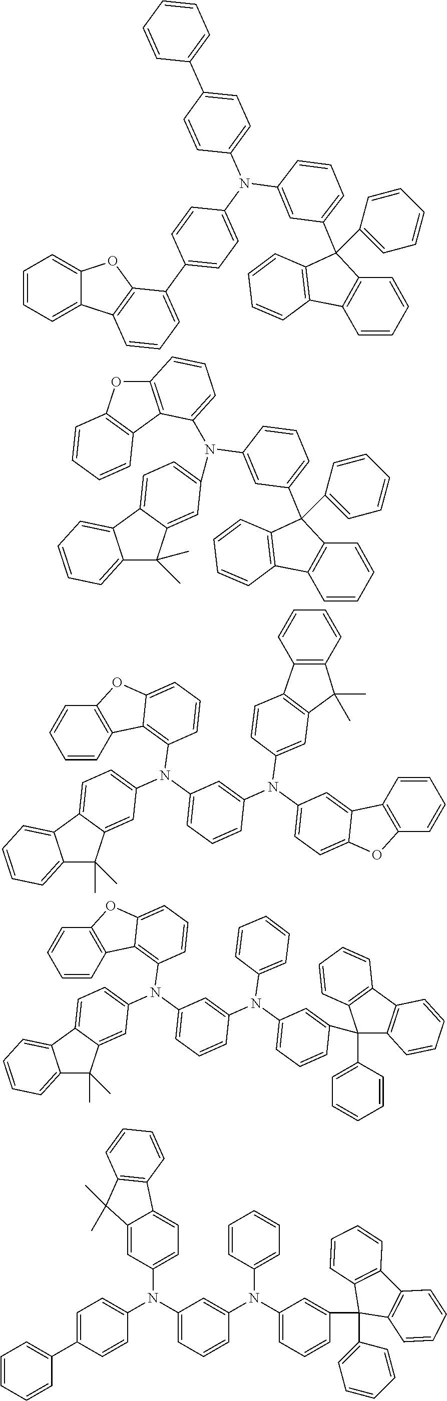

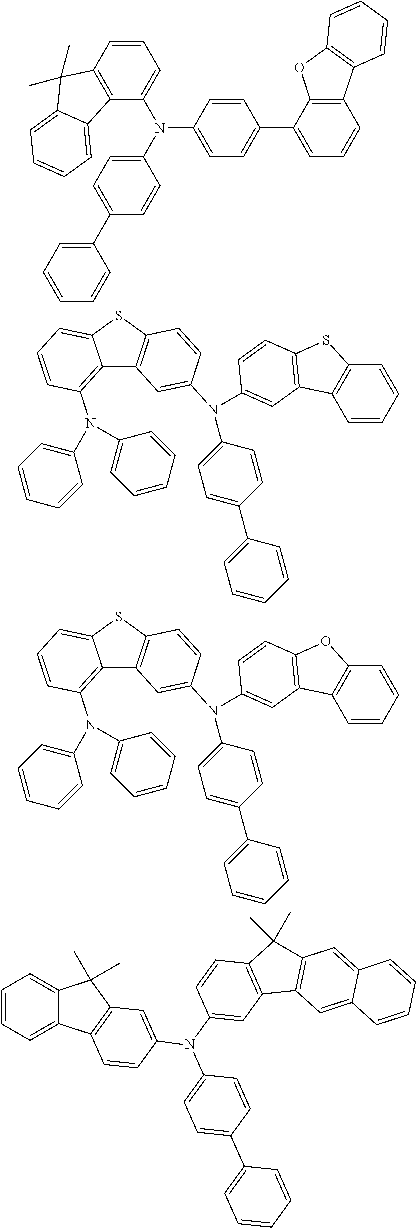

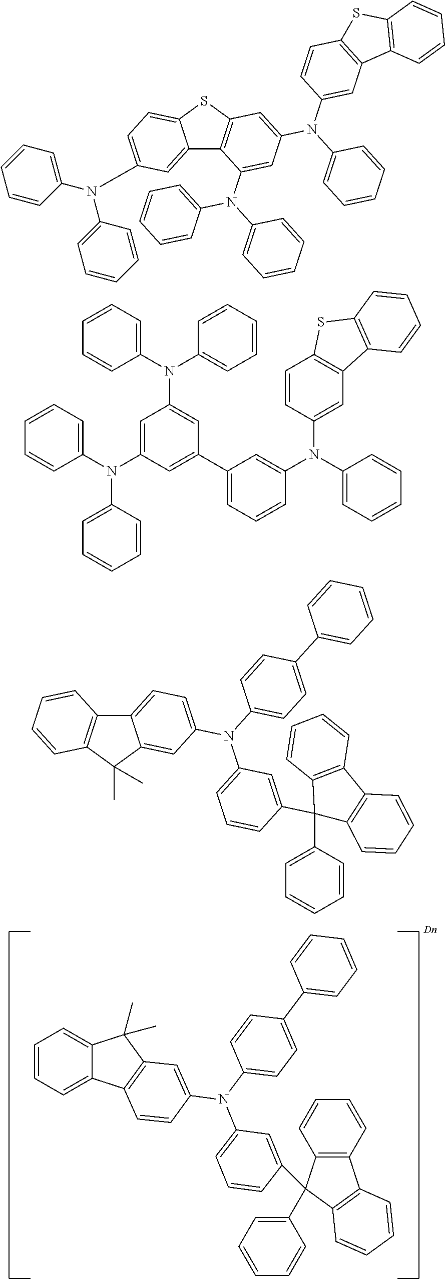

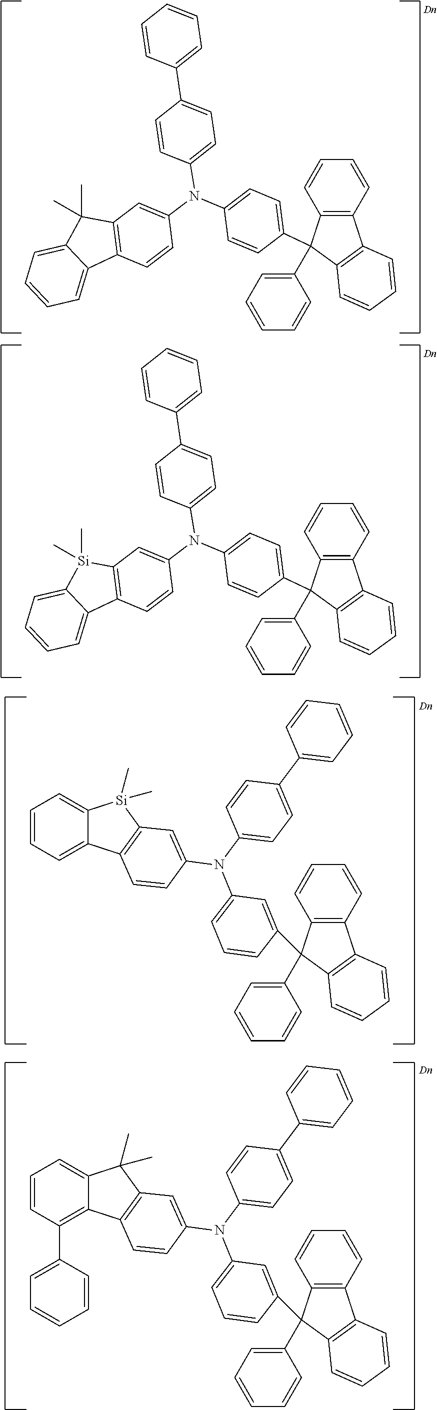

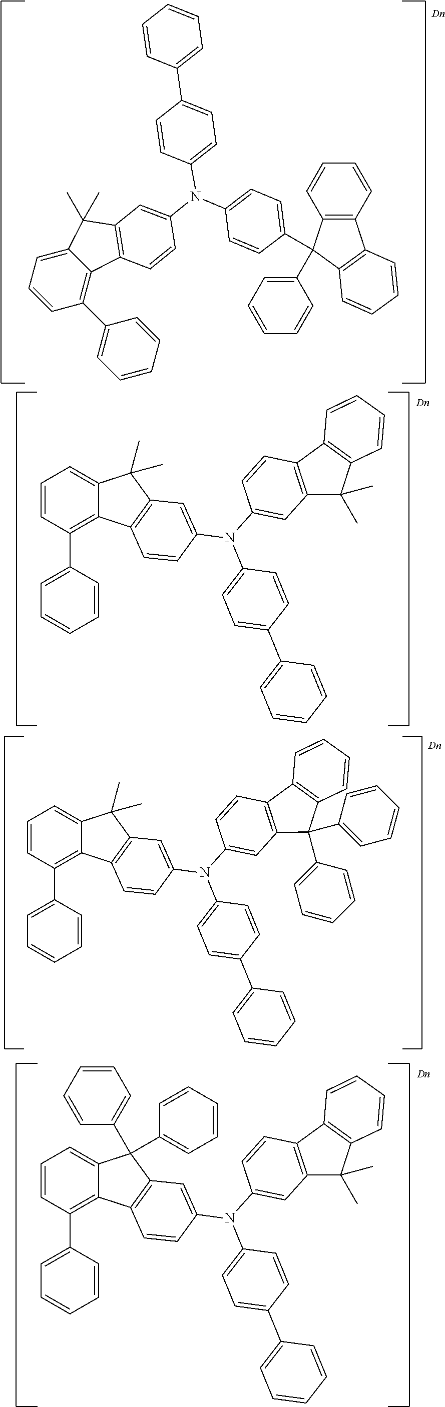

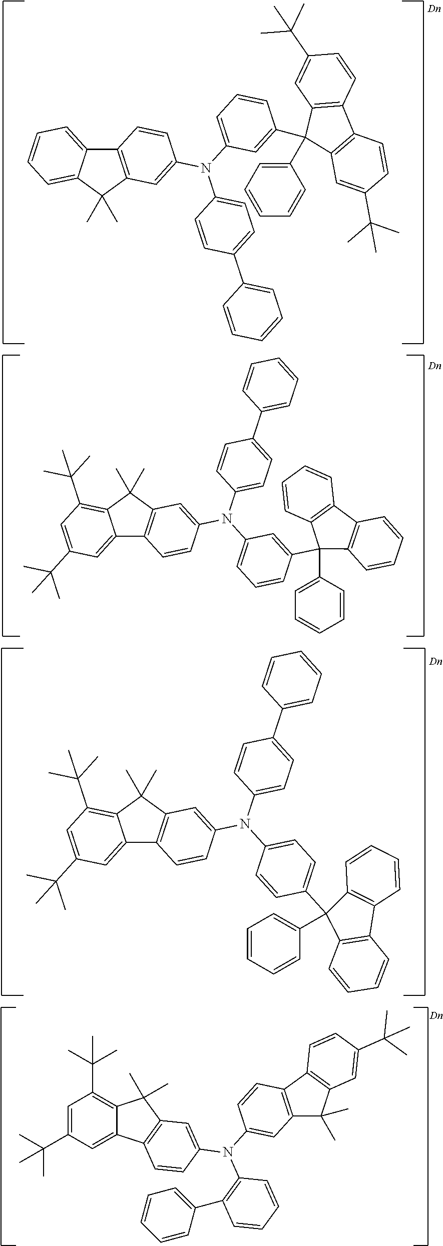

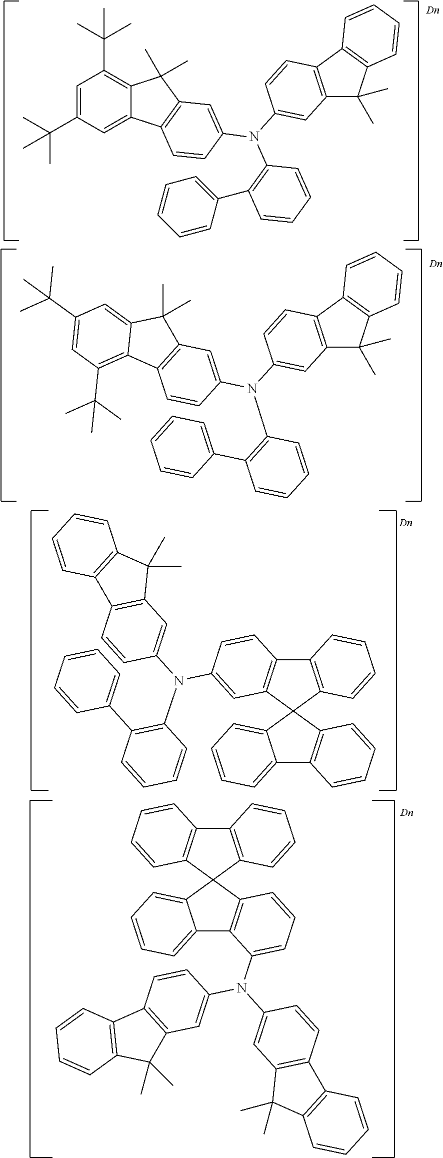

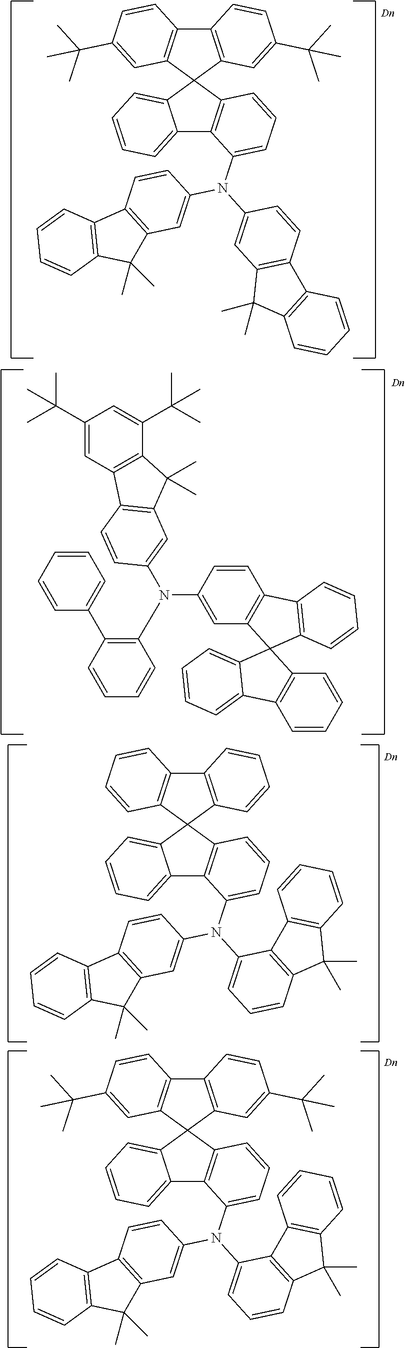

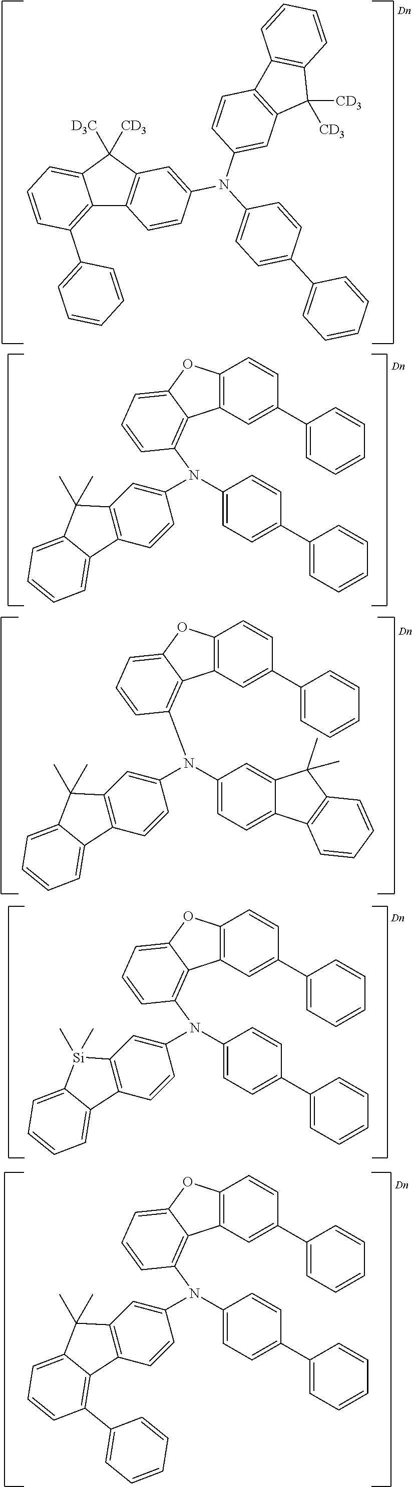

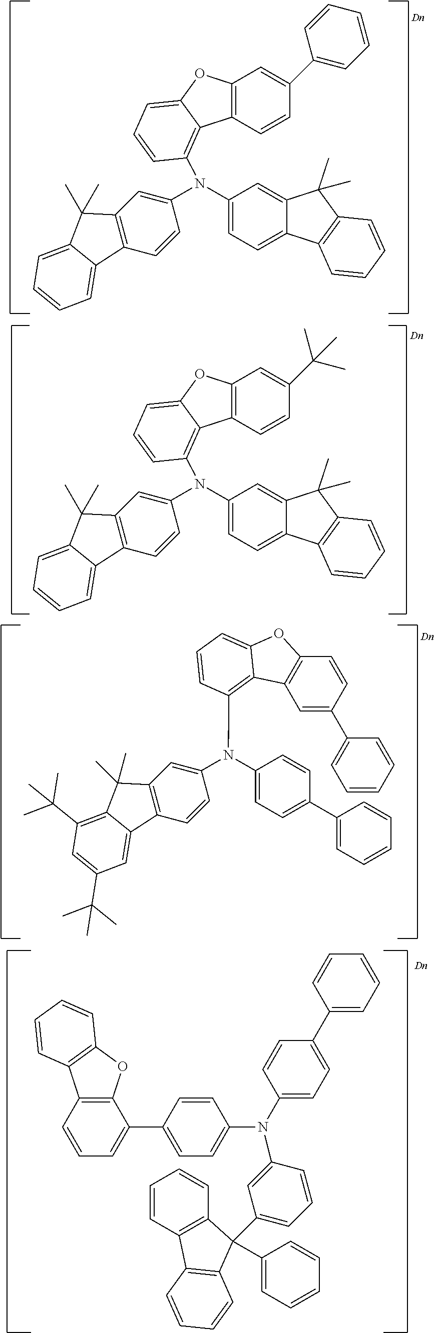

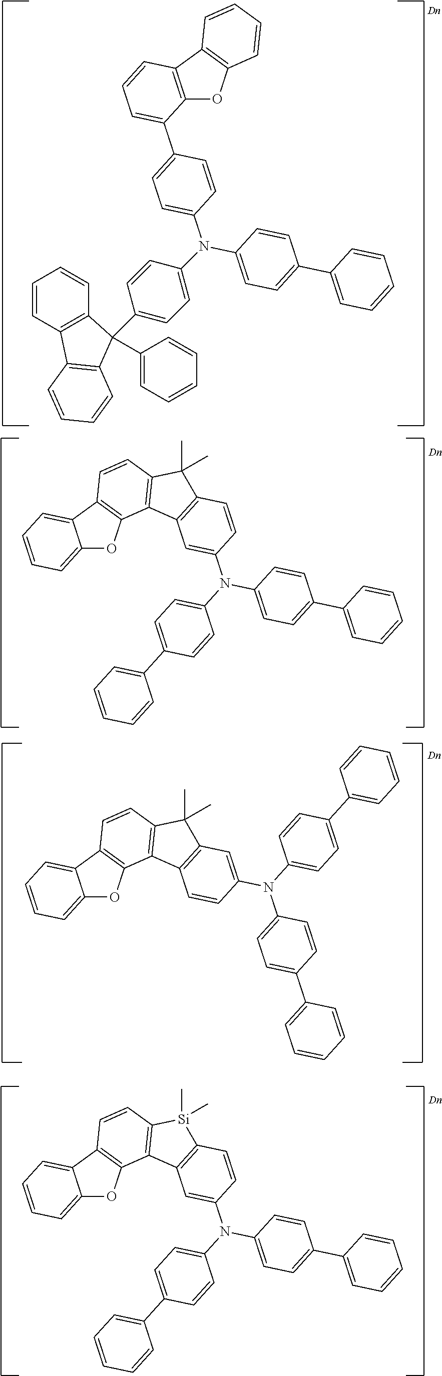

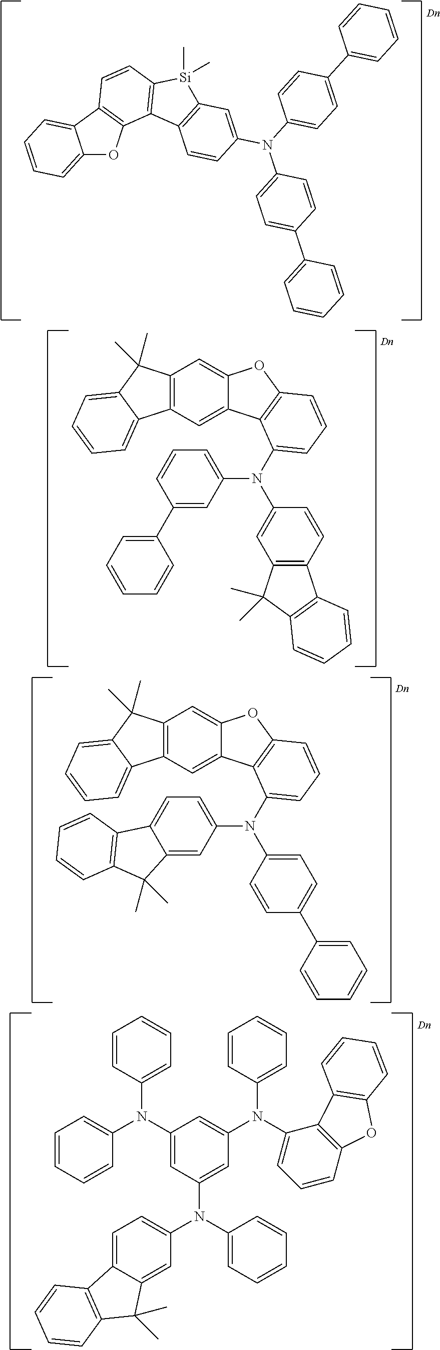

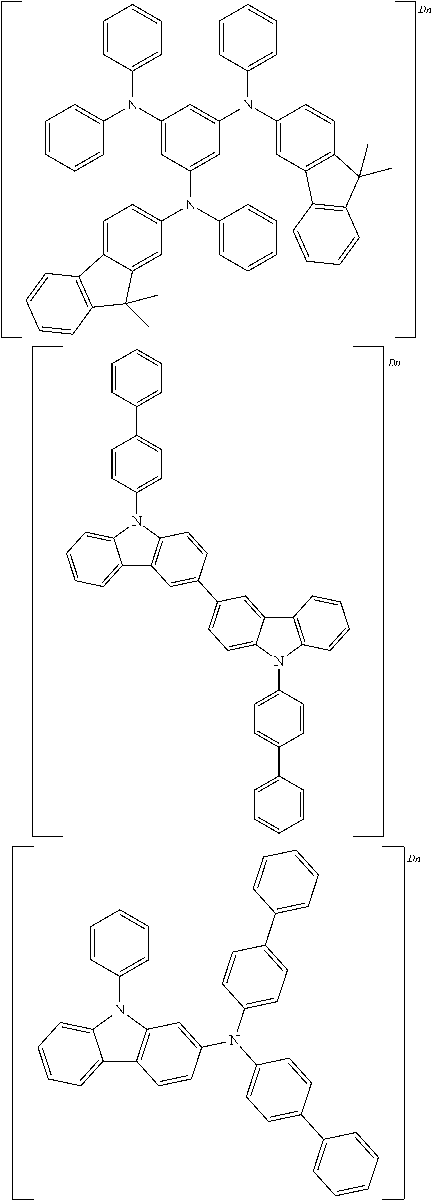

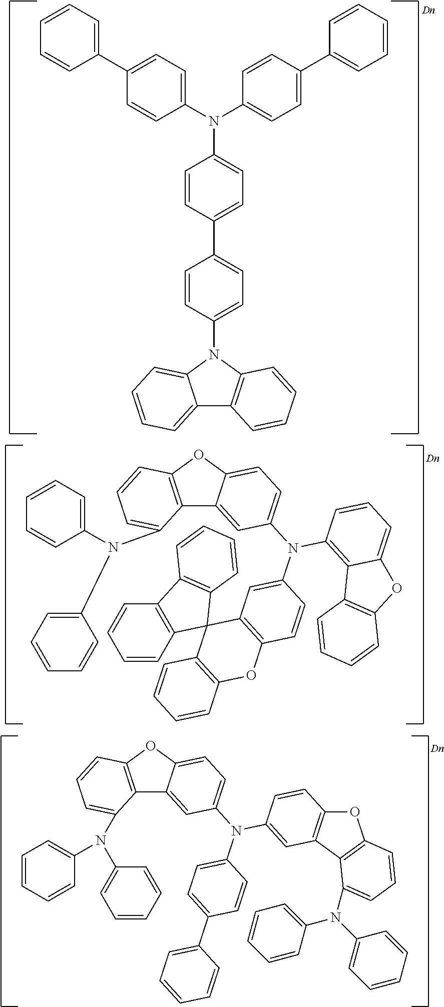

(Dn refers to the number of deuterium atoms substituted and indicates a structure in which one or more deuterium atoms are substituted)

In the hole transport region 140, in addition to the compounds described above, known compounds disclosed in other suitable compounds having a similar structure may also be used.

In an implementation, the charge transport region may be, e.g., the electron transport region 150.

The electron transport region 150 may help further increase electron injection or electron mobility and help block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

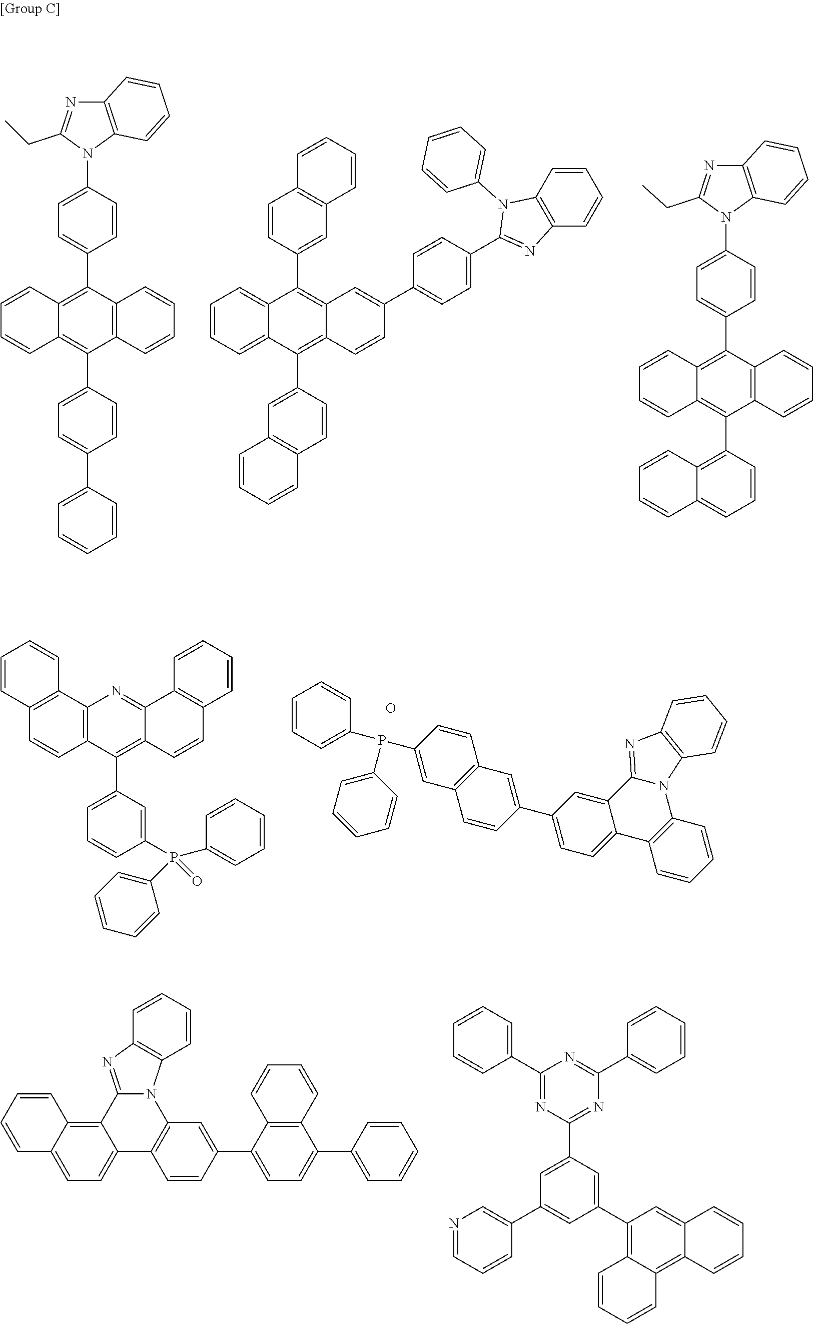

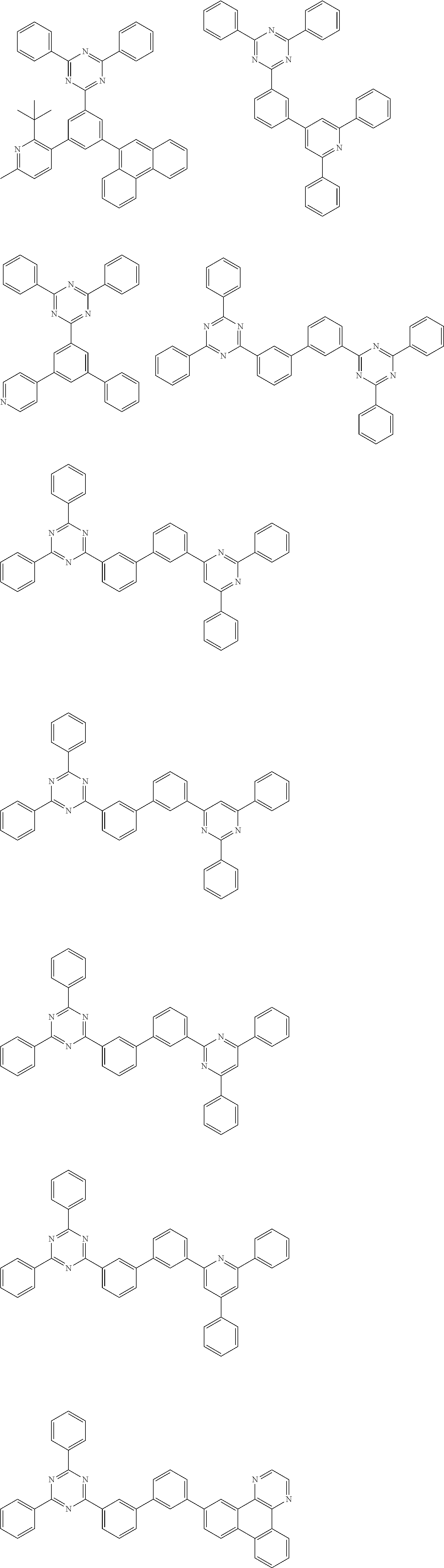

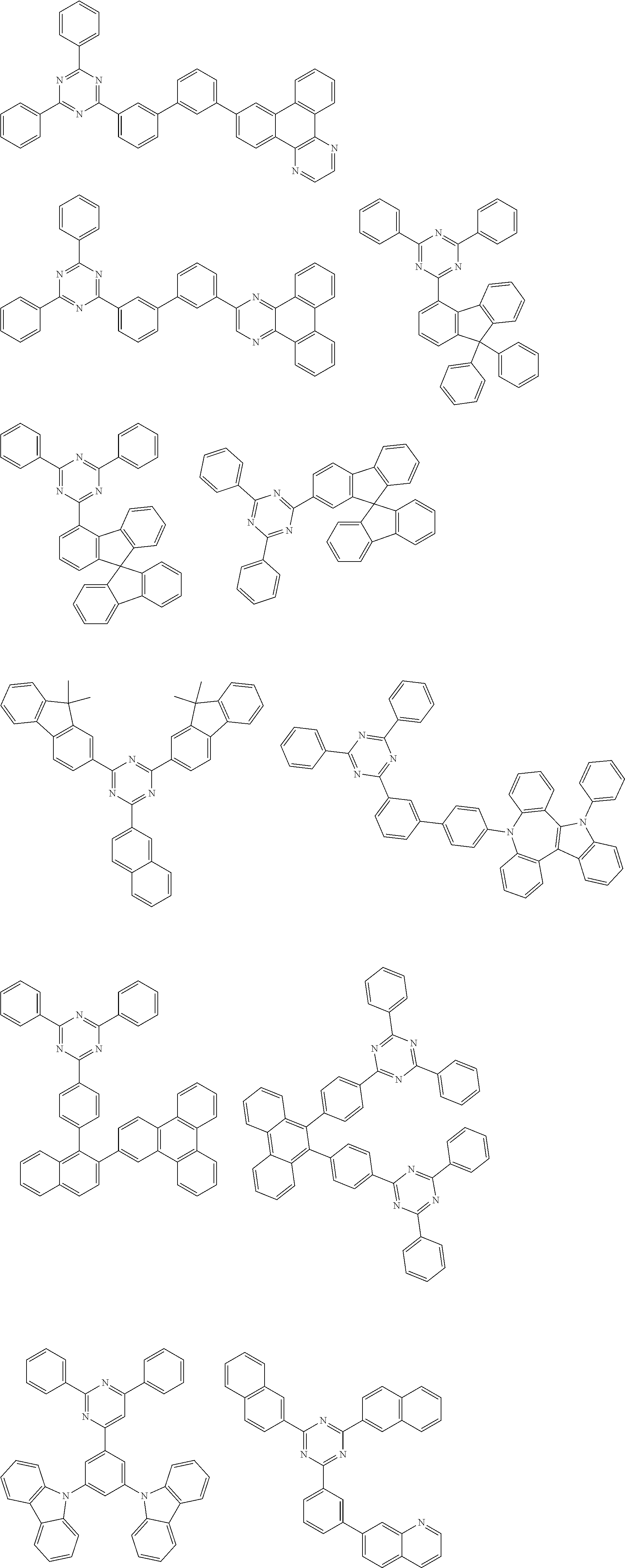

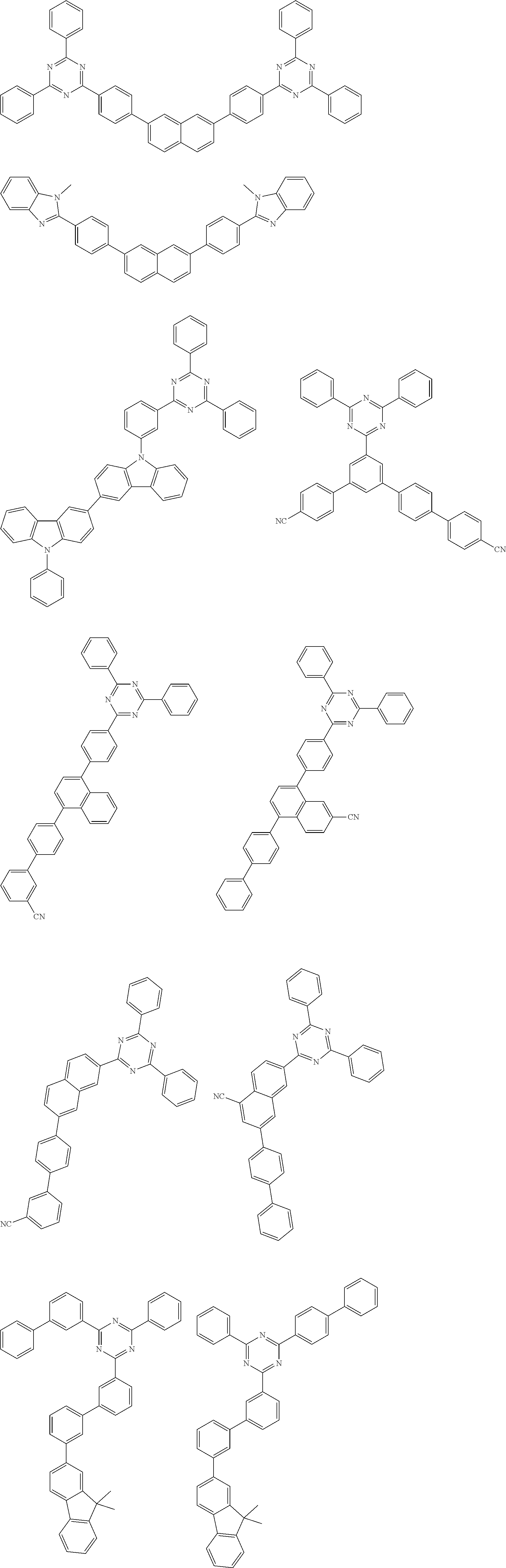

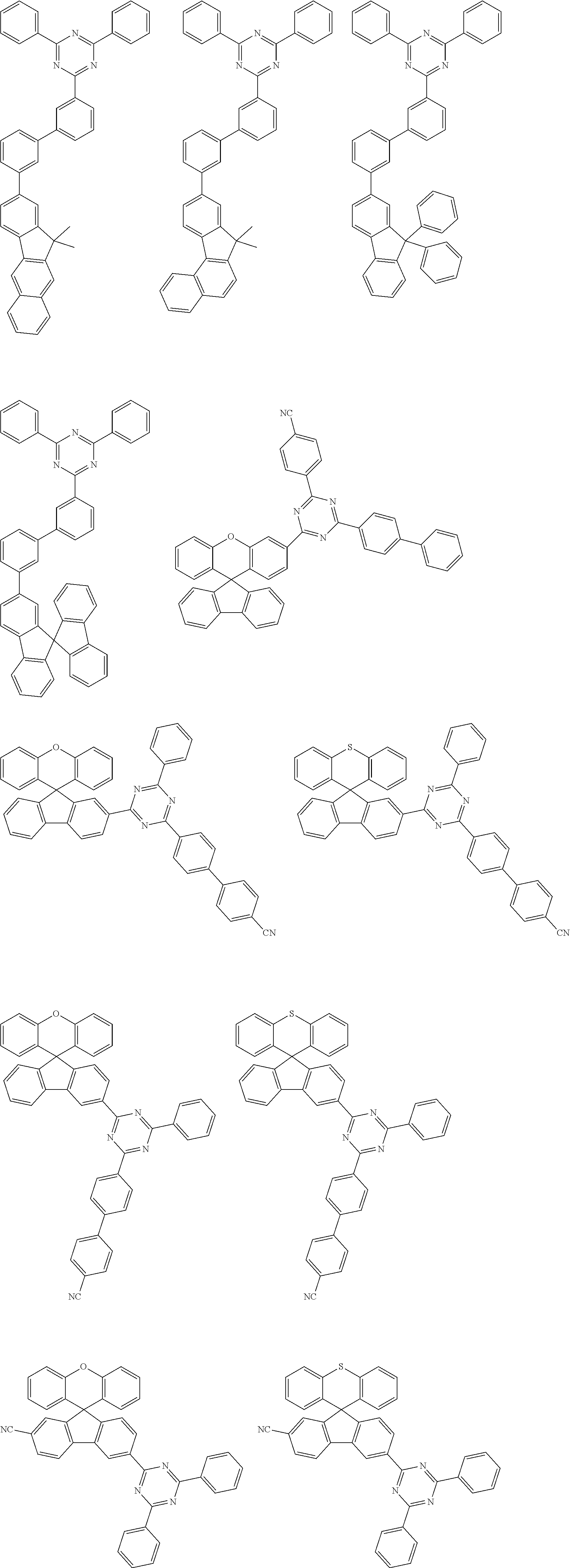

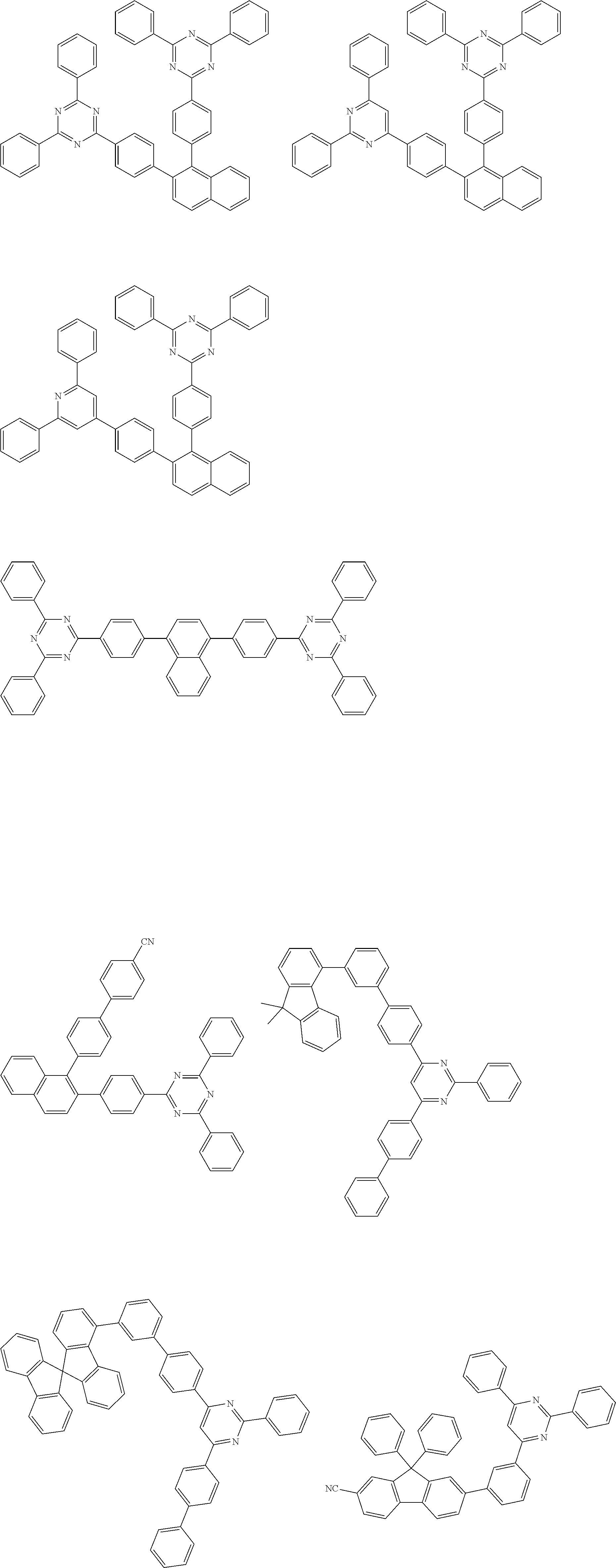

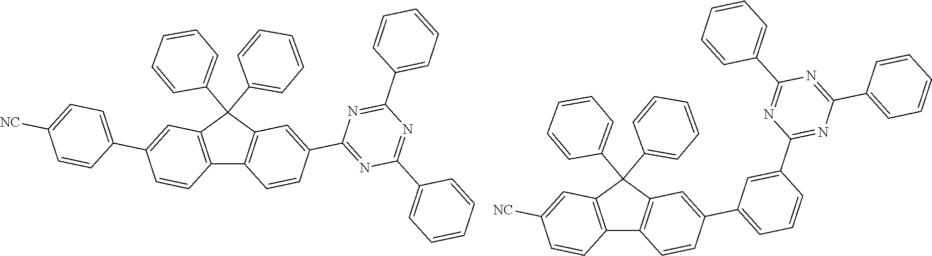

Some example embodiments may be an organic light emitting diode including the light emitting layer as the organic layer.

Some example embodiments may be an organic light emitting diode including a light emitting layer and a hole transport region as the organic layer.

Some example embodiments may be an organic light emitting diode including a light emitting layer and an electron transport region as the organic layer.

An organic light emitting diode according to some example embodiments may include, e.g., a hole transport region 140 and an electron transport region 150 in addition to the light emitting layer 130 as the organic layer 105, as shown in the FIGURE.

In an implementation, an organic light emitting diode may further include, e.g., an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method, e.g., vacuum deposition, sputtering, plasma plating and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### (Synthesis of Compound for Organic Optoelectronic Device)

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by suitable methods.

### Synthesis Example 1: Synthesis of Compound A-1

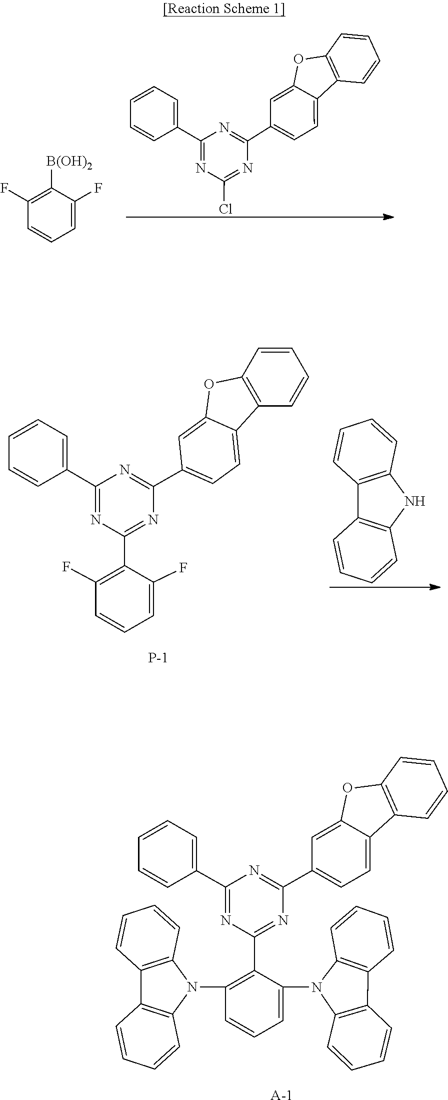

### 1st Step: Synthesis of Intermediate P-1

2-chloro-4-(dibenzo[b,d]furan-3-yl)-6-phenyl-1,3,5-triazine (50 g/1.0 eq.), (2,6-difluorophenyl)boronic acid (1.1 eq.), Pd(PPh₃)₄ (0.05 eq.), and K₂CO₃ (3.0 eq.) along with tetrahydrofuran (THF) (750 mL) and distilled water (250 mL) were added to a flask and then, refluxed at 80° C. After 12 hours, a reaction was terminated, and a resultant therefrom was diluted with dichloromethane (DCM), three times washed with brine, and dried with MgSO₄. Subsequently, column chromatography was performed to obtain 30 g of Intermediate P-1.

### 2nd Step: Synthesis of Compound A-1

Intermediate P-1 (10 g/1.0 eq.), 9H-carbazole (2.5 eq.), and K₃PO₄ (3.0 eq.) along with dimethylformamide (DMF) (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 7 g of Compound A-1.

### Synthesis Example 2: Synthesis of Compound A-33

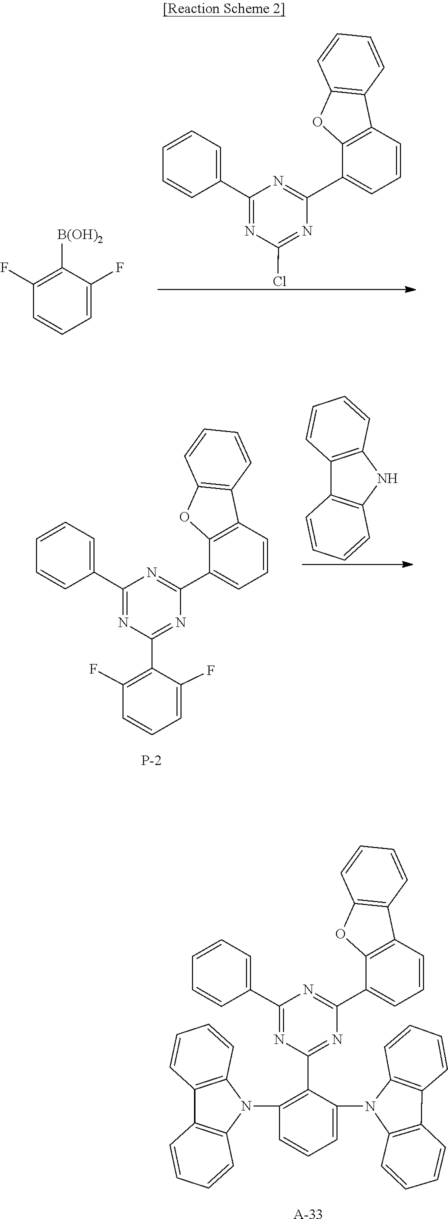

### 1st Step: Synthesis of Intermediate P-2

2-chloro-4-(dibenzo[b,d]furan-4-yl)-6-phenyl-1,3,5-triazine (30 g/1.0 eq.), (2,6-difluorophenyl)boronic acid (1.1 eq.), Pd(PPh₃)₄ (0.05 eq.), and K₂CO₃ (3.0 eq.) along with THF (750 mL) and distilled water (250 mL) were added to a flask and then, refluxed at 80° C. After 12 hours, a reaction was terminated, and a resultant therefrom was diluted with DCM, three times washed with brine, and dried with MgSO₄. Subsequently, column chromatography was performed to obtain 15 g of Intermediate P-2.

### 2nd Step: Synthesis of Compound A-33

Intermediate P-2 (15 g/1.0 eq.), 9H-carbazole (2.5 eq.), and K₃PO₄ (3.0 eq.) along with DMF (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 9 g of Compound A-33.

### Synthesis Example 3: Synthesis of Compound A-49

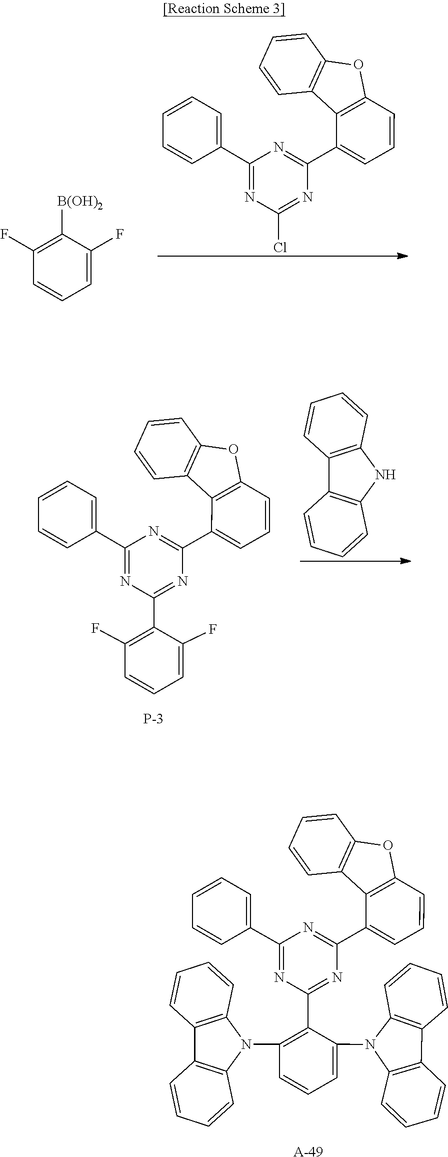

### 1st Step: Synthesis of Intermediate P-3

2-chloro-4-(dibenzo[b,d]furan-1-yl)-6-phenyl-1,3,5-triazine (30 g/1.0 eq.), (2,6-difluorophenyl)boronic acid (1.1 eq.), Pd(PPh₃)₄ (0.05 eq.), and K₂CO₃ (3.0 eq.) along with THF (750 mL) and distilled water (250 mL) were added to a flask and then, refluxed at 80° C. After 12 hours, a reaction was terminated, and a resultant therefrom was diluted with DCM, three times washed with brine, and dried with MgSO₄. Subsequently, column chromatography was performed to obtain 20 g of Intermediate P-3.

### 2nd Step: Synthesis of Compound A-49

Intermediate P-3 (20 g/1.0 eq.), 9H-carbazole (2.5 eq.), and K₃PO₄ (3.0 eq.) along with DMF (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 13 g of Compound A-49.

### Synthesis Example 4: Synthesis of Compound A-70

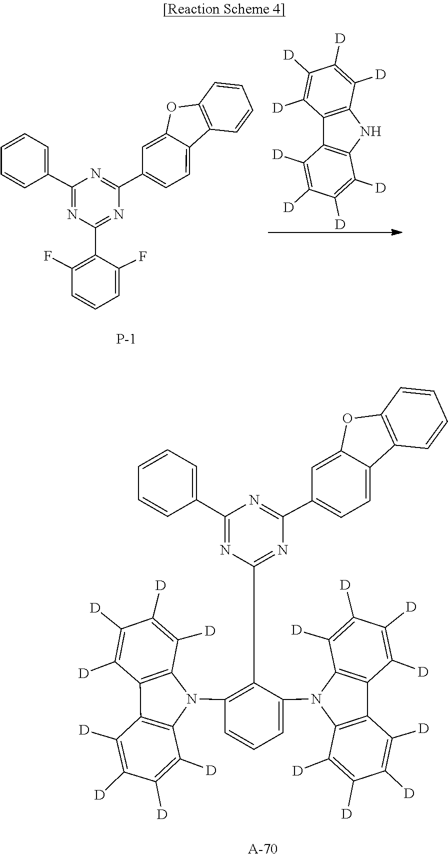

Intermediate P-1 (10 g/1.0 eq.), 9H-carbazole-d8 (2.5 eq.), and K₃PO₄ (3.0 eq.) along with DMF (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 6 g of Compound A-70.

### Synthesis Example 5: Synthesis of Compound C-4

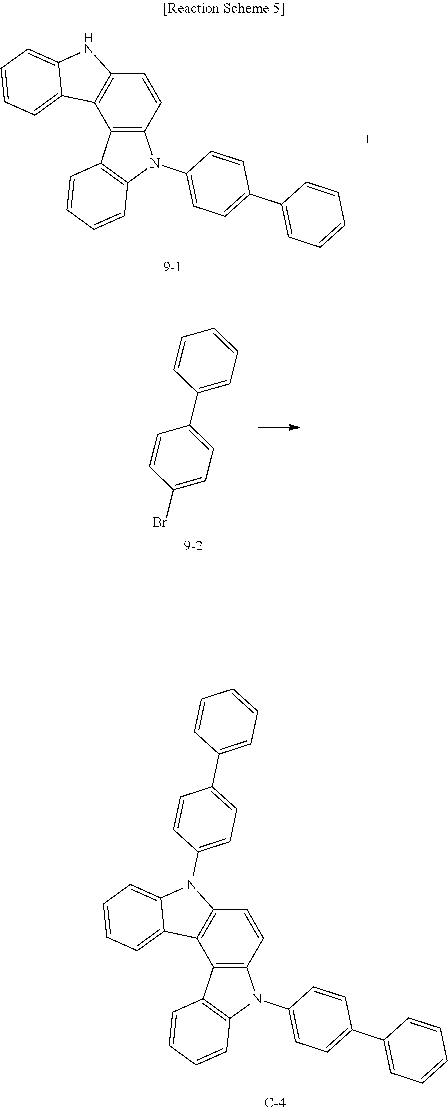 [SMILES: c1ccc(-c2ccc(-n3c4ccccc4c4c5c(ccc43)[nH]c3ccccc35)cc2)cc1 ; Brc1ccc(-c2ccccc2)cc1 ; c1ccc(-c2ccc(-n3c4ccccc4c4c5c6ccccc6n(-c6ccc(-c7ccccc7)cc6)c5ccc43)cc2)cc1]

10.0 g (24.5 mmol) of Intermediate 9-1, 6.3 g (26.9 mmol) of Intermediate 9-2, 1.1 g (1.2 mmol) of Pd₂(dba)₃, 3.5 g (36.7 mmol) of NaOtBu, and 0.7 g (3.7 mmol) of P(t-Bu)₃ were added to a round-bottomed flask, and 122 m1 of xylene was added thereto and then, stirred under reflux at 140° C. for 12 hours. When a reaction was completed, after adding distilled water thereto and then, stirring the mixture, an organic layer obtained after removing an aqueous layer therefrom was filtered with silica gel and recrystallized to obtain 10.3 g (75%) of Compound C-4.

(LC/MS theoretical value: 560.23 g/mol, measured value: M+=561.54 g/mol)

### Synthesis Example 6: Synthesis of Compound B-136

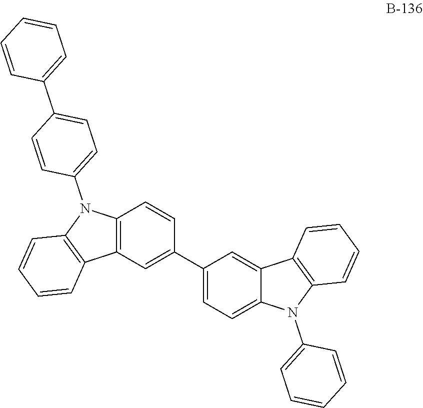 [SMILES: c1ccc(-c2ccc(-n3c4ccccc4c4cc(-c5ccc6c(c5)c5ccccc5n6-c5ccccc5)ccc43)cc2)cc1]

Compound B-136 was obtained by purchase from GemChem Inc.

HRMS (70 eV, EI+): m/z calcd for C42H28N2: 560.2252, found: 560.

Elemental Analysis: C, 90%; H, 5%

### Synthesis Example 7: Synthesis of Compound R-1

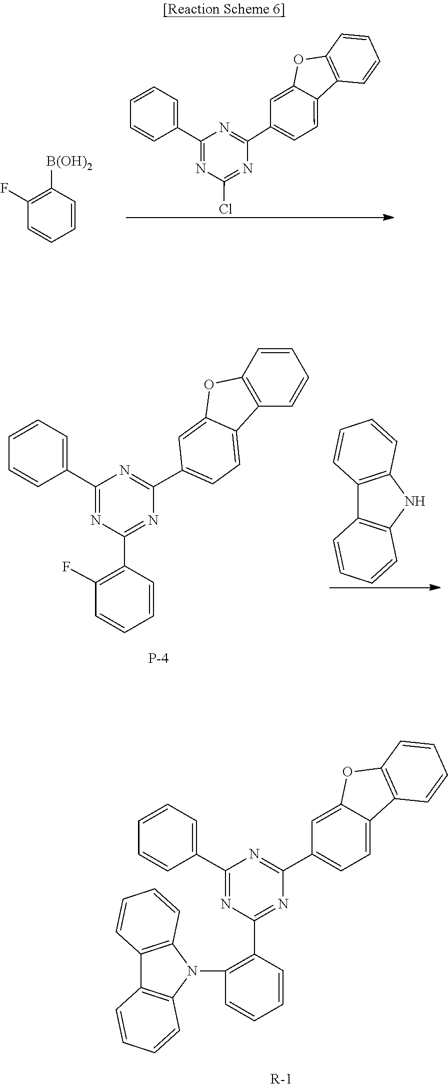

### 1st Step: Synthesis o Intermediate P-4

2-chloro-4-(dibenzo[b,d]furan-3-yl)-6-phenyl-1,3,5-triazine (20 g/1.0 eq.), (2-fluorophenyl)boronic acid (1.1 eq.), Pd(PPh₃)₄ (0.05 eq.), and K₂CO₃ (3.0 eq.) along with THF (750 mL) and distilled water (250 mL) were added to a flask and then, refluxed at 80° C. After 12 hours, a reaction was terminated, and a resultant therefrom was diluted with DCM, three times washed with brine, and dried with MgSO₄. Subsequently, column chromatography was performed to obtain 15 g of Intermediate P-4.

### 2nd Step: Synthesis of Compound R-1

Intermediate P-4 (15 g/1.0 eq.), 9H-carbazole (1.2 eq.), and K₃PO₄ (2.0 eq.) along with DMF (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 12 g of Compound R-1.

### Synthesis Example 8: Synthesis of Compound R-2

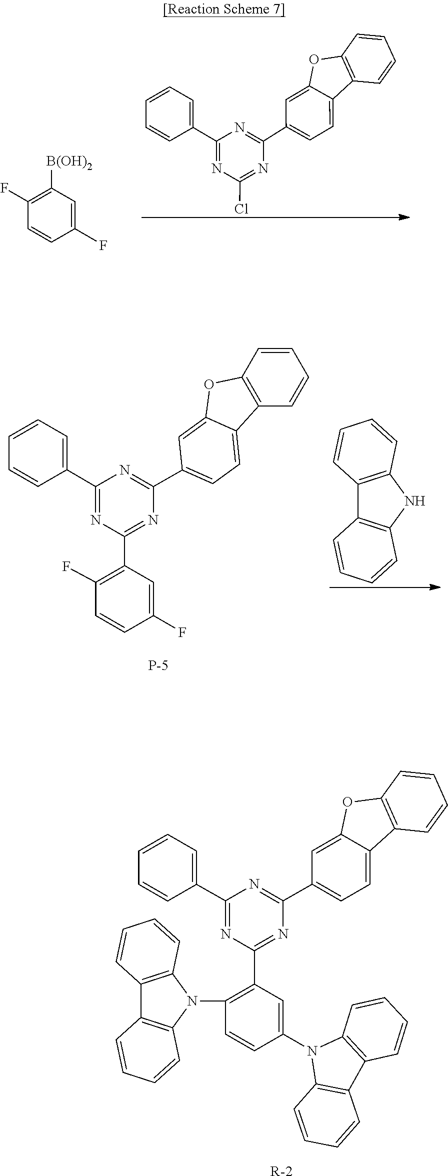

### 1 st Step: Synthesis of Intermediate P-5

2-chloro-4-(dibenzo[b,d]furan-3-yl)-6-phenyl-1,3,5-triazine (20 g/1.0 eq.), (2,5-difluorophenyl)boronic acid (1.1 eq.), Pd(PPh₃)₄ (0.05 eq.), and K₂CO₃ (3.0 eq.) along with THF (750 mL) and distilled water (250 mL) were added to a flask and then, refluxed at 80° C. After 12 hours, a reaction was terminated, and a resultant therefrom was diluted with DCM, three times washed with brine, and dried with MgSO₄. Subsequently, column chromatography was performed to obtain 16 g of Intermediate P-5.

### 2nd Step: Synthesis of Compound R-2

Intermediate P-5 (16 g/1.0 eq.), 9H-carbazole (2.5 eq.), and K₃PO₄ (3.0 eq.) along with DMF (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 10 g of Compound R-2.

### Synthesis Example 9: Synthesis of Compound R-3

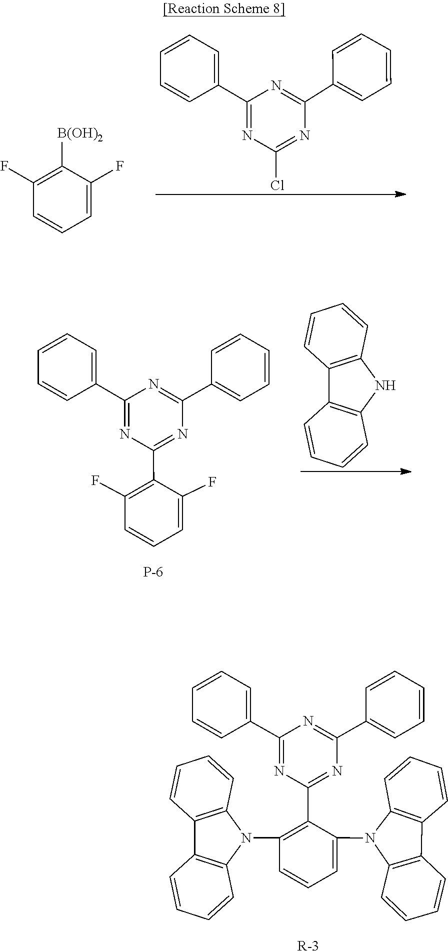

### 1st Step: Synthesis of Intermediate P-6

2-chloro-4,6-diphenyl-1,3,5-triazine (30 g/1.0 eq.), (2,6-difluorophenyl)boronic acid (1.1 eq.), Pd(PPh₃)₄ (0.05 eq.), and K₂CO₃ (3.0 eq.) along with THF (750 mL) and distilled water (250 mL) were added to a flask and then, refluxed at 80° C. After 12 hours, a reaction was terminated, and a resultant therefrom was diluted with DCM, three times washed with brine, and dried with MgSO₄. Subsequently, column chromatography was performed to obtain 14 g of Intermediate P-6.

### 2nd Step: Synthesis of Compound R-3

Intermediate P-6 (14 g/1.0 eq.), 9H-carbazole (2.5 eq.), and K₃PO₄ (3.0 eq.) along with DMF (200 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 7 g of Compound R-3.

### Synthesis Example 10: Synthesis of Compound R-4

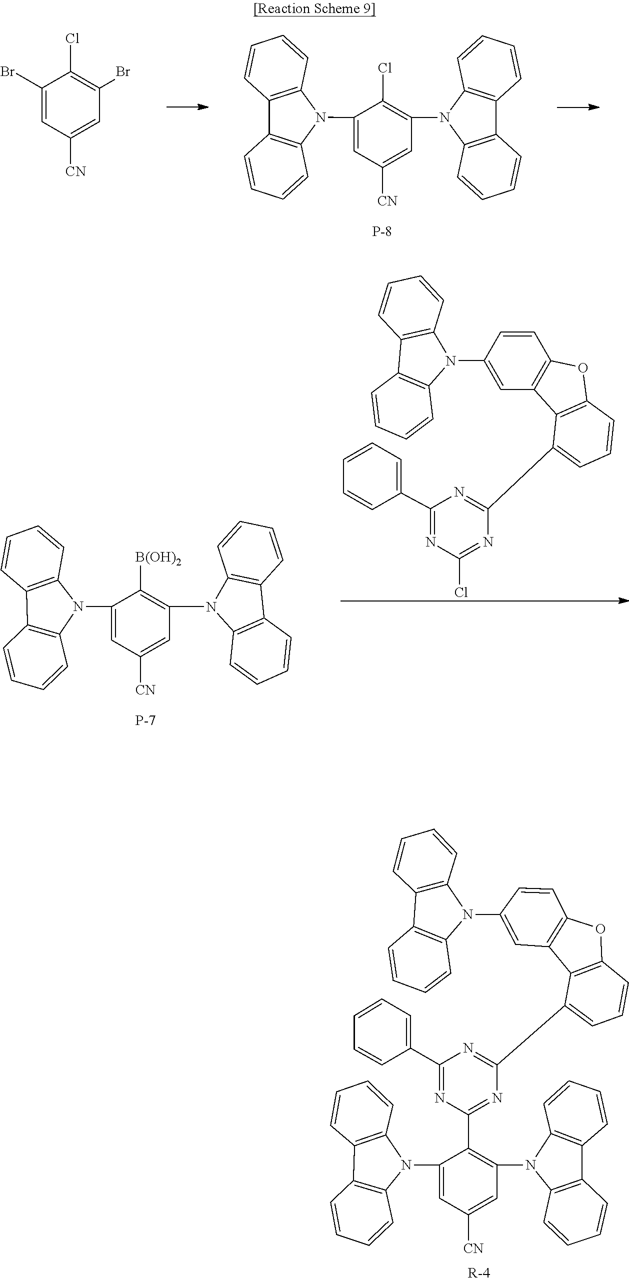

### 1st Step: Synthesis of Intermediate P-8

3,5-dibromo-4-chlorobenzonitrile (30 g/1.0 eq.), 9H-carbazole (2.5 eq.), Pd(OAc)₂ (0.05 eq.), P(t-Bu)₃ (0.2 eq.), and K₂CO₃ (3.0 eq.) along with xylene (400 mL) were added to a flask and then, refluxed at 150° C. After 12 hours, a reaction was terminated, and a resultant therefrom was diluted with DCM, three times washed with brine, and dried with MgSO₄. Subsequently, column chromatography was performed to obtain 24 g of Compound P-8.

### 2nd Step: Synthesis of Intermediate P-7

Intermediate P-8 (24 g/1.0 eq.) along with THF (300 mL) was added to a flask and then, stirred by cooling to −78° C. under N₂ atmosphere. After 30 minutes, t-BuLi (1.1 eq.) was slowly injected thereinto at −78° C. After stirring at −78° C. for 1 hour, trimethyl borate (2.0 eq.) was injected thereinto at the same temperature. After stirring at −78° C. for 1 hour, hydrochloric acid (0.1N) (100 mL) was slowly injected thereinto. The obtained mixture was stirred for 12 hours by naturally increasing the temperature of −78° C. to ambient temperature after removing an ice bath. After completing a reaction, a resultant therefrom was diluted with DCM, three times washed with brine, and dried with MgSO₄. Subsequently, column chromatography was performed to obtain 12 g of Intermediate P-7.

### 3rd Step: Synthesis of Compound R-4

Intermediate P-7 (12 g/1.0 eq.), 9-(9-(4-chloro-6-phenyl-1,3,5-triazin-2-yl)dibenzo[b,d]furan-2-yl)-9H-carbazole (1.1 eq.), Pd(PPh₃)₄ (0.05 eq.), and K₂CO₃ (3.0 eq.) along with THF (750 mL) and distilled water (250 mL) were added to a flask and then, refluxed at 80° C. After 12 hours, a reaction was terminated, and a resultant therefrom was diluted with DCM, three times washed with brine, and dried with MgSO₄. Subsequently, column chromatography was performed to obtain 8 g of Compound R-4.

### Example 1: Manufacturing of Green Organic Light Emitting Diode (Single Host)

A glass substrate coated with ITO (Indium tin oxide) was ultrasonically washed with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1,350 Å-thick hole transport layer. Compound B was deposited on the hole transport layer to a thickness of 350 Å to form a hole transport auxiliary layer. Compound A-1 was used as a host on the hole transport auxiliary layer, and 7 wt % of PhGD was doped as a dopant to form a 400 Å thick light emitting layer by vacuum deposition. Next, Compound C was deposited on the light emitting layer to form a 50 Å-thick electron transport auxiliary layer, and Compound D and LiQ were simultaneously vacuum deposited at a weight ratio of 1:1 to form a 300 Å-thick electron transport layer. LiQ (15 Å) and Al (1,200 Å) were sequentially vacuum-deposited on the electron transport layer to form a cathode, thereby manufacturing an organic light emitting diode.

The organic light emitting diode has a structure of ITO/Compound A (3% NDP-9 doping, 100 Å)/Compound A (1,350 Å)/Compound B (350 Å)/EML [Host (Compound A-1):PhGD=93 wt %:7 wt %](400 Å)/Compound C (50 Å)/Compound D:LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′:2′,1″:3″,1″′:3′″,1″″-quinquephenyl]-3″″-yl)-1,3,5-triazine
Compound D: 2-(1,1′-Biphenyl-4-yl)-4-(9,9-diphenylfluoren-4-yl)-6-phenyl-1,3,5-triazine

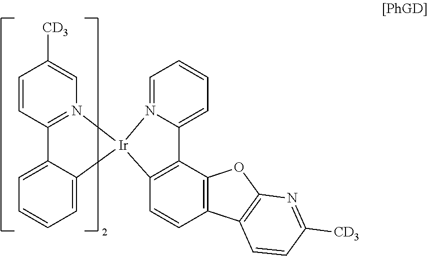

### Examples 2 to 4 and Comparative Examples 1 to 4

Organic light emitting diodes were manufactured in the same manner as in Example 1, except that the composition was changed to those shown in Table 1.

### Example 5: Manufacturing of Green Organic Light Emitting Diode (Mixed Host)

A glass substrate coated with ITO was ultrasonically washed with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. This obtained ITO transparent electrode was used as an anode, Compound E doped with 3% NDP-9 (available from Novaled) was vacuum-deposited on the ITO substrate to form a 100 Å-thick hole injection layer, and Compound E was deposited to on the hole injection layer to form a 1,350 Å-thick hole transport layer. Compound F was deposited on the hole transport layer to a thickness of 320 Å to form a hole transport auxiliary layer. On the hole transport auxiliary layer, Compound A-1 and Compound C-4 were simultaneously used as hosts at a weight ratio of 4:6 and PhGD was doped as a dopant at 10 wt % to form a 380 Å-thick light emitting layer by vacuum deposition. Next, Compound G was deposited on the light emitting layer to form a 50 Å-thick electron transport auxiliary layer, and Compound H and LiQ were simultaneously vacuum deposited at a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The organic light emitting diode has a structure of ITO/Compound E (3% NDP-9 doping, (100 Å))/Compound E (1,350 Å)/Compound F (320 Å)/EML [Host (Compound A-1:Compound C-4=4:6 wt %/wt %):PhGD=90 wt %:10 wt %](380 Å)/Compound G (50 Å)/Compound H:LiQ (300 Å)/LiQ (15 Å)/Al (1,200 Å).
Compound E: N-(9,9-diphenyl-9H-fluoren-2-yl)-N,9-diphenyl-9H-carbazol-2-amine
Compound F: 9,9-dimethyl-N-[3-(9-phenyl-9H-fluoren-9-yl)phenyl]-4-(4-phenylphenyl)-9H-fluoren-2-amine
Compound G: 4-{4-[4-(9,9-dimethyl-9H-fluoren-4-yl)phenyl]phenyl}-2-phenyl-6-(4-phenylphenyl)pyrimidine
Compound H: 2-(4-{1-[4-(diphenyl-1,3,5-triazin-2-yl)phenyl]naphthalene-2-yl}-4,6-diphenyl-1,3,5-triazine

### Examples 5 to 8 and Comparative Examples 5 to 8

Organic light emitting diodes were manufactured in the same manner as in Example 5, except that the composition was changed to those shown in Table 2.

### Evaluation

The driving voltages, luminous efficiency, and life-span characteristics of the organic light emitting diodes according to Example 1 to 8 and Comparative Example 1 to 8 were evaluated.

Specific measurement methods are as follows, and the results are shown in Tables 1 and 2.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

The luminous efficiency (cd/A) at the same current density (10 mA/cm²) was calculated using the luminance and current density measured from (1) and (2) above and the voltage.

The luminous efficiency values of Examples 1 to 4 and Comparative Examples 1 to 4 were calculated as relative values based on Comparative Example 1 and are listed in Table 1.

The luminous efficiency values of Examples 5 to 8 and Comparative Examples 5 to 8 were calculated as relative values based on Comparative Example 5 and are listed in Table 2.

### (4) Measurement of Life-Span

Time when each current efficiency (cd/A) was reduced to 97%, while maintaining luminance (cd/m²) at 24000 cd/m², was measured as a life-span.

The life-span measurement values of Examples 1 to 4 and Comparative Examples 1 to 4 were calculated as relative values based on Comparative Example 1 and are listed in Table 1.

The life-span measurement values of Examples 5 to 8 and Comparative Examples 5 to 8 were calculated as relative values based on Comparative Example 5 and are listed in Table 2.

### (5) Measurement of Driving Voltage

The driving voltages of each diode were measured at 15 mA/cm² using a current-voltage meter (Keithley 2400) to obtain results.

The driving voltages of Examples 1 to 4 and Comparative Examples 1 to 4 were calculated as relative values based on Comparative Example 1 and are listed in Table 1.

The driving voltages of Examples 5 to 8 and Comparative Examples 5 to 8 were calculated as relative values based on Comparative Example 5 and are listed in Table 2.

TABLE 1

| | | Driving | Luminous | Life-span |
|---|---|---|---|---|
| Nos. | Host | voltage (%) | efficiency (%) | (%) |
| Example 1 | A-1 | 100 | 105 | 130 |
| Example 2 | A-33 | 101 | 106 | 120 |
| Example 3 | A-49 | 101 | 105 | 115 |
| Example 4 | A-70 | 100 | 105 | 200 |
| Comparative | R-1 | 100 | 100 | 100 |
| Example 1 | | | | |
| Comparative | R-2 | 102 | 100 | 110 |
| Example 2 | | | | |
| Comparative | R-3 | 110 | 96 | 80 |
| Example 3 | | | | |
| Comparative | R-4 | 115 | 92 | 60 |
| Example 4 | | | | |

TABLE 2

| | | Driving | Luminous | Life-span |
|---|---|---|---|---|
| Nos. | Host | voltage (%) | efficiency (%) | (%) |
| Example 5 | A-1/C-4 | 99 | 108 | 150 |
| Example 6 | A-33/C-4 | 100 | 108 | 140 |
| Example 7 | A-49/C-4 | 101 | 107 | 135 |
| Example 8 | A-70/C-4 | 98 | 108 | 220 |
| Comparative | R-1/C-4 | 100 | 100 | 100 |
| Example 5 | | | | |
| Comparative | R-2/C-4 | 102 | 100 | 120 |
| Example 6 | | | | |
| Comparative | R-3/C-4 | 109 | 93 | 70 |
| Example 7 | | | | |
| Comparative | R-4/C-4 | 112 | 85 | 50 |
| Example 8 | | | | |

Referring to Table 1 and Table 2, it can be confirmed that the organic light emitting diodes according to Examples 1 to 8 exhibit improved luminous efficiency under an equivalent or higher driving voltage compared to the organic light emitting diodes according to Comparative Examples 1 to 8, and in particular, the life-span characteristics are significantly improved.

In particular, when a cyano group is substituted at the R⁸ position (e.g., Comparative Examples 4 and 8), the driving voltages are greatly increased and the efficiency is very low because they have very deep LUMO energy and thus have low exciplex energy.

On the other hand, in the Examples, because the cyano group is not substituted at the R⁸ position, low operating voltage and high efficiency characteristics are exhibited.

In addition, when the dibenzofuranyl group is not substituted (e.g., Comparative Examples 3 and 7), it can be confirmed that the driving voltages are greatly increased due to insufficient electronic characteristics and the efficiency is very low due to imbalance of electrons and holes.

On the other hand, in the Examples, the dibenzofuranyl group is substituted, thereby exhibiting low operating voltage and high efficiency characteristics. In addition, the life-span is greatly improved by a good balance of electrons and holes, and it can be confirmed that the life-span is further improved when D is substituted.

By way of summation and review, some example embodiments may provide a compound for an organic optoelectronic device that may help lower the driving voltage and help realize a high-efficiency and long life-span organic optoelectronic device. Some example embodiments may provide a composition for an organic optoelectronic device including the compound for the organic optoelectronic device. Some example embodiments may provide an organic optoelectronic device including the compound for the organic optoelectronic device or the composition for an organic optoelectronic device. Some example embodiments may provide a display device including the organic optoelectronic device.

Through some embodiments, high-efficiency, long life-span organic optoelectronic devices may be realized while lowering the operating voltage.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device represented by Chemical Formula 1: 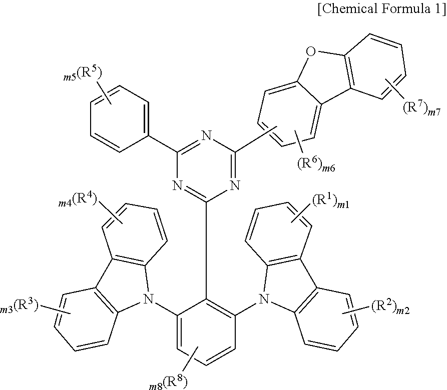
wherein, in Chemical Formula 1,
R¹ to R⁷ are each independently hydrogen, deuterium, a cyano group, a substituted or unsubstituted C1 to C10 alkyl group, a substituted or unsubstituted C1 to C10 alkylsilyl group, or a substituted or unsubstituted C6 to C12 aryl group,
R⁸ is hydrogen, deuterium, a substituted or unsubstituted C1 to C10 alkyl group, or a substituted or unsubstituted C6 to C12 aryl group,
m1 to m4 and m7 are each independently an integer of 1 to 4,
m5 is an integer of 1 to 5,
m6 and m8 are each independently an integer of 1 to 3,
when m1 is 2, 3, or 4, each R¹ is the same or different from each other,
when m2 is 2, 3, or 4, each R² is the same or different from each other,
when m3 is 2, 3, or 4, each R³ is the same or different from each other,
when m4 is 2, 3, or 4, each R⁴ is the same or different from each other,
when m5 is 2, 3, 4, or 5, each R⁵ is the same or different from each other,
when m6 is 2 or 3, each R⁶ is the same or different from each other,
when m7 is 2, 3, or 4, each R⁷ is the same or different from each other, and
when m8 is 2 or 3, each R⁸ is the same or different from each other.

2. The compound for the organic optoelectronic device as claimed in claim 1, wherein:
Chemical Formula 1 is represented by one of Chemical Formula 1-1 to Chemical Formula 1-4: 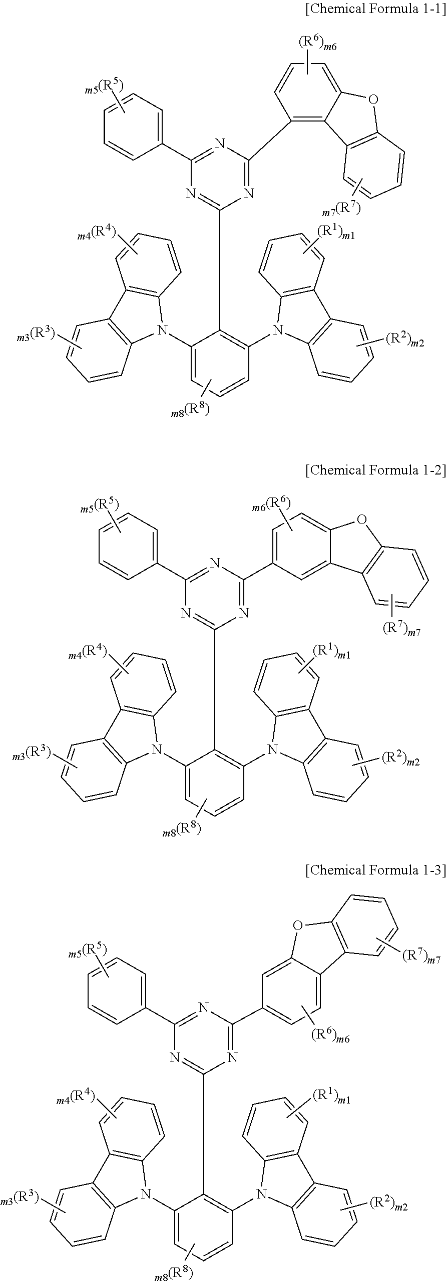 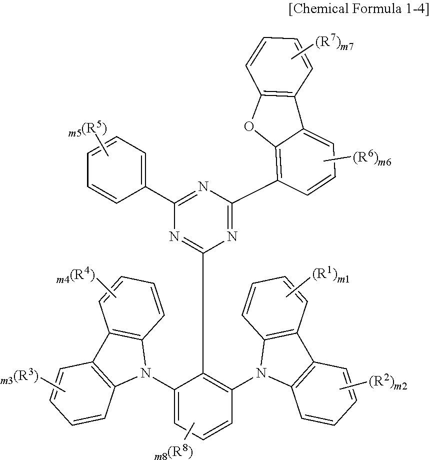
in Chemical Formula 1-1 to Chemical Formula 1-4, R¹ to R⁸ and m1 to m8 are each defined the same as those of Chemical Formula 1.

3. The compound for the organic optoelectronic device as claimed in claim 1, wherein:
R¹ to R⁷ are each independently hydrogen, deuterium, a cyano group, a substituted or unsubstituted tert-butyl group, a substituted or unsubstituted trimethylsilyl group, or a substituted or unsubstituted phenyl group, and
R⁸ is hydrogen, deuterium, or a substituted or unsubstituted phenyl group.

4. The compound for the organic optoelectronic device as claimed in claim 1, wherein:
the compound is a compound of Group 1: 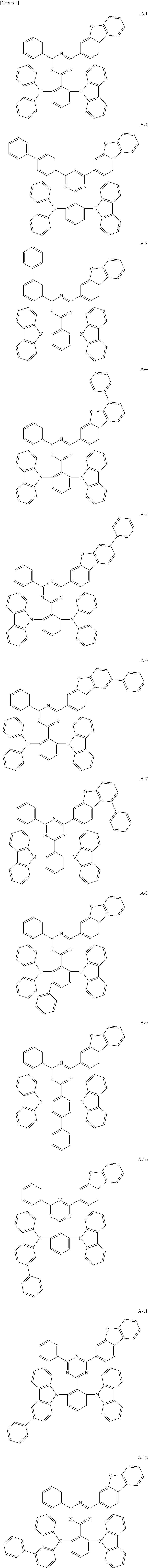 [chemical structure] [chemical structure] [chemical structure] 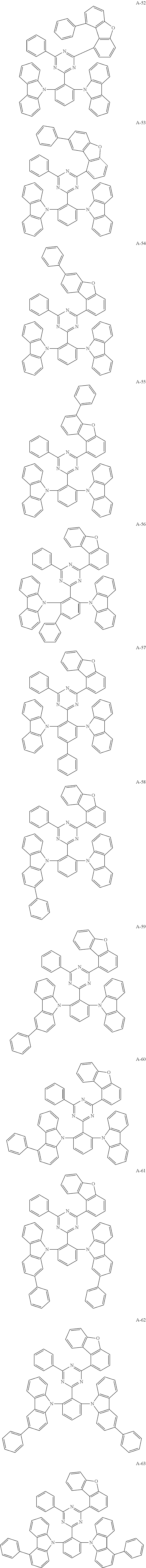 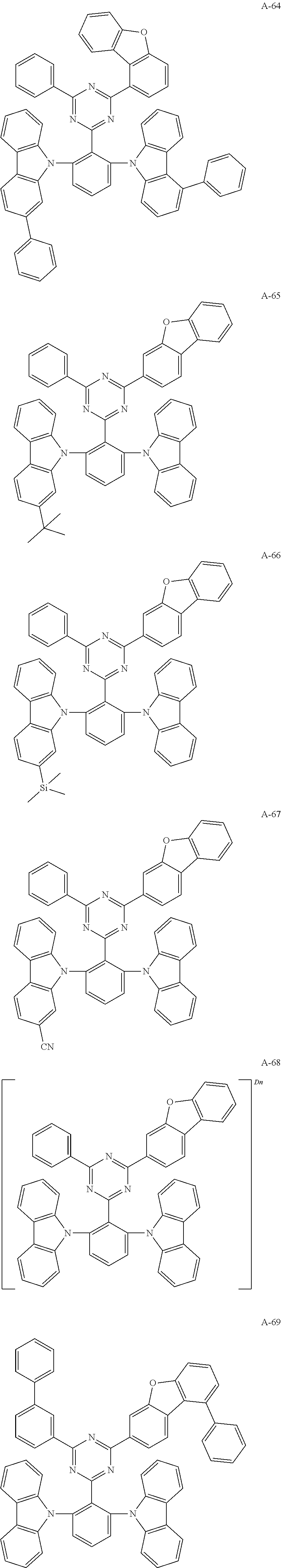
and, in the above chemical formulas, Dn represents the number of deuterium substitutions and is an integer between 1 and 30.

5. The compound for the organic optoelectronic device as claimed in claim 4, wherein the compound A-68 is a compound of Group 1-1:

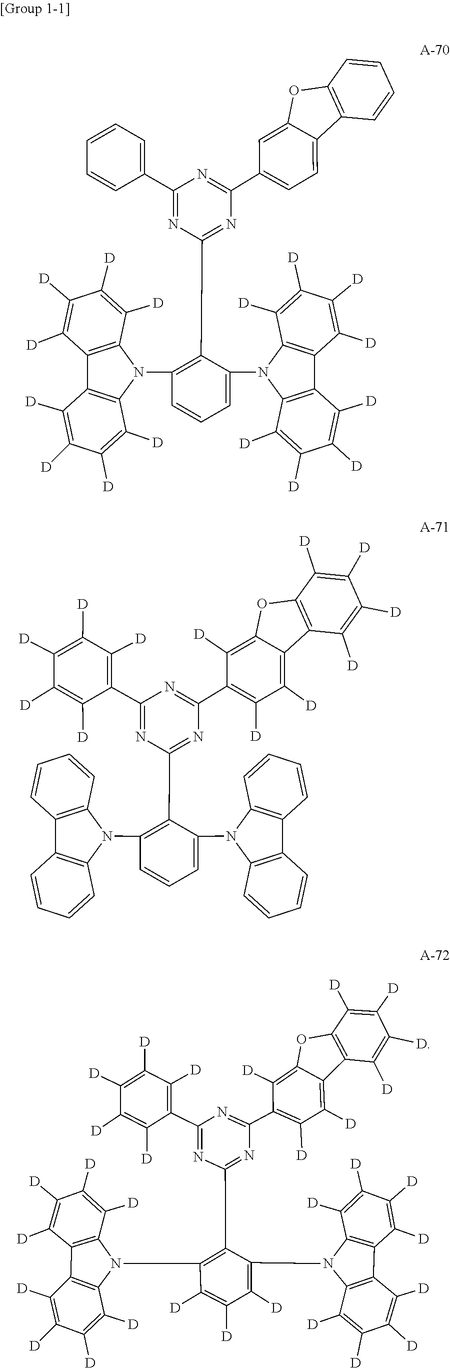

6. A composition for an organic optoelectronic device, the composition comprising:
a first compound; and
a second compound,
wherein:
the first compound is the compound for the organic optoelectronic device as claimed in claim 1, and
the second compound is represented by Chemical Formula 2, a combination of Chemical Formula 3 and Chemical Formula 4, or Chemical Formula 5: 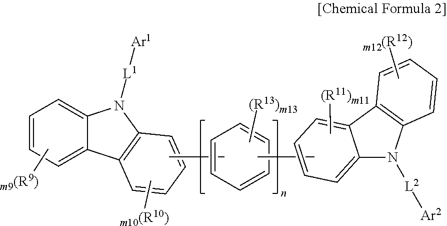
in Chemical Formula 2,
R⁹ to R¹³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar¹ and Ar² are each independently a substituted or unsubstituted C6 to C20 aryl group or a substituted or unsubstituted C2 to C30 heterocyclic group,
L¹ and L² are each independently a single bond or a substituted or unsubstituted C6 to C20 arylene group,
m9, m12, and m13 are each independently integers of 1 to 4,
m10 and m11 are each independently integers of 1 to 3,
when m9 is 2, 3, or 4, each R⁹ is the same or different from each other,
when m10 is 2 or 3, each R¹⁰ is the same or different from each other,
when m11 is 2 or 3, each R¹¹ is the same or different from each other,
when m12 is 2, 3, or 4, each R¹² is the same or different from each other,
when m13 is 2, 3, or 4, each R¹³ is the same or different from each other, and
n is an integer of 0 to 2; 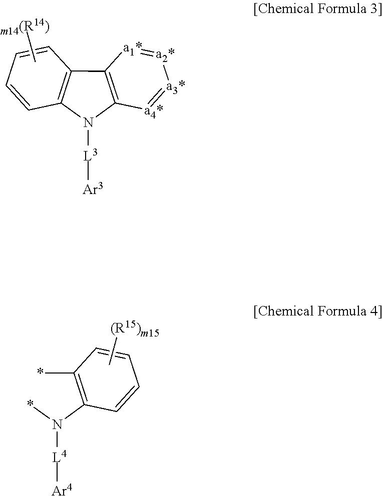
in Chemical Formula 3 and Chemical Formula 4, two adjacent ones of a₁* to a₄* of Chemical Formula 3 are linking carbons linked at * of Chemical Formula 4,
the remaining two of a₁* to a₄* of Chemical Formula 3, not linked at * of Chemical Formula 4, are each independently C-L^{a}-R^{a},
L^{a}, L³, and L⁴ are each independently a single bond or a substituted or unsubstituted C6 to C20 arylene group,
R^{a}, R¹⁴, and R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar³ and Ar⁴ are each independently a substituted or unsubstituted C6 to C20 aryl group or a substituted or unsubstituted C2 to C30 heterocyclic group,
m14 and m15 are each independently an integer of 1 to 4,
when m14 is 2, 3, or 4, each R¹⁴ is the same or different from each other, and
when m15 is 2, 3, or 4, each R¹⁵ is the same or different from each other; 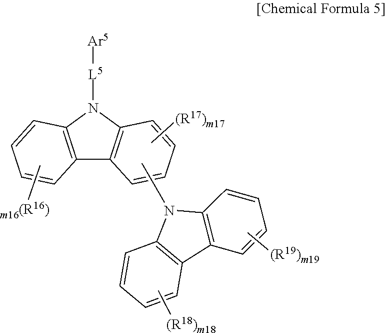
in Chemical Formula 5,
L⁵ is a single bond or a substituted or unsubstituted C6 to C20 arylene group,
R¹⁶ to R¹⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ is a substituted or unsubstituted C6 to C20 aryl group or a substituted or unsubstituted C2 to C30 heterocyclic group,
m16, m18, and m19 are each independently an integer of 1 to 4,
m17 is an integer of 1 to 3,
when m16 is 2, 3, or 4, each R¹⁶ is the same or different from each other,
when m17 is 2 or 3, each R¹⁷ is the same or different from each other,
when m18 is 2, 3, or 4, each R¹⁸ is the same or different from each other, and
when m19 is 2, 3, or 4, each R¹⁹ is the same or different from each other.

7. The composition for an organic optoelectronic device as claimed in claim 6, wherein:
Chemical Formula 2 is represented by Chemical Formula 2-8: 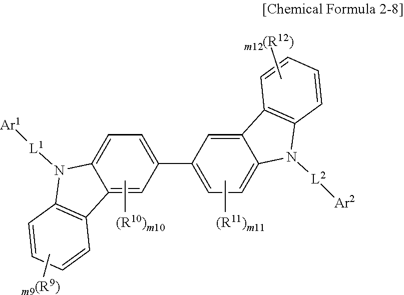
in Chemical Formula 2-8,
R⁹ to R¹² are each independently hydrogen, deuterium, or a substituted or unsubstituted C6 to C12 aryl group,
m9 and m12 are each independently an integer of 1 to 4,
m10 and m11 are each independently an integer of 1 to 3, and
moieties *-L¹-Ar^{l} and *-L²-Ar² are each independently a moiety Group I, 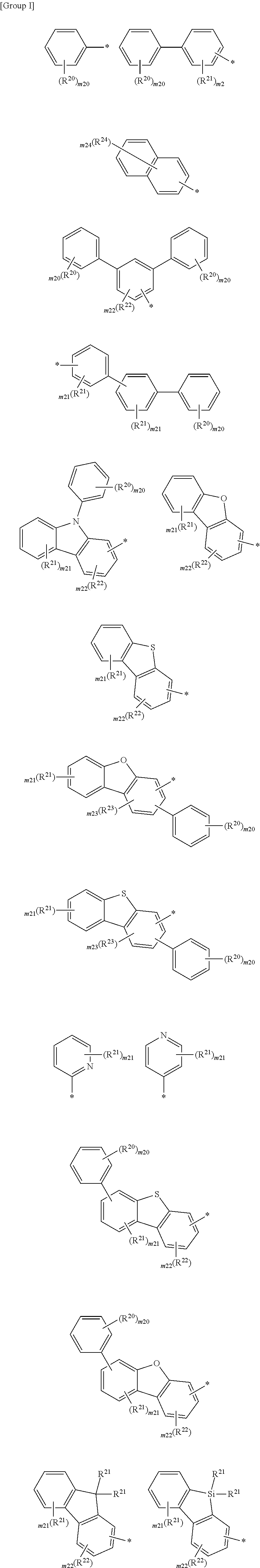 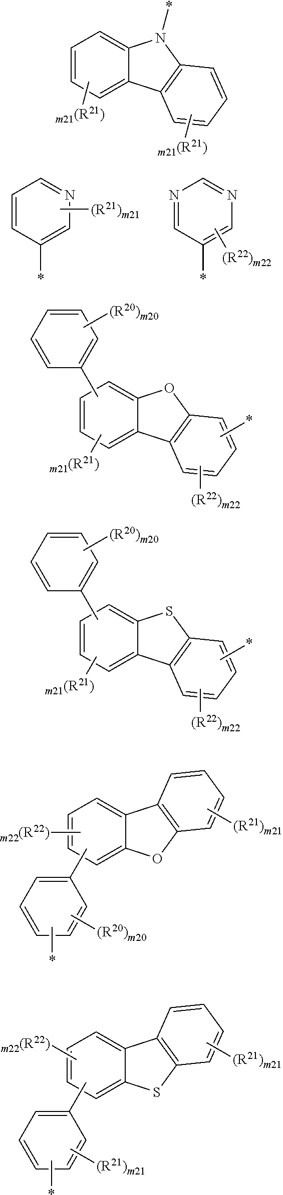 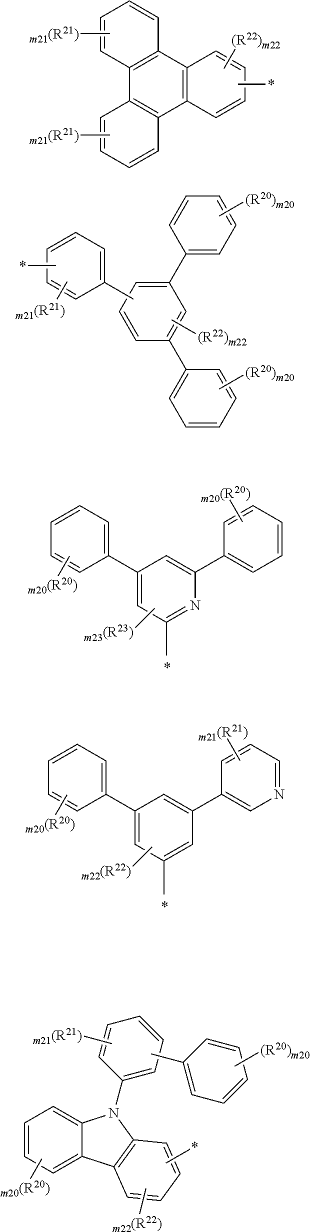 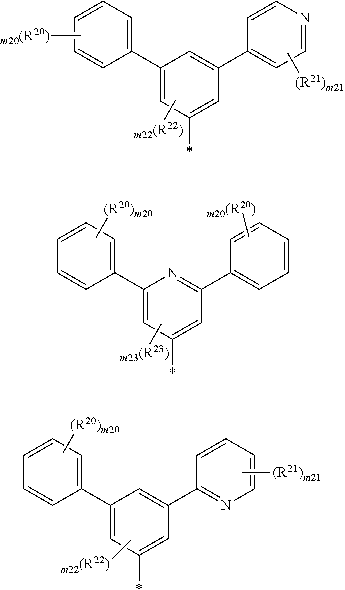
in Group I,
R²⁰ to R²⁴ are each independently hydrogen, deuterium, a cyano group, a substituted or unsubstituted C1 to C10 alkyl group, or a substituted or unsubstituted C6 to C12 aryl group,
m20 is an integer of 1 to 5,
m21 is an integer of 1 to 4,
m22 is an integer of 1 to 3,
m23 is an integer of 1 or 2,
m24 is an integer of 1 to 7, and
* is a linking point.

8. The composition for an organic optoelectronic device as claimed in claim 6, wherein:
the combination of Chemical Formula 3 and Chemical Formula 4 is represented by Chemical Formula 3C: 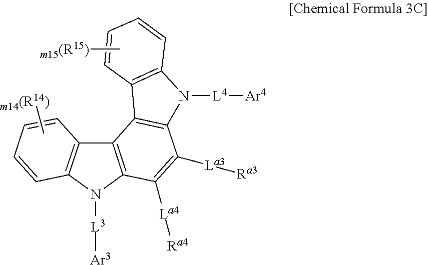
in Chemical Formula 3C,
L^{a3} and L^{a4} are each a single bond,
R¹⁴, R¹⁵, R^{a3}, and R^{a4} are each independently hydrogen, deuterium, or a C6 to C12 aryl group,
m14 and m15 are each independently an integer of 1 to 4, and
moieties *-L³-Ar³ and *-L⁴-Ar⁴ are each independently a moiety of Group I, 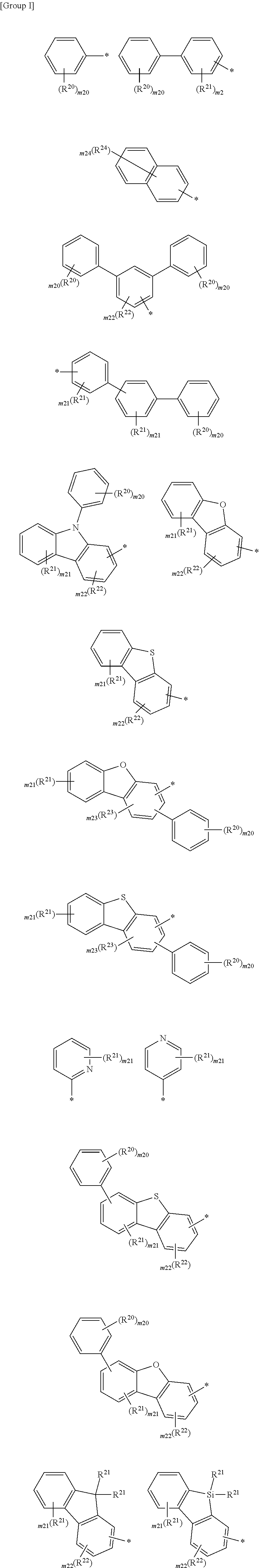 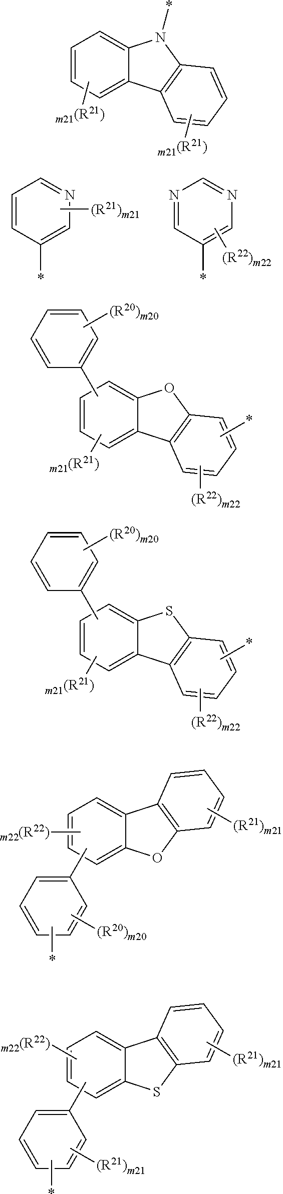 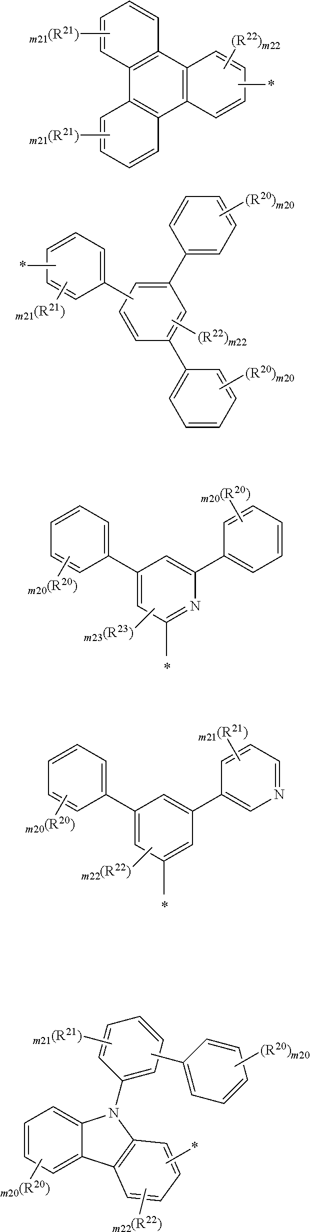 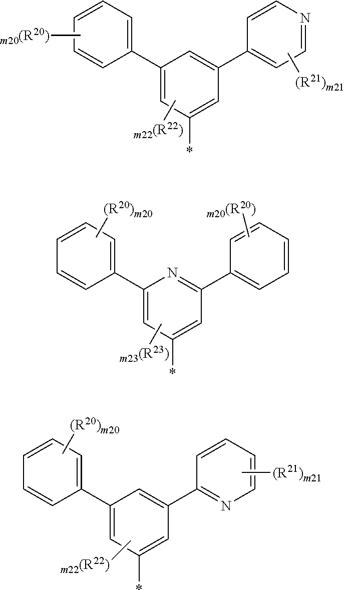
in Group I,
R²⁰ to R²⁴ are each independently hydrogen, deuterium, a cyano group, a substituted or unsubstituted C1 to C10 alkyl group, or a substituted or unsubstituted C6 to C12 aryl group,
m20 is an integer of 1 to 5,
m21 is an integer of 1 to 4,
m22 is an integer of 1 to 3,
m23 is an integer of 1 or 2,
m24 is an integer of 1 to 7, and
* is a linking point.

9. An organic optoelectronic device, comprising:
an anode and a cathode facing each other, and
at least one organic layer between the anode and the cathode,
wherein the at least one organic layer includes the compound for the organic optoelectronic device as claimed in claim 1.

10. The organic optoelectronic device as claimed in claim 9, wherein:
the organic layer includes a light emitting layer, and
the light emitting layer includes the compound.

11. A display device comprising the organic optoelectronic device as claimed in claim 9.

12. An organic optoelectronic device, comprising:
an anode and a cathode facing each other, and
at least one organic layer between the anode and the cathode,
wherein the at least one organic layer includes the composition for an organic optoelectronic device as claimed in claim 6.

13. The organic optoelectronic device as claimed in claim 12, wherein
the organic layer includes a light emitting layer, and
the light emitting layer includes the compound for the composition.

14. A display device comprising the organic optoelectronic device as claimed in claim 12.
